(19) United States
(12) Patent Application Publication
(10) Pub. No.: US 2011/0123556 A1
(43) Pub. Date: May 26, 2011

(54) IMMUNOGEN PRIORITIZATION FOR VACCINE DESIGN

(76) Inventors: Sanjay Phogat, Frederick, MD (US); Christopher Marshall, Brooklyn, NY (US)

(21) Appl. No.: 12/951,675

(22) Filed: Nov. 22, 2010

Related U.S. Application Data
(60) Provisional application No. 61/263,892, filed on Nov. 24, 2009.
(60) Provisional application No. 61/377,649, filed on Aug. 27, 2010.

(51) Int. Cl. G01N 33/53; A61K 39/00; A61P 37/04; A61P 31/00; A61P 31/04; A61P 33/02; A61P 31/12; A61P 31/18; B82Y 15/00

(52) U.S. Cl. 424/184.1; 435/7.24; 977/773; 977/920

(57) ABSTRACT

The present invention provides a method of prioritizing vaccine immunogens, using human iNKT-cell and B-cells, that enables screening of large numbers of immunogens simultaneously in-vitro for diseases and any other vaccine related application, that may then be further tested and improved upon in iterative application of the present technology and other methods of analyzing immunogens. The present invention encompasses the preparation and purification of immunogenic compositions which are formulated into the vaccines of the present invention.

### INCORPORATION BY REFERENCE

This application claims priority to U.S. provisional patent application Ser. Nos. 61/263,892 filed Nov. 24, 2009 and 61/377,649 filed Aug. 27, 2010.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

The present invention provides a method of prioritizing vaccine immunogens, using human iNKT-cell and B-cells, that enables screening of large numbers of immunogens simultaneously in-vitro for infectious diseases (viral, bacterial and protozoal) and any other vaccine related application, that may then be further tested and improved upon in iterative application of the present technology and other methods of analyzing immunogens' potential for vaccine development, in pre-clinical animals testing, and in clinical trials.

### BACKGROUND OF THE INVENTION

B-cell receptor (BCR)-mediated antigen uptake, processing and presentation of peptides along with MHC II molecules on the surface are known antigenic determinants for initiating T-cell responses. The ability of B-cells to internalize and process particulate antigen has been described by several groups (Vidard et al., 1996. J. Immunol. 156:2809-2818; Batista & Neuberger, 2000. EMBO J. 19:513-520; Lin et al., 2008. Immunity 28:75-87.). BCR-mediated internalization of particulate antigen can occur in vivo even in response to a low-affinity antigen, provided a specific avidity threshold is exceeded, implying that a minimum degree of BCR clustering is necessary to trigger internalization of particulate antigenic lipids.

In recent years it has also become clear that T-cells respond to antigenic lipids and glycolipids, presented by cluster of differentiation 1 (CD1) molecules (Brigl and Brenner, 2004, Ann. Rev. Immunol. 22:817-90) on antigen presenting cells (APCs), including B-cells. The human CD1 gene family is composed of five distinct genes, CD1A, CD1B, CD1C, CD1D, and CD1E (Calabi and Milstein, 2000, Semin Immunol. 12(6):503-9), whereas mice express only CD1d molecules. CD1 proteins mediate the presentation of antigenic lipids on the surface of APCs, similar to presentation of peptides by MHC class II molecules (Moody et al. 2003, Nat. Rev. Immunol. 3(1):11-22).

A sub-population of T-cells are the invariant natural killer T (iNKT) cells that are defined by their expression of a restricted TCR repertoire, consisting of a canonical Vα14-Jα18 or Vα24-Jα18 chain in mice and humans, respectively. The iNKT-cells are activated by interaction with antigenic glycolipids presented by CD1d molecules expressed on the surface of antigen presenting cells (Brigl and Brenner, 2004, Ann. Rev. Immunol.). If the required avidity threshold is surpassed, B-cell receptor-mediated uptake allows efficient presentation of particulate antigenic lipids to, and activation of, iNKT-cells. Activated iNKT-cells provide help for specific B-cell proliferation, extra-folicular plasma B-cell differentiation, and production of high titers of specific IgM and early class-switched antibodies.

α-galactosylceramide (αGalCer) strongly activates both murine and human iNKT-cells (Kawano et a/0.1997, Science 278(5343):1626-9), and activated iNKT-cells stimulate B-cell proliferation and antibody production in vitro independent of iNKT-cell ligands and BCR specificity (Galli et al. 2003, J. Exp. Med. 197(8):1051-7). Although αGalCer can be used effectively to characterize of iNKT-cells, both in vitro and in vivo, it is not a natural ligand for

iNKT-cells. iNKT-cells are activated by glycolipids from LPS-negative bacteria (Mattner et al., 2005. Nature 434:525-529; Kinjo et al., 2005. Nature 434:520-525; Kinjo et al., 2006. Nat Immunol 7:978-986). Alternatively, iNKT-cells can recognize an endogenous lipidic ligand, via CD1d presentation (Mattner et al., 2005. Nature 434:525-529.; Brigl et al., 2003. Nat Immunol 4:1230-1237; Paget et al., 2007. Immunity 27:597-609).

All B-cells express CD1d; this expression, however, is enhanced in marginal zone B-cells, which present a CD21high CD23low CD1dhigh phenotype and recruit iNKT-cell help and generate specific antibody responses more efficiently (Barral et al., 2008. Proc. Natl. Acad. Sci 105(24): 8345-50).

Strategies that use invariant Natural killer T (iNKT) cell dependent B-cell activation in presence of nano-particles containing specific glycolipids (aGalCer) have been developed for analyzing immune responses in mice (Banal et al., 2008. Proc. Natl. Acad. Sci 105(24): 8345-50). Recent studies in mice also show that using nano-particles that combine CD1-effective lipids and immunoglobulinspecific immunogen, BCR-mediated internalization achieves the selectivity in delivery and efficient internalization of particulate antigenic lipids, and enhances B-cell presentation of particulate lipid antigens to iNKT-cells in vivo. Subsequently, activated iNKT-cells provide help for immunogen-specific B-cell proliferation, B-cell differentiation to extra-follicular plasma cells, and secretion of high titers of specific IgM and early class-switched antibodies (Barral et al., 2008. Proc. Natl. Acad. Sci 105(24): 8345-50).

Citation or identification of any document in this application is not an admission that such document is available as prior art to the present invention.

### SUMMARY OF THE INVENTION

The present invention is based, in part, on Applicants' discovery that effective vaccine immunogens may be identified with activated iNKT-cells and B-cells by measuring the binding and functional activity of the resulted secreted antibodies.

The present invention provides a method of prioritizing vaccine immunogens, using human iNKT-cell and B-cells, that enables screening of large numbers of immunogens simultaneously in-vitro for infectious diseases (viral, bacterial and protozoal) and any other vaccine related application, that can then be further tested and improved upon in iterative application of the present technology and other methods of analyzing immunogens' potential for vaccine development, in pre-clinical animals testing, and in clinical trials.

The present invention relates to a method of identifying vaccine immunogens which may comprise:
(i) engineering nano-particles to contain CD-1-effective glycolipids and disease-, disorder-, or vaccine-specific immunogens,
(ii) exposing iNKT-cells in vitro to the engineered nano-particles, thereby activating the iNKT-cells,
(iii) exposing naïve B-cells in vitro to the above engineered nano-particles in the presence of activated iNKT-cells,
(iv) assaying secreted antibodies for binding and functional activity, and
(v) identifying the immunogens that bind the secreted antibodies, thereby identifying vaccine immunogens.

The immunogens identified by the present invention may be utilized for immunogenic compositions for vaccine formulation.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings, in which:

FIG. 1 depicts a human DNA sequence from clone RP11-101J8 on chromosome 1q21.2-22 and the CD1A gene for CD1A antigen, a polypeptide, a high-mobility group nucleosome binding domain 1 (HMGN1) pseudogene, the CD1C gene for CD1C antigen, c polypeptide, the CD1B gene for CD1B antigen, b polypeptide, the CD1E gene for CD1E antigen, e polypeptide, the OR10T2 gene for olfactory receptor, family 10, subfamily T, member 2 and the OR10K2 gene for olfactory receptor, family 10, subfamily K, member 2, complete sequence (GenBank Accession No. AL121986.

### DETAILED DESCRIPTION

The present invention provides a method of prioritizing vaccine immunogens, using human iNKT-cell and B-cells, that enables screening of large numbers of immunogens simultaneously in-vitro for infectious diseases (viral, bacterial and protozoal) and any other vaccine related application, that can then be further tested and improved upon in iterative application of the present technology and other methods of analyzing immunogens' potential for vaccine development, in pre-clinical animals testing, and in clinical trials. Methods of the present invention may be practiced using high throughput techniques.

The invention may involve:
(i) engineering nano-particles to contain human CD-1-effective glycolipids and disease-, disorder-, or vaccine-specific immunogens,
(ii) exposing iNKT-cells in vitro to the engineered nanoparticles, and thereby activating the iNKT-cells,
(iii) exposing naïve B-cells in vitro from human donor/s to the above engineered nano-particles (containing glycolipids and immunogen) in the presence of activated iNKT-cells and
(iv) assaying secreted antibodies for binding and functional activity, such as, for example, virus neutralizing capacity.

Exposing and activating iNKT-cells to the engineered nanoparticles, and exposing naïve B-cells from human donor/s to the engineered nano-particles in the presence of activated iNKT-cells may be carried out as separate steps, or in a single, combined reaction.

BCR-specific binding and subsequent internalization of particulate antigen is dependent on antigen affinity and avidity. Use of a support, such as a bead, provides control of surface antigen density, and thereby provides control of the avidity of the interaction. This enables the practitioner of the instant invention to generate beads with surface antigen that binds a BCR with low affinity that nonetheless stimulate specific immune responses. An immunostimulant, also attached to the support, is internalized by the cell together with the antigen, which enhances the specific immune response by recruiting other cellular factors that stimulate activation.

A nanoparticle capable of BCR-mediated internalization may comprise:
(i) a support, and
(ii) a BCR-binding antigen attached to the support.

Such a nanoparticle may further comprise an immunostimulant attached to the support. Upon BCR-mediated internalization the product elicits an antigen-specific immune response.

Any method known in the art may be used to prepare and use nanoparticles that meet the above criteria. For example, PCT/GB2009/001111 (Facundo et al., 2009) describes the preparation and use of nanoparticles containing a BCR-binding antigen and an immunostimulant (e.g., α-Galactosyl Ceramide) for eliciting an antigen-specific immune response; US2007/0104776 (Ishii et al.) describes the use of liposomes containing ovalbumin and α-Galactosyl Ceramide, whereby the ovalbumin is encapsulated in the liposome. Both references are incorporated herein by reference. The liposomes show an inhibitory effect on antibody production.

Any suitable support may be used. The support must be suitable for attaching an antigen and/or an immunostimulant thereto and must be of a suitable size to be internalized and processed by a BCR-expressing cell. In some embodiments, the support may be a particle, for example a bead. The support may be a microsphere. The term “microsphere” refers to a spherical shell made of any material that has a very small diameter, usually in the micron or nanometer range. The support may be made of any suitable material known in the art, for example, but not limited to, polymer or silica supports, such as, for example, polymer or silica beads or microspheres.

The particle further may be a liposome which is a vesicle structure made up of one or more lipid bilayers surrounding an aqueous core. Bangham et al. (Bangham et al., 1965, J MoI Biol, 13:238) describe methods for making liposomes; WO2006/002642 describes compositions and methods for stabilizing liposome suspensions. Both references are incorporated herein by reference.

Liposome coated beads may be prepared by methods known to one of ordinary skill in the art, and used to practice the instant invention.

Any method of attaching the immunostimulant and/or the antigen to the support known to one of ordinary skill in the art that preserves the immunostimulant's and the antigen's capability of eliciting (a) desirable immune response(s) may be employed. Any method of using liposomes to coat silica microspheres with particular immunostimulant lipids known to one of ordinary skill in the art may also be employed.

More or less antigen may either be attached to the support surface or incorporated in a liposome, thereby increasing or decreasing the antigen density (and therefore avidity); methods of assaying and adjusting the amount of antigen on a support surface or liposome are known to one of ordinary skill in the art.

The terms “protein”, “peptide”, “polypeptide”, and “amino acid sequence” are used interchangeably herein to refer to polymers of amino acid residues of any length. The polymer may be linear or branched, it may comprise modified amino acids or amino acid analogs, and it may be interrupted by chemical moieties other than amino acids. The terms also encompass an amino acid polymer that has been modified naturally or by intervention; for example disulfide bond formation, glycosylation, lipidation, acetylation, phosphorylation, or any other manipulation or modification, such as conjugation with a labeling or bioactive component.

As used herein, the terms “antigen” or “immunogen” are used interchangeably to refer to a substance, typically a protein, which is capable of inducing an immune response in a subject. The term also refers to proteins that are immunologically active in the sense that once administered to a subject (either directly or by administering to the subject a nucleotide sequence or vector that encodes the protein) is able to evoke an immune response of the humoral and/or cellular type directed against that protein.

The term “antibody” includes intact molecules as well as fragments thereof, such as Fab, F(ab′)₂, Fv and scFv which are capable of binding the epitope determinant. These antibody fragments retain some ability to selectively bind with its antigen or receptor and include, for example:
(i) Fab, the fragment which contains a monovalent antigen-binding fragment of an antibody molecule can be produced by digestion of whole antibody with the enzyme papain to yield an intact light chain and a portion of one heavy chain;
(ii) Fab′, the fragment of an antibody molecule can be obtained by treating whole antibody with pepsin, followed by reduction, to yield an intact light chain and a portion of the heavy chain; two Fab′ fragments are obtained per antibody molecule;
(iii) F(ab′)₂, the fragment of the antibody that can be obtained by treating whole antibody with the enzyme pepsin without subsequent reduction; F(ab′)2 is a dimer of two Fab′ fragments 30 held together by two disulfide bonds;
(iv) scFv, including a genetically engineered fragment containing the variable region of a heavy and a light chain as a fused single chain molecule.

General methods of making these fragments are known in the art. (See for example, Harlow and Lane, Antibodies: A Laboratory Manual, Cold Spring Harbor Laboratory, New York (1988), which is incorporated herein by reference).

Broad and potent neutralizing antibodies may neutralize greater than about 50% of HIV-1 viruses (from diverse clades and different strains within a Glade) in a neutralization assay. The inhibitory concentration of the monoclonal antibody may be less than about 25 ug/ml to neutralize about 50% of the input virus in the neutralization assay.

It should be understood that the proteins, including the antibodies and/or antigens of the invention may differ from the exact sequences illustrated and described herein. Thus, the invention contemplates deletions, additions and substitutions to the sequences shown, so long as the sequences function in accordance with the methods of the invention. In this regard, particularly preferred substitutions will generally be conservative in nature, i.e., those substitutions that take place within a family of amino acids. For example, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. It is reasonably predictable that an isolated replacement of leucine with isoleucine or valine, or vice versa; an aspartate with a glutamate or vice versa; a threonine with a serine or vice versa; or a similar conservative replacement of an amino acid with a structurally related amino acid, will not have a major effect on the biological activity. Proteins having substantially the same amino acid sequence as the sequences illustrated and described but possessing minor amino acid substitutions that do not substantially affect the immunogenicity of the protein are, therefore, within the scope of the invention.

As used herein the terms “nucleotide sequences” and “nucleic acid sequences” refer to deoxyribonucleic acid (DNA) or ribonucleic acid (RNA) sequences, including, without limitation, messenger RNA (mRNA), DNA/RNA hybrids, or synthetic nucleic acids. The nucleic acid can be single-stranded, or partially or completely double-stranded (duplex). Duplex nucleic acids can be homoduplex or heteroduplex.

As used herein the term “transgene” may used to refer to “recombinant” nucleotide sequences that may be derived from any of the nucleotide sequences encoding the proteins of the present invention. The term “recombinant” means a nucleotide sequence that has been manipulated “by man” and which does not occur in nature, or is linked to another nucleotide sequence or found in a different arrangement in nature. It is understood that manipulated “by man” means manipulated by some artificial means, including by use of machines, codon optimization, restriction enzymes, etc.

For example, in one embodiment the nucleotide sequences may be mutated such that the activity of the encoded proteins in vivo is abrogated. In another embodiment the nucleotide sequences may be codon optimized, for example the codons may be optimized for human use. In preferred embodiments the nucleotide sequences of the invention are both mutated to abrogate the normal in vivo function of the encoded proteins, and codon optimized for human use. For example, each of the Gag, Pol, Env, Nef, RT, and Int sequences of the invention may be altered in these ways.

As regards codon optimization, the nucleic acid molecules of the invention have a nucleotide sequence that encodes the antigens of the invention and can be designed to employ codons that are used in the genes of the subject in which the antigen is to be produced. Many viruses, including HIV and other lentiviruses, use a large number of rare codons and, by altering these codons to correspond to codons commonly used in the desired subject, enhanced expression of the antigens can be achieved. In a preferred embodiment, the codons used are “humanized” codons, i.e., the codons are those that appear frequently in highly expressed human genes (Andre et al., J. Virol. 72:1497-1503, 1998) instead of those codons that are frequently used by HIV. Such codon usage provides for efficient expression of the transgenic HIV proteins in human cells. Any suitable method of codon optimization may be used. Such methods, and the selection of such methods, are well known to those of skill in the art. In addition, there are several companies that will optimize codons of sequences, such as Geneart (geneart.com). Thus, the nucleotide sequences of the invention can readily be codon optimized.

The invention further encompasses nucleotide sequences encoding functionally and/or antigenically equivalent variants and derivatives of the antigens of the invention and functionally equivalent fragments thereof. These functionally equivalent variants, derivatives, and fragments display the ability to retain antigenic activity. For instance, changes in a DNA sequence that do not change the encoded amino acid sequence, as well as those that result in conservative substitutions of amino acid residues, one or a few amino acid deletions or additions, and substitution of amino acid residues by amino acid analogs are those which will not significantly affect properties of the encoded polypeptide. Conservative amino acid substitutions are glycine/alanine; valine/isoleucine/leucine; asparagine/glutamine; aspartic acid/glutamic acid; serine/threonine/methionine; lysine/arginine; and phenylalanine/tyrosine/tryptophan. In one embodiment, the variants have at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% homology or identity to the antigen, epitope, immunogen, peptide or polypeptide of interest.

For the purposes of the present invention, sequence identity or homology is determined by comparing the sequences when aligned so as to maximize overlap and identity while minimizing sequence gaps. In particular, sequence identity may be determined using any of a number of mathematical algorithms. A nonlimiting example of a mathematical algorithm used for comparison of two sequences is the algorithm of Karlin & Altschul, Proc. Natl. Acad. Sci. USA 1990; 87: 2264-2268, modified as in Karlin & Altschul, Proc. Natl. Acad. Sci. USA 1993; 90: 5873-5877.

Another example of a mathematical algorithm used for comparison of sequences is the algorithm of Myers & Miller, CABIOS 1988; 4: 11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson & Lipman, Proc. Natl. Acad. Sci. USA 1988; 85: 2444-2448.

Advantageous for use according to the present invention is the WU-BLAST (Washington University BLAST) version 2.0 software. WU-BLAST version 2.0 executable programs for several UNIX platforms can be downloaded from ftp://blast.wustl.edu/blast/executables. This program is based on WU-BLAST version 1.4, which in turn is based on the public domain NCBI-BLAST version 1.4 (Altschul & Gish, 1996, Local alignment statistics, Doolittle ed., Methods in Enzymology 266: 460-480; Altschul et al., Journal of Molecular Biology 1990; 215: 403-410; Gish & States, 1993; Nature Genetics 3: 266-272; Karlin & Altschul, 1993; Proc. Natl. Acad. Sci. USA 90: 5873-5877; all of which are incorporated by reference herein).

The various recombinant nucleotide sequences and antibodies and/or antigens of the invention are made using standard recombinant DNA and cloning techniques. Such techniques are well known to those of skill in the art. See for example, “Molecular Cloning: A Laboratory Manual”, second edition (Sambrook et al. 1989).

The nucleotide sequences of the present invention may be inserted into “vectors.” The term “vector” is widely used and understood by those of skill in the art, and as used herein the term “vector” is used consistent with its meaning to those of skill in the art. For example, the term “vector” is commonly used by those skilled in the art to refer to a vehicle that allows or facilitates the transfer of nucleic acid molecules from one environment to another or that allows or facilitates the manipulation of a nucleic acid molecule.

Any vector that allows expression of the antibodies and/or antigens of the present invention may be used in accordance with the present invention. In certain embodiments, the antigens and/or antibodies of the present invention may be used in vitro (such as using cell-free expression systems) and/or in cultured cells grown in vitro in order to produce the encoded HIV-antigens and/or antibodies which may then be used for various applications such as in the production of proteinaceous vaccines. For such applications, any vector that allows expression of the antigens and/or antibodies in vitro and/or in cultured cells may be used.

For applications where it is desired that the antibodies and/or antigens be expressed in vivo, for example when the transgenes of the invention are used in DNA or DNA-containing vaccines, any vector that allows for the expression of the antibodies and/or antigens of the present invention and is safe for use in vivo may be used. In preferred embodiments the vectors used are safe for use in humans, mammals and/or laboratory animals.

For the antibodies and/or antigens of the present invention to be expressed, the protein coding sequence should be “operably linked” to regulatory or nucleic acid control sequences that direct transcription and translation of the protein. As used herein, a coding sequence and a nucleic acid control sequence or promoter are said to be “operably linked” when they are covalently linked in such a way as to place the expression or transcription and/or translation of the coding sequence under the influence or control of the nucleic acid control sequence. The “nucleic acid control sequence” can be any nucleic acid element, such as, but not limited to promoters, enhancers, IRES, introns, and other elements described herein that direct the expression of a nucleic acid sequence or coding sequence that is operably linked thereto. The term “promoter” will be used herein to refer to a group of transcriptional control modules that are clustered around the initiation site for RNA polymerase II and that when operationally linked to the protein coding sequences of the invention lead to the expression of the encoded protein. The expression of the transgenes of the present invention can be under the control of a constitutive promoter or of an inducible promoter, which initiates transcription only when exposed to some particular external stimulus, such as, without limitation, antibiotics such as tetracycline, hormones such as ecdysone, or heavy metals. The promoter can also be specific to a particular cell type, tissue or organ. Many suitable promoters and enhancers are known in the art, and any such suitable promoter or enhancer may be used for expression of the transgenes of the invention. For example, suitable promoters and/or enhancers can be selected from the Eukaryotic Promoter Database (EPDB).

The immunogen may be a viral immunogen, wherein the viruses include, but are not limited to, those caused by infection with hepatitis B, hepatitis C, rotavirus, human immunodeficiency virus type I (HIV-I), human immunodeficiency virus type II (HIV-II), human T-cell lymphotropic virus type I (HTLV-I), human T-cell lymphotropic virus type II (HTLV-II), AIDS, DNA viruses such as hepatitis type B and hepatitis type C virus; parvoviruses, such as adeno-associated virus and cytomegalovirus; papovaviruses such as papilloma virus, polyoma viruses, and SV40; adenoviruses; herpes viruses such as herpes simplex type I (HSV-I), herpes simplex type II (HSV-II), and Epstein-Ban virus; poxviruses, such as variola (smallpox) and vaccinia virus; and RNA viruses, such as human immunodeficiency virus type I (HIV-I), human immunodeficiency virus type II (HIV-II), human T-cell lymphotropic virus type I (HTLV-I), human T-cell lymphotropic virus type II (HTLV-II), influenza virus, measles virus, rabies virus, Sendai virus, picornaviruses such as poliomyelitis virus, coxsackieviruses, rhinoviruses, reoviruses, togaviruses such as rubella virus (German measles) and Semliki forest virus, arboviruses, and hepatitis type A virus.

The present invention relates to a recombinant vector expressing a foreign epitope. Advantageously, the epitope is an HIV epitope. In an advantageous embodiment, the HIV epitope is a protein fragment of the present invention, however, the present invention may encompass additional HIV antigens, epitopes or immunogens. Advantageously, the HIV epitope is an HIV antigen, HIV epitope or an HIV immunogen, such as, but not limited to, the HIV antigens, HIV epitopes or HIV immunogens of U.S. patent Nos. is an HIV antigen, HIV epitope or an HIV immunogen, such as, but not limited to, the HIV antigens, HIV epitopes or HIV immunogens of U.S. Pat. Nos. 7,341,731, 7,335,364, 7,329,807, 7,323,553, 7,320,859, 7,311,920, 7,306,798; 7,285,646, 7,285,289, 7,285,271, 7,282,364, 7,273,695, 7,270,997, 7,262,270, 7,244,819; 7,244,575, 7,232,567, 7,232,566, 7,223,844, 7,223,739, 7,223,534, 7,223,368, 7,220,554; 7,214,530, 7,211,659, 7,211,432, 7,205,159, 7,198,934, 7,195,768, 7,192,555, 7,189,826; 7,189,522, 7,186,507, 7,179,645, 7,175,843, 7,172,761, 7,169,550, 7,157,083, 7,153,509; 7,147,862, 7,141,550, 7,129,219, 7,122,188, 7,118,859, 7,118,855, 7,118,751, 7,118,742; 7,105,655, 7,101,552, 7,097,971, 7,097,842, 7,094,405, 7,091,049, 7,090,648, 7,087,377, 7,083,787, 7,070,787, 7,070,781, 7,060,273, 7,056,521, 7,056,519, 7,049,136, 7,048,929; 7,033,593, 7,030,094, 7,022,326, 7,009,037, 7,008,622, 7,001,759, 6,997,863, 6,995,008; 6,979,535, 6,974,574, 6,972,126, 6,969,609, 6,964,769, 6,964,762, 6,958,158, 6,956,059; 6,953,689, 6,951,648, 6,946,075, 6,927,031, 6,919,319, 6,919,318, 6,919,077, 6,913,752; 6,911,315, 6,908,617, 6,908,612, 6,902,743, 6,900,010, 6,893,869, 6,884,785, 6,884,435; 6,875,435, 6,867,005, 6,861,234, 6,855,539, 6,841,381 6,841,345, 6,838,477, 6,821,955, 6,818,392, 6,818,222, 6,815,217, 6,815,201, 6,812,026, 6,812,025, 6,812,024, 6,808,923; 6,806,055, 6,803,231, 6,800,613, 6,800,288, 6,797,811, 6,780,967, 6,780,598, 6,773,920; 6,764,682, 6,761,893, 6,753,015, 6,750,005, 6,737,239, 6,737,067, 6,730,304, 6,720,310; 6,716,823, 6,713,301, 6,713,070, 6,706,859, 6,699,722, 6,699,656, 6,696,291, 6,692,745; 6,670,181, 6,670,115, 6,664,406, 6,657,055, 6,657,050, 6,656,471, 6,653,066, 6,649,409; 6,649,372, 6,645,732, 6,641,816, 6,635,469, 6,613,530, 6,605,427, 6,602,709 6,602,705, 6,600,023, 6,596,477, 6,596,172, 6,593,103, 6,593,079, 6,579,673, 6,576,758, 6,573,245; 6,573,040, 6,569,418, 6,569,340, 6,562,800, 6,558,961, 6,551,828, 6,551,824, 6,548,275; 6,544,780, 6,544,752, 6,544,728, 6,534,482, 6,534,312, 6,534,064, 6,531,572, 6,531,313; 6,525,179, 6,525,028, 6,524,582, 6,521,449, 6,518,030, 6,518,015, 6,514,691, 6,514,503; 6,511,845, 6,511,812, 6,511,801, 6,509,313, 6,506,384, 6,503,882, 6,495,676, 6,495,526; 6,495,347, 6,492,123, 6,489,131, 6,489,129, 6,482,614, 6,479,286, 6,479,284, 6,465,634, 6,461,615 6,458,560, 6,458,527, 6,458,370, 6,451,601, 6,451,592, 6,451,323, 6,436,407; 6,432,633, 6,428,970, 6,428,952, 6,428,790, 6,420,139, 6,416,997, 6,410,318, 6,410,028; 6,410,014, 6,407,221, 6,406,710, 6,403,092, 6,399,295, 6,392,013, 6,391,657, 6,384,198; 6,380,170, 6,376,170, 6,372,426, 6,365,187, 6,358,739, 6,355,248, 6,355,247, 6,348,450; 6,342,372, 6,342,228, 6,338,952, 6,337,179, 6,335,183, 6,335,017, 6,331,404, 6,329,202; 6,329,173, 6,328,976, 6,322,964, 6,319,666, 6,319,665, 6,319,500, 6,319,494, 6,316,205; 6,316,003, 6,309,633, 6,306,625 6,296,807, 6,294,322, 6,291,239, 6,291,157, 6,287,568, 6,284,456, 6,284,194, 6,274,337, 6,270,956, 6,270,769, 6,268,484, 6,265,562, 6,265,149; 6,262,029, 6,261,762, 6,261,571, 6,261,569, 6,258,599, 6,258,358, 6,248,332, 6,245,331; 6,242,461, 6,241,986, 6,235,526, 6,235,466, 6,232,120, 6,228,361, 6,221,579, 6,214,862; 6,214,804, 6,210,963, 6,210,873, 6,207,185, 6,203,974, 6,197,755, 6,197,531, 6,197,496; 6,194,142, 6,190,871, 6,190,666, 6,168,923, 6,156,302, 6,153,408, 6,153,393, 6,153,392; 6,153,378, 6,153,377, 6,146,635, 6,146,614, 6,143,876 6,140,059, 6,140,043, 6,139,746, 6,132,992, 6,124,306, 6,124,132, 6,121,006, 6,120,990, 6,114,507, 6,114,143, 6,110,466; 6,107,020, 6,103,521, 6,100,234, 6,099,848, 6,099,847, 6,096,291, 6,093,405, 6,090,392; 6,087,476, 6,083,903, 6,080,846, 6,080,725, 6,074,650, 6,074,646, 6,070,126, 6,063,905; 6,063,564, 6,060,256, 6,060,064, 6,048,530, 6,045,788, 6,043,347, 6,043,248, 6,042,831; 6,037,165, 6,033,672, 6,030,772, 6,030,770, 6,030,618, 6,025,141, 6,025,125, 6,020,468; 6,019,979, 6,017,543, 6,017,537, 6,015,694, 6,015,661, 6,013,484, 6,013,432 6,007,838, 6,004,811, 6,004,807, 6,004,763, 5,998,132, 5,993,819, 5,989,806, 5,985,926, 5,985,641; 5,985,545, 5,981,537, 5,981,505, 5,981,170, 5,976,551, 5,972,339, 5,965,371, 5,962,428; 5,962,318, 5,961,979, 5,961,970, 5,958,765, 5,958,422, 5,955,647, 5,955,342, 5,951,986; 5,951,975, 5,942,237, 5,939,277, 5,939,074, 5,935,580, 5,928,930, 5,928,913, 5,928,644; 5,928,642, 5,925,513, 5,922,550, 5,922,325, 5,919,458, 5,916,806, 5,916,563, 5,914,395; 5,914,109, 5,912,338, 5,912,176, 5,912,170, 5,906,936, 5,895,650, 5,891,623, 5,888,726, 5,885,580 5,885,578, 5,879,685, 5,876,731, 5,876,716, 5,874,226, 5,872,012, 5,871,747; 5,869,058, 5,866,694, 5,866,341, 5,866,320, 5,866,319, 5,866,137, 5,861,290, 5,858,740; 5,858,647, 5,858,646, 5,858,369, 5,858,368, 5,858,366, 5,856,185, 5,854,400, 5,853,736; 5,853,725, 5,853,724, 5,852,186, 5,851,829, 5,851,529, 5,849,475, 5,849,288, 5,843,728; 5,843,723, 5,843,640, 5,843,635, 5,840,480, 5,837,510, 5,837,250, 5,837,242, 5,834,599; 5,834,441, 5,834,429, 5,834,256, 5,830,876, 5,830,641, 5,830,475, 5,830,458, 5,830,457; 5,827,749, 5,827,723, 5,824,497 5,824,304, 5,821,047, 5,817,767, 5,817,754, 5,817,637, 5,817,470, 5,817,318, 5,814,482, 5,807,707, 5,804,604, 5,804,371, 5,800,822, 5,795,955; 5,795,743, 5,795,572, 5,789,388, 5,780,279, 5,780,038, 5,776,703, 5,773,260, 5,770,572; 5,766,844, 5,766,842, 5,766,625, 5,763,574, 5,763,190, 5,762,965, 5,759,769, 5,756,666; 5,753,258, 5,750,373, 5,747,641, 5,747,526, 5,747,028, 5,736,320, 5,736,146, 5,733,760; 5,731,189, 5,728,385, 5,721,095, 5,716,826, 5,716,637, 5,716,613, 5,714,374, 5,709,879; 5,709,860, 5,709,843, 5,705,331, 5,703,057, 5,702,707 5,698,178, 5,688,914, 5,686,078, 5,681,831, 5,679,784, 5,674,984, 5,672,472, 5,667,964, 5,667,783, 5,665,536, 5,665,355; 5,660,990, 5,658,745, 5,658,569, 5,643,756, 5,641,624, 5,639,854, 5,639,598, 5,637,677; 5,637,455, 5,633,234, 5,629,153, 5,627,025, 5,622,705, 5,614,413, 5,610,035, 5,607,831; 5,606,026, 5,601,819, 5,597,688, 5,593,972, 5,591,829, 5,591,823, 5,589,466, 5,587,285; 5,585,254, 5,585,250, 5,580,773, 5,580,739, 5,580,563, 5,573,916, 5,571,667, 5,569,468; 5,558,865, 5,556,745, 5,550,052, 5,543,328, 5,541,100, 5,541,057, 5,534,406 5,529,765, 5,523,232, 5,516,895, 5,514,541, 5,510,264, 5,500,161, 5,480,967, 5,480,966, 5,470,701; 5,468,606, 5,462,852, 5,459,127, 5,449,601, 5,447,838, 5,447,837, 5,439,809, 5,439,792; 5,418,136, 5,399,501, 5,397,695, 5,391,479, 5,384,240, 5,374,519, 5,374,518, 5,374,516; 5,364,933, 5,359,046, 5,356,772, 5,354,654, 5,344,755, 5,335,673, 5,332,567, 5,320,940; 5,317,009, 5,312,902, 5,304,466, 5,296,347, 5,286,852, 5,268,265, 5,264,356, 5,264,342; 5,260,308, 5,256,767, 5,256,561, 5,252,556, 5,230,998, 5,230,887, 5,227,159, 5,225,347, 5,221,610 5,217,861, 5,208,321, 5,206,136, 5,198,346, 5,185,147, 5,178,865, 5,173,400; 5,173,399, 5,166,050, 5,156,951, 5,135,864, 5,122,446, 5,120,662, 5,103,836, 5,100,777; 5,100,662, 5,093,230, 5,077,284, 5,070,010, 5,068,174, 5,066,782, 5,055,391, 5,043,262; 5,039,604, 5,039,522, 5,030,718, 5,030,555, 5,030,449, 5,019,387, 5,013,556, 5,008,183; 5,004,697, 4,997,772, 4,983,529, 4,983,387, 4,965,069, 4,945,082, 4,921,787, 4,918,166; 4,900,548, 4,888,290, 4,886,742, 4,885,235, 4,870,003, 4,869,903, 4,861,707, 4,853,326; 4,839,288, 4,833,072 and 4,795,739.

In another embodiment, HIV, or immunogenic fragments thereof, may be utilized as the HIV epitope. For example, the HIV nucleotides of U.S. Pat. Nos. 7,393,949, 7,374,877, 7,306,901, 7,303,754, 7,173,014, 7,122,180, 7,078,516, 7,022,814, 6,974,866, 6,958,211, 6,949,337, 6,946,254, 6,896,900, 6,887,977, 6,870,045, 6,803,187, 6,794,129, 6,773,915, 6,768,004, 6,706,268, 6,696,291, 6,692,955, 6,656,706, 6,649,409, 6,627,442, 6,610,476, 6,602,705, 6,582,920, 6,557,296, 6,531,587, 6,531,137, 6,500,623, 6,448,078, 6,429,306, 6,420,545, 6,410,013, 6,407,077, 6,395,891, 6,355,789, 6,335,158, 6,323,185, 6,316,183, 6,303,293, 6,300,056, 6,277,561, 6,270,975, 6,261,564, 6,225,045, 6,222,024, 6,194,391, 6,194,142, 6,162,631, 6,114,167, 6,114,109, 6,090,392, 6,060,587, 6,057,102, 6,054,565, 6,043,081, 6,037,165, 6,034,233, 6,033,902, 6,030,769, 6,020,123, 6,015,661, 6,010,895, 6,001,555, 5,985,661, 5,980,900, 5,972,596, 5,939,538, 5,912,338, 5,869,339, 5,866,701, 5,866,694, 5,866,320, 5,866,137, 5,864,027, 5,861,242, 5,858,785, 5,858,651, 5,849,475, 5,843,638, 5,840,480, 5,821,046, 5,801,056, 5,786,177, 5,786,145, 5,773,247, 5,770,703, 5,756,674, 5,741,706, 5,705,612, 5,693,752, 5,688,637, 5,688,511, 5,684,147, 5,665,577, 5,585,263, 5,578,715, 5,571,712, 5,567,603, 5,554,528, 5,545,726, 5,527,895, 5,527,894, 5,223,423, 5,204,259, 5,144,019, 5,051,496 and 4,942,122 are useful for the present invention.

Any epitope recognized by an HIV antibody may be used in the present invention. For example, the anti-HIV antibodies of U.S. Pat. Nos. 6,949,337, 6,900,010, 6,821,744, 6,768,004, 6,613,743, 6,534,312, 6,511,830, 6,489,131, 6,242,197, 6,114,143, 6,074,646, 6,063,564, 6,060,254, 5,919,457, 5,916,806, 5,871,732, 5,824,304, 5,773,247, 5,736,320, 5,637,455, 5,587,285, 5,514,541, 5,317,009, 4,983,529, 4,886,742, 4,870,003 and 4,795,739 are useful for the present invention. Furthermore, monoclonal anti-HIV antibodies of U.S. Pat. Nos. 7,074,556, 7,074,554, 7,070,787, 7,060,273, 7,045,130, 7,033,593, RE39,057, 7,008,622, 6,984,721, 6,972,126, 6,949,337, 6,946,465, 6,919,077, 6,916,475, 6,911,315, 6,905,680, 6,900,010, 6,825,217, 6,824,975, 6,818,392, 6,815,201, 6,812,026, 6,812,024, 6,797,811, 6,768,004, 6,703,019, 6,689,118, 6,657,050, 6,608,179, 6,600,023, 6,596,497, 6,589,748, 6,569,143, 6,548,275, 6,525,179, 6,524,582, 6,506,384, 6,498,006, 6,489,131, 6,465,173, 6,461,612, 6,458,933, 6,432,633, 6,410,318, 6,406,701, 6,395,275, 6,391,657, 6,391,635, 6,384,198, 6,376,170, 6,372,217, 6,344,545, 6,337,181, 6,329,202, 6,319,665, 6,319,500, 6,316,003, 6,312,931, 6,309,880, 6,296,807, 6,291,239, 6,261,558, 6,248,514, 6,245,331, 6,242,197, 6,241,986, 6,228,361, 6,221,580, 6,190,871, 6,177,253, 6,146,635, 6,146,627, 6,146,614, 6,143,876, 6,132,992, 6,124,132, RE36,866, 6,114,143, 6,103,238, 6,060,254, 6,039,684, 6,030,772, 6,020,468, 6,013,484, 6,008,044, 5,998,132, 5,994,515, 5,993,812, 5,985,545, 5,981,278, 5,958,765, 5,939,277, 5,928,930, 5,922,325, 5,919,457, 5,916,806, 5,914,109, 5,911,989, 5,906,936, 5,889,158, 5,876,716, 5,874,226, 5,872,012, 5,871,732, 5,866,694, 5,854,400, 5,849,583, 5,849,288, 5,840,480, 5,840,305, 5,834,599, 5,831,034, 5,827,723, 5,821,047, 5,817,767, 5,817,458, 5,804,440, 5,795,572, 5,783,670, 5,776,703, 5,773,225, 5,766,944, 5,753,503, 5,750,373, 5,747,641, 5,736,341, 5,731,189, 5,707,814, 5,702,707, 5,698,178, 5,695,927, 5,665,536, 5,658,745, 5,652,138, 5,645,836, 5,635,345, 5,618,922, 5,610,035, 5,607,847, 5,604,092, 5,601,819, 5,597,896, 5,597,688, 5,591,829, 5,558,865, 5,514,541, 5,510,264, 5,478,753, 5,374,518, 5,374,516, 5,344,755, 5,332,567, 5,300,433, 5,296,347, 5,286,852, 5,264,221, 5,260,308, 5,256,561, 5,254,457, 5,230,998, 5,227,159, 5,223,408, 5,217,895, 5,180,660, 5,173,399, 5,169,752, 5,166,050, 5,156,951, 5,140,105, 5,135,864, 5,120,640, 5,108,904, 5,104,790, 5,049,389, 5,030,718, 5,030,555, 5,004,697, 4,983,529, 4,888,290, 4,886,742 and 4,853,326, are also useful for the present invention.

The immunogen may be a cancer immunogen, wherein the cancers include, but are not limited to, leukemias, including but not limited to acute leukemia, acute lymphocytic leukemia, acute myelocytic leukemia, myeloblastic, promyelocytic, myelomonocytic, monocytic, erythroleukemia, chronic leukemia, chronic myelocytic (granulocytic) leukemia, chronic lymphocytic leukemia, Polycythemia vera, Lymphomas including but not limited to Hodgkin's disease, non-Hodgkin's disease, Multiple myeloma, Waldenstrom's macroglobulinemia, Heavy chain disease, Solid tumors including but not limited to sarcomas and carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, and neuroblastomaretinoblastoma.

The immunogen may be an inflammatory immunogen wherein the inflammatory disease includes, but is not limited to, eczema, inflammatory bowel disease, rheumatoid arthritis, asthma, psoriasis, ischemia/reperfusion injury, ulcerative colitis and acute respiratory distress syndrome.

The immunogen may be a cardiovascular disease immunogen, wherein the cardiovascular diseases include, but are not limited to, hypertension, heart failure, pulmonary hypertension and renal diseases.

Antigens may be prepared by any method known to one of ordinary skill in the art, including, but not limited to, antigen biotinylation by sulfo-NHS-LC-LC-biotin (commercially available from Pierce). An antigenic/immunogenic protein is produced or purified by any methods known to one of ordinary skill in the art.

Fusion polypeptides between the antigen/immunogen and other homologous or heterologous proteins are also provided. Heterologous fusions may be constructed which would exhibit a combination of properties or activities of the derivative proteins. Typical examples are fusions of a reporter polypeptide, e.g., luciferase, with a segment or domain of a receptor, e.g., a ligand-binding segment, so that the presence or location of a desired ligand may be easily determined. See, e.g., Dull, et al., U.S. Pat. No. 4,859,609, which is hereby incorporated herein by reference. Other gene fusion partners include glutathione-S-transferase (GST), bacterial.beta.-galactosidase, trpE, Protein A, .beta.-lactamase, alpha amylase, alcohol dehydrogenase, and yeast alpha mating factor. See, e.g., Godowski, et al., Science 241, 812 (1988).

Such polypeptides may also have amino acid residues which have been chemically modified by phosphorylation, sulfonation, biotinylation, or the addition or removal of other moieties, particularly those which have molecular shapes similar to phosphate groups. In some embodiments, the modifications will be useful labeling reagents, or serve as purification targets, e.g., affinity ligands.

Fusion proteins are typically made by either recombinant nucleic acid methods or by synthetic polypeptide methods. Techniques for nucleic acid manipulation and expression are described generally, for example, in Sambrook, et al., Molecular Cloning: A Laboratory Manual (2d ed.), Vols. 1-3, Cold Spring Harbor Laboratory (1989), and Ausubel, et al., Current Protocols in Molecular Biology, Greene/Wiley, New York (1987), which are each incorporated herein by reference. Techniques for synthesis of polypeptides are described, for example, in Merrifield, J. Amer. Chem. Soc. 85, 2149 (1963); Merrifield, Science 232, 341 (1986); and Atherton, et al., Solid Phase Peptide Synthesis: A Practical Approach, IRL Press, Oxford (1989); each of which is incorporated herein by reference. See also Dawson, et al., Science 266,776 (1994) for methods to make larger polypeptides.

In an advantageous embodiment, the antigens are recombinant antigens. The vectors used in accordance with the present invention should typically be chosen such that they contain a suitable gene regulatory region, such as a promoter or enhancer, such that the antigens and/or antibodies of the invention can be expressed.

For example, when the aim is to express the antibodies and/or antigens of the invention in vitro, or in cultured cells, or in any prokaryotic or eukaryotic system for the purpose of producing the protein(s) encoded by that antibody and/or antigen, then any suitable vector can be used depending on the application. For example, plasmids, viral vectors, bacterial vectors, protozoal vectors, insect vectors, baculovirus expression vectors, yeast vectors, mammalian cell vectors, and the like, can be used. Suitable vectors can be selected by the skilled artisan taking into consideration the characteristics of the vector and the requirements for expressing the antibodies and/or antigens under the identified circumstances.

In a particularly advantageous embodiment of the present invention, the protein fragments of the present invention are expressed in a system that produces non-glycosylated versions of the protein fragments. In particular, a bacterial system is utilized to express the protein fragments of the present invention. Advantageously, the bacteria is E. coli, in particular B121(DE3) cells. The vector is advantageously a bacterial expression vector, in particular a bacterial expression vector with a T7 promoter.

When the aim is to express the antibodies and/or antigens of the invention in vivo in a subject, for example in order to generate an immune response and/or protective immunity, expression vectors that are suitable for expression on that subject, and that are safe for use in vivo, should be chosen. For example, in some embodiments it may be desired to express the antibodies and/or antigens of the invention in a laboratory animal, such as for pre-clinical testing of the HIV-1 immunogenic compositions and vaccines of the invention. In other embodiments, it will be desirable to express the antibodies and/or antigens of the invention in human subjects, such as in clinical trials and for actual clinical use of the immunogenic compositions and vaccine of the invention. Any vectors that are suitable for such uses can be employed, and it is well within the capabilities of the skilled artisan to select a suitable vector. In some embodiments it may be preferred that the vectors used for these in vivo applications are attenuated to vector from amplifying in the subject. For example, if plasmid vectors are used, preferably they will lack an origin of replication that functions in the subject so as to enhance safety for in vivo use in the subject. If viral vectors are used, preferably they are attenuated or replication-defective in the subject, again, so as to enhance safety for in vivo use in the subject.

In preferred embodiments of the present invention viral vectors are used. Viral expression vectors are well known to those skilled in the art and include, for example, viruses such as adenoviruses, adeno-associated viruses (AAV), alphaviruses, herpesviruses, retroviruses and poxviruses, including avipox viruses, attenuated poxviruses, vaccinia viruses, and particularly, the modified vaccinia Ankara virus (MVA; ATCC Accession No. VR-1566). Such viruses, when used as expression vectors are innately non-pathogenic in the selected subjects such as humans or have been modified to render them non-pathogenic in the selected subjects. For example, replication-defective adenoviruses and alphaviruses are well known and can be used as gene delivery vectors.

The nucleotide sequences and vectors of the invention can be delivered to cells, for example if aim is to express the antigens in cells in order to produce and isolate the expressed proteins, such as from cells grown in culture. For expressing the antibodies and/or antigens in cells any suitable transfection, transformation, or gene delivery methods can be used. Such methods are well known by those skilled in the art, and one of skill in the art would readily be able to select a suitable method depending on the nature of the nucleotide sequences, vectors, and cell types used. For example, transfection, transformation, microinjection, infection, electroporation, lipofection, or liposome-mediated delivery could be used. Expression of the antibodies and/or antigens can be carried out in any suitable type of host cells, such as bacterial cells, yeast, insect cells, and mammalian cells. The antibodies and/or antigens of the invention can also be expressed using including in vitro transcription/translation systems. All of such methods are well known by those skilled in the art, and one of skill in the art would readily be able to select a suitable method depending on the nature of the nucleotide sequences, vectors, and cell types used.

Liposomes may be prepared by any method known to one of ordinary skill in the art, including, but not limited to, use of liposomes containing 1,2-Dioleoyl-sn-Glycero-3-phosphocholine (DOPC) and N-Cap 1-oleoyl-2-(12-biotinyl(amino)auroyl))-sn-glycero-3-phosphoethanolamine (PE-biotin) (both commercially available from Avanti Polar Lipids), DOPC/PE-biotin (e.g. 98/2, m/m) or DOPC/PE-biotin/αGalCer (e.g. 88/2/10, m/m/m); any lipid known to activate iNKT-cells may be incorporated in the liposomes. Lipids may, for example, be dried under argon, and resuspended in buffers, such as, for example, but not limited to, 25 mMTris, 150 mM NaCl, pH 7.0 with vigorous mixing.

αGalCer is commercially available from Alexis Biochemical. The synthesis of αGalCer-Alexa 488 may be synthesized by any method known to one of ordinary skill in the art, including, but not limited to, methods described in the literature, such as for example, the methods described by Yuan et al. (Yuan et al., 2007. Proc Natl Acad Sci USA 104:5551-5556).

Any method known to one of ordinary skill in the art to quantify the amount of αGalCer bound to particles may be used, including, but not limited to, use of αGalCer-Alexa 488-containing liposomes and using an EnVision Multilabel Reader to record relative fluorescence intensity.

Any glycolipid may be used as long as it is known to activate iNKT-cells because of their expression of NK surface markers such as CD161. NKT-cells are activated by CD1d-presented antigens, and rapidly produce Th1 and Th2 cytokines, typically represented by interferon-gamma and IL-4 production. Advantageously, the glycolipids are CD1-effective glycolipids. The natural antigens of group 2 CD1 are not well-characterized, but synthetic glycolipid αGalCer originally isolated from a compound found in a marine sponge, has strong biologic activity.

The amount of the glycoside contained in the liposomal composition of the present invention may be 0.3 to 2.0 mol, preferably 0.8 to 1.5 mol, on the basis of 1 mol of a phospholipid.

The liposomal composition of the present invention may contain at least a phospholipid as a membrane component. Examples of the phospholipid include phosphatidylcholines such as dilauroylphosphatidylcholine, dimyristoylphosphatidylcholine, dipalmitoylphosphatidylcholine, distearoylphosphatidylcholine, dioleoylphosphatidylcholine, dilinoleoylphosphatidylcholine, myristoylpalmitoylphosphatidylcholine, myristoylstearoylphosphatidylcholine, and palmitoylarachidoylphosphatidylcholine; phosphatidylethanolamine; phosphatidylserine; phosphatidylinositol; and phosphatidic acid. The phospholipid may be a naturally occurring product, or may be obtained through semisynthesis or total synthesis. The phospholipid may be a processed phospholipid such as a hydrogenated phospholipid. These phospholipids may be employed singly or in combination of two or more species.

The positive-charge-providing substance is added for positively charging the surface of a lipid membrane. When the surface of liposomes is positively charged, the liposomes are expected to come into natural contact with cells having negatively charged membrane surfaces. Examples of the positive-charge-providing substance include aliphatic amines such as stearylamine and oleylamine; and aromatic amines such as fluoreneethylamine. Of these, aliphatic amines are preferred, and stearylamine is particularly preferably employed. The amount of the positive-charge-providing substance contained in the liposomal composition is 0.04 to 0.15 mol, preferably 0.1 to 0.15 mol, on the basis of 1 mol of the phospholipid.

If desired, the liposomal composition of the present invention may contain, in addition to the aforementioned components, a membrane structure stabilizer such as cholesterol, a fatty acid, or diacetyl phosphate.

The aqueous solution employed for dispersing the membrane component is preferably water, saline, a buffer, an aqueous solution of a sugar, or a mixture thereof. The buffer to be employed is preferably an organic or inorganic buffer which has buffering action in the vicinity of the hydrogen ion concentration of body fluids. For example, a phosphate buffer can be employed.

No particular limitation is imposed on the method for preparing the liposomal composition of the present invention, and the composition can be prepared through a customary method. For example, the liposomal composition can be prepared through the method described in JP-A-57-82310, JP-A-60-12127, JP-A-60-58915, JP-A-1-117824, JP-A-1-167218, JP-A-4-29925, or JP-A-9-87168, the method described in Methods of Biochemical Analysis (1988) 33, p 337, or the method described in “Liposome” (Nankodo).

Preparation of the Liposomal Composition of the Present Invention May be Through the method described in Japanese Patent Application Laid-Open (kokai) No. 9-87168. First, an organic solvent and water are added to and mixed with a glycoside, a phospholipid, and a positive-charge-providing substance, and subsequently the organic solvent is completely removed by means of a rotary evaporator or a similar apparatus, followed by removal of the water. In this case, the mixing proportions of the membrane component, the positive-charge-providing substance, and the glycoside may be, for example, 52:8:20 (by mole). However, so long as the mixing proportions fall within a range nearly equal to the above range, particular problems do not arise. When the mixing proportion of the glycoside is low, if desired, a membrane structure stabilizer such as cholesterol may be added. However, when the mixing proportion of the glycoside is high, addition of such a membrane structure stabilizer is not necessarily required. No particular limitation is imposed on the organic solvent to be employed, so long as it is a volatile organic solvent which is insoluble in water. Examples of the organic solvent which may be employed include chloroform, chloromethane, benzene, and hexane. In consideration of solubility, an organic solvent having relatively high polarity (e.g., ethanol or methanol) may be appropriately added to such a water-insoluble solvent, and the thus-prepared organic solvent mixture may be employed. No particular limitation is imposed on the mixing proportions of the organic solvent mixture and water, so long as a uniform solvent mixture is obtained.

In the case where water is added for preparation of the liposomal composition, removal of the water is generally carried out through freeze-drying. However, removal of the water is not necessarily performed through freeze-drying, and may be performed through drying in a reduced-pressure desiccator. After removal of the water, the aforementioned aqueous solution for dispersion is added, followed by impregnation by means of, for example, a Vortex mixer, to thereby form the liposomal composition.

Liposomes having a uniform particle size can be prepared through, for example, ultrasonic treatment, extrusion treatment by use of a porous membrane filter, treatment by use of a high-pressure injection emulsifier, or combination of such treatments. Smaller liposome particles can be prepared by, for example, performing ultrasonic treatment for a long period of time.

For coating the nano-particles, any method known to one of ordinary skill in the art, including, but not limited to, incubation of silica microspheres (e.g. 100 nm; available from Kisker GbR) with liposomes followed by streptavidin and biotinylated proteins.

An example of suitable nanoparticles for the present invention are silica particles. These particles are commercially available (Polysicence Laboratories). It was observed that silica particles having a sedimentation rate of at least 0.5 mm/hour were suitable for the present invention. This corresponds to silica beads of radius 50 nm having a density of 2.6 g/L. In a preferred embodiment, the radius of the beads is between about 50 nm to about 1 μm. In a more preferred embodiment, the radius of the beads is about 225 nm. Silica beads are generally available commercially in water. These can be sterilized or may be used directly. The beads may be diluted in water or in a suitable buffer.

Those skilled in the art will recognize that the reactive groups on the silica beads may be modified, such as by oxidation. Those skilled in the art will also recognize that any polymer beads with similar diameter and density as the silica beads used herein can be used for the present invention. The reactive groups on the polymer beads can also be modified so as to be similar to those (such as silanol groups) on silica beads.

Human B-cell purification from peripheral blood mononuclear cells (PBMCs) or any other source may carried out by any method known to one of ordinary skill in the art, such as, for example, but not limited to, enrichment by negative selection by using a B-cell purification kit (Miltenyi Biotec). In another embodiment, B-cells may be isolated directly from whole blood by negative selection, from mononuclear cells by negative selection or by immunomagnetic isolation by positive selection (see, e.g., Sims and Lipsky, Isolation of B-cell Populations, Current Protocols in Immunology ed. by Judy B. Splawski, Peter E. Lipsky, Eli M. Eisenstein, Kevin S. Chua, December 2006).

In another embodiment, PBMC may be isolated from blood samples via Histopaque layering. About thirty ml of human blood samples may be stored at room temperature for the same period of time from pre-vaccination and post-vaccination of the same volunteers were collected and stored at 25° C. for up to 3 days while remaining in the original vacutainer tubes containing anticoagulant. The total number of viable cells may be counted using a microscope, ahemocytometer, and trypan blue staining.

Following incubation/exposure of B-cells with/to the above nano-particles by standard methods known to one of ordinary skill in the art, analysis of CD-1 effective lipid presentation may be carried out by any method known to one of ordinary skill in the art, such as, for example, but not limited to, incubation of B-cells with particles, followed by washing and culturing the cells at an appropriate concentration, such as, for example, 5×10⁴ cells per well with an appropriate number, such as, for example, an equal number of iNKT-cells, such as, for example, a human CD 1-restricted NKT-cell hybridoma and IL-2 is measured in the supernatant of the co-cultures by any method known to one of ordinary skill in the art, such as, for example, but not limited to, ELISA assays (e.g. using a capture agent and, for example, biotinylated or otherwise labeled antibodies for detection).

In a preferred embodiment, B-cells are transduced by a vector that directs enhanced or overexpression of human CD1A, B, C, D, and/or E. This enhanced expression increases the effectiveness with which B-cell present to, and activate iNKT-cells. Any method of B-cell transduction known to one of ordinary skill in the art may be used, including, for example, but not limited to, transduction with lentiviral vectors that have surface measles or canine distemper H and F glycoproteins, or related virus proteins, and that are reported to transduce a high percentage of quiescent B-cells and do not affect cells' ability to enter the cycle or B-cell activation; in addition these vectors maintain the naive and memory phenotypes of resting B-cells (Frecha et al., 2009. Blood 114(15):3173-80). Other methods involving lentiviral transduction of B-cells may be found in Methods in Molecular Biology, Volume: 415, Pub. Date: Nov. 19, 2007, Page Range: 301-320.

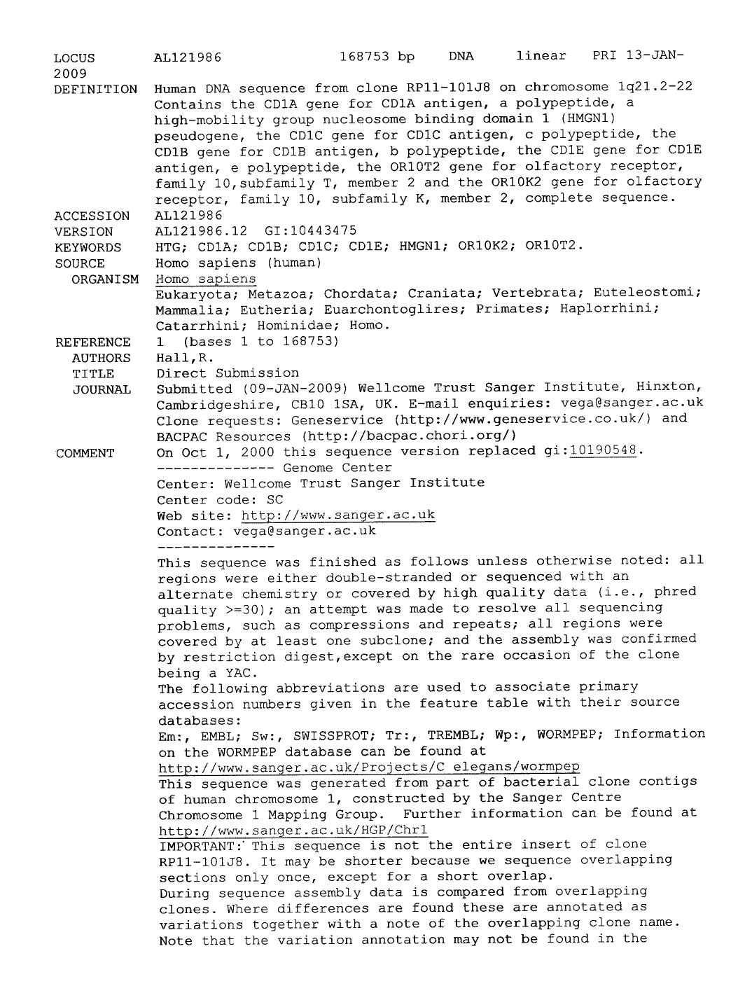

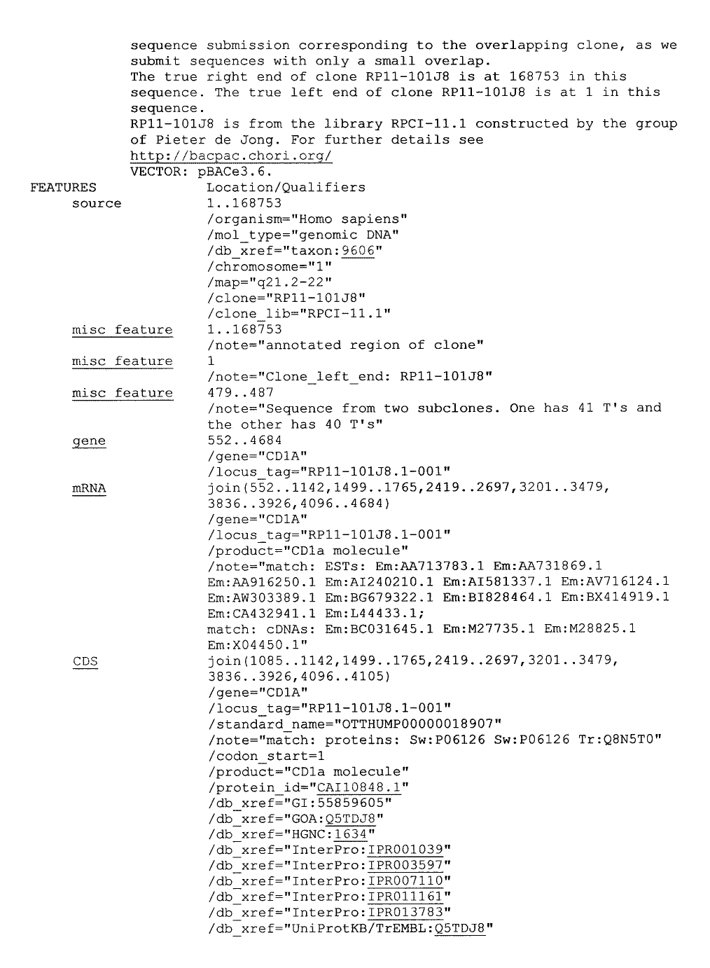

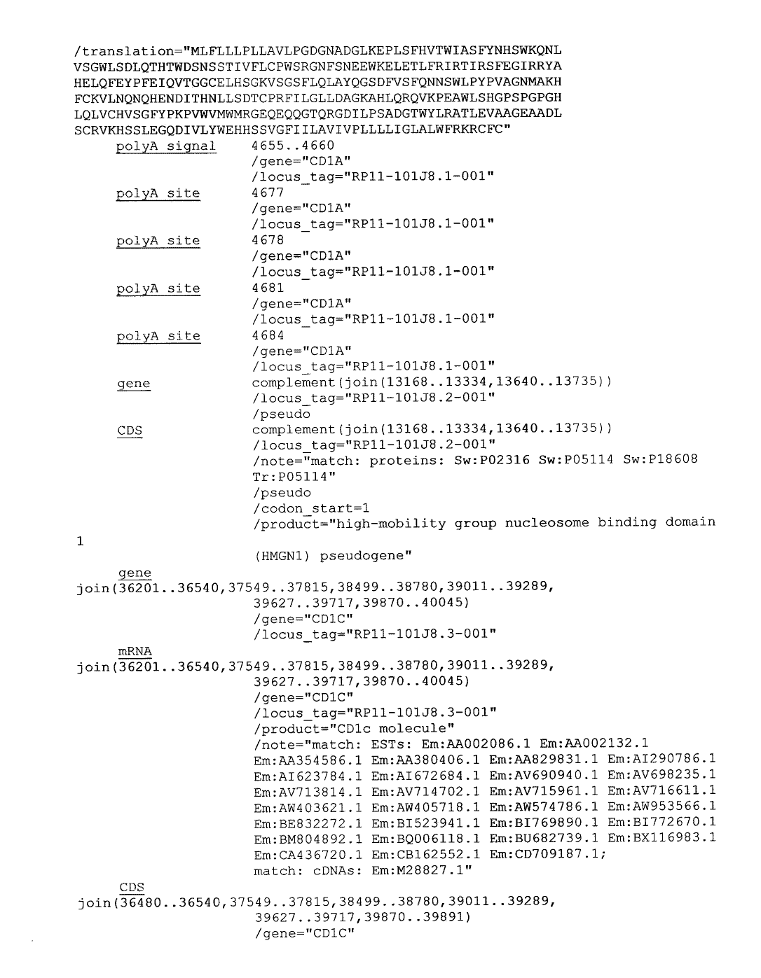

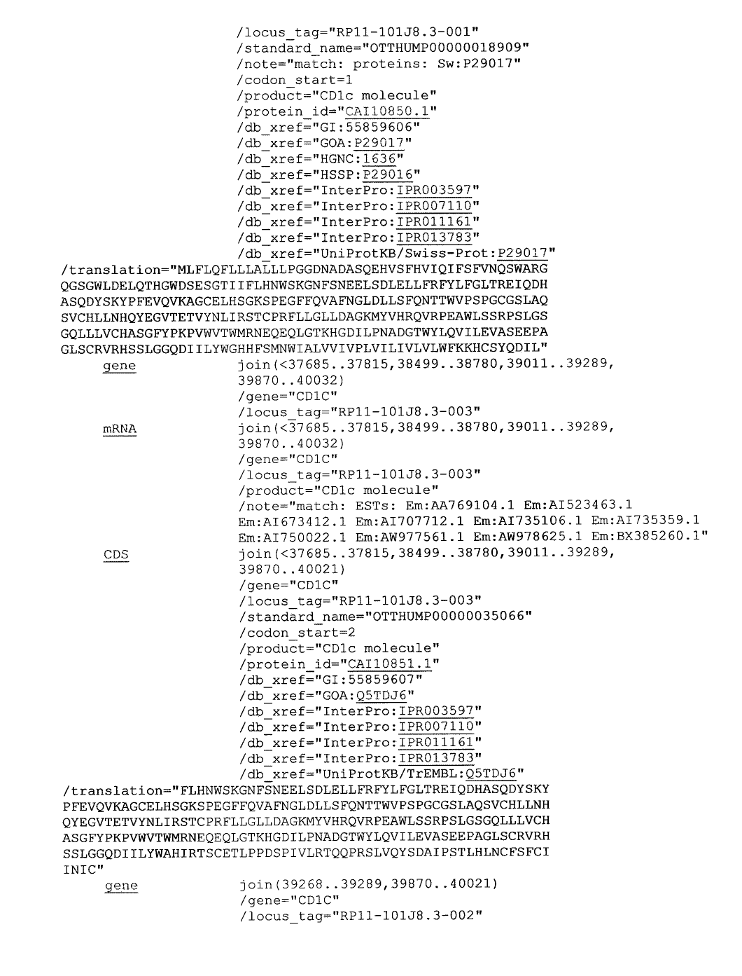

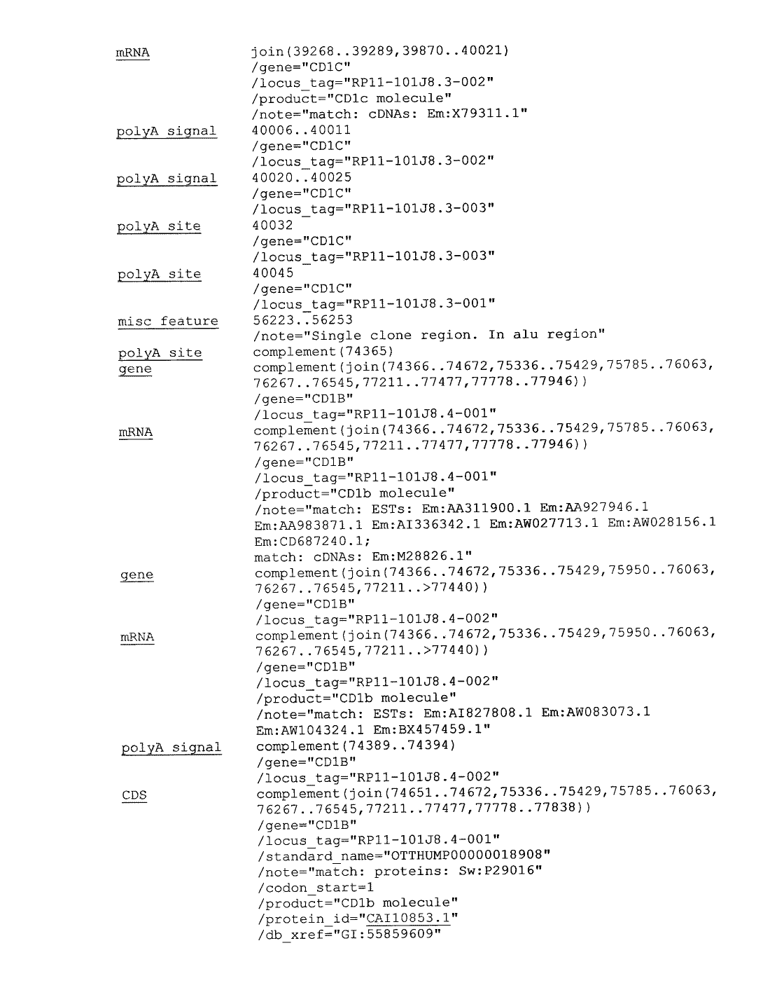

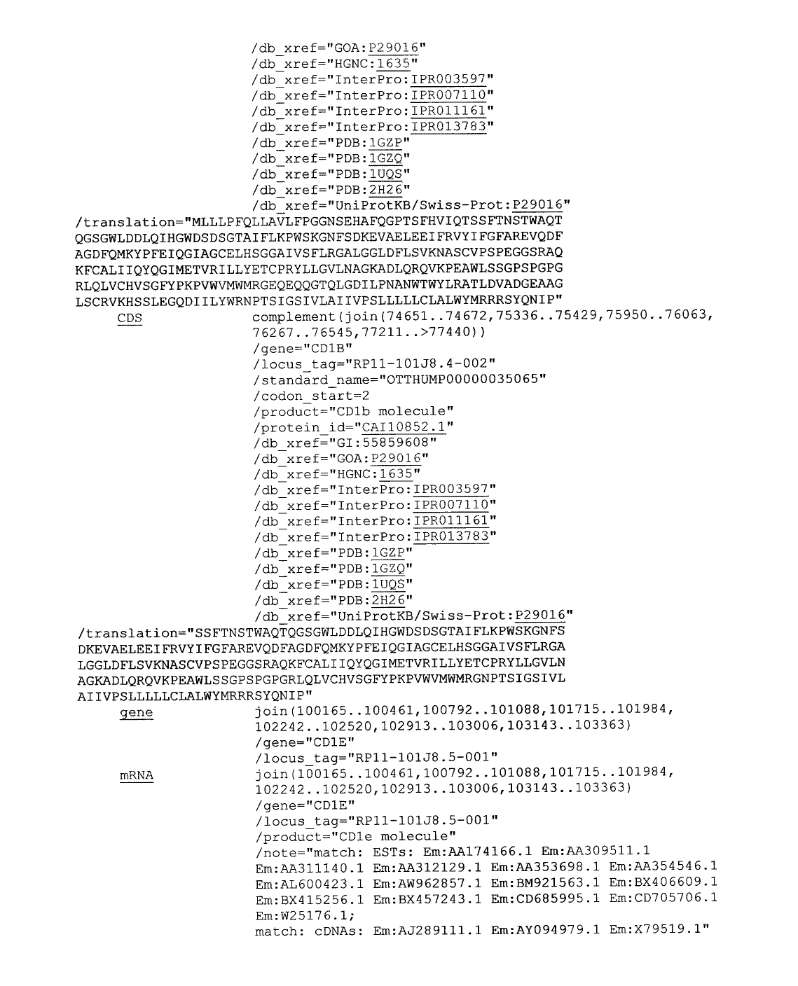

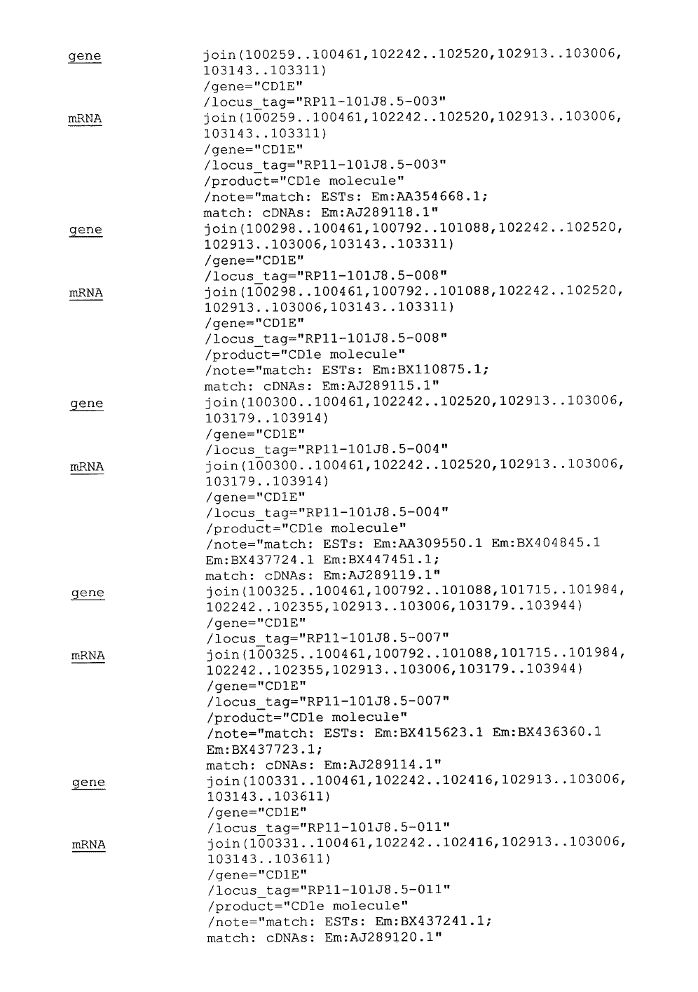

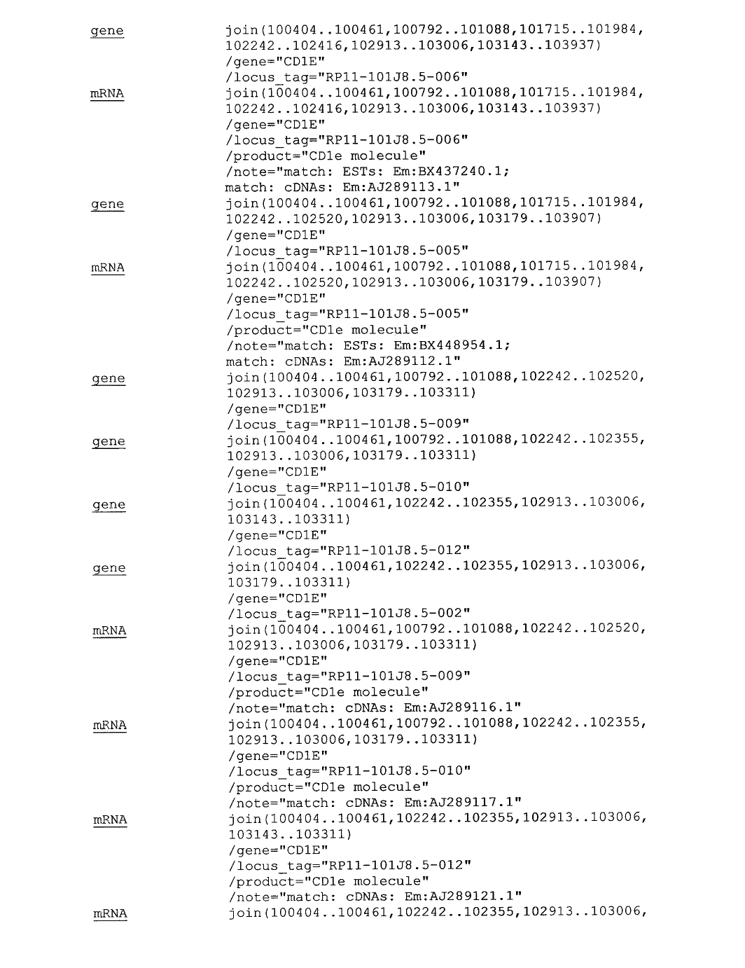

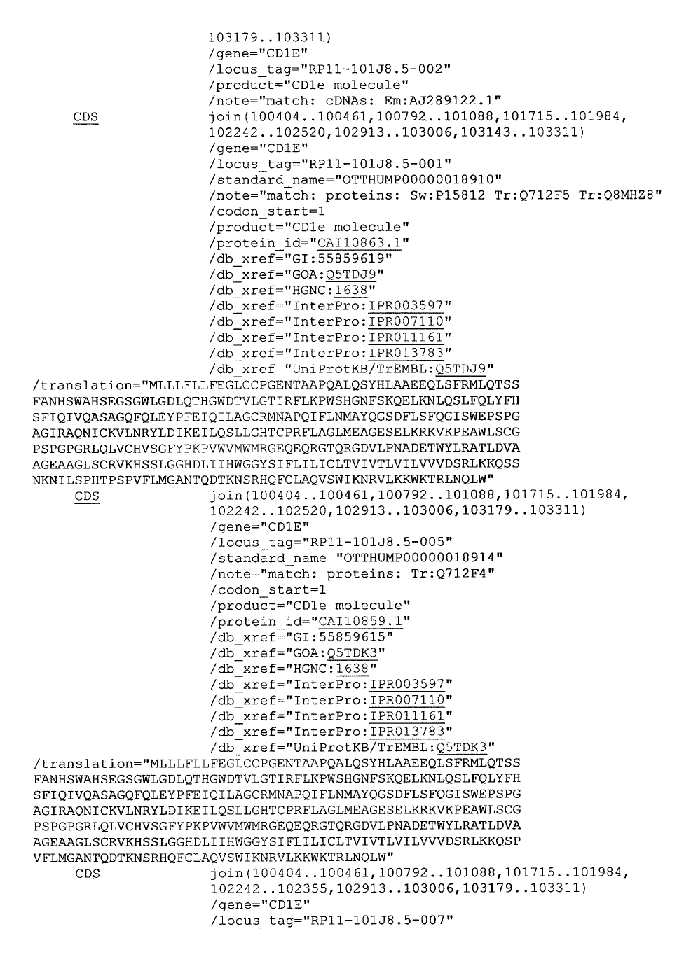

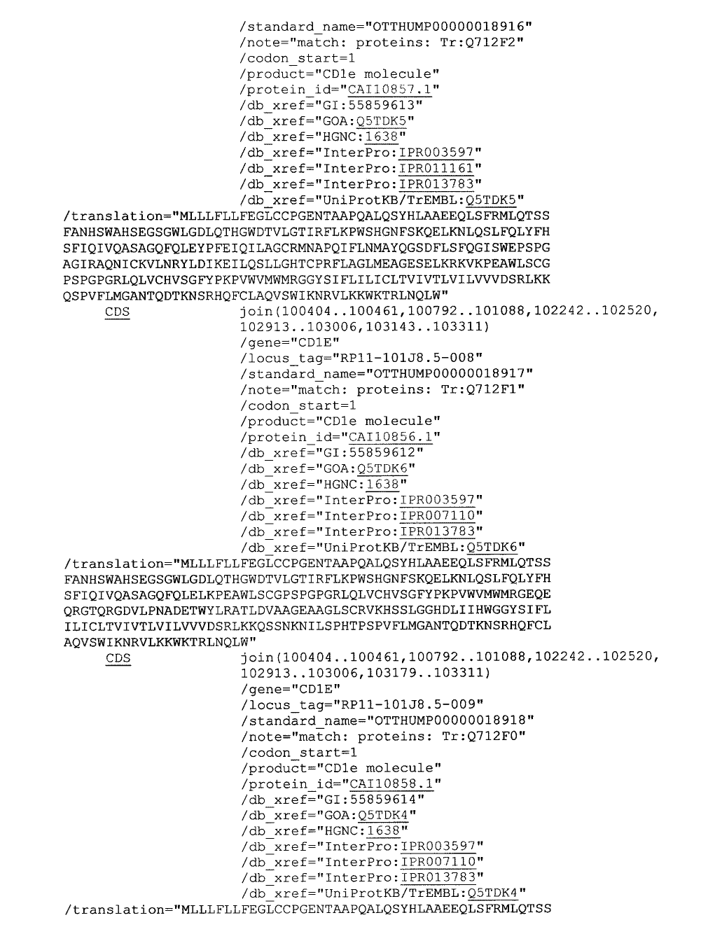

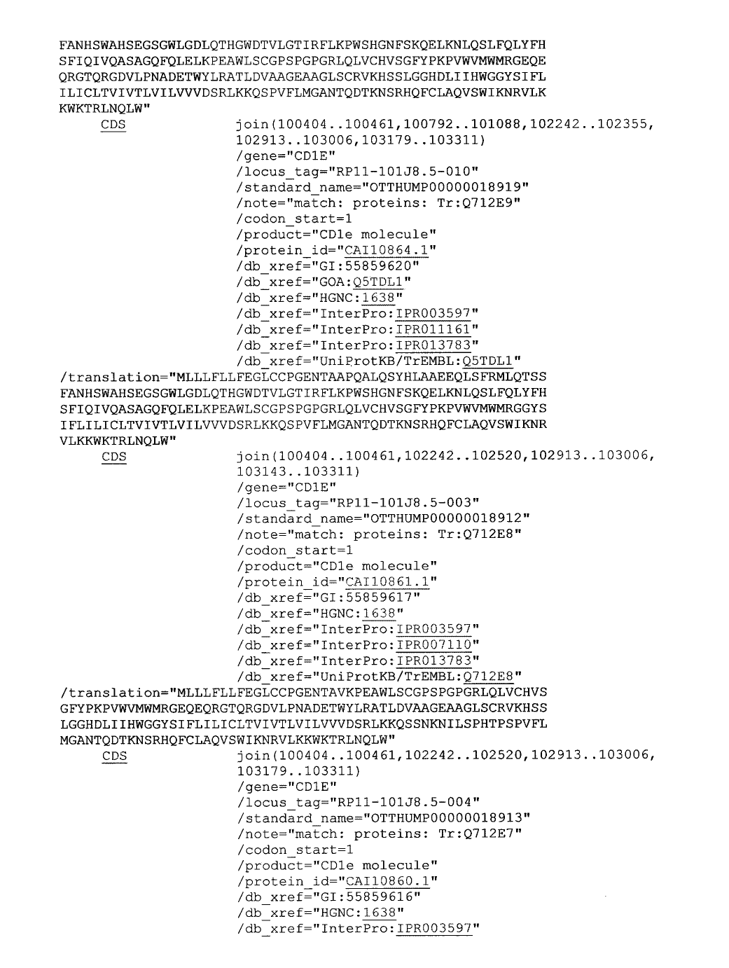

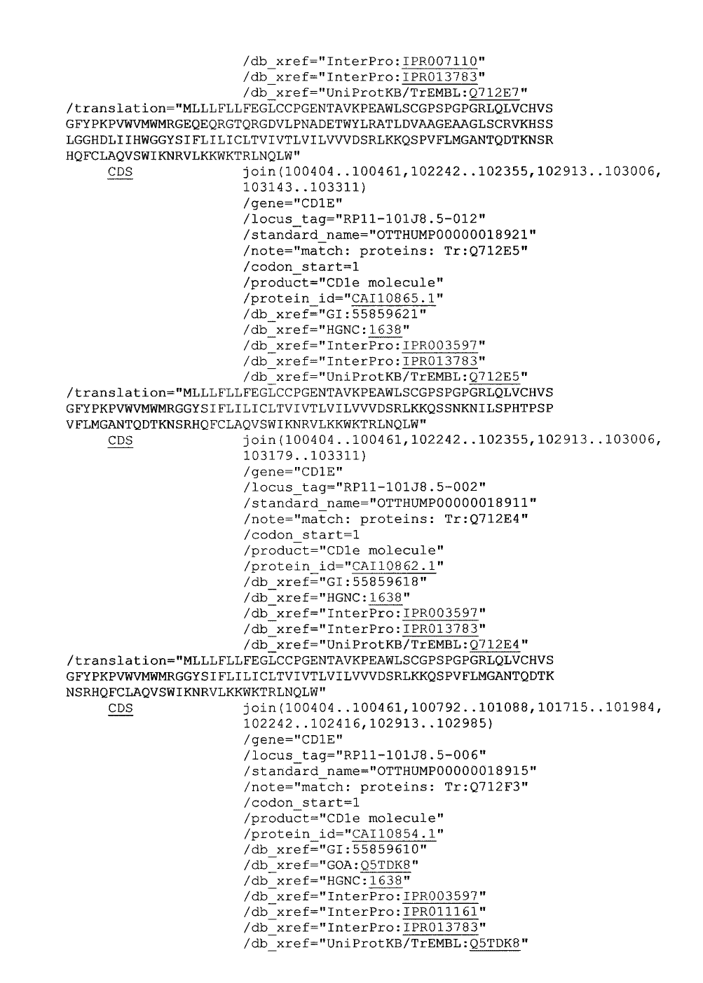

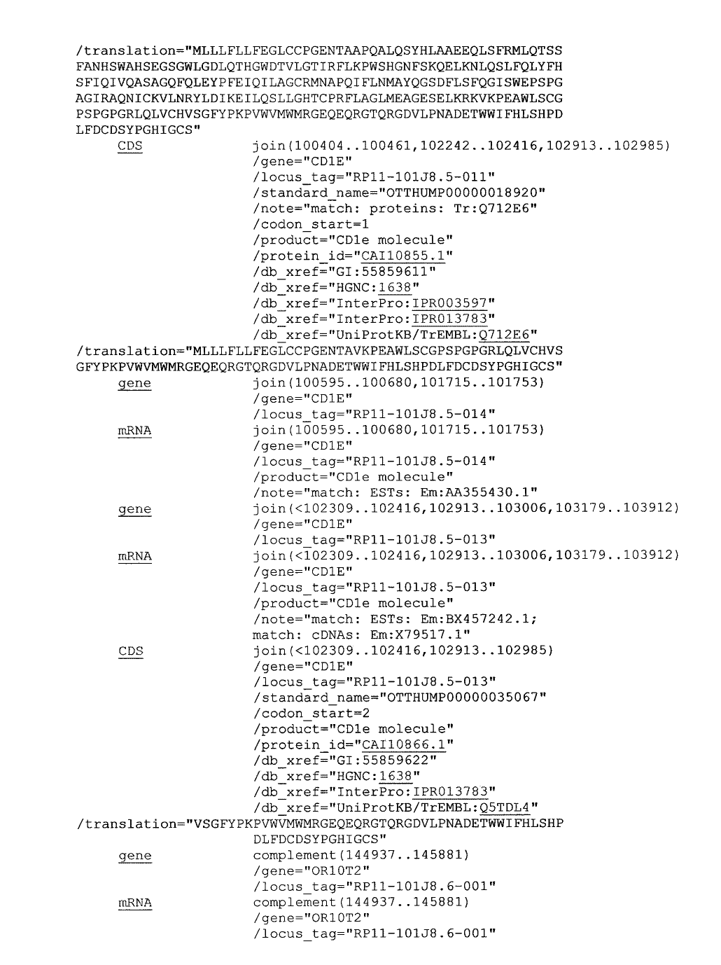

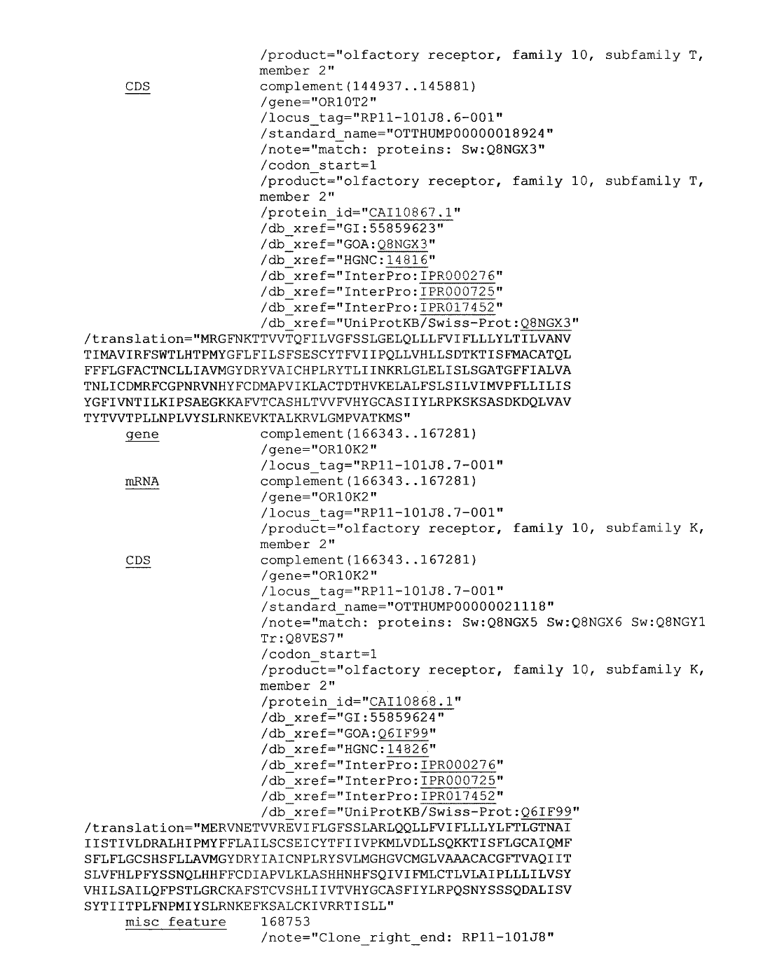

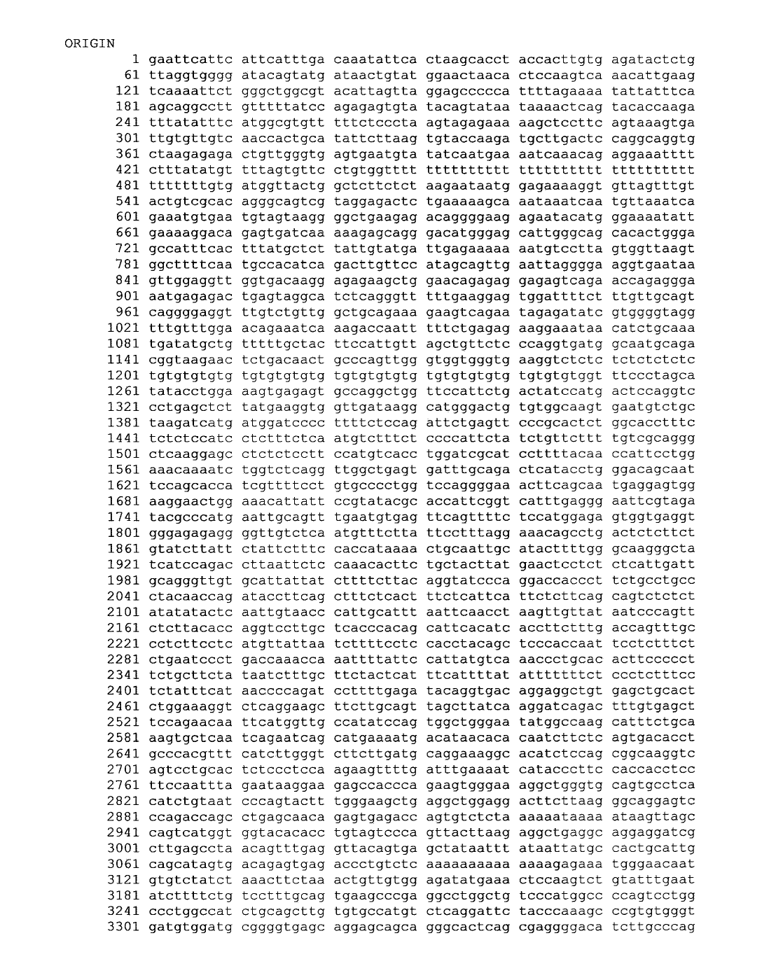

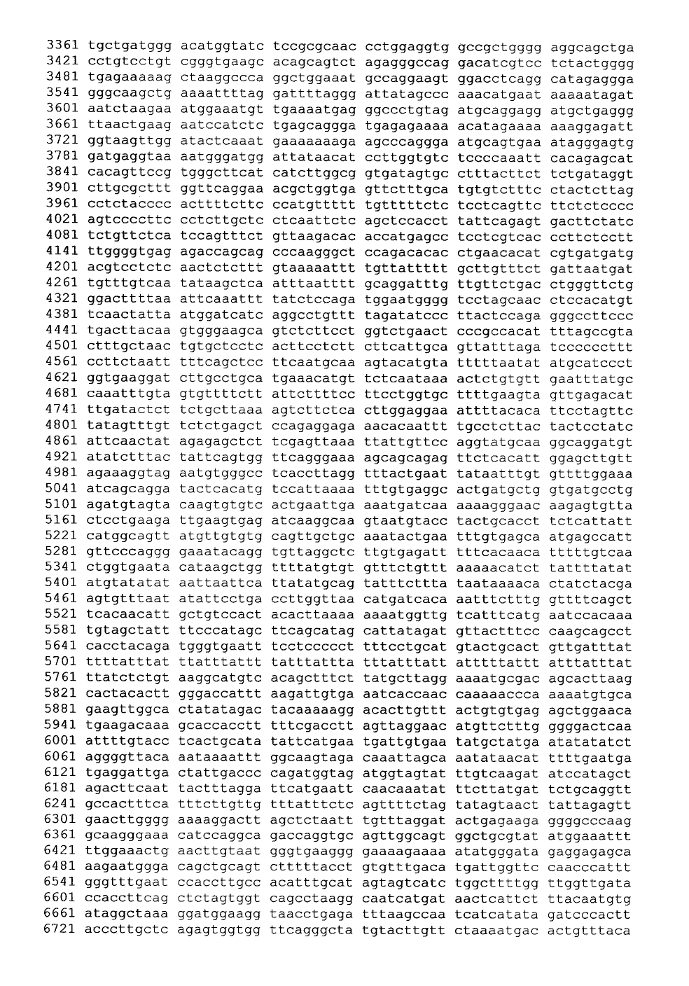

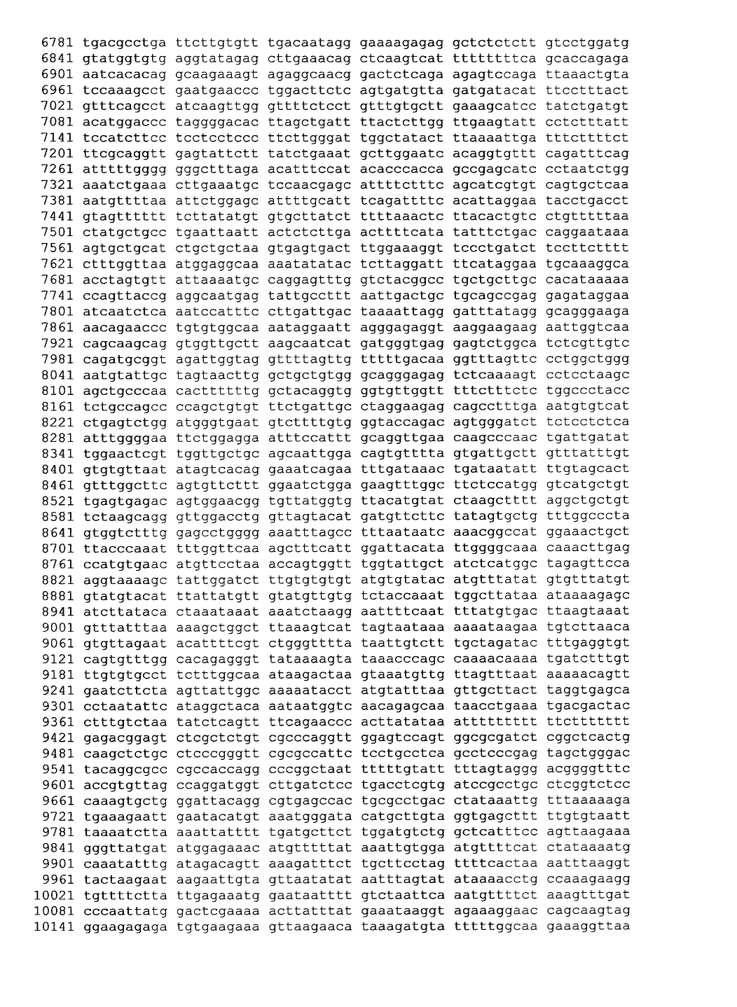

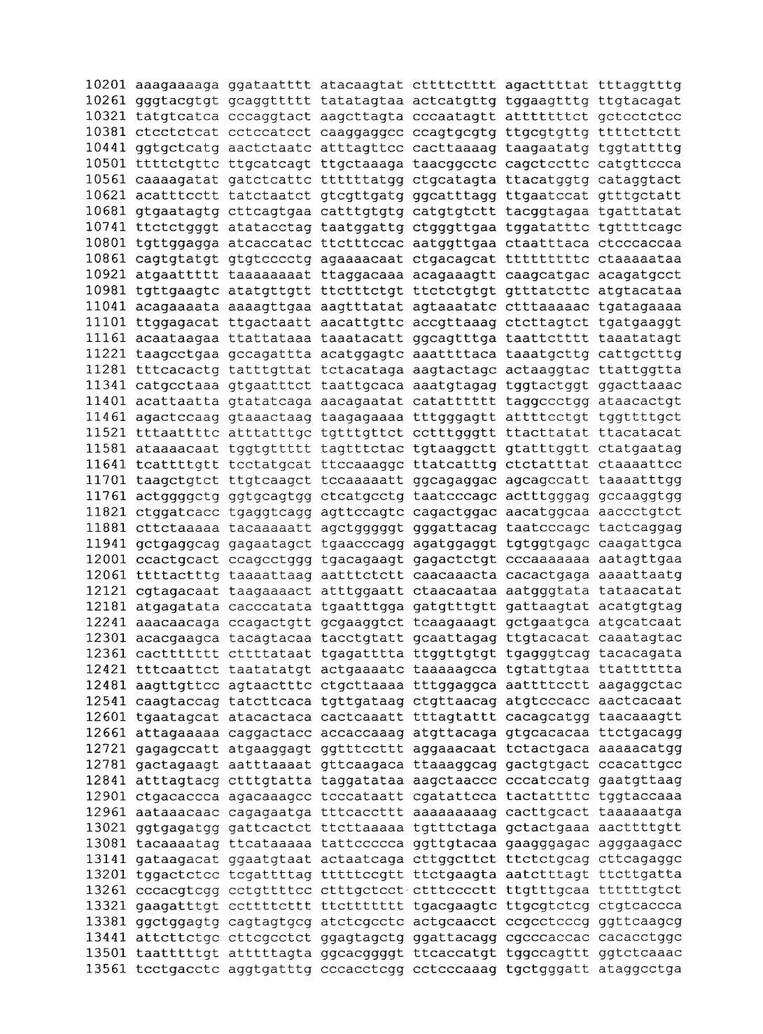

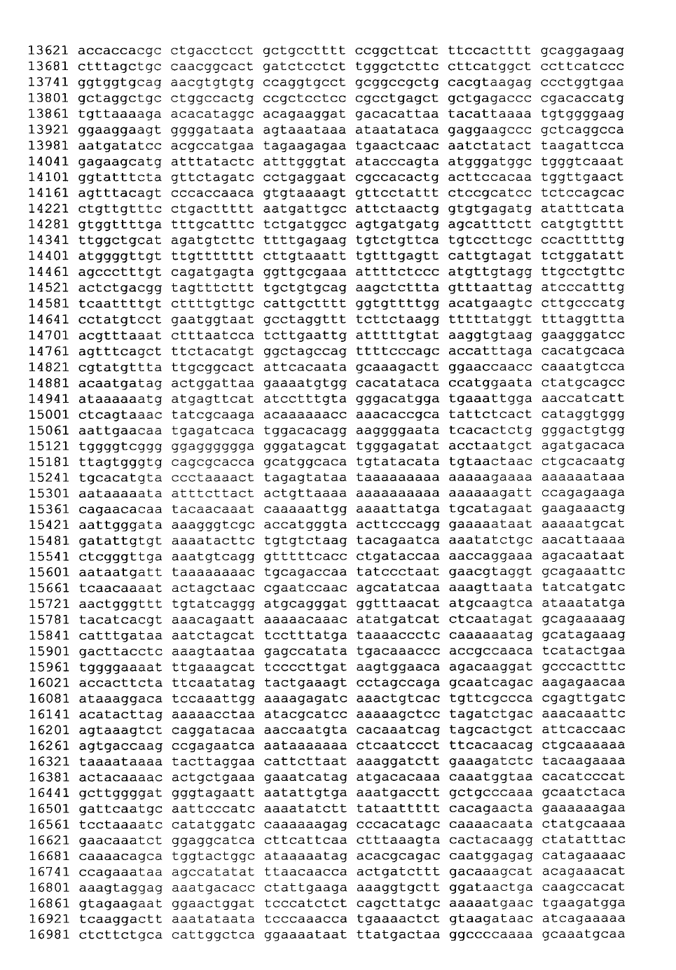

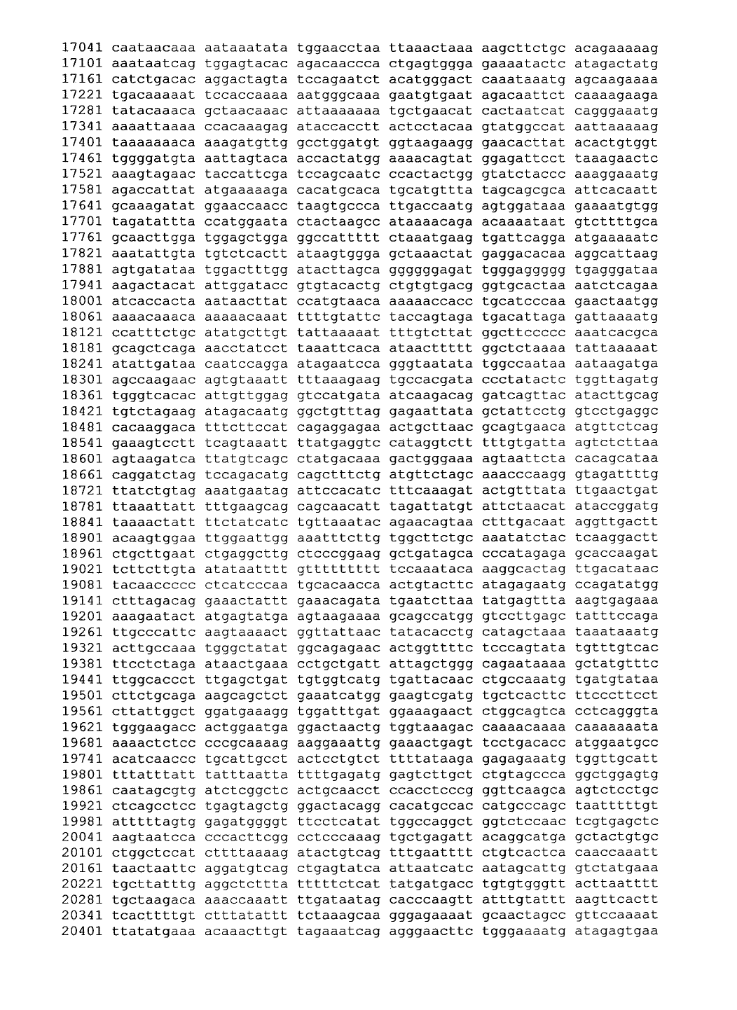

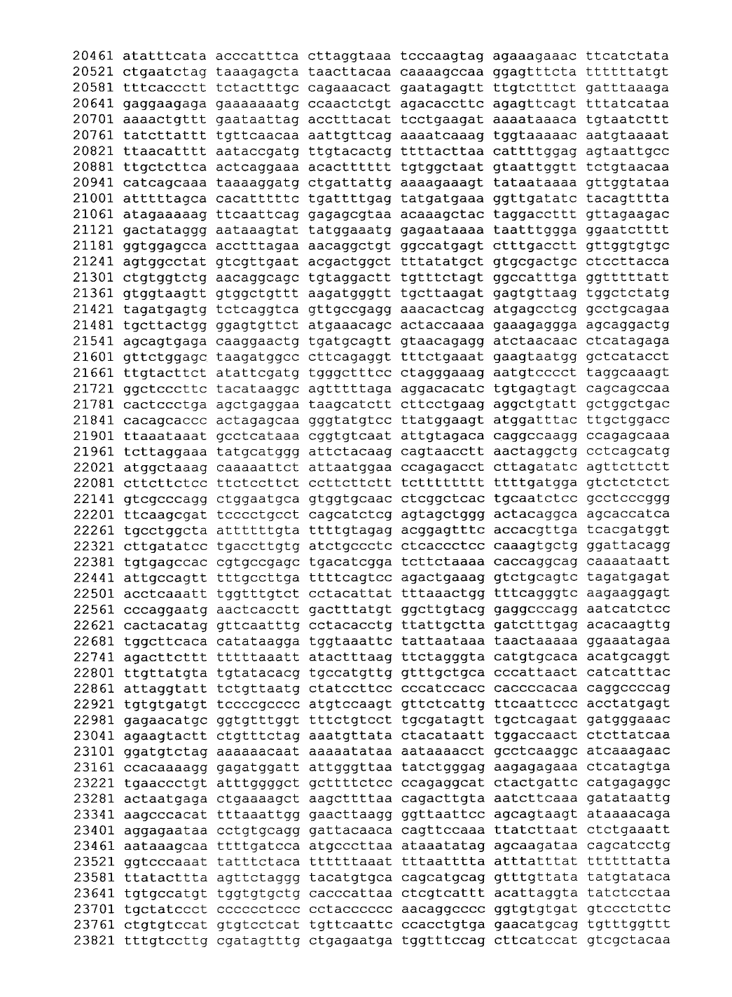

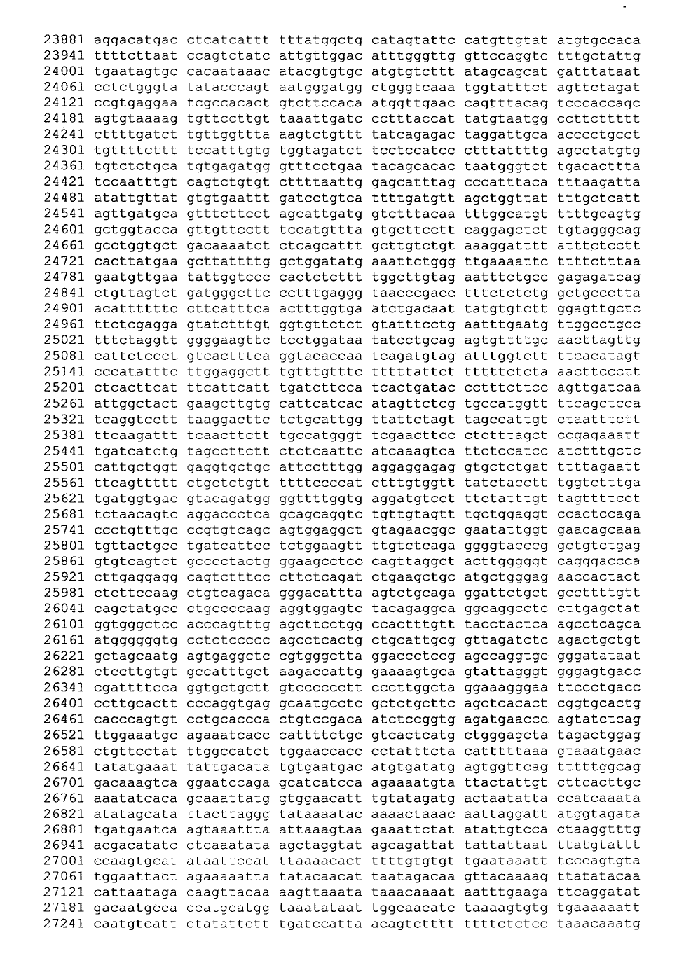

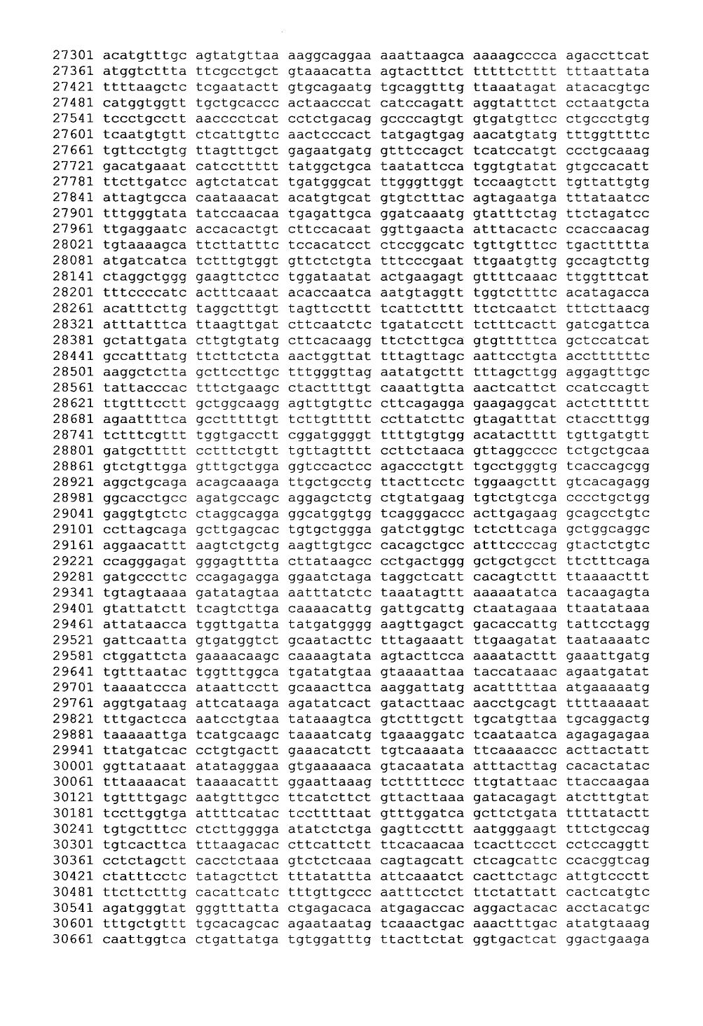

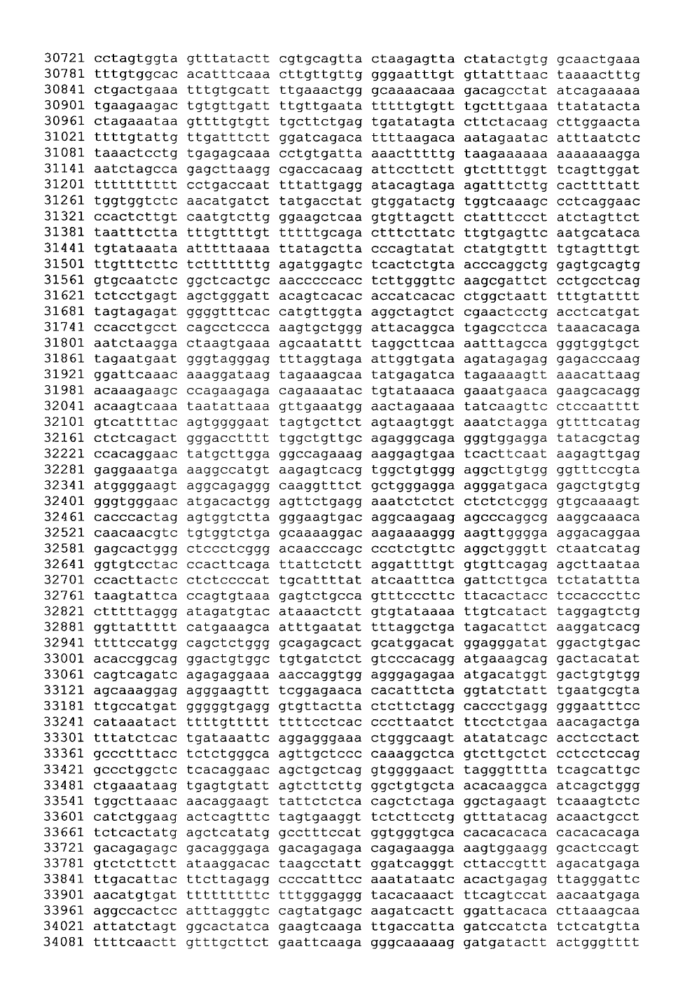

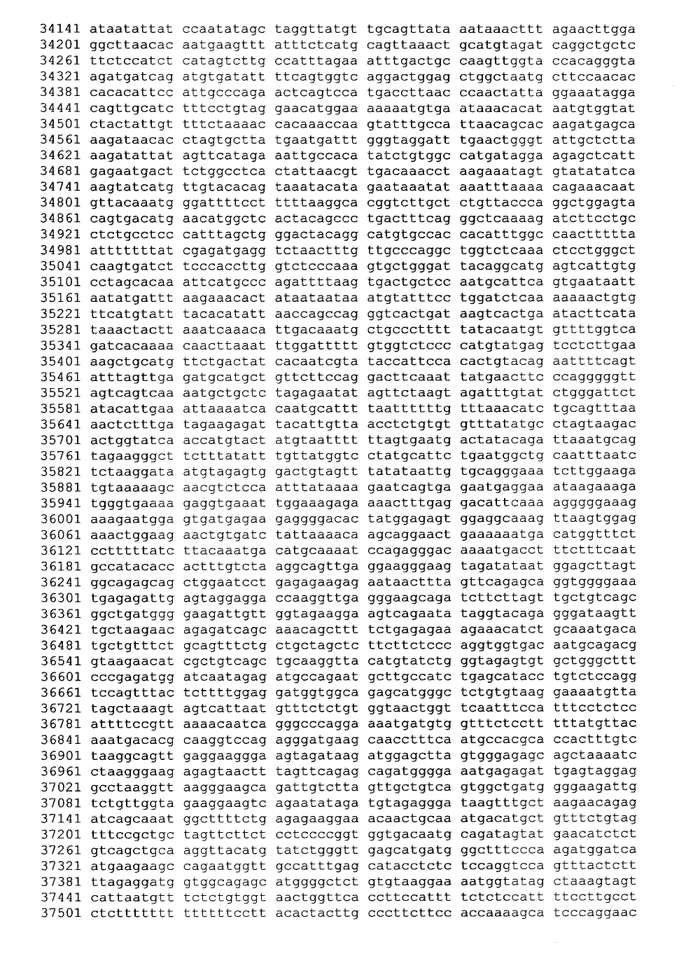

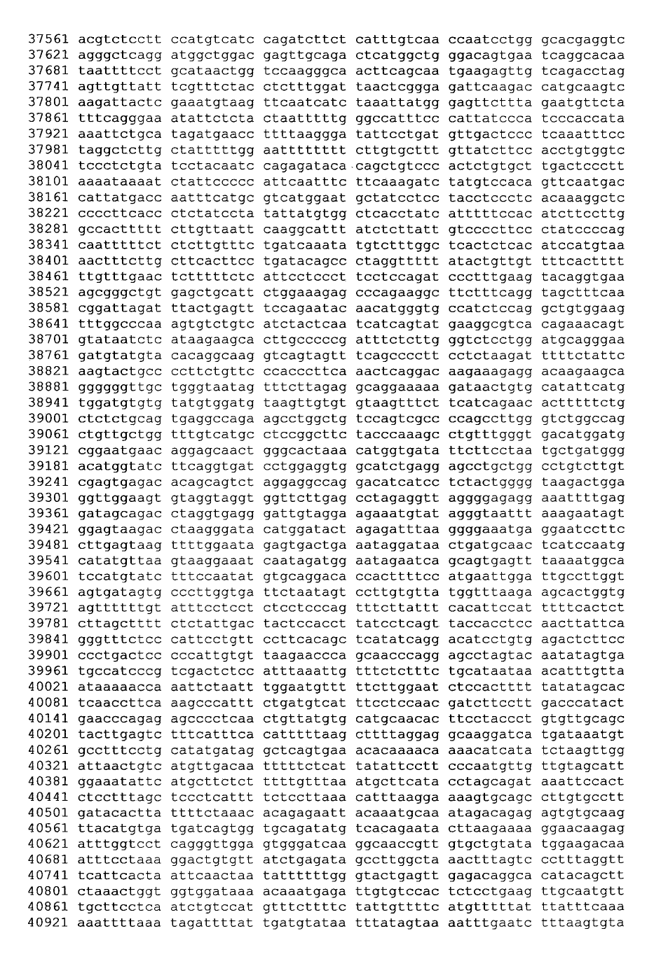

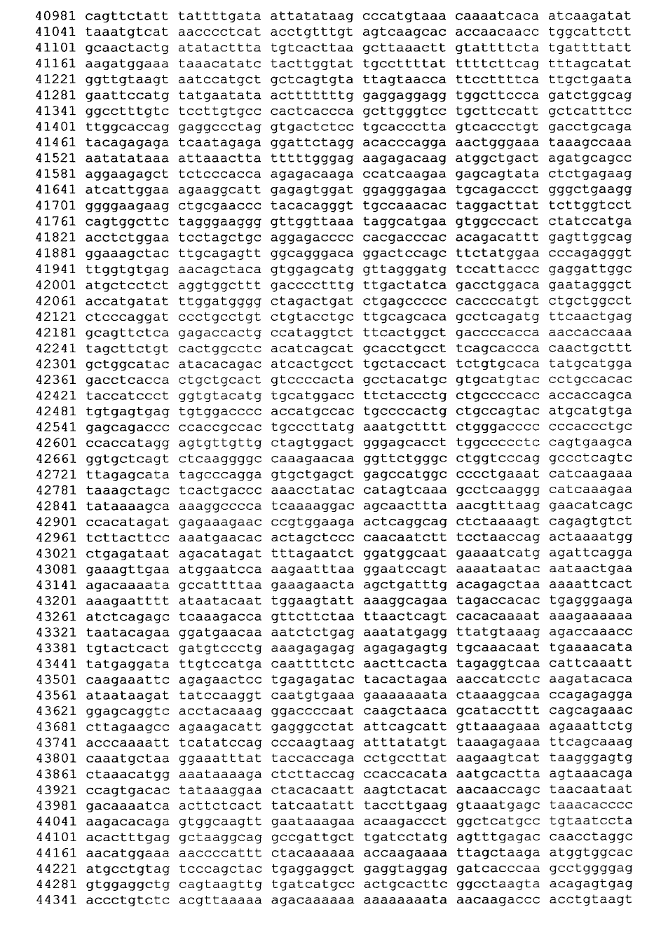

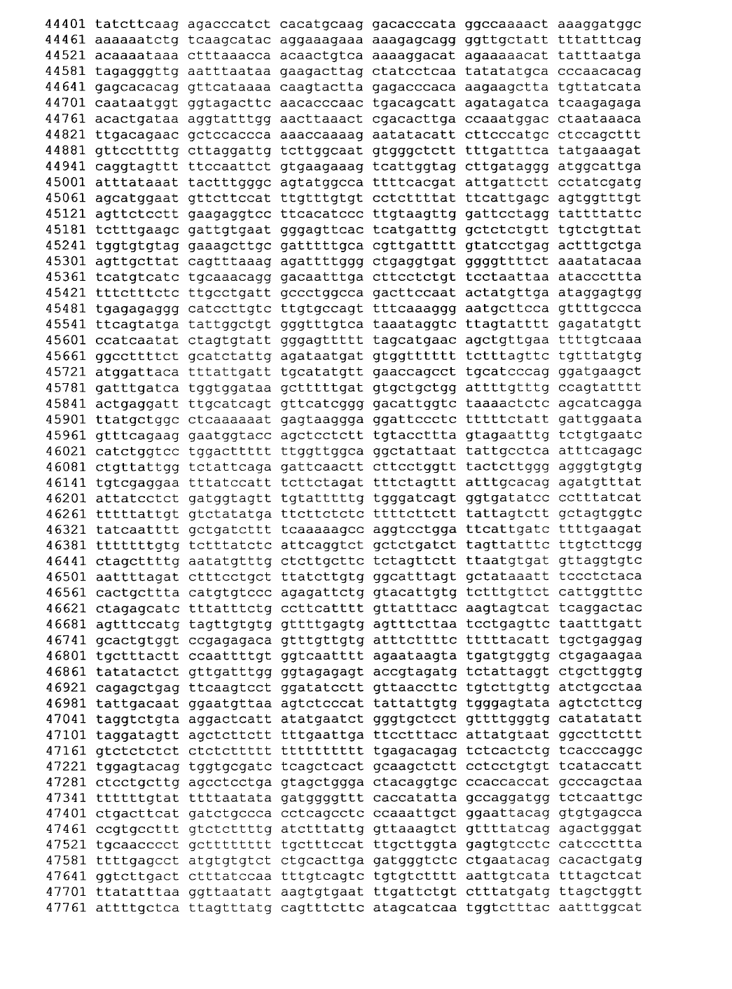

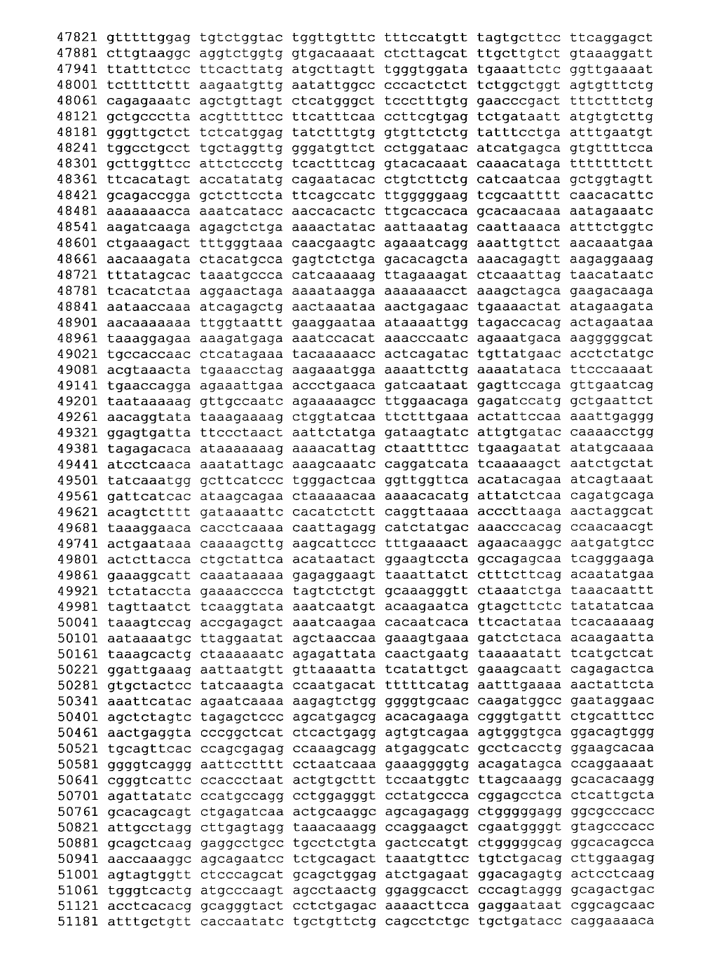

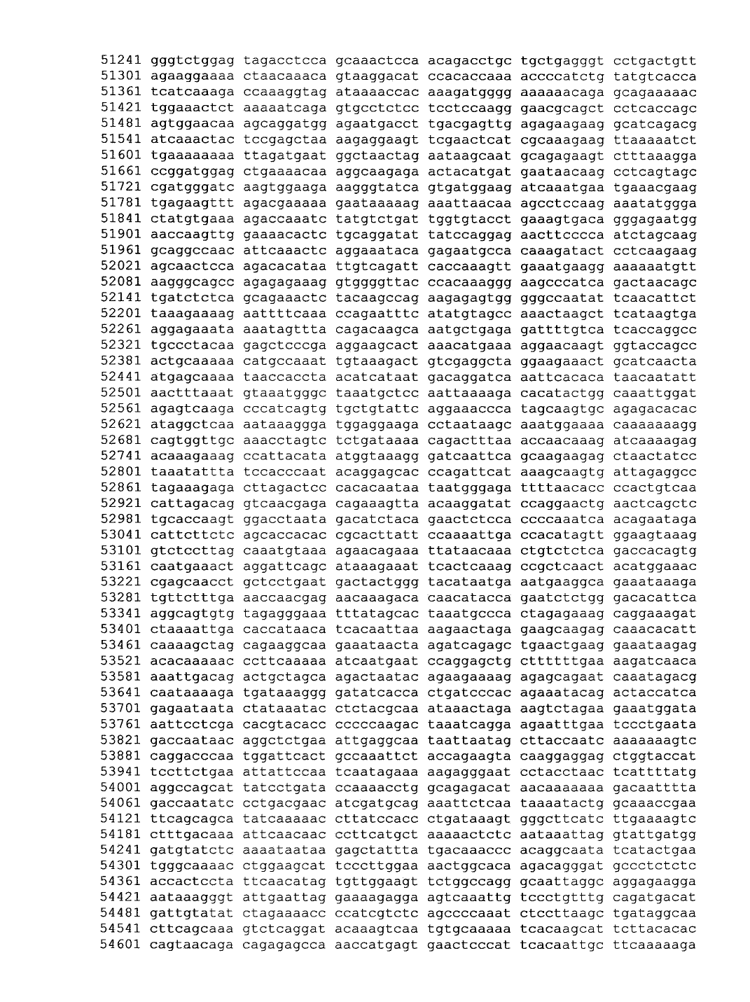

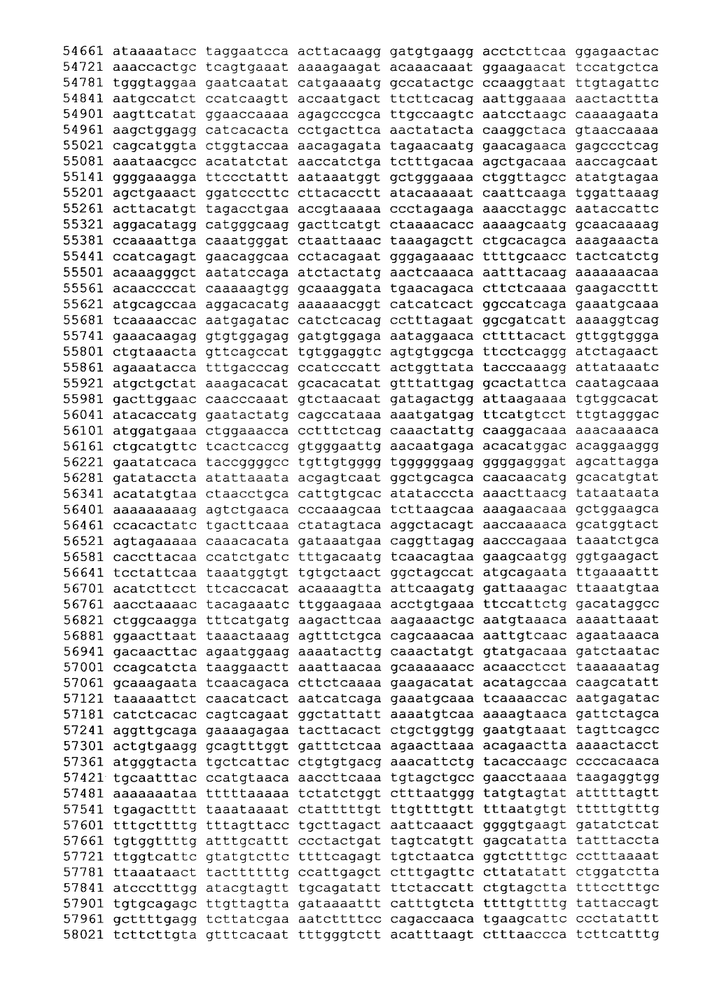

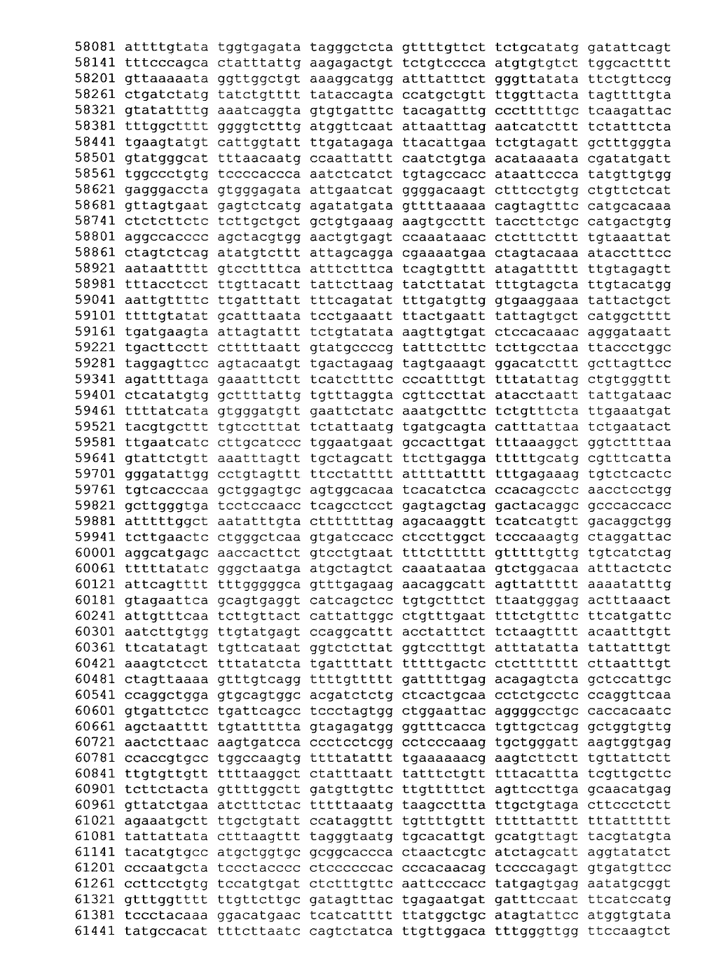

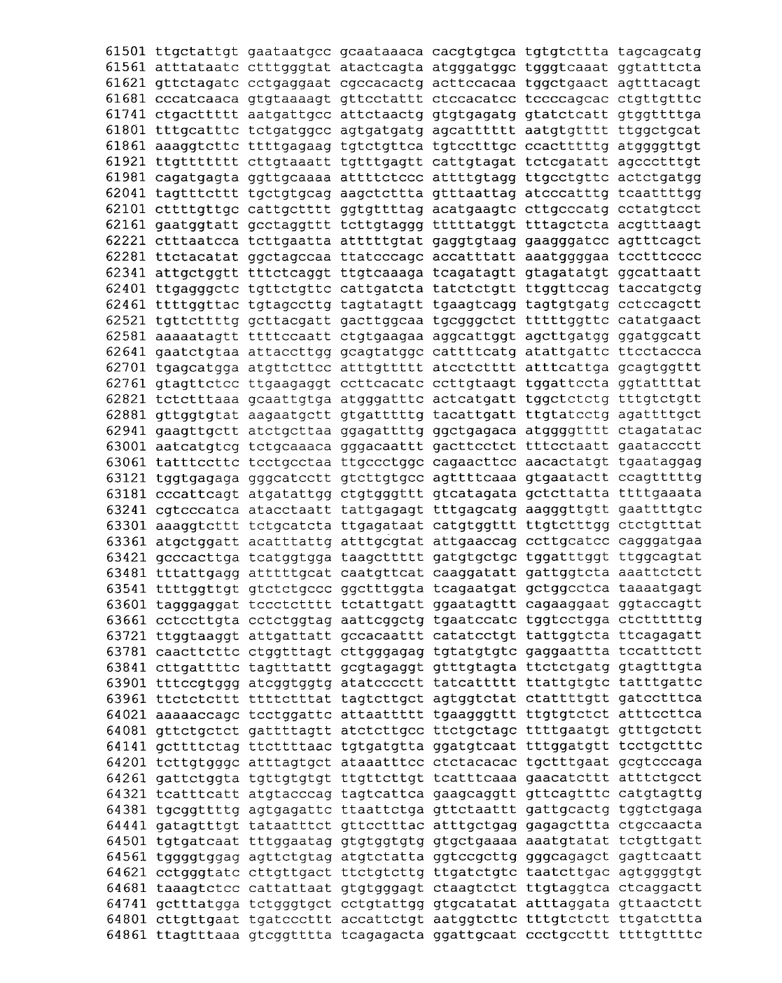

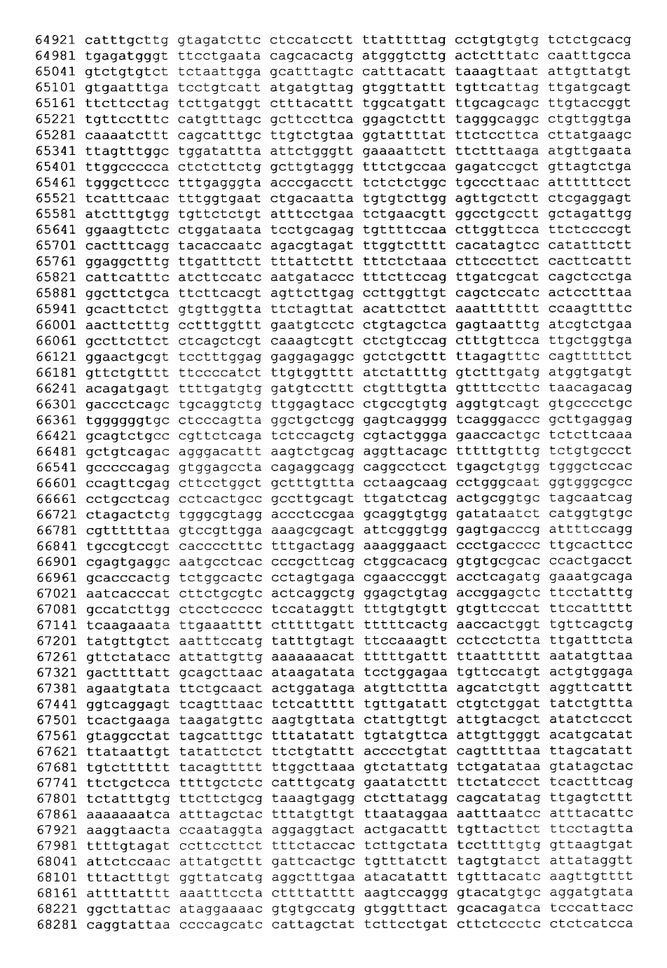

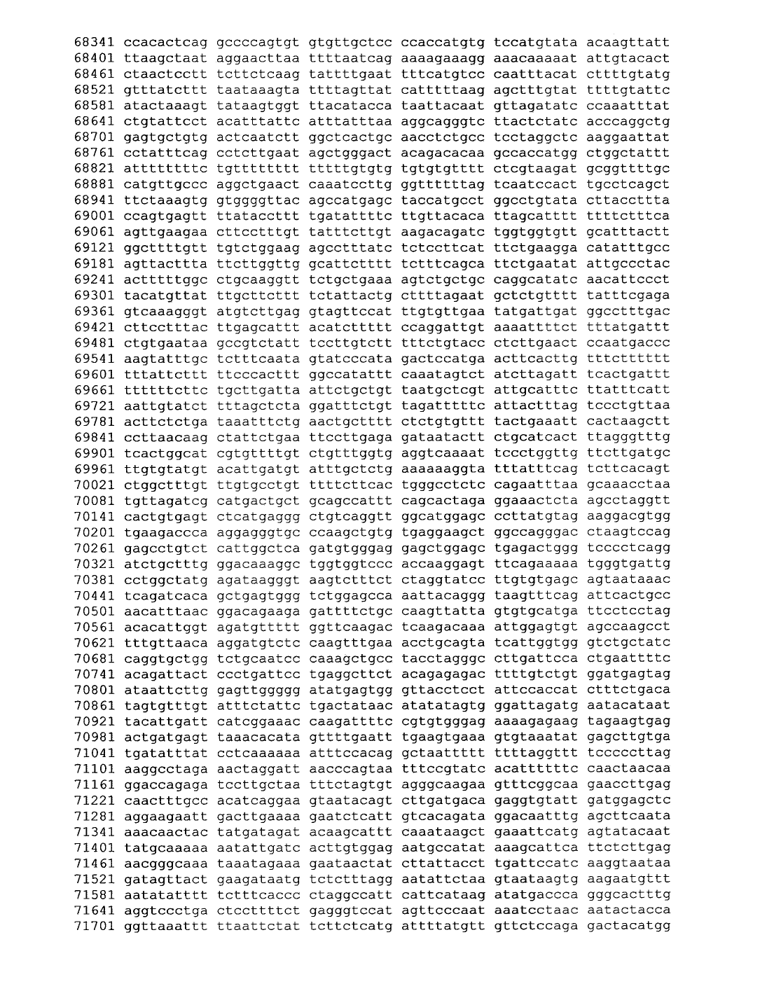

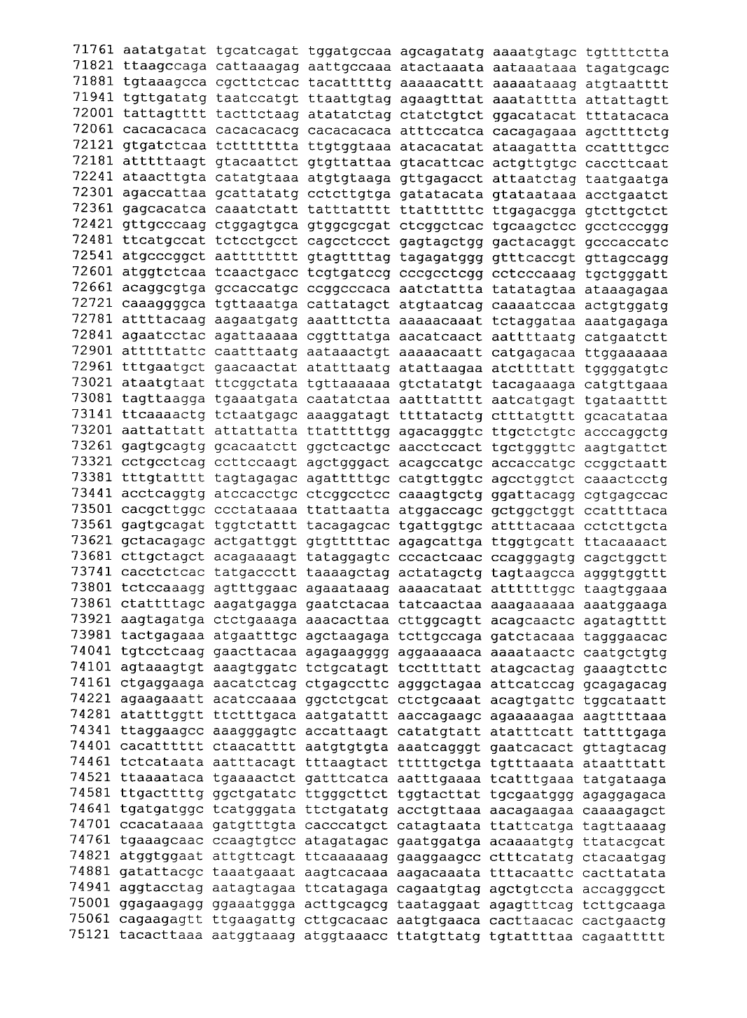

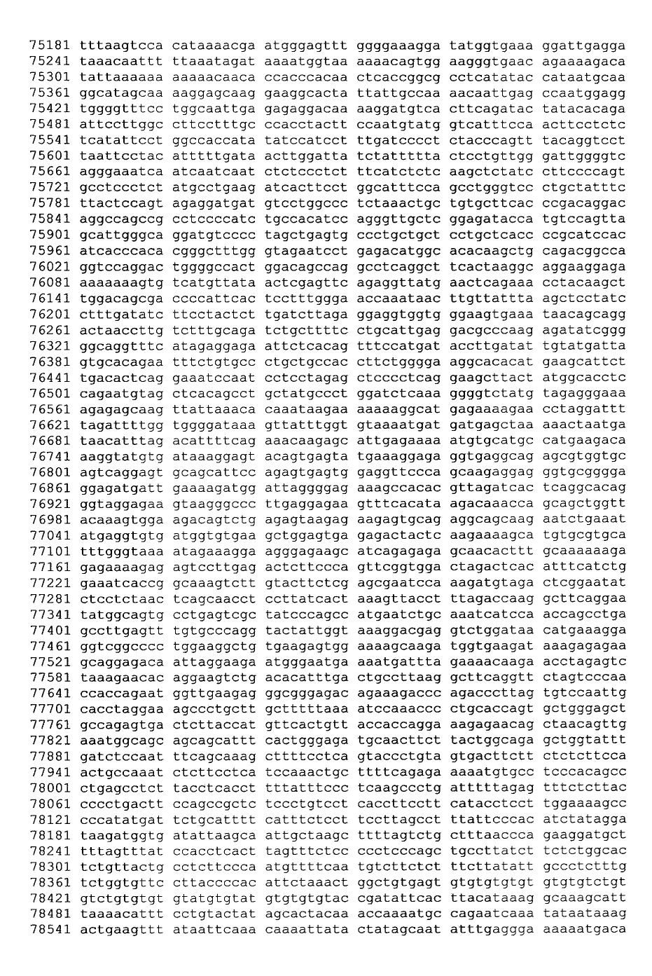

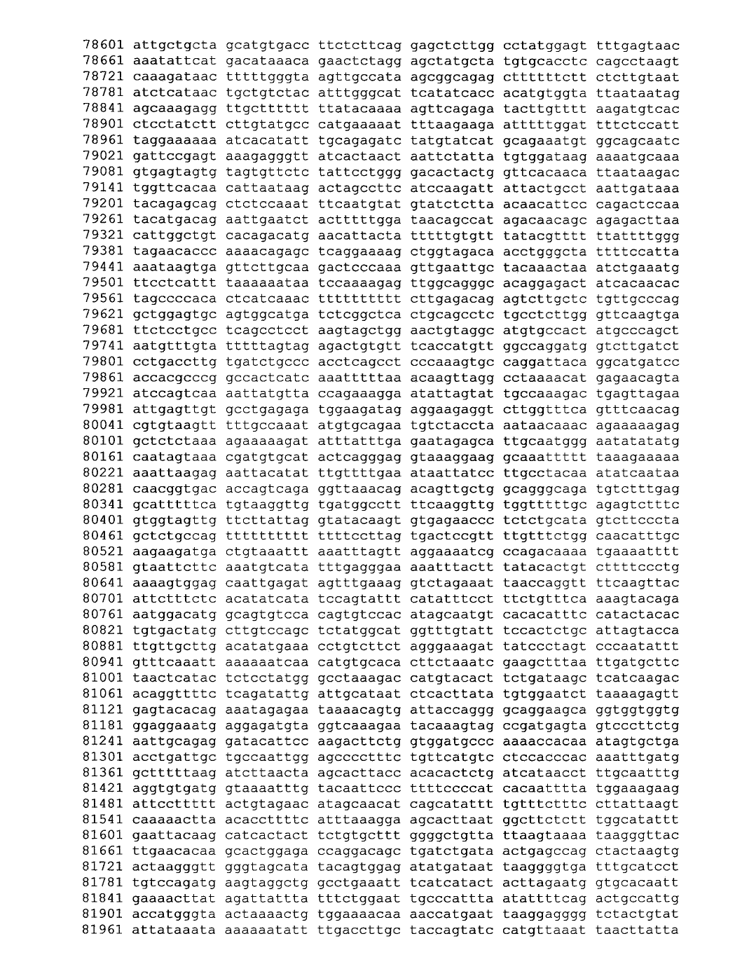

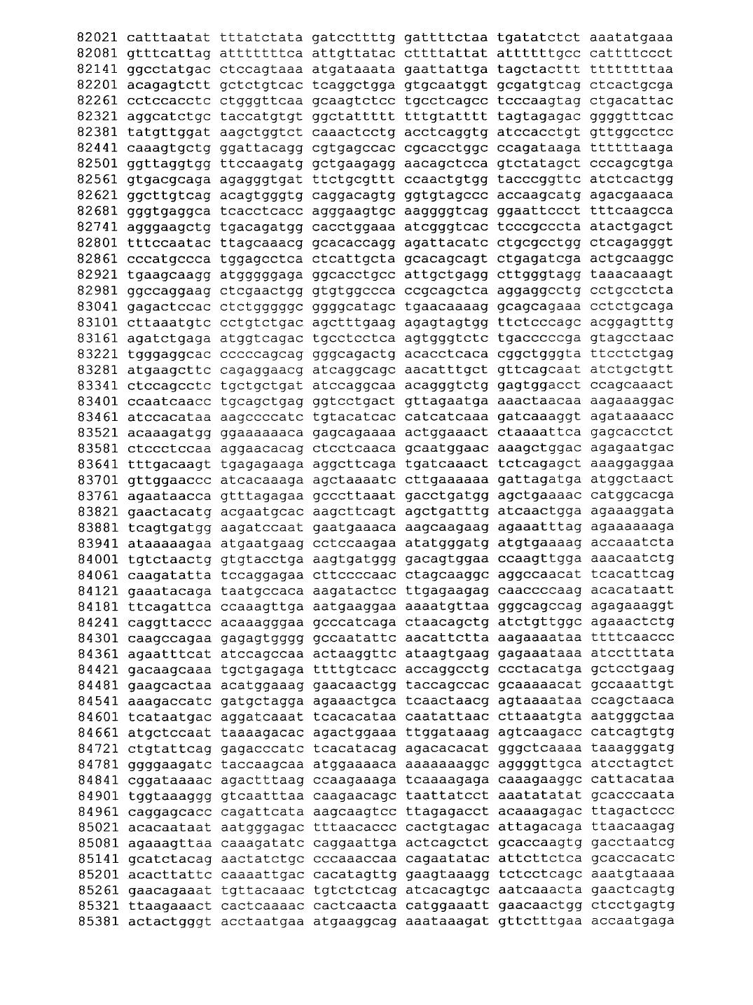

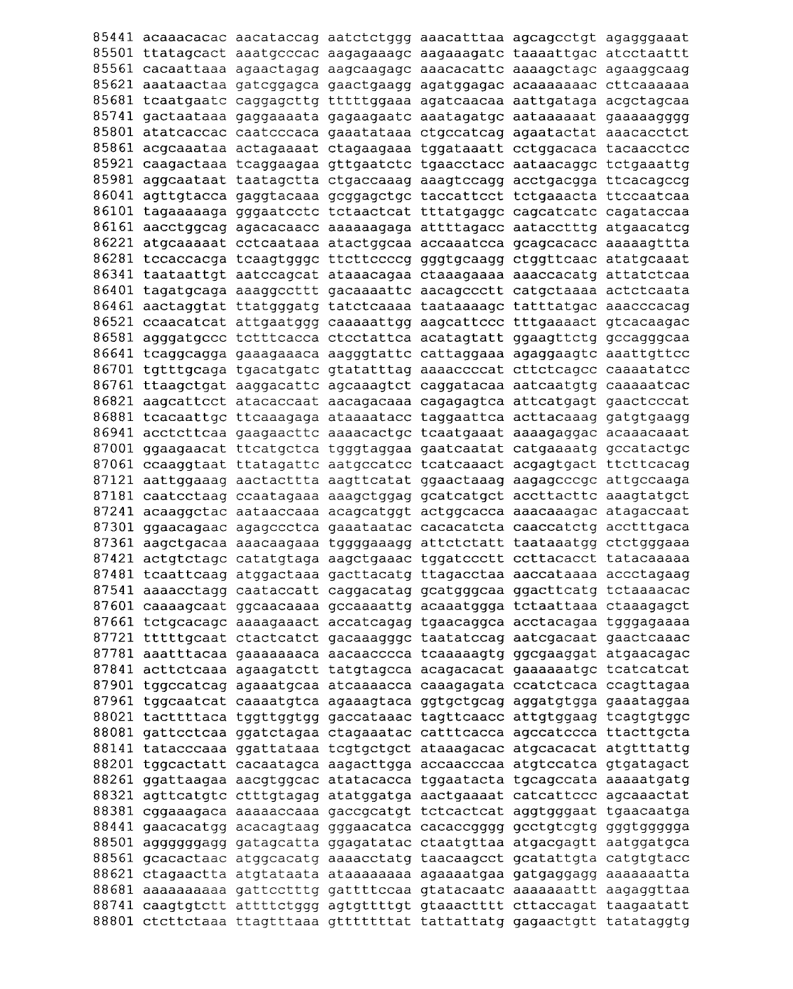

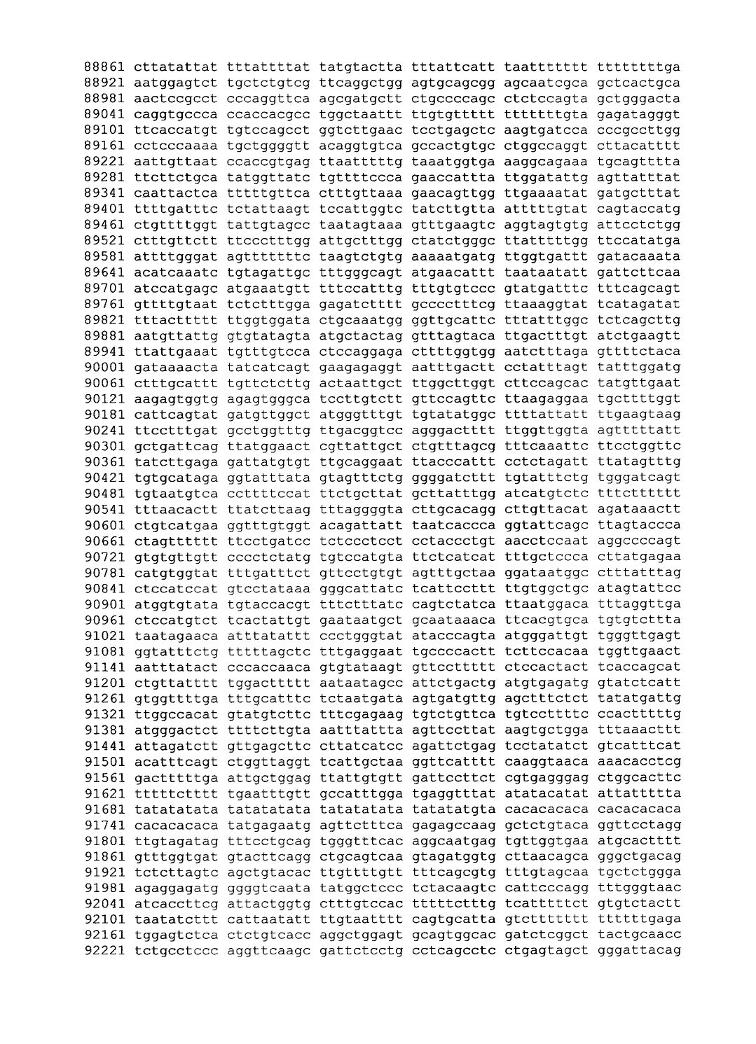

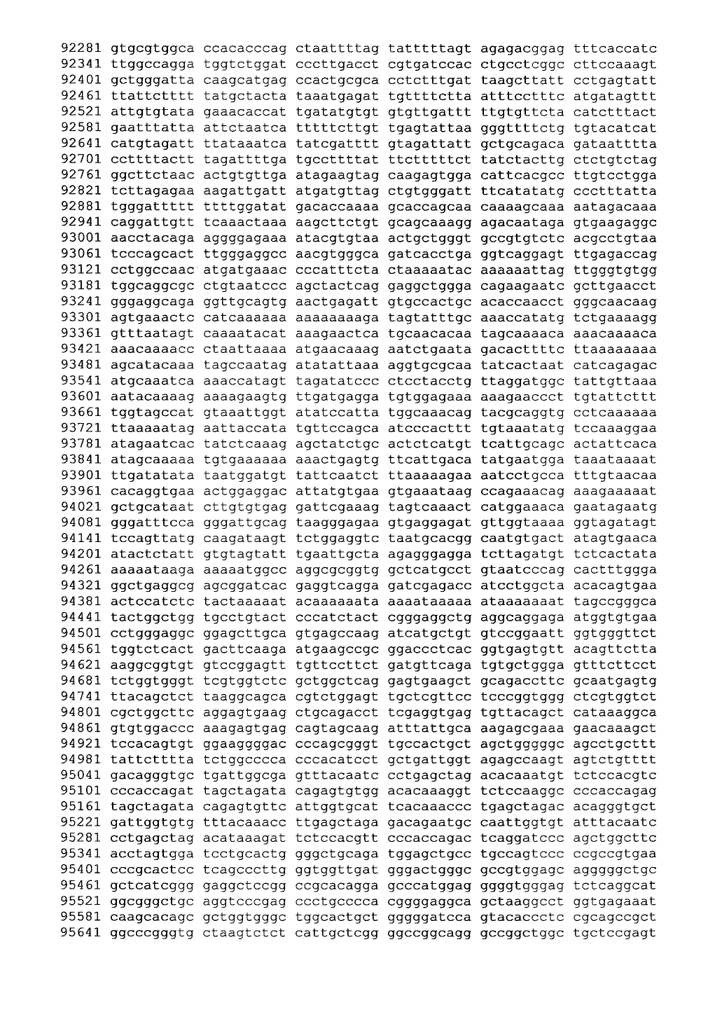

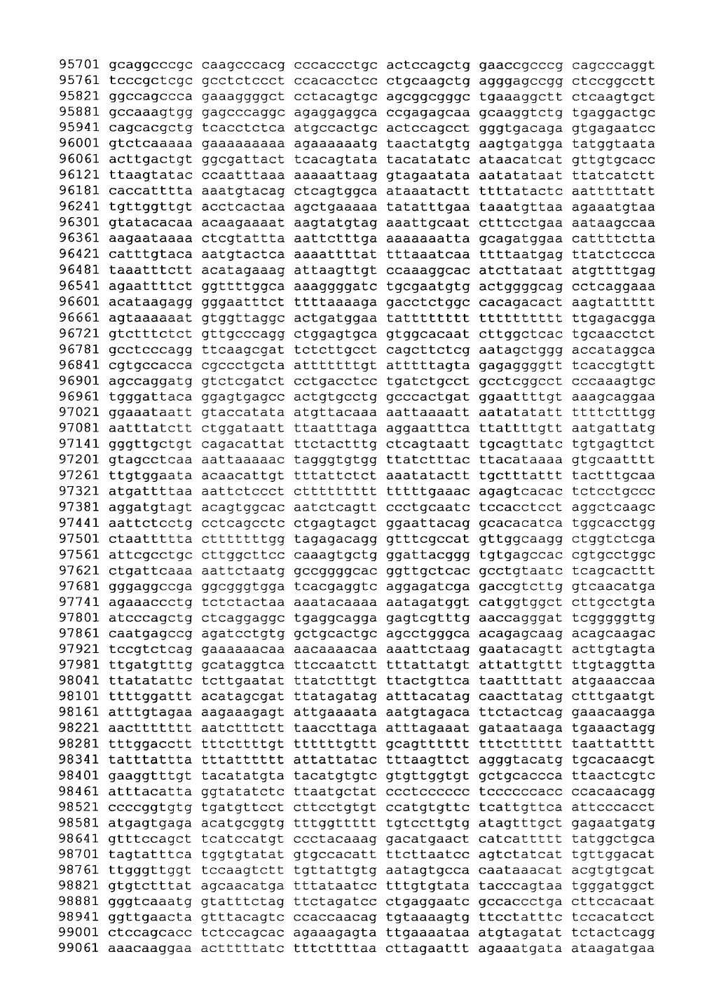

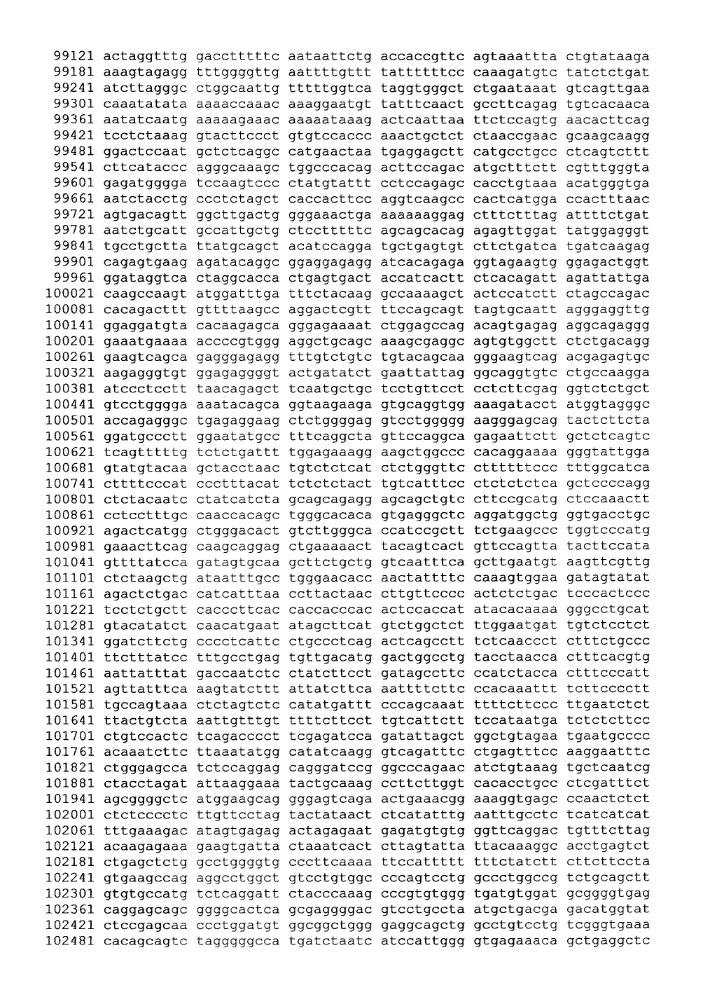

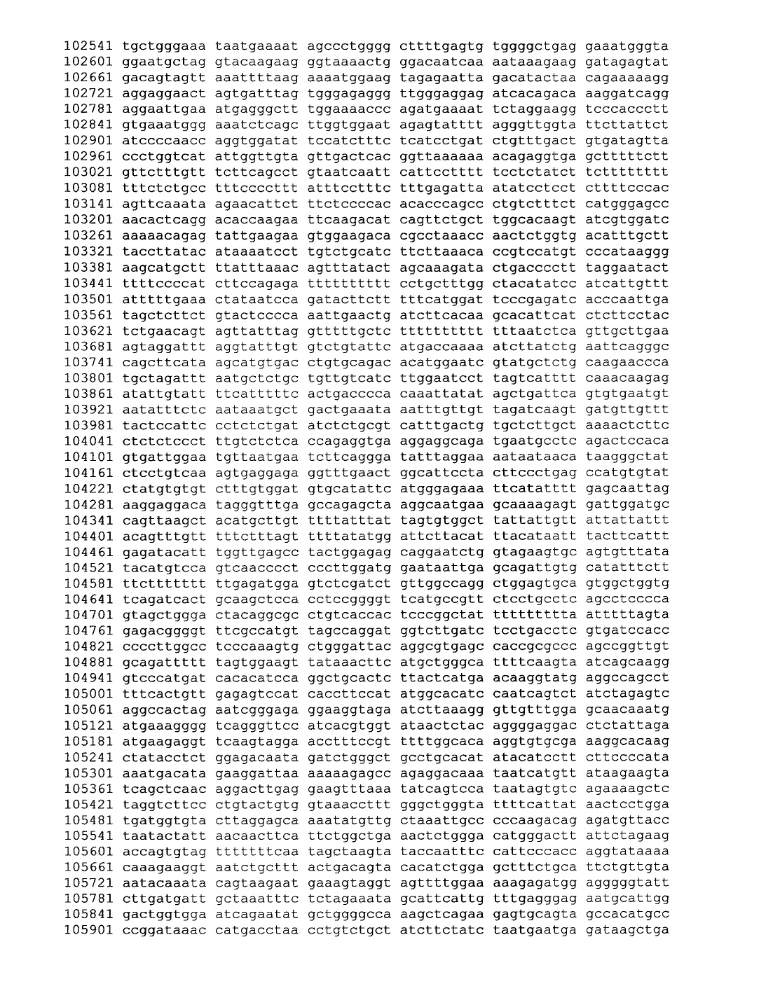

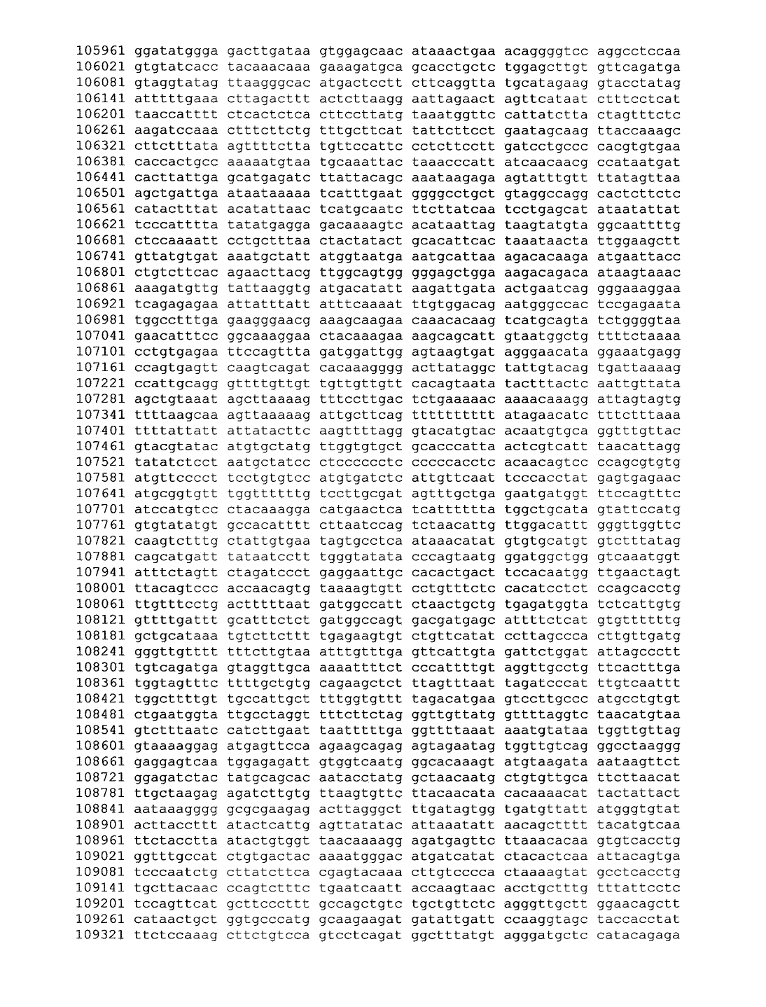

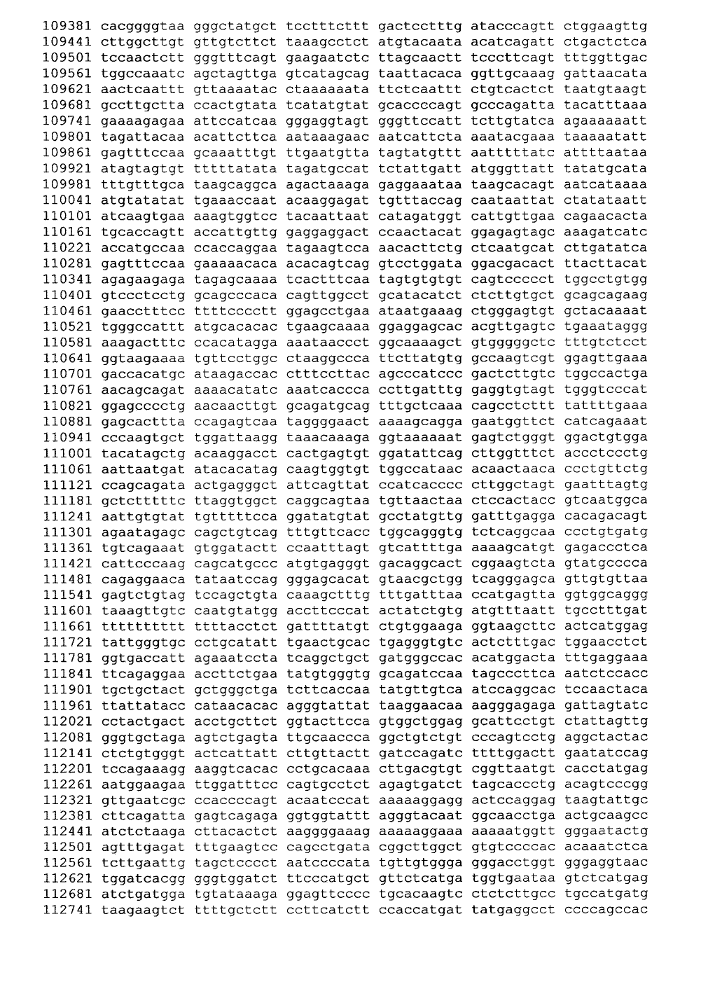

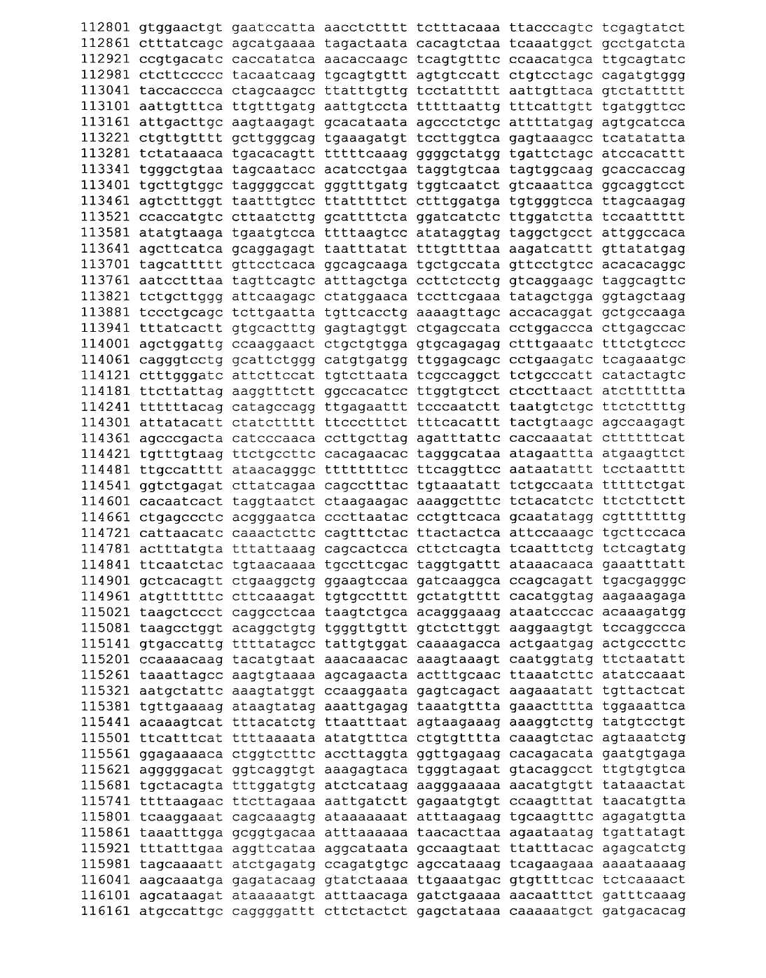

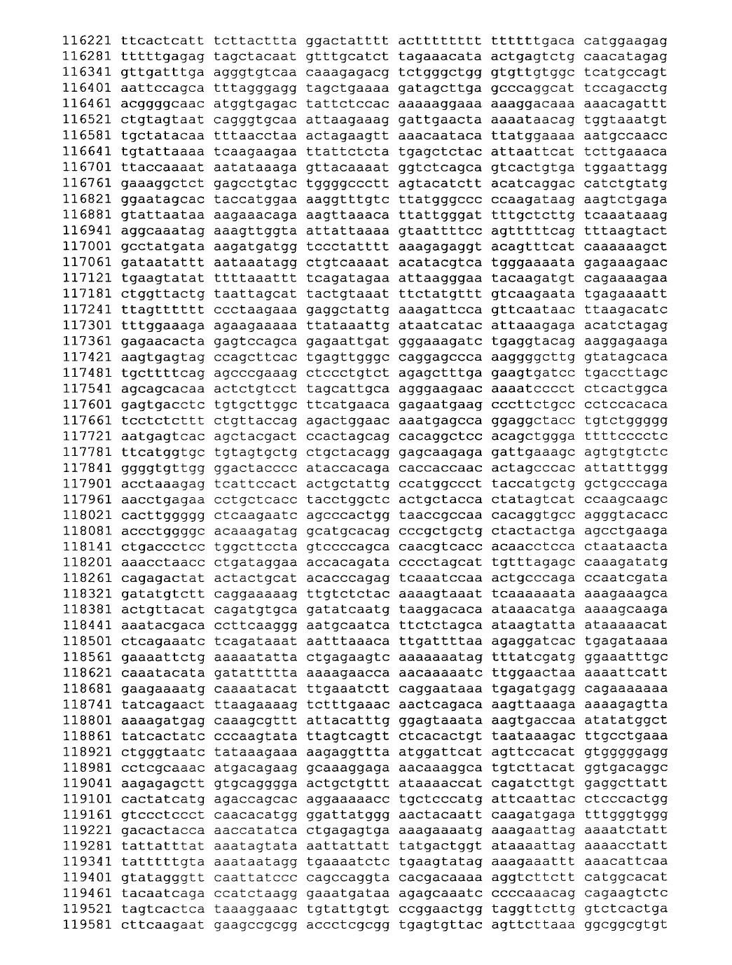

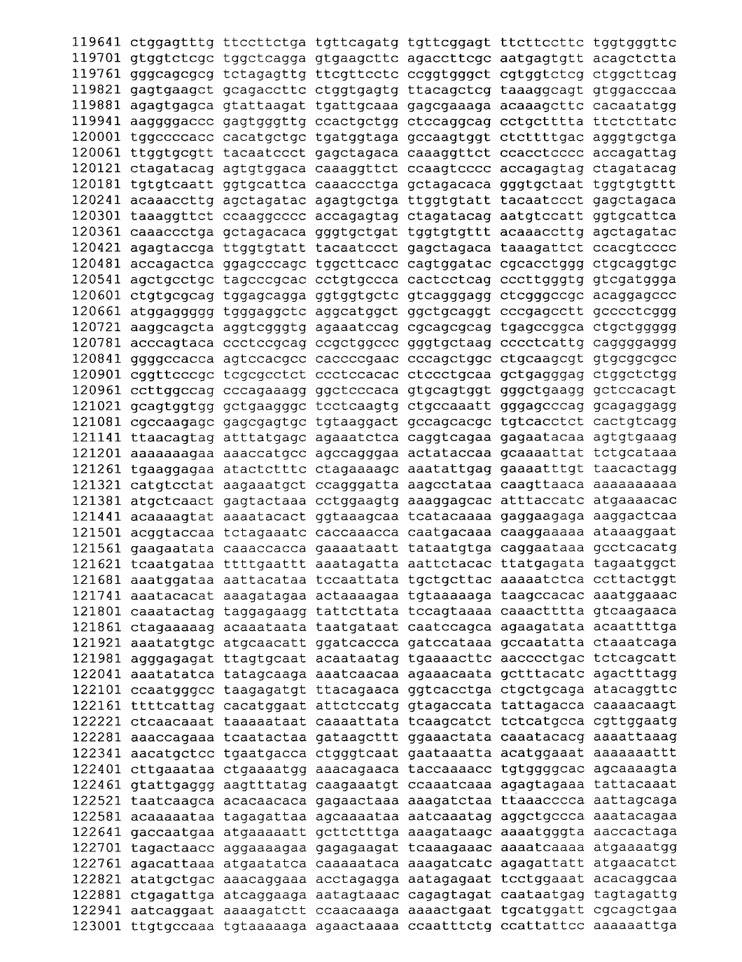

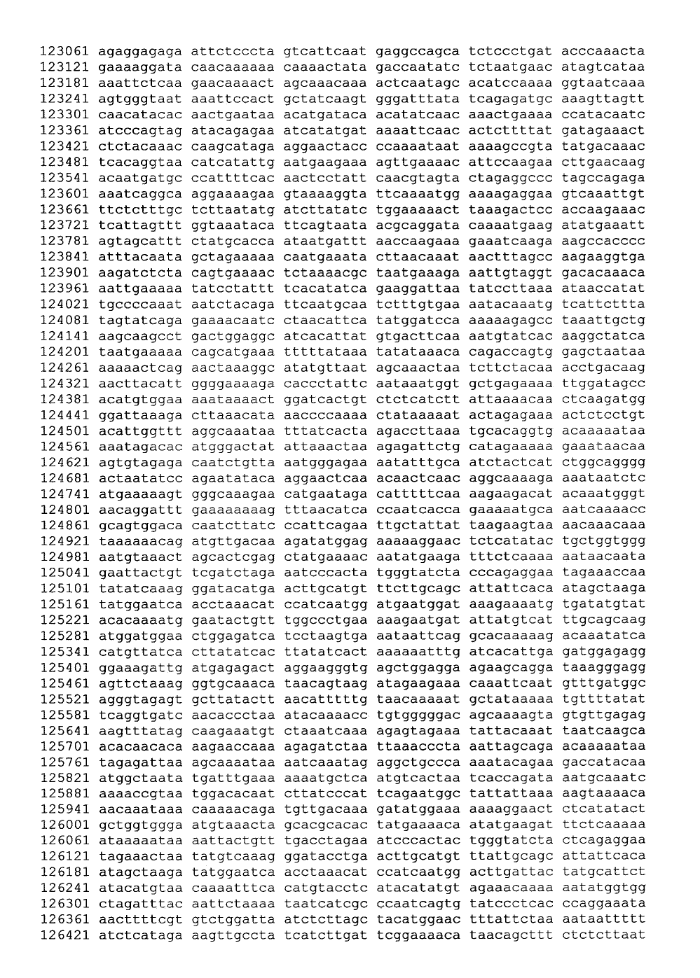

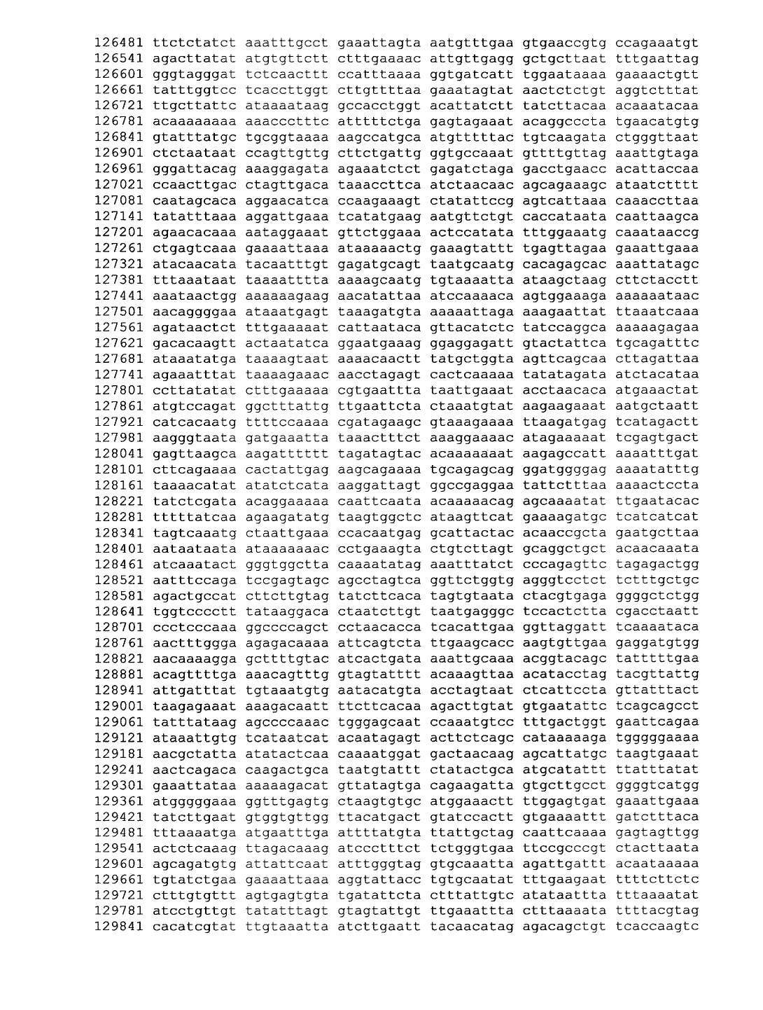

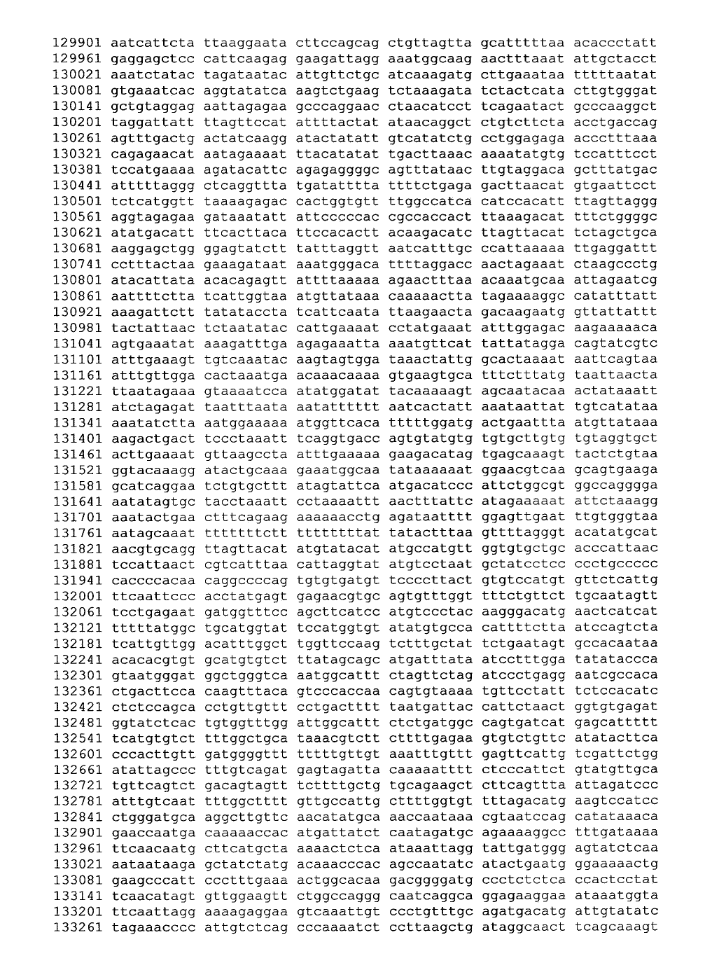

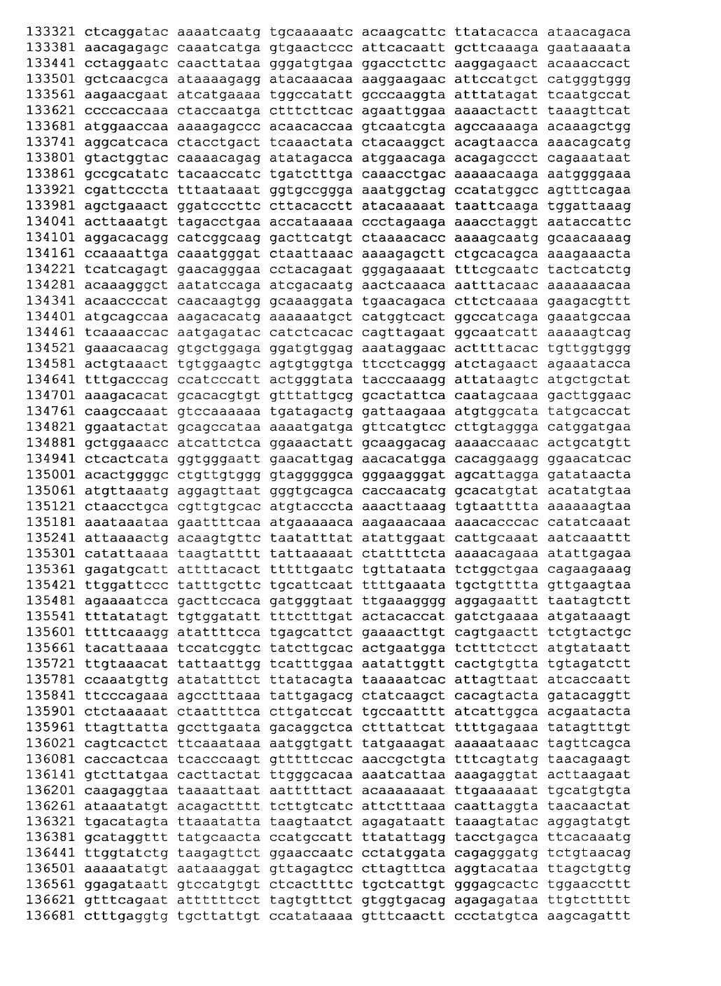

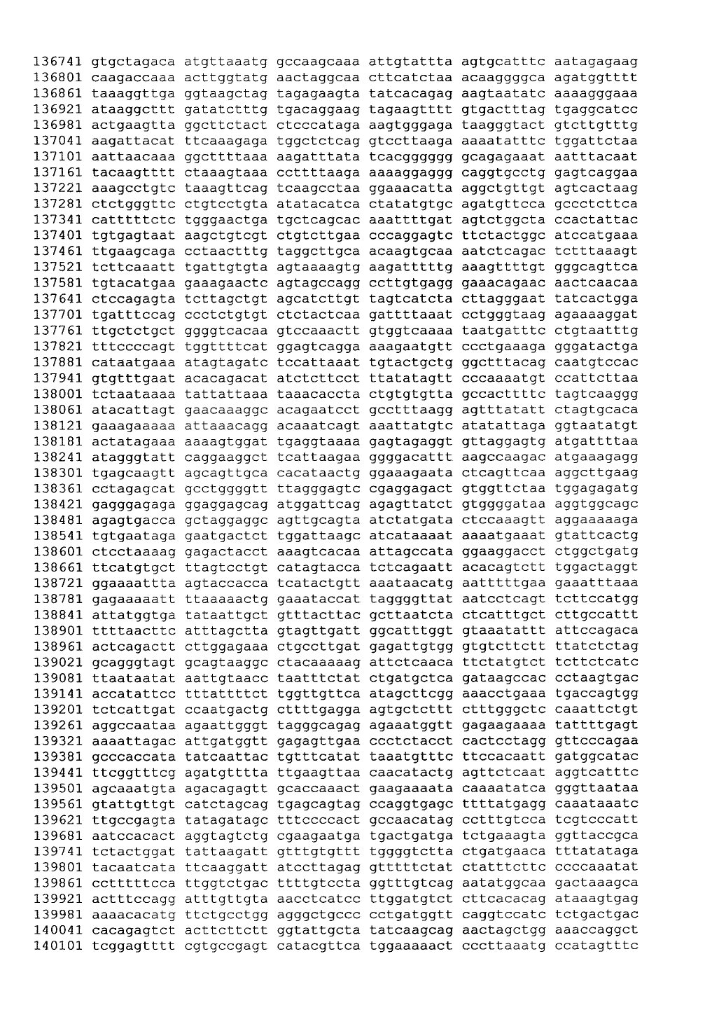

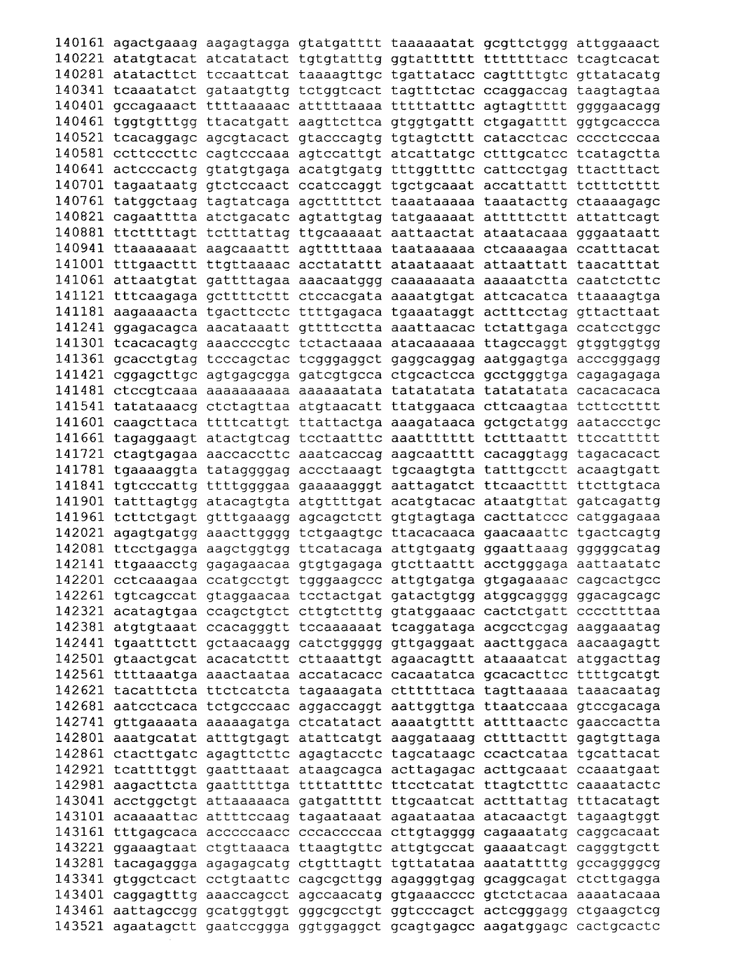

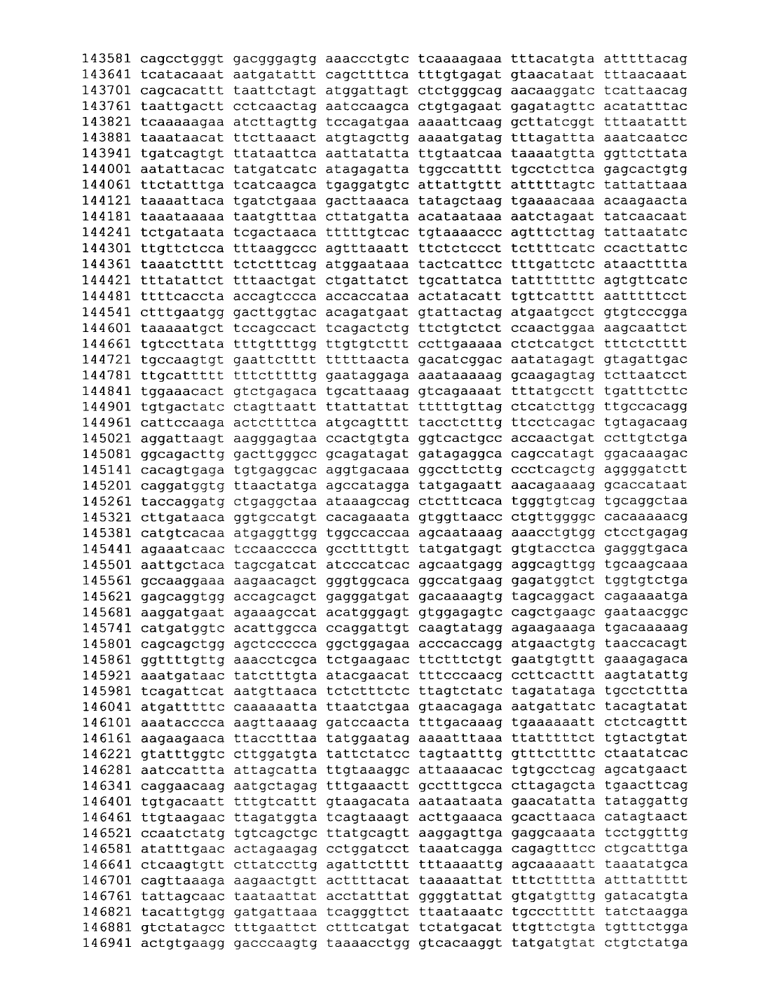

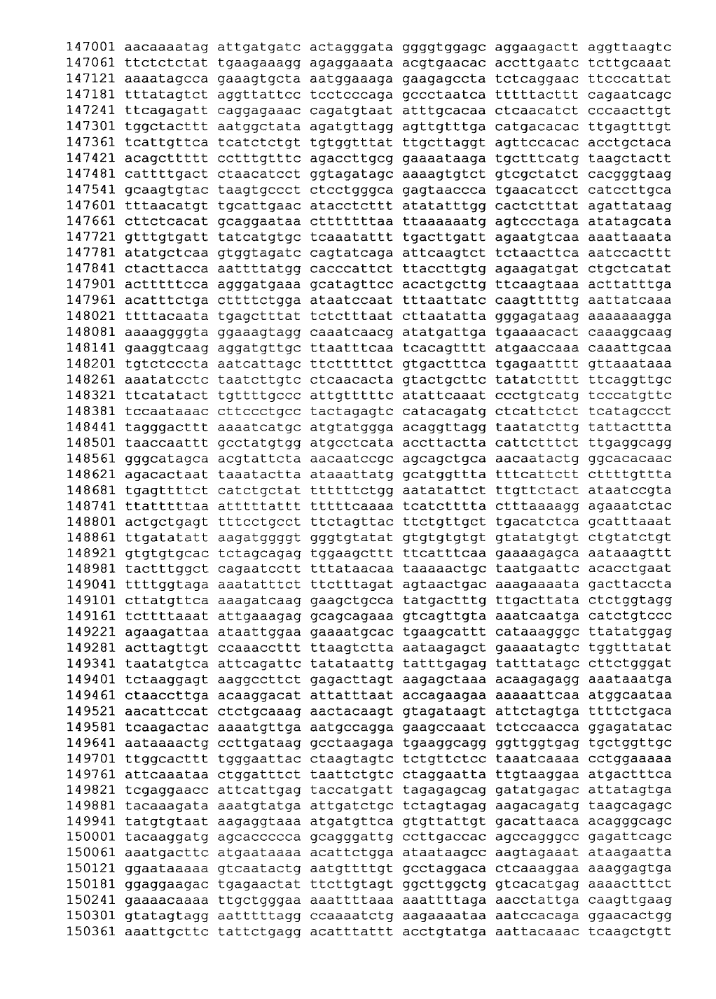

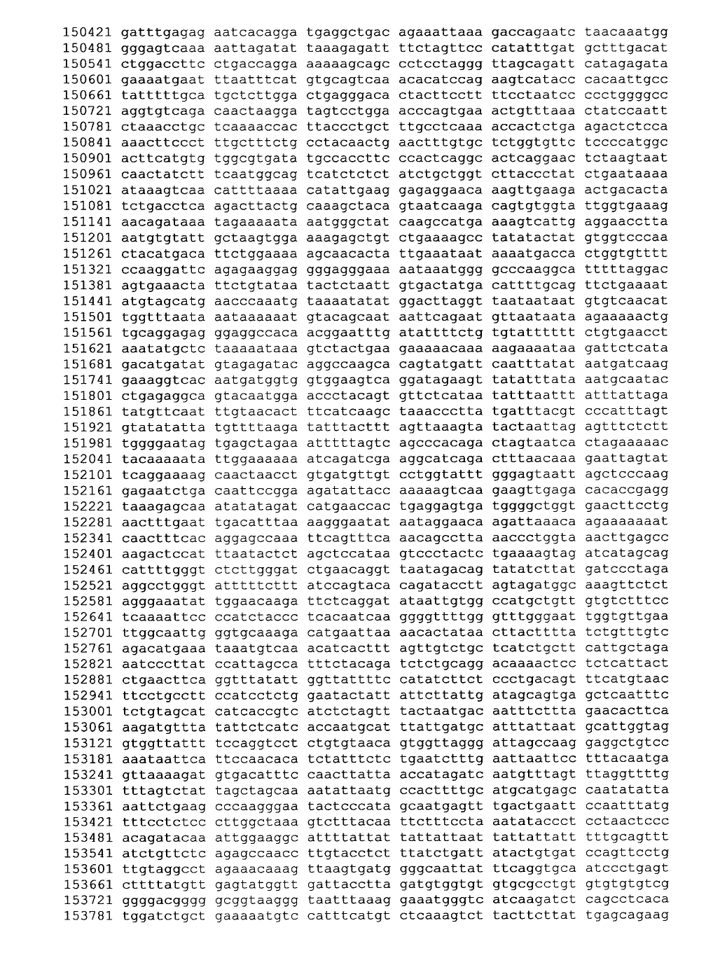

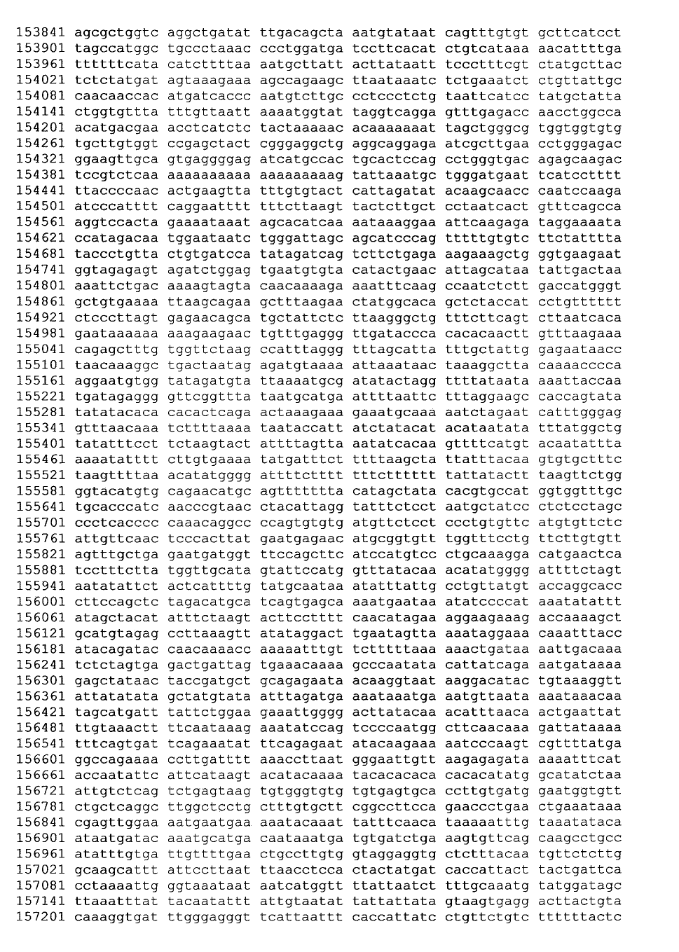

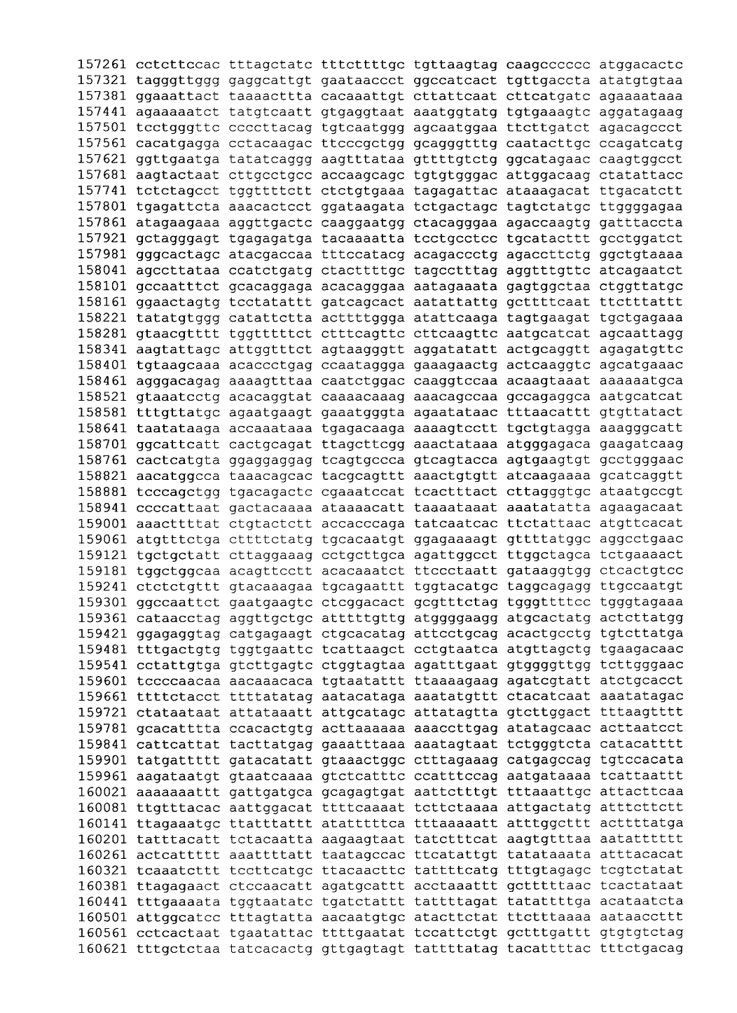

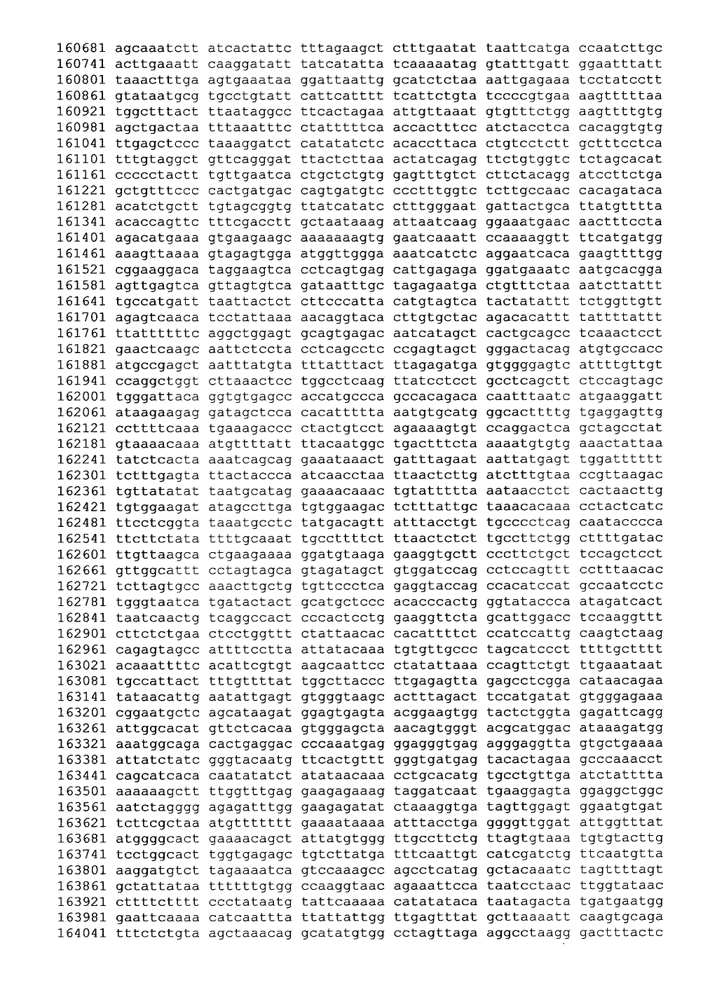

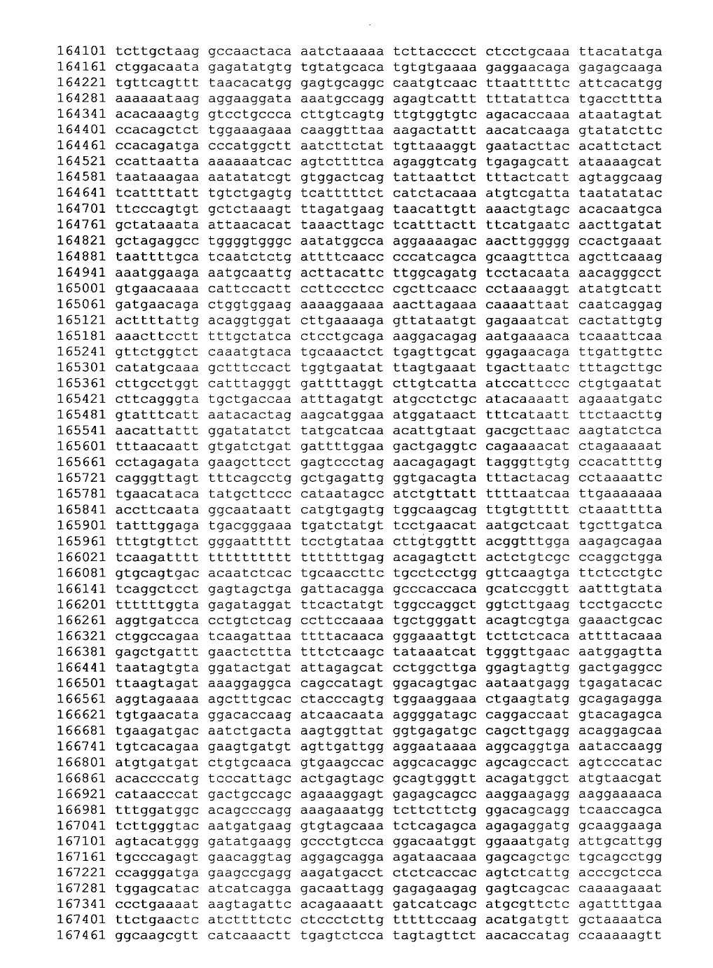

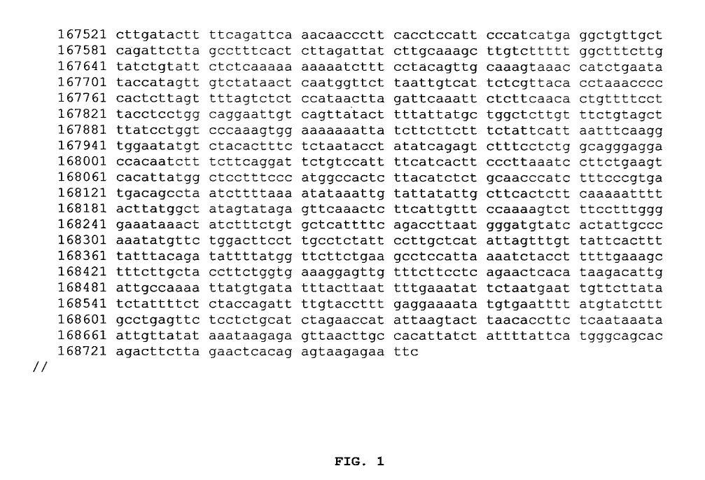

DNA encoding the CD1A, B, C, D, and/or E genes and vector DNA is isolated by standard methods known to one of ordinary skill in the art. CD1 (cluster of differentiation 1) is a family of glycoproteins expressed on the surface of various human antigen-presenting cells. They are related to the class I MHC molecules, and are involved in the presentation of lipid antigens to T-cells. CD1 glycoproteins can be classified primarily into two groups which differ in their lipid anchoring. CD1A, CD1B and CD1C (group 1 CD1 molecules) are expressed on cells specialized for antigen presentation. CD1D (group 2 CD1) is expressed in a wider variety of cells. CD1E is an intermediate form, expressed intracellularly, the role of which is currently unclear. FIG. 1 depicts representative CD1 sequences that may be utilized for the present invention.

Vectors are designed, with or without reporter genes for facile isolation of transduced cells, e.g. by FACS, by standard methods known to one of ordinary skill in the art in such a way that CD1A, B, C, D, and/or E gene expression levels are optimized for iNKT-cell activation efficiency.

Immunoglobulin secreted as result of the primary response to disease/disorder-specific potential vaccine immunogens are analyzed using binding and functional assays. Secreted antibodies are measured by any method known to one of ordinary skill in the art, such as, for example, but not limited to, ELISA assays, for example by using antigen for capture and labeled (e.g. biotin-labeled) anti-human immunoglobulin for detection, such as, for example, commercially available anti-IgM, -IgG, -IgG1, IgG2, IgG3, -IgG4, -IgG1a, -IgG1b, -IgG2a, -IgG2b, IgG2c, etc.

To develop a sensitive surrogate for assaying local immunity, the lymphocytes traveling from local mucosal areas to the systemic blood circulation are used by methods for in vitro laboratory evaluations such as ELISPOT (6-10, 12, 15, 21; P. W. Lowry, L. M. McFarland, and H. K. Threefoot, Letter, J. Infect. Dis. 154:730, 1986). In its final step, ELISPOT measures the results of specific antibody-secreting cells (ASC) on a spot-forming gel. ELISPOT measures the number of antibody producing cells per 10⁶ PBMC following oral vaccination. The quantification of antibodies secreted by a fixed concentration of PBMC is as important as the enumeration of ASC.

An ELISA may be utilized for measuring IgA and IgG antibodies. Antitoxin and anti-lipopolysaccharide (LPS)-specific IgA and IgG titers may be measured by the enzyme-linked immunosorbent assay (ELISA) method using Gm1 and LPS as capture antigens. Microtiter 96-well, low-binding plates may be first coated with a 100 μl of either 50 μg of Gm1 (Sigma) or 50 μg of V. cholerae LPS (Inaba 569B; Sigma) per ml in PBS overnight. The plates may then be washed twice with 1×PBS and blocked with 100 μl of 0.1% bovine serum albumin (BSA)-PBS for 30 min at 37° C. The plates may be washed three times with PBS-0.05% Tween 20.

Test samples from serum may be diluted in the plates using 0.1% BSA-PBS-Tween solution as a diluent. After 30 min of incubation, the plates may be washed twice with PBS-Tween. Then, 100 μl of anti-human IgG or anti-human IgA conjugated with horseradish peroxidase (Jackson Laboratories) may be diluted in 0.1% BSA-PBS-Tween and added to each well. After the mixtures are washed, 100 μl of o-phenylenediamine (OPD; 1 mg/ml; Sigma) in 0.1 M sodium citrate buffer (pH 4.5) with 30% H₂O₂ (4 μl/10 ml) may be added to each well. After 20 min, the plates may be read at 450 nm in an automated ELISA reader. Titers may be calculated using a computer program to interpolate the dilution of serum that yielded an optical density of 0.4 above baseline. Pre-vaccine and post-vaccine sera may be tested simultaneously in the same plate.

ELISA for total human IgA and IgG: Total IgA and IgG levels may be measured by using conjugate antibodies of goat anti-human IgA and IgG in ELISA. DynTech Immunolon I plates were coated with 100 ρl of 2-μg/ml concentration of goat anti-humanIgA α-chain-specific (Jackson Laboratories) or goat anti-human IgG Fc-specific (Jackson Laboratories). The plates may be incubated overnight at 4° C. Each plate was washed with 1×PBS and blocked with 100 μl of 1% BSA-1×PBS at 37° C. for 30 min. The plate may then be washed with PBS again. A 100-μl portion of ALS supernatant sample with the desired dilutions may be added to the plate. Standard human IgA and IgG with twofold dilutions may be added to each plate as a standard.

For total IgA measurement, the plates may be incubated for 60 min at 37° C. and washed with PBS-0.05% Tween 20. The plate may then be conjugated with goat anti-human IgA (peroxidase conjugated, α-chain specific; Jackson Laboratories). For total IgG measurement, the plates may be conjugated with goat anti-human IgG heavy and light chain (peroxidase conjugated; Jackson Laboratories). The plates may be incubated at 37° C. for 60 min and washed with PBS-0.05% Tween 20. Then, 1001 μl of OPD substrate (Sigma) may be added. Finally, the plates may be read in an ELISA reader at 450 nm.

Measurement of total IgA and IgG secretion abilities may be processed at days 0, 1, 2, and 3 at room temperature: Titration of the effect of blood storage on the ability of PBMC to secrete the antibodies may be done with blood samples from healthy adults. The blood may be stored at day 0, day 1, day 2, and day 3 at room temperature. These same aliquots of blood may be processed for PBMC isolation and adjusted to 10⁷ cells per ml in complete RPMI 1640 medium. Then, 1 ml of each sample in the 24-well tissue culture plate was inoculated and incubated at 37° C. with 5% CO₂ for 48 h. Cell supernatants may be collected for the measurement of total IgA and IgG by ELISA.

Any functional analyses may be carried out by methods known to one of ordinary skill in the art, such as, for example, but not limited to, viral neutralization (e.g. HIV immunogen) and hemaglutinin inhibition (e.g. influenza immunogen) assays. Viral neutralization assays are carried out by any methods known to one of ordinary skill in the art, such as, for example, but not limited to, the methods described by Dey et al., 2007 (Dey et al., 2007. J Virol 81(11): 5579-5593) and Beddows et al., 2007 (Beddows et al., 2007. Virol 360: 329-340).

As used herein, a neutralizing antibody may inhibit the entry of HIV-1 virus with a neutralization index>1.5 or >2.0. Broad and potent neutralizing antibodies may neutralize greater than about 50% of HIV-1 viruses (from diverse clades and different strains within a Glade) in a neutralization assay. The inhibitory concentration of the monoclonal antibody may be less than about 25 ug/ml to neutralize about 50% of the input virus in the neutralization assay.

Assays for screening for neutralizing antibodies are known in the art. A neutralization assay approach has been described previously (Binley J M, et al., (2004). Comprehensive Cross-Clade Neutralization Analysis of a Panel of Anti-Human Immunodeficiency Virus Type 1 Monoclonal Antibodies. J. Virol. 78: 13232-13252). Pseudotyped viruses may be generated by co-transfecting cells with at least two plasmids encoding a protein fragment of the present invention cDNA of the present invention and the rest of the HIV genome separately. In the HIV genome encoding vector, the Env gene may be replaced by the firefly luciferase gene. Transfectant supernatants containing pseudotyped virus may be co-incubated overnight with B-cell supernatants derived from activation of an infected donor's primary peripheral blood mononuclear cells (PBMCs). Cells stably transfected with and expressing CD4 plus the CCR5 and CXCR4 coreceptors may be added to the mixture and incubated for 3 days at 37° C. Infected cells may be quantified by luminometry.

The method of U.S. Pat. No. 7,386,232 may also be utilized for the screening of broad neutralizing antibodies. A fusion protein may be constructed by attaching an enzyme to the C-terminal end of a protein fragment of the present invention. Virus particles comprising of the fusion protein and wild type and/or protein fragments of the present invention may be generated and used to infect target cells in the presence of a patient's sera. Activities of enzyme measured in such infected cells are measures of virus binding and entry to the target cells that are mediated by the wild type viral protein fragments of the present invention. Examples of enzymes that can be used to generate the fusion protein include, but are not limited to, luciferase, bacterial or placental alkaline phosphatase, β-galactosidase, and fluorescent proteins such as Green fluorescent protein or toxins. The assay, in general, can also be carried out in 96-well plate. Decreased enzyme activities in the presence of the sera indicate that there are neutralizing antibodies in the sera.

For secondary responses, IgG+ memory B-cells isolated from either vaccine/immunogen or pathogen exposed donors and iNKT-cells are exposed in vitro to nano-particle as described above. IgG secreted as a result is tested for disease specific binding responses and functional activity, for example, neutralization assays for HIV, or hemaglutinin inhibition assay for influenza, as described above.

The present invention also encompasses identifying the immunogens that bind the secreted antibodies. Upon identification, the immunogens may be formulated into vaccines.

The nucleotide sequences and vectors of the invention can be delivered to cells, for example if aim is to express and the antigens in cells in order to produce and isolate the expressed proteins, such as from cells grown in culture. For expressing the antibodies and/or antigens in cells any suitable transfection, transformation, or gene delivery methods can be used. Such methods are well known by those skilled in the art, and one of skill in the art would readily be able to select a suitable method depending on the nature of the nucleotide sequences, vectors, and cell types used. For example, transfection, transformation, microinjection, infection, electroporation, lipofection, or liposome-mediated delivery could be used. Expression of the antibodies and/or antigens can be carried out in any suitable type of host cells, such as bacterial cells, yeast, insect cells, and mammalian cells. The antibodies and/or antigens of the invention can also be expressed using including in vitro transcription/translation systems. All of such methods are well known by those skilled in the art, and one of skill in the art would readily be able to select a suitable method depending on the nature of the nucleotide sequences, vectors, and cell types used.

In preferred embodiments, the nucleotide sequences, antibodies and/or antigens of the invention are administered in vivo, for example where the aim is to produce an immunogenic response in a subject. A “subject” in the context of the present invention may be any animal. For example, in some embodiments it may be desired to express the transgenes of the invention in a laboratory animal, such as for pre-clinical testing of the immunogenic compositions and vaccines of the invention. In other embodiments, it will be desirable to express the antibodies and/or antigens of the invention in human subjects, such as in clinical trials and for actual clinical use of the immunogenic compositions and vaccine of the invention. In preferred embodiments the subject is a human, for example a human that is infected with, or is at risk of infection with, HIV-1.

For such in vivo applications the nucleotide sequences, antibodies and/or antigens of the invention are preferably administered as a component of an immunogenic composition

comprising the nucleotide sequences and/or antigens of the invention in admixture with a pharmaceutically acceptable carrier. In an advantageous embodiment, the immunogenic compositions of the invention are useful to stimulate an immune response against HIV-1 and may be used as one or more components of a prophylactic or therapeutic vaccine against HIV-1 for the prevention, amelioration or treatment of AIDS. The nucleic acids and vectors of the invention are particularly useful for providing genetic vaccines, i.e. vaccines for delivering the nucleic acids encoding the antibodies and/or antigens of the invention to a subject, such as a human, such that the antibodies and/or antigens are then expressed in the subject to elicit an immune response.

The compositions of the invention may be injectable suspensions, solutions, sprays, lyophilized powders, syrups, elixirs and the like. Any suitable form of composition may be used. To prepare such a composition, a nucleic acid or vector of the invention, having the desired degree of purity, is mixed with one or more pharmaceutically acceptable carriers and/or excipients. The carriers and excipients must be “acceptable” in the sense of being compatible with the other ingredients of the composition. Acceptable carriers, excipients, or stabilizers are nontoxic to recipients at the dosages and concentrations employed, and include, but are not limited to, water, saline, phosphate buffered saline, dextrose, glycerol, ethanol, or combinations thereof, buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

An immunogenic or immunological composition can also be formulated in the form of an oil-in-water emulsion. The oil-in-water emulsion can be based, for example, on light liquid paraffin oil (European Pharmacopea type); isoprenoid oil such as squalane, squalene, EICOSANE™ or tetratetracontane; oil resulting from the oligomerization of alkene(s), e.g., isobutene or decene; esters of acids or of alcohols containing a linear alkyl group, such as plant oils, ethyl oleate, propylene glycol di(caprylate/caprate), glyceryl tri(caprylate/caprate) or propylene glycol dioleate; esters of branched fatty acids or alcohols, e.g., isostearic acid esters. The oil advantageously is used in combination with emulsifiers to form the emulsion. The emulsifiers can be nonionic surfactants, such as esters of sorbitan, mannide (e.g., anhydromannitol oleate), glycerol, polyglycerol, propylene glycol, and oleic, isostearic, ricinoleic, or hydroxystearic acid, which are optionally ethoxylated, and polyoxypropylene-polyoxyethylene copolymer blocks, such as the Pluronic® products, e.g., L121. The adjuvant can be a mixture of emulsifier(s), micelle-forming agent, and oil such as that which is commercially available under the name Provax® (IDEC Pharmaceuticals, San Diego, Calif.).

The immunogenic compositions of the invention can contain additional substances, such as wetting or emulsifying agents, buffering agents, or adjuvants to enhance the effectiveness of the vaccines (Remington's Pharmaceutical Sciences, 18th edition, Mack Publishing Company, (ed.) 1980).

Adjuvants may also be included. Adjuvants include, but are not limited to, mineral salts (e.g., AlK(SO₄)₂, AlNa(SO₄)₂, AlNH(SO₄)₂, silica, alum, Al(OH)₃, Ca₃(PO₄)₂, kaolin, or carbon), polynucleotides with or without immune stimulating complexes (ISCOMs) (e.g., CpG oligonucleotides, such as those described in Chuang, T. H. et al., (2002) J. Leuk. Biol. 71(3): 538-44; Ahmad-Nejad, P. et al. (2002) Eur. J. Immunol. 32(7): 1958-68; poly IC or poly AU acids, polyarginine with or without CpG (also known in the art as IC31; see Schellack, C. et al. (2003) Proceedings of the 34th Annual Meeting of the German Society of Immunology; Lingnau, K. et al. (2002) Vaccine 20(29-30): 3498-508), JuvaVax™ (U.S. Pat. No. 6,693,086), certain natural substances (e.g., wax D from Mycobacterium tuberculosis, substances found in Cornyebacterium parvum, Bordetella pertussis, or members of the genus Brucella), flagellin (Toll-like receptor 5 ligand; see McSorley, S. J. et al. (2002) J. Immunol. 169(7): 3914-9), saponins such as QS21, QS17, and QS7 (U.S. Pat. Nos. 5,057,540; 5,650,398; 6,524,584; 6,645,495), monophosphoryl lipid A, in particular, 3-de-O-acylated monophosphoryl lipid A (3D-MPL), imiquimod (also known in the art as IQM and commercially available as Aldara®; U.S. Pat. Nos. 4,689,338; 5,238,944; Zuber, A. K. et al. (2004) 22(13-14): 1791-8), and the CCR5 inhibitor CMPD167 (see Veazey, R. S. et al. (2003) J. Exp. Med. 198: 1551-1562).

Aluminum hydroxide or phosphate (alum) are commonly used at 0.05 to 0.1% solution in phosphate buffered saline. Other adjuvants that can be used, especially with DNA vaccines, are cholera toxin, especially CTA1-DD/ISCOMs (see Mowat, A. M. et al. (2001) J. Immunol. 167(6): 3398-405), polyphosphazenes (Allcock, H.R. (1998) App. Organometallic Chem. 12(10-11): 659-666; Payne, L. G. et al. (1995) Pharm. Biotechnol. 6: 473-93), cytokines such as, but not limited to, IL-2, IL-4, GM-CSF, IL-12, IL-15 IGF-1, IFN-α, IFN-β, and IFN-γ (Boyer et al., (2002) J. Liposome Res. 121:137-142; WO01/095919), immunoregulatory proteins such as CD40L (ADX40; see, for example, WO03/063899), and the CD1a ligand of natural killer cells (also known as CRONY or α-galactosyl ceramide; see Green, T. D. et al., (2003) J. Virol. 77(3): 2046-2055), immunostimulatory fusion proteins such as IL-2 fused to the Fc fragment of immunoglobulins (Barouch et al., Science 290:486-492, 2000) and co-stimulatory molecules B7.1 and B7.2 (Boyer), all of which can be administered either as proteins or in the form of DNA, on the same expression vectors as those encoding the antigens of the invention or on separate expression vectors.

In an advantageous embodiment, the adjuvants may be lecithin is combined with an acrylic polymer (Adjuplex-LAP), lecithin coated oil droplets in an oil-in-water emulsion (Adjuplex-LE) or lecithin and acrylic polymer in an oil-in-water emulsion (Adjuplex-LAO) (Advanced BioAdjuvants (ABA)).

The immunogenic compositions can be designed to introduce the nucleic acids or expression vectors to a desired site of action and release it at an appropriate and controllable rate. Methods of preparing controlled-release formulations are known in the art. For example, controlled release preparations can be produced by the use of polymers to complex or absorb the immunogen and/or immunogenic composition. A controlled-release formulation can be prepared using appropriate macromolecules (for example, polyesters, polyamino acids, polyvinyl, pyrrolidone, ethylenevinylacetate, methylcellulose, carboxymethylcellulose, or protamine sulfate) known to provide the desired controlled release characteristics or release profile. Another possible method to control the duration of action by a controlled-release preparation is to incorporate the active ingredients into particles of a polymeric material such as, for example, polyesters, polyamino acids, hydrogels, polylactic acid, polyglycolic acid, copolymers of these acids, or ethylene vinylacetate copolymers. Alternatively, instead of incorporating these active ingredients into polymeric particles, it is possible to entrap these materials into microcapsules prepared, for example, by coacervation techniques or by interfacial polymerization, for example, hydroxymethylcellulose or gelatin-microcapsule and poly-(methylmethacrylate) microcapsule, respectively, in colloidal drug delivery systems (for example, liposomes, albumin microspheres, microemulsions, nano-particles and nanocapsules) or in macroemulsions. Such techniques are disclosed in New Trends and Developments in Vaccines, Voller et al. (eds.), University Park Press, Baltimore, Md., 1978 and Remington's Pharmaceutical Sciences, 16th edition.

Suitable dosages of the nucleic acids and expression vectors of the invention (collectively, the immunogens) in the immunogenic composition of the invention can be readily determined by those of skill in the art. For example, the dosage of the immunogens can vary depending on the route of administration and the size of the subject. Suitable doses can be determined by those of skill in the art, for example by measuring the immune response of a subject, such as a laboratory animal, using conventional immunological techniques, and adjusting the dosages as appropriate. Such techniques for measuring the immune response of the subject include, but are not limited to, chromium release assays, tetramer binding assays, IFN-γ ELISPOT assays, IL-2 ELISPOT assays, intracellular cytokine assays, and other immunological detection assays, e.g., as detailed in the text “Antibodies: A Laboratory Manual” by Ed Harlow and David Lane.

When provided prophylactically, the immunogenic compositions of the invention are ideally administered to a subject in advance of HIV infection, or evidence of HIV infection, or in advance of any symptom due to AIDS, especially in high-risk subjects. The prophylactic administration of the immunogenic compositions can serve to provide protective immunity of a subject against HIV-1 infection or to prevent or attenuate the progression of AIDS in a subject already infected with HIV-1. When provided therapeutically, the immunogenic compositions can serve to ameliorate and treat AIDS symptoms and are advantageously used as soon after infection as possible, preferably before appearance of any symptoms of AIDS but may also be used at (or after) the onset of the disease symptoms.

The immunogenic compositions can be administered using any suitable delivery method including, but not limited to, intramuscular, intravenous, intradermal, mucosal, and topical delivery. Such techniques are well known to those of skill in the art. More specific examples of delivery methods are intramuscular injection, intradermal injection, and subcutaneous injection. However, delivery need not be limited to injection methods. Further, delivery of DNA to animal tissue has been achieved by cationic liposomes (Watanabe et al., (1994) Mol. Reprod. Dev. 38:268-274; and WO 96/20013), direct injection of naked DNA into animal muscle tissue (Robinson et al., (1993) Vaccine 11:957-960; Hoffman et al., (1994) Vaccine 12: 1529-1533; Xiang et al., (1994) Virology 199: 132-140; Webster et al., (1994) Vaccine 12: 1495-1498; Davis et al., (1994) Vaccine 12: 1503-1509; and Davis et al., (1993) Hum. Mol. Gen. 2: 1847-1851), or intradermal injection of DNA using “gene gun” technology (Johnston et al., (1994) Meth. Cell Biol. 43:353-365). Alternatively, delivery routes can be oral, intranasal or by any other suitable route. Delivery also be accomplished via a mucosal surface such as the anal, vaginal or oral mucosa.

Immunization schedules (or regimens) are well known for animals (including humans) and can be readily determined for the particular subject and immunogenic composition. Hence, the immunogens can be administered one or more times to the subject. Preferably, there is a set time interval between separate administrations of the immunogenic composition. While this interval varies for every subject, typically it ranges from 10 days to several weeks, and is often 2, 4, 6 or 8 weeks. For humans, the interval is typically from 2 to 6 weeks. In a particularly advantageous embodiment of the present invention, the interval is longer, advantageously about 10 weeks, 12 weeks, 14 weeks, 16 weeks, 18 weeks, 20 weeks, 22 weeks, 24 weeks, 26 weeks, 28 weeks, 30 weeks, 32 weeks, 34 weeks, 36 weeks, 38 weeks, 40 weeks, 42 weeks, 44 weeks, 46 weeks, 48 weeks, 50 weeks, 52 weeks, 54 weeks, 56 weeks, 58 weeks, 60 weeks, 62 weeks, 64 weeks, 66 weeks, 68 weeks or 70 weeks. In a most advantageous embodiment, the interval is about 16 weeks or about 53 weeks.

The immunization regimes typically have from 1 to 6 administrations of the immunogenic composition, but may have as few as one or two or four. The methods of inducing an immune response can also include administration of an adjuvant with the immunogens. In some instances, annual, biannual or other long interval (5-10 years) booster immunization can supplement the initial immunization protocol.

The present methods also include a variety of prime-boost regimens, for example DNA prime-Adenovirus boost regimens. In these methods, one or more priming immunizations are followed by one or more boosting immunizations. The actual immunogenic composition can be the same or different for each immunization and the type of immunogenic composition (e.g., containing protein or expression vector), the route, and formulation of the immunogens can also be varied. For example, if an expression vector is used for the priming and boosting steps, it can either be of the same or different type (e.g., DNA or bacterial or viral expression vector). One useful prime-boost regimen provides for two priming immunizations, four weeks apart, followed by two boosting immunizations at 4 and 8 weeks after the last priming immunization. It should also be readily apparent to one of skill in the art that there are several permutations and combinations that are encompassed using the DNA, bacterial and viral expression vectors of the invention to provide priming and boosting regimens.

A specific embodiment of the invention provides methods of inducing an immune response against HIV in a subject by administering an immunogenic composition of the invention, preferably comprising an adenovirus vector containing DNA encoding one or more of the epitopes of the invention, one or more times to a subject wherein the epitopes are expressed at a level sufficient to induce a specific immune response in the subject. Such immunizations can be repeated multiple times at time intervals of at least 2, 4 or 6 weeks (or more) in accordance with a desired immunization regime.

The immunogenic compositions of the invention can be administered alone, or can be co-administered, or sequentially administered, with other immunogens and/or immunogenic compositions, e.g., with “other” immunological, antigenic or vaccine or therapeutic compositions thereby providing multivalent or “cocktail” or combination compositions of the invention and methods of employing them. Again, the ingredients and manner (sequential or co-administration) of administration, as well as dosages can be determined taking into consideration such factors as the age, sex, weight, species and condition of the particular subject, and the route of administration.

When used in combination, the other immunogens can be administered at the same time or at different times as part of an overall immunization regime, e.g., as part of a prime-boost regimen or other immunization protocol. For example, in an advantageous embodiment, the other HIV immunogen is env, preferably the HIV env trimer.

Many other HIV immunogens are known in the art, one such preferred immunogen is HIVA (described in WO 01/47955), which can be administered as a protein, on a plasmid (e.g., pTHr.HIVA) or in a viral vector (e.g., MVA.HIVA). Another such HIV immunogen is RENTA (described in PCT/US2004/037699), which can also be administered as a protein, on a plasmid (e.g., pTHr.RENTA) or in a viral vector (e.g., MVA.RENTA).

For example, one method of inducing an immune response against HIV in a human subject comprises administering at least one priming dose of an HIV immunogen and at least one boosting dose of an HIV immunogen, wherein the immunogen in each dose can be the same or different, provided that at least one of the immunogens is an epitope of the present invention, a nucleic acid encoding an epitope of the invention or an expression vector, preferably a VSV vector, encoding an epitope of the invention, and wherein the immunogens are administered in an amount or expressed at a level sufficient to induce an HIV-specific immune response in the subject. The HIV-specific immune response can include an HIV-specific T-cell immune response or an HIV-specific B-cell immune response. Such immunizations can be done at intervals, preferably of at least 2-6 or more weeks.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A method of identifying vaccine immunogens comprising:
(i) engineering nano-particles to contain CD-1-effective glycolipids and disease-, disorder-, or vaccine-specific immunogens,
(ii) exposing iNKT-cells in vitro to the engineered nano-particles, thereby activating the iNKT-cells,
(iii) exposing naïve B-cells in vitro to the above engineered nano-particles in the presence of activated iNKT-cells,
(iv) assaying secreted antibodies for binding and functional activity, and
(v) identifying the immunogens that bind the secreted antibodies, thereby identifying vaccine immunogens.

2. The method of claim 1, wherein the immunogen is an infectious disease immunogen.

3. The method of claim 2, wherein the infectious disease is caused by a virus.

4. The method of claim 3, wherein the virus is human immunodeficiency virus (HIV).

5. The method of claim 2, wherein the infectious disease is caused by a bacteria.

6. The method of claim 2, wherein the infectious disease is caused by a protozoa.

7. A method of prioritizing vaccine immunogens comprising iterative application of the method of claim 1 and analyzing immunogens' potential for vaccine development.

8. The method of claim 7, wherein the immunogen is an infectious disease immunogen.

9. The method of claim 8, wherein the infectious disease is caused by a virus.

10. The method of claim 9, wherein the virus is human immunodeficiency virus (HIV).

11. The method of claim 8, wherein the infectious disease is caused by a bacteria.

12. The method of claim 8, wherein the infectious disease is caused by a protozoa.

13. The method of claim 7, wherein the analyzing immunogens' potential for vaccine development is in pre-clinical animal testing.

14. The method of claim 7, wherein the analyzing immunogens' potential for vaccine development is in clinical trials.

15. A method of producing an immune response or eliciting an immune response comprising administering to a mammal the immunogen identified by the method of claim 1.

16. A method of producing an immune response or eliciting an immune response comprising administering to a mammal the immunogen identified by the method of claim 7.


SEQUENCE LISTING (21 sequences)

SEQ ID NO: 1 - protein, 327 aa, Homo sapiens
MLFLLLPLLAVLPGDGNADGLKEPLSFHVTWIASFYNHSWKQNLVSGWLSDLQTHTWDSNSSTIVFLCPWSRGNFSNEEWKELETLFRIRTIRSFEGIRRYAHELQFEYPFEIQVTGGCELHSGKVSGSFLQLAYQGSDFVSFQNNSWLPYPVAGNMAKHFCKVLNQNQHENDITHNLLSDTCPRFILGLLDAGKAHLQRQVKPEAWLSHGPSPGPGHLQLVCHVSGFYPKPVWVMWMRGEQEQQGTQRGDILPSADGTWYLRATLEVAAGEAADLSCRVKHSSLEGQDIVLYWEHHSSVGFIILAVIVPLLLLIGLALWFRKRCFC

SEQ ID NO: 2 - protein, 333 aa, Homo sapiens
MLFLQFLLLALLLPGGDNADASQEHVSFHVIQIFSFVNQSWARGQGSGWLDELQTHGWDSESGTIIFLHNWSKGNFSNEELSDLELLFRFYLFGLTREIQDHASQDYSKYPFEVQVKAGCELHSGKSPEGFFQVAFNGLDLLSFQNTTWVPSPGCGSLAQSVCHLLNHQYEGVTETVYNLIRSTCPRFLLGLLDAGKMYVHRQVRPEAWLSSRPSLGSGQLLLVCHASGFYPKPVWVTWMRNEQEQLGTKHGDILPNADGTWYLQVILEVASEEPAGLSCRVRHSSLGGQDIILYWGHHFSMNWIALVVIVPLVILIVLVLWFKKHCSYQDIL

SEQ ID NO: 3 - protein, 280 aa, Homo sapiens
FLHNWSKGNFSNEELSDLELLFRFYLFGLTREIQDHASQDYSKYPFEVQVKAGCELHSGKSPEGFFQVAFNGLDLLSFQNTTWVPSPGCGSLAQSVCHLLNHQYEGVTETVYNLIRSTCPRFLLGLLDAGKMYVHRQVRPEAWLSSRPSLGSGQLLLVCHASGFYPKPVWVTWMRNEQEQLGTKHGDILPNADGTWYLQVILEVASEEPAGLSCRVRHSSLGGQDIILYWAHIRTSCETLPPDSPIVLRTQQPRSLVQYSDAIPSTLHLNCFSFCIINIC

SEQ ID NO: 4 - protein, 333 aa, Homo sapiens
MLLLPFQLLAVLFPGGNSEHAFQGPTSFHVIQTSSFTNSTWAQTQGSGWLDDLQIHGWDSDSGTAIFLKPWSKGNFSDKEVAELEEIFRVYIFGFAREVQDFAGDFQMKYPFEIQGIAGCELHSGGAIVSFLRGALGGLDFLSVKNASCVPSPEGGSRAQKFCALIIQYQGIMETVRILLYETCPRYLLGVLNAGKADLQRQVKPEAWLSSGPSPGPGRLQLVCHVSGFYPKPVWVMWMRGEQEQQGTQLGDILPNANWTWYLRATLDVADGEAAGLSCRVKHSSLEGQDIILYWRNPTSIGSIVLAIIVPSLLLLLCLALWYMRRRSYQNIP

SEQ ID NO: 5 - protein, 245 aa, Homo sapiens
SSFTNSTWAQTQGSGWLDDLQIHGWDSDSGTAIFLKPWSKGNFSDKEVAELEEIFRVYIFGFAREVQDFAGDFQMKYPFEIQGIAGCELHSGGAIVSFLRGALGGLDFLSVKNASCVPSPEGGSRAQKFCALIIQYQGIMETVRILLYETCPRYLLGVLNAGKADLQRQVKPEAWLSSGPSPGPGRLQLVCHVSGFYPKPVWVMWMRGNPTSIGSIVLAIIVPSLLLLLCLALWYMRRRSYQNIP

SEQ ID NO: 6 - protein, 388 aa, Homo sapiens
MLLLFLLFEGLCCPGENTAAPQALQSYHLAAEEQLSFRMLQTSSFANHSWAHSEGSGWLGDLQTHGWDTVLGTIRFLKPWSHGNFSKQELKNLQSLFQLYFHSFIQIVQASAGQFQLEYPFEIQILAGCRMNAPQIFLNMAYQGSDFLSFQGISWEPSPGAGIRAQNICKVLNRYLDIKEILQSLLGHTCPRFLAGLMEAGESELKRKVKPEAWLSCGPSPGPGRLQLVCHVSGFYPKPVWVMWMRGEQEQRGTQRGDVLPNADETWYLRATLDVAAGEAAGLSCRVKHSSLGGHDLIIHWGGYSIFLILICLTVIVTLVILVVVDSRLKKQSSNKNILSPHTPSPVFLMGANTQDTKNSRHQFCLAQVSWIKNRVLKKWKTRLNQLW

SEQ ID NO: 7 - protein, 376 aa, Homo sapiens
MLLLFLLFEGLCCPGENTAAPQALQSYHLAAEEQLSFRMLQTSSFANHSWAHSEGSGWLGDLQTHGWDTVLGTIRFLKPWSHGNFSKQELKNLQSLFQLYFHSFIQIVQASAGQFQLEYPFEIQILAGCRMNAPQIFLNMAYQGSDFLSFQGISWEPSPGAGIRAQNICKVLNRYLDIKEILQSLLGHTCPRFLAGLMEAGESELKRKVKPEAWLSCGPSPGPGRLQLVCHVSGFYPKPVWVMWMRGEQEQRGTQRGDVLPNADETWYLRATLDVAAGEAAGLSCRVKHSSLGGHDLIIHWGGYSIFLILICLTVIVTLVILVVVDSRLKKQSPVFLMGANTQDTKNSRHQFCLAQVSWIKNRVLKKWKTRLNQLW

SEQ ID NO: 8 - protein, 321 aa, Homo sapiens
MLLLFLLFEGLCCPGENTAAPQALQSYHLAAEEQLSFRMLQTSSFANHSWAHSEGSGWLGDLQTHGWDTVLGTIRFLKPWSHGNFSKQELKNLQSLFQLYFHSFIQIVQASAGQFQLEYPFEIQILAGCRMNAPQIFLNMAYQGSDFLSFQGISWEPSPGAGIRAQNICKVLNRYLDIKEILQSLLGHTCPRFLAGLMEAGESELKRKVKPEAWLSCGPSPGPGRLQLVCHVSGFYPKPVWVMWMRGGYSIFLILICLTVIVTLVILVVVDSRLKKQSPVFLMGANTQDTKNSRHQFCLAQVSWIKNRVLKKWKTRLNQLW

SEQ ID NO: 9 - protein, 298 aa, Homo sapiens
MLLLFLLFEGLCCPGENTAAPQALQSYHLAAEEQLSFRMLQTSSFANHSWAHSEGSGWLGDLQTHGWDTVLGTIRFLKPWSHGNFSKQELKNLQSLFQLYFHSFIQIVQASAGQFQLELKPEAWLSCGPSPGPGRLQLVCHVSGFYPKPVWVMWMRGEQEQRGTQRGDVLPNADETWYLRATLDVAAGEAAGLSCRVKHSSLGGHDLIIHWGGYSIFLILICLTVIVTLVILVVVDSRLKKQSSNKNILSPHTPSPVFLMGANTQDTKNSRHQFCLAQVSWIKNRVLKKWKTRLNQLW

SEQ ID NO: 10 - protein, 286 aa, Homo sapiens
MLLLFLLFEGLCCPGENTAAPQALQSYHLAAEEQLSFRMLQTSSFANHSWAHSEGSGWLGDLQTHGWDTVLGTIRFLKPWSHGNFSKQELKNLQSLFQLYFHSFIQIVQASAGQFQLELKPEAWLSCGPSPGPGRLQLVCHVSGFYPKPVWVMWMRGEQEQRGTQRGDVLPNADETWYLRATLDVAAGEAAGLSCRVKHSSLGGHDLIIHWGGYSIFLILICLTVIVTLVILVVVDSRLKKQSPVFLMGANTQDTKNSRHQFCLAQVSWIKNRVLKKWKTRLNQLW

SEQ ID NO: 11 - protein, 231 aa, Homo sapiens
MLLLFLLFEGLCCPGENTAAPQALQSYHLAAEEQLSFRMLQTSSFANHSWAHSEGSGWLGDLQTHGWDTVLGTIRFLKPWSHGNFSKQELKNLQSLFQLYFHSFIQIVQASAGQFQLELKPEAWLSCGPSPGPGRLQLVCHVSGFYPKPVWVMWMRGGYSIFLILICLTVIVTLVILVVVDSRLKKQSPVFLMGANTQDTKNSRHQFCLAQVSWIKNRVLKKWKTRLNQLW

SEQ ID NO: 12 - protein, 199 aa, Homo sapiens
MLLLFLLFEGLCCPGENTAVKPEAWLSCGPSPGPGRLQLVCHVSGFYPKPVWVMWMRGEQEQRGTQRGDVLPNADETWYLRATLDVAAGEAAGLSCRVKHSSLGGHDLIIHWGGYSIFLILICLTVIVTLVILVVVDSRLKKQSSNKNILSPHTPSPVFLMGANTQDTKNSRHQFCLAQVSWIKNRVLKKWKTRLNQLW

SEQ ID NO: 13 - protein, 187 aa, Homo sapiens
MLLLFLLFEGLCCPGENTAVKPEAWLSCGPSPGPGRLQLVCHVSGFYPKPVWVMWMRGEQEQRGTQRGDVLPNADETWYLRATLDVAAGEAAGLSCRVKHSSLGGHDLIIHWGGYSIFLILICLTVIVTLVILVVVDSRLKKQSPVFLMGANTQDTKNSRHQFCLAQVSWIKNRVLKKWKTRLNQLW

SEQ ID NO: 14 - protein, 144 aa, Homo sapiens
MLLLFLLFEGLCCPGENTAVKPEAWLSCGPSPGPGRLQLVCHVSGFYPKPVWVMWMRGGYSIFLILICLTVIVTLVILVVVDSRLKKQSSNKNILSPHTPSPVFLMGANTQDTKNSRHQFCLAQVSWIKNRVLKKWKTRLNQLW

SEQ ID NO: 15 - protein, 132 aa, Homo sapiens
MLLLFLLFEGLCCPGENTAVKPEAWLSCGPSPGPGRLQLVCHVSGFYPKPVWVMWMRGGYSIFLILICLTVIVTLVILVVVDSRLKKQSPVFLMGANTQDTKNSRHQFCLAQVSWIKNRVLKKWKTRLNQLW

SEQ ID NO: 16 - protein, 290 aa, Homo sapiens
MLLLFLLFEGLCCPGENTAAPQALQSYHLAAEEQLSFRMLQTSSFANHSWAHSEGSGWLGDLQTHGWDTVLGTIRFLKPWSHGNFSKQELKNLQSLFQLYFHSFIQIVQASAGQFQLEYPFEIQILAGCRMNAPQIFLNMAYQGSDFLSFQGISWEPSPGAGIRAQNICKVLNRYLDIKEILQSLLGHTCPRFLAGLMEAGESELKRKVKPEAWLSCGPSPGPGRLQLVCHVSGFYPKPVWVMWMRGEQEQRGTQRGDVLPNADETWWIFHLSHPDLFDCDSYPGHIGCS

SEQ ID NO: 17 - protein, 101 aa, Homo sapiens
MLLLFLLFEGLCCPGENTAVKPEAWLSCGPSPGPGRLQLVCHVSGFYPKPVWVMWMRGEQEQRGTQRGDVLPNADETWWIFHLSHPDLFDCDSYPGHIGCS

SEQ ID NO: 18 - protein, 59 aa, Homo sapiens
VSGFYPKPVWVMWMRGEQEQRGTQRGDVLPNADETWWIFHLSHPDLFDCDSYPGHIGCS

SEQ ID NO: 19 - protein, 314 aa, Homo sapiens
MRGFNKTTVVTQFILVGFSSLGELQLLLFVIFLLLYLTILVANVTIMAVIRFSWTLHTPMYGFLFILSFSESCYTFVIIPQLLVHLLSDTKTISFMACATQLFFFLGFACTNCLLIAVMGYDRYVAICHPLRYTLIINKRLGLELISLSGATGFFIALVATNLICDMRFCGPNRVNHYFCDMAPVIKLACTDTHVKELALFSLSILVIMVPFLLILISYGFIVNTILKIPSAEGKKAFVTCASHLTVVFVHYGCASIIYLRPKSKSASDKDQLVAVTYTVVTPLLNPLVYSLRNKEVKTALKRVLGMPVATKMS

SEQ ID NO: 20 - protein, 312 aa, Homo sapiens
MERVNETVVREVIFLGFSSLARLQQLLFVIFLLLYLFTLGTNAIIISTIVLDRALHIPMYFFLAILSCSEICYTFIIVPKMLVDLLSQKKTISFLGCAIQMFSFLFLGCSHSFLLAVMGYDRYIAICNPLRYSVLMGHGVCMGLVAAACACGFTVAQIITSLVFHLPFYSSNQLHHFFCDIAPVLKLASHHNHFSQIVIFMLCTLVLAIPLLLILVSYVHILSAILQFPSTLGRCKAFSTCVSHLIIVTVHYGCASFIYLRPQSNYSSSQDALISVSYTIITPLFNPMIYSLRNKEFKSALCKIVRRTISLL

SEQ ID NO: 21 - DNA, 168753 bp, Homo sapiens
gaattcattcattcatttgacaaatattcactaagcacctaccacttgtgagatactctgttaggtggggatacagtatgataactgtatggaactaacactccaagtcaaacattgaagtcaaaattctgggctggcgtacattagttaggagcccccattttagaaaatattatttcaagcaggccttgtttttatccagagagtgtatacagtataataaaactcagtacaccaagatttatatttcatggcgtgtttttctccctaagtagagaaaaagctccttcagtaaagtgattgtgttgtcaaccactgcatattcttaagtgtaccaagatgcttgactccaggcaggtgctaagagagactgttgggtgagtgaatgtatatcaatgaaaatcaaacagaggaaattttctttatatgttttagtgttcctgtggtttttttttttttttttttttttttttttttttttttttttgtgatggttactggctcttctctaagaataatggagaaaaggtgttagtttgtactgtcgcacagggcagtcgtaggagactctgaaaaagcaaataaatcaatgttaaatcagaaatgtgaatgtagtaaggggctgaagagacaggggaagagaatacatgggaaaatattgaaaaggacagagtgatcaaaaagagcagggacatgggagcattgggcagcacactgggagccatttcactttatgctcttattgtatgattgagaaaaaaatgtccttagtggttaagtggcttttcaatgccacatcagacttgttccatagcagttgaattaggggaaggtgaataagttggaggttggtgacaaggagagaagctggaacagagaggagagtcagaaccagagggaaatgagagactgagtaggcatctcagggtttttgaaggagtggattttctttgttgcagtcaggggaggtttgtctgttggctgcagaaagaagtcagaatagagatatcgtggggtaggtttgtttggaacagaaatcaaagaccaatttttctgagagaaggaaataacatctgcaaatgatatgctgtttttgctacttccattgttagctgttctcccaggtgatggcaatgcagacggtaagaactctgacaactgcccagttgggtggtgggtgaaggtctctctctctctctctgtgtgtgtgtgtgtgtgtgtgtgtgtgtgtgtgtgtgtgtgtgtgtggtttccctagcatatacctggaaagtgagagtgccaggctggttccattctgactatccatgactccaggtccctgagctcttatgaaggtggttgataaggcatgggactgtgtggcaagtgaatgtctgctaagatcatgatggatccccttttctccagattctgagttcccgcactctggcacctttctctctccatcctctttctcaatgtctttctccccattctatctgttcttttgtcgcagggctcaaggagcctctctccttccatgtcacctggatcgcatccttttacaaccattcctggaaacaaaatctggtctcaggttggctgagtgatttgcagactcatacctgggacagcaattccagcaccatcgttttcctgtgcccctggtccaggggaaacttcagcaatgaggagtggaaggaactggaaacattattccgtatacgcaccattcggtcatttgagggaattcgtagatacgcccatgaattgcagtttgaatgtgagttcagttttctccatggagagtggtgaggtgggagagaggggttgtctcaatgtttcttattcctttaggaaacagcctgactctcttctgtatcttattctattctttccaccataaaactgcaattgcatacttttgggcaagggctatcatccagaccttaattctccaaacacttctgctacttatgaactcctctctcattgattgcagggttgtgcattattatcttttcttacaggtatcccaggaccacccttctgcctgccctacaaccagataccttcagctttctcactttctcattcattctcttcagcagtctctctatatatactcaattgtaacccattgcatttaattcaacctaagttgttataatcccagttctcttacaccaggtccttgctcacccacagcattcacatcaccttctttgaccagtttgccctcttcctcatgttattaatcttttcctccacctacagctcccaccaattcctctttctctgaatccctgaccaaaccaaattttattccattatgtcaaaccctgcacacttcccccttctgcttctataatctttgcttctactcatttcattttatatttttttctccctctttcctctatttcataaccccagatccttttgagatacaggtgacaggaggctgtgagctgcactctggaaaggtctcaggaagcttcttgcagttagcttatcaaggatcagactttgtgagcttccagaacaattcatggttgccatatccagtggctgggaatatggccaagcatttctgcaaagtgctcaatcagaatcagcatgaaaatgacataacacacaatcttctcagtgacacctgcccacgtttcatcttgggtcttcttgatgcaggaaaggcacatctccagcggcaaggtcagtcctgcactctccctccaagaagttttgatttgaaaatcatacccttccaccacctccttccaatttagaataaggaagagccacccagaagtgggaaaggctgggtgcagtgcctcacatctgtaatcccagtactttgggaagctgaggctggaggacttcttaagggcaggagtcccagaccagcctgagcaacagagtgagaccagtgtctctaaaaaataaaaataagttagccagtcatggtggtacacacctgtagtcccagttacttaagaggctgaggcaggaggatcgcttgagcctaacagtttgaggttacagtgagctataatttataattatgccactgcattgcagcatagtgacagagtgagaccctgtctcaaaaaaaaaaaaaagagaaatgggaacaatgtgtctatctaaacttctaaactgttgtggagatatgaaactccaagtctgtatttgaatatcttttctgtcctttgcagtgaagcccgaggcctggctgtcccatggccccagtcctggccctggccatctgcagcttgtgtgccatgtctcaggattctacccaaagcccgtgtgggtgatgtggatgcggggtgagcaggagcagcagggcactcagcgaggggacatcttgcccagtgctgatgggacatggtatctccgcgcaaccctggaggtggccgctggggaggcagctgacctgtcctgtcgggtgaagcacagcagtctagagggccaggacatcgtcctctactggggtgagaaaaagctaaggcccaggctggaaatgccaggaagtggacctcaggcatagagggagggcaagctgaaaattttaggattttagggattatagcccaaacatgaataaaaatagataatctaagaaatggaaatgttgaaaatgagggccctgtagatgcaggaggatgctgagggttaactgaagaatccatctctgagcagggatgagagaaaaacatagaaaaaaaggagattggtaagttggatactcaaatgaaaaaaagaagcccagggaatgcagtgaaatagggagtggatgaggtaaaatgggatggattataacatccttggtgtctccccaaattcacagagcatcacagttccgtgggcttcatcatcttggcggtgatagtgcctttacttcttctgataggtcttgcgctttggttcaggaaacgctggtgagttctttgcatgtgtctttcctactcttagcctctaccccacttttcttcccatgttttttgtttttctctcctcagttcttctctccccagtccccttccctcttgctcctcaattctcagctccaccttattcagagtgacttctatctctgttctcatccagtttctgttaagacacaccatgagcctcctcgtcacccttctccttttggggtgagagaccagcagcccaagggctccagacacacctgaacacatcgtgatgatgacgtcctctcaactctctttgtaaaaattttgttatttttgcttgtttctgattaatgattgtttgtcaatataagctcaatttaattttgcaggatttgttgttctgacctgggttctgggacttttaaattcaaattttatctccagatggaatggggtcctagcaacctccacatgttcaactattaatggatcatcaggcctgttttagatatcccttactccagagggccttccctgacttacaagtgggaagcagtctcttcctggtctgaactcccgccacattttagccgtactttgctaactgtgctcctcacttcctcttcttcattgcagttatttagatcccccctttccttctaatttttcagctccttcaatgcaaagtacatgtatttttaatatatgcatccctggtgaaggatcttgcctgcatgaaacatgttctcaataaaactctgtgttgaatttatgccaaatttgtagtgttttcttattcttttccttcctggtgcttttgaagtagttgagacatttgatactcttctgcttaaaagtcttctcacttggaggaaattttacacattcctagttctatagtttgttctctgagctccagaggagaaacacaattttgcctcttactactcctatcattcaactatagagagctcttcgagttaaattattgttccaggtatgcaaggcaggatgtatatctttactattcagtggttcagggaaaagcagcagagttctcacattggagcttgttagaaaggtagaatgtgggcctcaccttaggtttactgaattataatttgtgttttggaaaatcagcaggatactcacatgtccattaaaatttgtgaggcactgatgctggtgatgcctgagatgtagtacaagtgtgtcactgaattgaaaatgatcaaaaaagggaacaagagtgttactcctgaagattgaagtgagatcaaggcaagtaatgtacctactgcaccttctcattattcatggcagttatgttgtgtgcagttgctgcaaatactgaatttgtgagcaatgagccattgttcccaggggaaatacaggtgttaggctcttgtgagatttttcacaacatttttgtcaactggtgaatacataagctggttttatgtgtgtttctgtttaaaaacatcttattttatatatgtatatataattaattcattatatgcagtatttctttataataaaacactatctacgaagtgtttaatatattcctgaccttggttaacatgatcacaaatttctttggttttcagcttcacaacattgctgtccactacacttaaaaaaaatggttgtcatttcatgaatccacaaatgtagctattttcccatagcttcagcatagcattatagatgttactttcccaagcagcctcacctacagatgggtgaatttcctcccccttttcctgcatgtactgcactgttgatttatttttatttatttatttattttatttatttatttatttattatttttatttatttatttatttatctctgtaaggcatgtcacagctttcttatgcttaggaaaatgcgacagcacttaagcactacacttgggaccatttaagattgtgaaatcaccaaccaaaaacccaaaaatgtgcagaagttggcactatatagactacaaaaaggacacttgtttactgtgtgagagctggaacatgaagacaaagcaccacctttttcgaccttagttaggaacatgttctttgggggactcaaattttgtacctcactgcatatattcatgaatgattgtgaatatgctatgaatatatatctaggggttacaaataaaatttggcaagtagacaaattagcaaatataacatttttgaatgatgaggattgactattgaccccagatggtagatggtagtatttgtcaagatatccatagctagacttcaattactttaggattcatgaattcaacaaatatttcttatgattctgcaggttgccactttcatttcttgttgtttatttctcagttttctagtatagtaacttattagagttgaacttggggaaaaggacttagctctaatttgtttaggatactgagaagaggggcccaaggcaagggaaacatccaggcagaccaggtgcagttggcagtggctgcgtatatggaaatttttggaaactgaacttgtaatgggtgaaggggaaaagaaaaatatgggatagaggagagcaaagaatgggacagctgcagtctttttacctgtgtttgacatgattggttccaacccatttgggtttgaatccaccttgccacatttgcatagtagtcatctggcttttggttggttgataccaccttcagctctagtggtcagcctaaggcaatcatgataactcattctttacaatgtgataggctaaaggatggaaggtaacctgagatttaagccaatcatcatatagatcccacttacccttgctcagagtggtggttcagggctatgtacttgttctaaaatgacactgtttacatgacgcctgattcttgtgtttgacaatagggaaaagagaggctctctcttgtcctggatggtatggtgtgaggtatagagcttgaaacagctcaagtcatttttttttcagcaccagagaaatcacacaggcaagaaagtagaggcaacggactctcagaagagtccagattaaactgtatccaaagcctgaatgaaccctggacttctcagtgatgttagatgatacatttcctttactgtttcagcctatcaagttgggttttctcctgtttgtgcttgaaagcatcctatctgatgtacatggaccctaggggacacttagctgattttactcttggttgaagtattcctctttatttccatcttcctcctcctcccttcttgggattggctatactttaaaattgatttcttttctttcgcaggttgagtattctttatctgaaatgcttggaatcacaggtgtttcagatttcagatttttgggggggctttagaacatttccatacacccaccagccgagcatccctaatctggaaatctgaaacttgaaatgctccaacgagcattttctttcagcatcgtgtcagtgctcaaaatgttttaaattctggagcattttgcatttcagattttcacattaggaatacctgacctgtagtttttttcttatatgtgtgcttatctttttaaactcttacactgtcctgtttttaactatgctgcctgaattaattactctcttgaacttttcatatatttctgaccaggaataaaagtgctgcatctgctgctaagtgagtgactttggaaaggttccctgatcttccttcttttctttggttaaatggaggcaaaaatatatactcttaggattttcataggaatgcaaaggcaacctagtgttattaaaatgccaggagtttggtctacggcctgctgcttgccacataaaaaccagttaccgaggcaatgagtattgcctttaattgactgctgcagccgaggagataggaaatcaatctcaaatccatttccttgattgactaaaattagggatttatagggcagggaagaaacagaaccctgtgtggcaaaataggaattagggagaggtaaggaagaagaattggtcaacagcaagcaggtggttgcttaagcaatcatgatgggtgaggagtctggcatctcgttgtccagatgcggtagattggtaggttttagttgtttttgacaaggtttagttccctggctgggaatgtattgctagtaacttggctgctgtgggcagggagagtctcaaaagtcctcctaagcagctgcccaacacttttttggctacaggtgggtgttggtttttctttctctggccctacctctgccagccccagctgtgtttctgattgcctaggaagagcagcctttgaaatgtgtcatctgagtctggatgggtgaatgtcttttgtgggtaccagacagtgggatcttctcctctcaatttggggaattctggaggaatttccatttgcaggttgaacaagcccaactgattgatattggaactcgttggttgctgcagcaattggacagtgttttagtgattgcttgtttatttgtgtgtgttaatatagtcacaggaaatcagaatttgataaactgataatattttgtagcactgtttggcttcagtgttctttggaatctggagaagtttggcttctccatgggtcatgctgttgagtgagacagtggaacggtgttatggtgttacatgtatctaagcttttaggctgctgttctaagcagggttggacctggttagtacatgatgttcttctatagtgctgtttggccctagtggtctttggagcctggggaaatttagcctttaataatcaaacggccatggaaactgctttacccaaattttggttcaaagctttcattggattacatattggggcaaacaaacttgagccatgtgaacatgttcctaaaccagtggtttggtattgctatctcatggctagagttccaaggtaaaagctattggatctttgtgtgtgtatgtgtatacatgtttatatgtgtttatgtgtatgtacatttattatgttgtatgttgtgtctaccaaattggcttataaataaaagagcatcttatacactaaataaataaatctaaggaattttcaattttatgtgacttaagtaaatgtttatttaaaaagctggctttaaagtcattagtaataaaaaaataagaatgtcttaacagtgttagaatacattttcgtctgggttttataattgtctttgctagatactttgaggtgtcagtgtttggcacagagggttataaaagtataaacccagccaaaacaaaatgatctttgtttgtgtgccttctttggcaaataagactaagtaaatgttgttagtttaataaaaacagttgaatcttctaagttattggcaaaaatacctatgtatttaagttgcttacttaggtgagcacctaatattcataggctacaaataatggtcaacagagcaataacctgaaatgacgactacctttgtctaatatctcagttttcagaacccacttatataaatttttttttttctttttttgagacggagtctcgctctgtcgcccaggttggagtccagtggcgcgatctcggctcactgcaagctctgcctcccgggttcgcgccattctcctgcctcagcctcccgagtagctgggactacaggcgcccgccaccaggcccggctaattttttgtatttttagtagggacggggtttcaccgtgttagccaggatggtcttgatctcctgacctcgtgatccgcctgcctcggtctcccaaagtgctgggattacaggcgtgagccactgcgcctgacctataaattgtttaaaaagatgaaagaattgaatacatgtaaatgggatacatgcttgtaggtgagctttttgtgtaatttaaaatcttaaaattatttttgatgcttcttggatgtctggctcatttccagttaagaaagggttatgatatggagaaacatgtttttataaattgtggaatgttttcatctataaaatgcaaatatttgatagacagttaaagatttcttgcttcctagttttcactaaaatttaaggttactaagaataagaattgtagttaatatataatttagtatataaaacctgccaaagaaggtgttttcttattgagaaatggaataattttgtctaattcaaatgttttctaaagtttgatcccaattatggactcgaaaaacttatttatgaaataaggtagaaaggaaccagcaagtagggaagagagatgtgaagaaagttaagaacataaagatgtatttttggcaagaaaggttaaaaagaaaagaggataattttatacaagtatcttttcttttagacttttattttaggtttggggtacgtgtgcaggttttttatatagtaaactcatgttgtggaagtttgttgtacagattatgtcatcacccaggtactaagcttagtacccaatagttatttttttctgctcctctccctcctctcatcctccatcctcaaggaggccccagtgcgtgttgcgtgttgttttcttcttggtgctcatgaactctaatcatttagttcccacttaaaagtaagaatatgtggtattttgttttctgttcttgcatcagtttgctaaagataacggcctccagctccttccatgttcccacaaaagatatgatctcattcttttttatggctgcatagtattacatggtgcataggtactacatttcctttatctaatctgtcgttgatgggcatttaggttgaatccatgtttgctattgtgaatagtgcttcagtgaacatttgtgtgcatgtgtctttacggtagaatgatttatatttctctgggtatatacctagtaatggattgctgggttgaatggatatttctgttttcagctgttggaggaatcaccatacttctttccacaatggttgaactaatttacactcccaccaacagtgtatgtgtgtcccctgagaaaacaatctgacagcattttttttttcctaaaaataaatgaattttttaaaaaaaatttaggacaaaacagaaagttcaagcatgacacagatgccttgttgaagtcatatgttgttttctttctgtttctctgtgtgtttatcttcatgtacataaacagaaaataaaaagttgaaaagtttatatagtaaatatcctttaaaaactgatagaaaattggagacatttgactaattaacattgttcaccgttaaagctcttagtcttgatgaaggtacaataagaattattataaataaatacattggcagtttgataattctttttaaatatagttaagcctgaagccagatttaacatggagtcaaattttacataaatgcttgcattgctttgtttcacactgtatttgttattctacatagaaagtactagcactaaggtacttattggttacatgcctaaagtgaatttcttaattgcacaaaatgtagagtggtactggtggacttaaacacattaattagtatatcagaaacagaatatcatatttttttaggccctggataacactgtagactccaaggtaaactaagtaagagaaaatttgggagttattttcctgttggttttgcttttaattttcatttatttgctgtttgttctcctttgggttttacttatatttacatacatataaaacaattggtgttttttagtttctactgtaaggcttgtatttggttctatgaatagtcattttgtttcctatgcatttccaaaggcttatcatttgctctatttatctaaaattcctaagctgtctttgtcaagcttccaaaaattggcagaggacagcagccatttaaaatttggactggggctgggtgcagtggctcatgcctgtaatcccagcactttgggaggccaaggtggctggatcacctgaggtcaggagttccagtccagactggacaacatggcaaaaccctgtctcttctaaaaatacaaaaattagctgggggtgggattacagtaatcccagctactcaggaggctgaggcaggagaatagcttgaacccaggagatggaggttgtggtgagccaagattgcaccactgcactccagcctgggtgacagaagtgagactctgtcccaaaaaaaaatagttgaattttactttgtaaaattaagaatttctcttcaacaaactacacactgagaaaaattaatgcgtagacaattaagaaaactatttggaattctaacaataaaatgggtatatataacatatatgagatatacacccatatatgaatttggagatgtttgttgattaagtatacatgtgtagaaacaacagaccagactgttgcgaaggtcttcaagaaagtgctgaatgcaatgcatcaatacacgaagcatacagtacaatacctgtattgcaattagagttgtacacatcaaatagtaccactttttttcttttataattgagattttattggttgtgttgagggtcagtacacagatatttcaattcttaatatatgtactgaaaatctaaaaagccatgtattgtaattattttttaaagttgttccagtaactttcctgcttaaaatttggaggcaaattttccttaagaggctaccaagtaccagtatcttcacatgttgataagctgttaacagatgtcccaccaactcacaattgaatagcatatacactacacactcaaatttttagtatttcacagcatggtaacaaagttattagaaaaacaggactaccaccaccaaagatgttacagagtgcacacaattctgacagggagagccattatgaaggagtggtttcctttaggaaacaattctactgacaaaaaacatgggactagaagtaatttaaaatgttcaagacattaaaggcaggactgtgactccacattgccatttagtacgctttgtattataggatataaaagctaaccccccatccatggaatgttaagctgacacccaagacaaagcctcccataattcgatattccatactattttctggtaccaaaaataaacaaccagagaatgatttcacctttaaaaaaaaagcacttgcacttaaaaaatgaggtgagatgggattcactctttcttaaaaatgtttctagagctactgaaaaacttttgtttacaaaatagttcataaaaatattcccccaggttgtacaagaagggagacagggaagaccgataagacatggaatgtaatactaatcagacttggcttctttctctgcagcttcagaggctggactctcctcgattttagtttttccgttttctgaagtaaatctttagtttcttgattacccacgtcggcctgttttccctttgctcctctttccccttttgtttgcaattttttgtctgaagatttgtccttttctttttcttttttttgacgaagtcttgcgtctcgctgtcacccaggctggagtgcagtagtgcgatctcgcctcactgcaacctccgcctcccgggttcaagcgattcttctgccttcgcctctggagtagctgggattacaggcgcccaccaccacacctggctaatttttgtatttttagtaggcacggggtttcaccatgttggccagtttggtctcaaactcctgacctcaggtgatttgcccacctcggcctcccaaagtgctgggattataggcctgaaccaccacgcctgacctcctgctgccttttccggcttcatttccacttttgcaggagaagctttagctgccaacggcactgatctcctcttgggctcttccttcatggctccttcatcccggtggtgcagaacgtgtgtgccaggtgcctgcggccgctgcacgtaagagccctggtgaagctaggctgcctggccactgccgctcctcccgcctgagctgctgagaccccgacaccatgtgttaaaagaacacataggcacagaaggatgacacattaatacattaaaatgtggggaagggaaggaagtggggataataagtaaataaaataatatacagaggaagcccgctcaggccaaatgatatccacgccatgaatagaagagaatgaactcaacaatctatacttaagattccagagaagcatgatttatactcatttgggtatatacccagtaatgggatggctgggtcaaatggtatttctagttctagatccctgaggaatcgccacactgacttccacaatggttgaactagtttacagtcccaccaacagtgtaaaagtgttcctatttctccgcatcctctccagcacctgttgtttcctgactttttaatgattgccattctaactggtgtgagatgatatttcatagtggttttgatttgcatttctctgatggccagtgatgatgagcatttcttcatgtgttttttggctgcatagatgtcttcttttgagaagtgtctgttcatgtccttcgcccactttttgatggggttgtttgtttttttcttgtaaatttgtttgagttcattgtagattctggatattagccctttgtcagatgagtaggttgcgaaaattttctcccatgttgtaggttgcctgttcactctgacggtagtttcttttgctgtgcagaagctctttagtttaattagatcccatttgtcaattttgtcttttgttgccattgcttttggtgttttggacatgaagtccttgcccatgcctatgtcctgaatggtaatgcctaggttttcttctaaggtttttatggttttaggtttaacgtttaaatctttaatccatcttgaattgatttttgtataaggtgtaaggaagggatccagtttcagctttctacatgtggctagccagttttcccagcaccatttagacacatgcacacgtatgtttattgcggcactattcacaatagcaaagacttggaaccaacccaaatgtccaacaatgatagactggattaagaaaatgtggcacatatacaccatggaatactatgcagccataaaaaatgatgagttcatatcctttgtagggacatggatgaaattggaaaccatcattctcagtaaactatcgcaagaacaaaaaaccaaacaccgcatattctcactcataggtgggaattgaacaatgagatcacatggacacaggaaggggaatatcacactctggggactgtggtggggtcgggggaggggggagggatagcattgggagatatacctaatgctagatgacacattagtgggtgcagcgcaccagcatggcacatgtatacatatgtaactaacctgcacaatgtgcacatgtaccctaaaacttagagtataataaaaaaaaaaaaaagaaaaaaaaaataaaaataaaaataatttcttactactgttaaaaaaaaaaaaaaaaaaaagattccagagaagacagaacacaatacaacaaatcaaaaattggaaaattatgatgcatagaatgaagaaactgaattgggataaaagggtcgcaccatgggtaacttcccagggaaaaataataaaaatgcatgatattgtgtaaaatacttctgtgtctaagtacagaatcaaaatatctgcaacattaaaactcgggttgaaaatgtcagggtttttcaccctgataccaaaaccaggaaaagacaataataataatgatttaaaaaaaactgcagaccaatatccctaatgaacgtaggtgcagaaattctcaacaaaatactagctaaccgaatccaacagcatatcaaaaagttaatatatcatgatcaactgggttttgtatcagggatgcagggatggtttaacatatgcaagtcaataaatatgatacatcacgtaaacagaattaaaaacaaacatatgatcatctcaatagatgcagaaaaagcatttgataaaatctagcattcctttatgataaaaccctccaaaaaataggcatagaaaggacttacctcaaagtaataagagccatatatgacaaacccaccgccaacatcatactgaatggggaaaatttgaaagcattccccttgataagtggaacaagacaaggatgcccactttcaccacttctattcaatatagtactgaaagtcctagccagagcaatcagacaagagaacaaataaaggacatccaaattggaaaagagatcaaactgtcactgttcgcccacgagttgatcacatacttagaaaaacctaaatacgcatccaaaaagctcctagatctgacaaacaaattcagtaaagtctcaggatacaaaaccaatgtacacaaatcagtagcactgctattcaccaacagtgaccaagccgagaatcaaataaaaaaactcaatccctttcacaacagctgcaaaaaataaaataaaatacttaggaacattcttaataaaggatcttgaaagatctctacaagaaaaactacaaaacactgctgaaagaaatcatagatgacacaaacaaatggtaacacatcccatgcttggggatgggtagaattaatattgtgaaaatgaccttgctgcccaaagcaatctacagattcaatgcaattcccatcaaaatatctttataatttttcacagaactagaaaaaagaatcctaaaatccatatggatccaaaaaagagcccacatagccaaaacaatactatgcaaaagaacaaatctggaggcatcacttcattcaactttaaagtacactacaaggctatatttaccaaaacagcatggtactggcataaaaatagacacgcagaccaatggagagcatagaaaacccagaaataaagccatatatttaacaaccaactgatctttgacaaagcatacagaaacataaagtaggagaaatgacaccctattgaagaaaaggtgcttggataactgacaagccacatgtagaagaatggaactggattcccatctctcagcttatgcaaaaatgaactgaagatggatcaaggacttaaatataatatcccaaaccatgaaaactctgtaagataacatcagaaaaactcttctgcacattggctcaggaaaataatttatgactaaggccccaaaagcaaatgcaacaataacaaaaataaatatatggaacctaattaaactaaaaagcttctgcacagaaaaagaaataatcagtggagtacacagacaacccactgagtgggagaaaatactcatagactatgcatctgacacaggactagtatccagaatctacatgggactcaaataaatgagcaagaaaatgacaaaaattccaccaaaaaatgggcaaagaatgtgaatagacaattctcaaaagaagatatacaaacagctaacaaacattaaaaaaatgctgaacatcactaatcatcagggaaatgaaaattaaaaccacaaagagataccaccttactcctacaagtatggccataattaaaaagtaaaaaaacaaaagatgttggcctggatgtggtaagaagggaacacttatacactgtggttggggatgtaaattagtacaaccactatggaaaacagtatggagattccttaaagaactcaaagtagaactaccattcgatccagcaatcccactactgggtatctacccaaaggaaatgagaccattatatgaaaaagacacatgcacatgcatgtttatagcagcgcaattcacaattgcaaagatatggaaccaacctaagtgcccattgaccaatgagtggataaagaaaatgtggtagatatttaccatggaatactactaagccataaaacagaacaaaataatgtcttttgcagcaacttggatggagctggaggccatttttctaaatgaagtgattcaggaatgaaaaatcaaatattgtatgtctcacttataagtgggagctaaactatgaggacacaaaggcattaagagtgatataatggactttggatacttagcaggggggagattgggagggggtgagggataaaagactacatattggataccgtgtacactgctgtgtgacgggtgcactaaaatctcagaaatcaccactaaataacttatccatgtaacaaaaaaccacctgcatcccaagaactaatggaaaacaaacaaaaaacaaatttttgtattctaccagtagatgacattagagattaaaatgccatttctgcatatgcttgttattaaaaattttgtcttatggcttcccccaaatcacgcagcagctcagaaacctatccttaaattcacaataactttttggctctaaaatattaaaaatatattgataacaatccaggaatagaatccagggtaatatatggccaataaaataagatgaagccaagaacagtgtaaatttttaaagaagtgccacgataccctatactctggttagatgtgggtcacacattgttggaggtccatgataatcaagacaggatcagttacatacttgcagtgtctagaagatagacaatgggctgtttaggagaattatagctattcctggtcctgaggccacaaggacatttcttccatcagaggagaaactgcttaacgcagtgaacaatgttctcaggaaagtcctttcagtaaattttatgaggtccataggtctttttgtgattaagtctcttaaagtaagatcattatgtcagcctatgacaaagactgggaaaagtaattctacacagcataacaggatctagtccagacatgcagctttctgatgttctagcaaacccaagggtagattttgttatctgtagaaatgaatagattccacatctttcaaagatactgtttatattgaactgatttaaattatttttgaagcagcagcaacatttagattatgtattctaacatataccggatgtaaaactattttctatcatctgttaaatacagaacagtaactttgacaataggttgacttacaagtggaattggaattggaaatttcttgtggcttctgcaaatatctactcaaggacttctgcttgaatctgaggcttgctcccggaaggctgatagcacccatagagagcaccaagattcttcttgtaatataattttgttttttttttccaaatacaaaggcactagttgacataactacaacccccctcatcccaatgcacaaccaactgtacttcatagagaatgccagatatggctttagacaggaaactatttgaaacagatatgaatcttaatatgagtttaaagtgagaaaaaagaatactatgagtatgaagtaagaaaagcagccatgggtccttgagctatttccagattgcccattcaagtaaaactggttattaactatacacctgcatagctaaataaataaatgacttgccaaatgggctatatggcagagaacactggttttctcccagtatatgtttgtcacttcctctagaataactgaaacctgctgattattagctgggcagaataaaagctatgtttcttggcaccctttgagctgattgtggtcatgtgattacaacctgccaaatgtgatgtataacttctgcagaaagcagctctgaaatcatgggaagtcgatgtgctcacttcttcccttcctcttattggctggatgaaaggtggatttgatggaaagaactctggcagtcacctcagggtatgggaagaccactggaatgaggactaactgtggtaaagaccaaaacaaaacaaaaaaataaaaactctcccccgcaaaagaaggaaattggaaactgagttcctgacaccatggaatgccacatcaaccctgcattgcctactcctgtctttttataagagagagaaatgtggttgcatttttatttatttatttaattattttgagatggagtcttgctctgtagcccaggctggagtgcaatagcgtgatctcggctcactgcaacctccacctcccgggttcaagcaagtctcctgcctcagcctcctgagtagctgggactacaggcacatgccaccatgcccagctaatttttgtatttttagtggagatggggtttcctcatattggccaggctggtctccaactcgtgagctcaagtaatccacccacttcggcctcccaaagtgctgagattacaggcatgagctactgtgcctggctccatcttttaaaagatactgtcagtttgaattttctgtcactcacaaccaaatttaactaattcaggatgtcagctgagtatcaattaatcatcaatagcattggtctatgaaatgcttatttgaggctctttatttttctcattatgatgacctgtgtgggttacttaatttttgctaagacaaaaccaaattttgataatagcacccaagttatttgtatttaagttcactttcacttttgtctttatattttctaaagcaagggagaaaatgcaactagccgttccaaaatttatatgaaaacaaacttgttagaaatcagagggaacttctgggaaaatgatagagtgaaatatttcataacccatttcacttaggtaaatcccaagtagagaaagaaacttcatctatactgaatctagtaaagagctataacttacaacaaaagccaaggagtttctattttttatgttttcaccctttctactttgccagaaacactgaatagagttttgtctttctgatttaaagagaggaagagagaaaaaaatgccaactctgtagacaccttcagagttcagttttatcataaaaaactgtttgaataattagacctttacattcctgaagataaaataaacatgtaatcttttatcttattttgttcaacaaaattgttcagaaaatcaaagtggtaaaaacaatgtaaaatttaacattttaataccgatgttgtacactgttttacttaacattttggagagtaattgccttgctcttcaactcaggaaaacactttttttgtggctaatgtaattggtttctgtaacaacatcagcaaataaaaggatgctgattattgaaaagaaagttataataaaagttggtataaatttttagcacacatttttctgattttgagtatgatgaaaggttgatatctacagttttaatagaaaaagttcaattcaggagagcgtaaacaaagctactaggacctttgttagaagacgactatagggaataaagtattatggaaatggagaataaaataatttgggaggaatcttttggtggagccaacctttagaaaacaggctgtggccatgagtctttgaccttgttggtgtgcagtggcctatgtcgttgaatacgactggcttttatatgctgtgcgactgcctccttaccactgtggtctgaacaggcagctgtaggactttgtttctagtggccatttgaggtttttattgtggtaagttgtggctgtttaagatgggtttgcttaagatgagtgttaagtggctctatgtagatgagtgtctcaggtcagttgccgaggaaacactcagatgagcctcggcctgcagaatgcttactggggagtgttctatgaaacagcactaccaaaagaaagagggaagcaggactgagcagtgagacaaggaactgtgatgcagttgtaacagaggatctaacaacctcatagagagttctggagctaagatggcccttcagaggttttctgaaatgaagtaatgggctcatacctttgtacttctatattcgatgtgggctttccctagggaaagaatgtccccttaggcaaagtggctcccttctacataaggcagtttttagaaggacacatctgtgagtagtcagcagccaacactccctgaagctgaggaataagcatcttcttcctgaagaggctgtattgctggctgaccacagcacccactagagcaagggtatgtccttatggaagtatggatttacttgctggaccttaaataaatgcctcataaacggtgtcaatattgtagacacaggccaaggccagagcaaatcttaggaaatatgcatgggattctacaagcagtaaccttaactaggctgcctcagcatgatggctaaagcaaaaattctattaatggaaccagagacctcttagatatcagttcttcttcttcttctccttctccttctccttcttctttcttttttttttttgatggagtctctctctgtcgcccaggctggaatgcagtggtgcaacctcggctcactgcaatctccgcctcccgggttcaagcgattcccctgcctcagcatctcgagtagctgggactacaggcaagcaccatcatgcctggctaattttttgtattttgtagagacggagtttcaccacgttgatcacgatggtcttgatatcctgaccttgtgatctgccctcctcaccctcccaaagtgctgggattacaggtgtgagccaccgtgccgagctgacatcggatcttctaaaacaccaggcagcaaaataattattgccagtttttgccttgattttcagtccagactgaaaggtctgcagtctagatgagatacctcaaatttggtttgtctcctacattattttaaactggtttcagggtcaagaaggagtcccaggaatgaactcaccttgactttatgtggcttgtacggaggcccaggaatcatctcccactacataggttcaatttgcctacacctgttattgcttagatctttgagacacaagttgtggcttcacacatataaggatggtaaattctattaataaataactaaaaaggaaatagaaagacttcttttttttaaattatactttaagttctagggtacatgtgcacaacatgcaggtttgttatgtatgtatacacgtgccatgttggtttgctgcacccattaactcatcatttacattaggtatttctgttaatgctatccttcccccatccacccaccccacaacaggccccagtgtgtgatgttccccgccccatgtccaagtgttctcattgttcaattcccacctatgagtgagaacatgcggtgtttggttttctgtccttgcgatagtttgctcagaatgatgggaaacagaagtacttctgtttctagaaatgttatactacataatttggaccaactctcttatcaaggatgtctagaaaaaacaataaaaatataaaataaaacctgcctcaaggcatcaaagaacccacaaaagggagatggattattgggttaatatctgggagaagagagaaactcatagtgatgaaccctgtatttggggctgcttttctccccagaggcatctactgattccatgagaggcactaatgagactgaaaagctaagcttttaacagacttgtaaatcttcaaagatataattgaagcccacattttaaattgggaacttaaggggttaattccagcagtaagtataaaacagaaggagaataacctgtgcagggattacaacacagttccaaattatcttaatctctgaaattaataaagcaattttgatccaatgcccttaaataaatatagagcaagataacagcatcctgggtcccaaattatttctacattttttaaattttaattttaatttatttatttttttattattatactttaagttctagggtacatgtgcacagcatgcaggtttgttatatatgtatacatgtgccatgttggtgtgctgcacccattaactcgtcatttacattaggtatatctcctaatgctatccctcccccctccccctaccccccaacaggccccggtgtgtgatgtccctcttcctgtgtccatgtgtcctcattgttcaattcccacctgtgagaacatgcagtgtttggttttttgtccttgcgatagtttgctgagaatgatggtttccagcttcatccatgtcgctacaaaggacatgacctcatcattttttatggctgcatagtattccatgttgtatatgtgccacattttcttaatccagtctatcattgttggacatttgggttggttccaggtctttgctattgtgaatagtgccacaataaacatacgtgtgcatgtgtctttatagcagcatgatttataatcctctgggtatatacccagtaatgggatggctgggtcaaatggtatttctagttctagatccgtgaggaatcgccacactgtcttccacaatggttgaaccagtttacagtcccaccagcagtgtaaaagtgttccttgttaaattgatccctttaccattatgtaatggccttctttttcttttgatcttgttggtttaaagtctgttttatcagagactaggattgcaacccctgccttgttttcttttccatttgtgtggtagatcttcctccatccctttattttgagcctatgtgtgtctctgcatgtgagatgggtttcctgaatacagcacactaatgggtcttgacactttatccaatttgtcagtctgtgtcttttaattggagcatttagcccatttacatttaagattaatattgttatgtgtgaatttgatcctgtcattttgatgttagctggttattttgctcattagttgatgcagtttcttcctagcattgatggtctttacaatttggcatgtttttgcagtggctggtaccagttgttcctttccatgtttagtgcttccttcaggagctcttgtagggcaggcctggtgctgacaaaatctctcagcatttgcttgtctgtaaaggattttatttctccttcacttatgaagcttattttggctggatatgaaattctgggttgaaaattcttttctttaagaatgttgaatattggtccccactctcttttggcttgtagaatttctgccgagagatcagctgttagtctgatgggcttccctttgagggtaacccgacctttctctctggctgcccttaacattttttccttcatttcaactttggtgaatctgacaattatgtgtcttggagttgctcttctcgaggagtatctttgtggtgttctctgtatttcctgaatttgaatgttggcctgcctttctaggttggggaagttctcctggataatatcctgcagagtgttttgcaacttagttgcattctccctgtcactttcaggtacaccaatcagatgtagatttggtcttttcacatagtcccatatttcttggaggctttgtttgtttctttttattcttttttctctaaacttcccttctcacttcatttcattcatttgatcttccatcactgataccctttcttccagttgatcaaattggctactgaagcttgtgcattcatcacatagttctcgtgccatggttttcagctccatcaggtcctttaaggacttctctgcattggttattctagttagccattgtctaatttcttttcaagattttcaacttctttgccatgggttcgaacttccctctttagctccgagaaatttgatcatctgtagccttcttctctcaattcatcaaagtcattctccatccatctttgctccattgctggtgaggtgctgcattcctttggaggaggagaggtgctctgatttttagaattttcagtttttctgctctgttttttccccatctttgtggtttatctacctttggtctttgatgatggtgacgtacagatggggttttggtgaggatgtcctttctatttgttagttttccttctaacagtcaggaccctcagcagcaggtctgttgtagtttgctggaggtccactccagaccctgtttgcccgtgtcagcagtggaggctgtagaacggcgaatattggtgaacagcaaatgttactgcctgatcattcctctggaagttttgtctcagaggggtacccggctgtctgaggtgtcagtctgcccctactgggaagcctcccagttaggctacttgggggtcagggacccacttgaggaggcagtctttcccttctcagatctgaagctgcatgctgggagaaccactactctcttccaagctgtcagacagggacatttaagtctgcagaggattctgctgccttttgttcagctatgccctgccccaagaggtggagtctacagaggcaggcaggcctccttgagctatggtgggctccacccagtttgagcttcctggccactttgtttacctactcaagcctcagcaatggggggtgcctctcccccagcctcactgctgcattgcggttagatctcagactgctgtgctagcaatgagtgaggctccgtgggcttaggaccctccgagccaggtgcgggatataatctccttgtgtgccatttgctaagaccattggaaaagtgcagtattagggtgggagtgacccgattttccaggtgctgcttgtccccccttcccttggctaggaaagggaattccctgaccccttgcacttcccaggtgaggcaatgcctcgctctgcttcagctcacactcggtgcactgcacccagtgtcctgcacccactgtccgacaatctccggtgagatgaacccagtatctcagttggaaatgcagaaatcacccattttctgcgtcactcatgctgggagctatagactggagctgttcctatttggccatcttggaaccacccctatttctacatttttaaagtaaatgaactatatgaaattattgacatatgtgaatgacatgtgatatgagtggttcagtttttggcaggacaaagtcaggaatccagagcatcatccaagaaaatgtattactattgtcttcacttgcaaatatcacagcaaattatggtggaacatttgtatagatgactaatattaccatcaaataatatagcatattacttagggtataaaatacaaaactaaacaattaggattatggtagatatgatgaatcaagtaaatttaattaaagtaagaaattctatatattgtccactaaggtttgacgacatatcctcaaatataagctaggtatagcagattattattattaatttatgtatttccaagtgcatataattccatttaaaacactttttgtgtgttgaataaatttcccagtgtatggaattactagaaaaattatatacaacattaatagacaagttacaaaagttatatacaacattaatagacaagttacaaaagttaaatataaacaaaataatttgaagattcaggatatgacaatgccaccatgcatggtaaatataattggcaacatctaaaagtgtgtgaaaaaattcaatgtcattctatattctttgatccattaacagtcttttttttctctcctaaacaaatgacatgtttgcagtatgttaaaaggcaggaaaaattaagcaaaaagccccaagaccttcatatggtctttattcgcctgctgtaaacattaagtactttcttttttctttttttaattatattttaagctctcgaatacttgtgcagaatgtgcaggtttgttaaatagatatacacgtgccatggtggtttgctgcacccactaacccatcatccagattaggtatttctcctaatgctatccctgccttaacccctcatcctctgacaggccccagtgtgtgatgttccctgccctgtgtcaatgtgttctcattgttcaactcccacttatgagtgagaacatgtatgtttggttttctgttcctgtgttagtttgctgagaatgatggtttccagcttcatccatgtccctgcaaaggacatgaaatcatccttttttatggctgcataatattccatggtgtatatgtgccacattttcttgatccagtctatcattgatgggcatttgggttggttccaagtctttgttattgtgattagtgccacaataaacatacatgtgcatgtgtctttacagtagaatgatttataatcctttgggtatatatccaacaatgagattgcaggatcaaatggtatttctagttctagatccttgaggaatcaccacactgtcttccacaatggttgaactaatttacactcccaccaacagtgtaaaagcattcttatttctccacatcctctccggcatctgttgtttcctgactttttaatgatcatcatctttgtggtgttctctgtatttcccgaatttgaatgttggccagtcttgctaggctggggaagttctcctggataatatactgaagagtgttttcaaacttggtttcattttccccatcactttcaaatacaccaatcaaatgtaggtttggtcttttcacatagaccaacatttcttgtaggctttgttagttccttttcattcttttttctcaatcttttcttaacgatttatttcattaagttgatcttcaatctctgatatcctttctttcacttgatcgattcagctattgatacttgtgtatgcttcacaaggttctcttgcagtgtttttcagctccatcatgccatttatgttcttctctaaactggttattttagttagcaattcctgtaaccttttttcaaggctcttagcttccttgctttgggttagaatatgcttttttagcttggaggagtttgctattacccactttctgaagcctacttttgtcaaattgttaaactcattctccatccagttttgtttccttgctggcaaggagttgtgttccttcagaggagaagaggcatactcttttttagaattttcagcctttttgttcttgtttttccttatcttcgtagatttatctacctttggtctttcgttttggtgaccttcggatggggtttttgtgtggacatactttttgttgatgttgatgctttttcctttctgtttgttagttttccttctaacagttaggcccctctgctgcaagtctgttggagtttgctggaggtccactccagaccctgtttgcctgggtgtcaccagcggaggctgcagaacagcaaagattgctgcctgttacttcctctggaagctttgtcacagaggggcacctgccagatgccagcaggagctctgctgtatgaagtgtctgtcgacccctgctgggaggtgtctcctaggcaggaggcatggtggtcagggacccacttgagaaggcagcctgtcccttagcagagcttgagcactgtgctgggagatctggtgctctcttcagagctggcaggcaggaacatttaagtctgctgaagttgtgcccacagctgccatttccccaggtactctgtcccagggagatgggagttttacttataagcccctgactggggctgctgcctttctttcagagatgcccttcccagagaggaggaatctagataggctcattcacagtctttttaaaacttttgtagtaaaagatatagtaaaatttatctctaaatagtttaaaaatatcatacaagagtagtattatctttcagtcttgacaaaacattggattgcattgctaatagaaattaatataaaattataaccatggttgattatatgatggggaagttgagctgacaccattgtattcctagggattcaattagtgatggtctgcaatacttctttagaaattttgaagatattaataaaatcctggattctagaaaacaagccaaaagtataagtacttccaaaaatactttgaaattgatgtgtttaatactggtttggcatgatatgtaagtaaaattaataccataaacagaatgatattaaaatcccaataattccttgcaaacttcaaaggattatgacatttttaaatgaaaaatgaggtgataagattcataagaagatatcactgatacttaacaacctgcagtttttaaaaattttgactccaaatcctgtaatataaagtcagtctttgctttgcatgttaatgcaggactgtaaaaattgatcatgcaagctaaaatcatgtgaaaggatctcaataatcaagagagagaattatgatcaccctgtgacttgaaacatctttgtcaaaatattcaaaacccacttactattggttataaatatatagggaagtgaaaaacagtacaatataatttacttagcacactatactttaaaacattaaaacatttggaattaaagtctttttcccttgtattaacttaccaagaatgttttgagcaatgtttgccttcatcttctgttacttaaagatacagagtatctttgtattccttggtgaattttcatactccttttaatgtttggatcagcttctgatattttatactttgtgctttccctcttggggaatatctctgagagttcctttaatgggaagttttctgccagtgtcacttcatttaagacaccttcattcttttcacaacaatcacttccctcctccaggttcctctagcttcacctctaaagtctctcaaacagtagcattctcagcattcccacggtcagctatttcctctatagcttcttttatatttaattcaaatctcacttctagcattgtcccttttcttctttgcacattcatctttgttgcccaatttcctctttctattattcactcatgtcagatgggtatgggtttattactgagacacaatgagaccacaggactacacacctacatgctttgctgttttgcacagcacagaataatagtcaaactgacaaactttgacatatgtaaagcaattggtcactgattatgatgtggatttgttacttctatggtgactcatggactgaagacctagtggtagtttatacttcgtgcagttactaagagttactatactgtggcaactgaaatttgtggcacacatttcaaacttgttgttggggaatttgtgttatttaactaaaactttgctgactgaaatttgtgcattttgaaactgggcaaaacaaagacagcctatatcagaaaaatgaagaagactgtgttgattttgttgaatatttttgtgtttgctttgaaattatatactactagaaataagttttgtgtttgcttctgagtgatatagtacttctacaagcttggaactattttgtattgttgatttcttggatcagacattttaagacaaatagaatacatttaatctctaaactcctgtgagagcaaacctgtgattaaaactttttgtaagaaaaaaaaaaaaaggaaatctagccagagcttaaggcgaccacaagattccttcttgtcttttggttcagttggatttttttttttcctgaccaattttattgaggatacagtagaagatttcttgcacttttatttggtggtctcaacatgatcttatgacctatgtggatactgtggtcaaagccctcaggaacccactcttgtcaatgtcttgggaagctcaagtgttagcttctatttccctatctagttcttaatttcttatttgttttgttttttgcagactttcttatcttgtgagttcaatgcatacatgtataaataatttttaaaattatagcttacccagtatatctatgtgttttgtagtttgtttgtttcttctctttttttgagatggagtctcactctgtaacccaggctggagtgcagtggtgcaatctcggctcactgcaacccccacctcttgggttcaagcgattctcctgcctcagtctcctgagtagctgggattacagtcacacaccatcacacctggctaatttttgtatttttagtagagatggggtttcaccatgttggtaaggctagtctcgaactcctgacctcatgatccacctgcctcagcctcccaaagtgctgggattacaggcatgagcctccataaacacagaaatctaaggactaagtgaaaagcaatattttaggcttcaaaatttagccagggtggtgcttagaatgaatgggtagggagtttaggtagaattggtgataagatagagaggagacccaagggattcaaacaaaggataagtagaaagcaatatgagatcatagaaaagttaaacattaagacaaagaagcccagaagagacagaaaatactgtataaacagaaatgaacagaagcacaggacaagtcaaataatattaaagttgaaatggaactagaaaatatcaagttcctccaattttgtcattttacagtggggaattagtgcttctagtaagtggtaaatctaggagttttcatagctctcagactgggacctttttggctgttgcagagggcagagggtggaggatatacgctagccacaggaactatgcttggaggccagaaagaaggagtgaatcacttcaataagagttgaggaggaaatgaaaggccatgtaagagtcacgtggctgtgggaggcttgtggggtttccgtaatggggaagtaggcagagggcaaggtttctgctgggaggaagggatgacagagctgtgtggggtgggaacatgacactggagttctgaggaaatctctctctctctcggggtgcaaaagtcacccactagagtggtcttagggaagtgacaggcaagaagagcccaggcgaaggcaaacacaacaacgtctgtggtctgagcaaaaggacaagaaaagggaagttggggaaggacaggaagagcactgggctccctcgggacaacccagcccctctgttcaggctgggttctaatcatagggtgtcctacccacttcagattattctcttaggattttgtgtgttcagagagcttaataaccacttactcctctccccattgcattttatatcaatttcagattcttgcatctatatttataagtattcaccagtgtaaagagtctgccagtttcccttcttacactacctccacccttcctttttagggatagatgtacataaactcttgtgtataaaattgtcatacttaggagtctgggttatttttcatgaaagcaatttgaatattttaggctgatagacattctaaggatcacgttttccatggcagctctggggcagagcactgcatggacatggagggatatggactgtgacacaccggcagggactgtggctgtgatctctgtcccacaggatgaaagcaggactacatatcagtcagatcagagaggaaaaaccaggtggagggagagaaatgacatggtgactgtgtggagcaaaggagagggaagttttcggagaacacacatttctaggtatctatttgaatgcgtattgccatgatgggggtgagggtgttacttactcttctaggcaccctgagggggaatttcccataaatactttttgtttttttttcctcaccccttaatctttcctctgaaaacagactgatttatctcactgataaattcaggagggaaactgggcaagtatatatcagcacctcctactgccctttacctctctgggcaagttgctccccaaaggctcagtcttgctctcctcctccaggccctggctctcacaggaacagctgctcaggtggggaacttagggttttatcagcattgcctgaaataagtgagtgtattagtcttcttgggctgtgctaacacaaggcaatcagctgggtggcttaaacaacaggaagttattctctcacagctctagaggctagaagttcaaagtctccatctggaagactcagtttctagtgaaggttctcttcctggtttatacagacaactgccttctcactatgagctcatatggcctttccatggtgggtgcacacacacacacacacacagagacagagagcgacagggagagacagagagacagagaaggaaagtggaagggcactccagtgtctcttcttataaggacactaagcctattggatcagggtcttaccgtttagacatgagattgacattacttcttagaggccccatttccaaatataatcacactgagagttagggattcaacatgtgattttttttttctttgggagggtacacaaactttcagtccataacaatgagaaggccactccatttagggtccagtatgagcaagatcacttggattacacacttaaagcaaattatctagtggcactatcagaagtcaagattgaccattagatccatctatctcatgttattttcaacttgtttgcttctgaattcaagagggcaaaaaggatgatacttactgggttttataatattatccaatatagctaggttatgttgcagttataaataaactttagaacttggaggcttaacacaatgaagtttatttctcatgcagttaaactgcatgtagatcaggctgctcttctccatctcatagtcttgccatttagaaatttgactgccaagttggtaccacagggtaagatgatcagatgtgatattttcagtggtcaggactggagctggctaatgcttccaacaccacacattccattgcccagaactcagtccatgaccttaacccaactattaggaaataggacagttgcatctttcctgtaggaacatggaaaaaaatgtgaataaacacataatgtggtatctactattgttttctaaaaccacaaaccaagtatttgccattaacagcacaagatgagcaaagataacacctagtgcttatgaatgatttgggtaggatttgaactgggtattgctcttaaagatattatagttcatagaaattgccacatatctgtggccatgataggaagagctcattgagaatgacttctggcctcactattaacgttgacaaacctaagaaatagtgtatatatcaaagtatcatgttgtacacagtaaatacatagaataaatataaatttaaaacagaaacaatgttacaaatgggattttcctttttaaggcacggtcttgctctgttacccaggctggagtacagtgacatgaacatggctcactacagccctgactttcagggctcaaaagatcttcctgcctctgcctcccatttagctgggactacaggcatgtgccaccacatttggccaactttttaatttttttatcgagatgaggtctaactttgttgcccaggctggtctcaaactcctgggctcaagtgatcttcccaccttggtctcccaaagtgctgggattacaggcatgagtcattgtgcctagcacaaattcatgcccagattttaagtgactgctccaatgcattcagtgaataattaatatgatttaagaaacactataataataaatgtatttcctggatctcaaaaaaactgtgttcatgtatttacacatattaaccagccagggtcactgataagtcactgaatacttcatataaactacttaaatcaaacattgacaaatgctgccctttttatacaatgtgttttggtcagatcacaaaacaacttaaatttggatttttgtggtctccccatgtatgagtcctcttgaaaagctgcatgttctgactatcacaatcgtataccattccacactgtacagaattttcagtatttagttgagatgcatgctgttcttccaggacttcaaattatgaacttcccagggggttagtcagtcaaaatgctgctctagagaatatagttctaagtagatttgtatctgggattctatacattgaaattaaaatcacaatgcattttaattttttgtttaaacatctgcagtttaaaactctttgatagaagagattacattgttaacctctgtgtgtttatatgcctagtaagacactggtatcaaccatgtactatgtaattttttagtgaatgactatacagattaaatgcagtagaagggcttctttatatttgttatggtcctatgcattctgaatggctgcaatttaatctctaaggataatgtagagtggactgtagtttatataattgtgcagggaaatcttggaagatgtaaaaagcaacgtctccaatttataaaagaatcagtgagaatgaggaaataagaaagatgggtgaaaagaggtgaaattggaaagagaaaactttgaggacattcaaaagggggaaagaaagaatggagtgatgagaagaggggacactatggagagtggaggcaaagttaagtggagaaactggaagaactgtgatctattaaaacaagcaggaactgaaaaaatgacatggtttctcctttttatcttacaaatgacatgcaaaatccagagggacaaaatgacctttctttcaatgccatacaccactttgtctaaggcagttgaggaagggaagtagatataatggagcttagtggcagagcagctggaatcctgagagaagagaataactttagttcagagcaggtggggaaatgagagattgagtaggaggaccaaggttgagggaagcagatcttcttagttgctgtcagcggctgatggggaagattgttggtagaaggaagtcagaatataggtacagagggataagtttgctaagaacagagatcagcaaacagcttttctgagagaaagaaacatctgcaaatgacatgctgtttctgcagtttctgctgctagctcttcttctcccaggtggtgacaatgcagacggtaagaacatcgctgtcagctgcaaggttacatgtatctgggtagagtgtgctgggctttcccgagatggatcaatagagatgccagaatgcttgccatctgagcatacctgtctccaggtccagtttactcttttggaggatggtggcagagcatgggctctgtgtaaggaaaatgttatagctaaagtagtcattaatgtttctctgtggtaactggttcaatttccatttcctctccattttccgttaaaacaatcagggcccaggaaaatgatgtggtttctcctttttatgttacaaatgacacgcaaggtccagagggatgaagcaacctttcaatgccacgcaccactttgtctaaggcagttgaggaagggaagtagataagatggagcttagtgggagagcagctaaaatcctaagggaagagagtaactttagttcagagcagatggggaaatgagagattgagtaggaggcctaaggttaagggaagcagattgtcttagttgctgtcagtggctgatggggaagattgtctgttggtagaaggaagtcagaatatagatgtagagggataagtttgctaagaacagagatcagcaaatggcttttctgagagaaggaaacaactgcaaatgacatgctgtttctgtagtttccgctgctagttcttctcctccccggtggtgacaatgcagatagtatgaacatctctgtcagctgcaaggttacatgtatctgggttgagcatgatgggctttcccaagatggatcaatgaagaagccagaatggttgccatttgagcatacctctctccaggtccagtttactcttttagaggatggtggcagagcatggggctctgtgtaaggaaaatggtatagctaaagtagtcattaatgtttctctgtggtaactggttcaccttccattttctctccattttccttgcctctctttttttttttttccttacactacttgcccttcttccaccaaaagcatcccaggaacacgtctccttccatgtcatccagatcttctcatttgtcaaccaatcctgggcacgaggtcagggctcaggatggctggacgagttgcagactcatggctgggacagtgaatcaggcacaataattttcctgcataactggtccaagggcaacttcagcaatgaagagttgtcagacctagagttgttatttcgtttctacctctttggattaactcgggagattcaagaccatgcaagtcaagattactcgaaatgtaagttcaatcatctaaattatgggagttctttagaatgttctatttcagggaaatattctctactaatttttgggccatttcccattatcccatcccaccataaaattctgcatagatgaaccttttaagggatattcctgatgttgactccctcaaatttcctaggctcttgctatttttggaattttttttcttgtgctttgttatcttccacctgtggtctccctctgtatcctacaatccagagatacacagctgtcccactctgtgcttgactcccttaaaataaaatctattcccccattcaatttcttcaaagatctatgtccacagttcaatgaccattatgaccaatttcatgcgtcatggaatgctatcctcctacctccctcacaaaggctcccccttcaccctctatcctatattatgtggctcacctatcatttttccacatcttccttggccactttttcttgttaattcaaggcatttatctcttattgtccccttccctatccccagcaatttttctctcttgtttctgatcaaatatgtctttggctcactctcacatccatgtaaaactttcttgcttcacttcctgatacagccctaggtttttatactgttgttttcacttttttgtttgaactctttttctcattcctcccttcctccagatccctttgaagtacaggtgaaagcgggctgtgagctgcattctggaaagagcccagaaggcttctttcaggtagctttcaacggattagatttactgagtttccagaatacaacatgggtgccatctccaggctgtggaagtttggcccaaagtgtctgtcatctactcaatcatcagtatgaaggcgtcacagaaacagtgtataatctcataagaagcacttgcccccgatttctcttgggtctcctggatgcagggaagatgtatgtacacaggcaaggtcagtagtttcagccccttcctctaagatttttctattcaagtactgccccttctgttcccacccttcaaactcaggacaagaaagaggacaagaagcaggggggttgctgggtaatagtttcttagaggcaggaaaaagataactgtgcatattcatgtggatgtgtgtatgtggatgtaagttgtgtgtaagtttcttcatcagaacactttttctgctctctgcagtgaggccagaagcctggctgtccagtcgccccagccttgggtctggccagctgttgctggtttgtcatgcctccggcttctacccaaagcctgtttgggtgacatggatgcggaatgaacaggagcaactgggcactaaacatggtgatattcttcctaatgctgatgggacatggtatcttcaggtgatcctggaggtggcatctgaggagcctgctggcctgtcttgtcgagtgagacacagcagtctaggaggccaggacatcatcctctactggggtaagactggaggttggaagtgtaggtaggtggttcttgagcctagaggttaggggagaggaaattttgaggatagcagacctaggtgagggattgtaggaagaaatgtatagggtaatttaaagaatagtggagtaagacctaagggatacatggatactagagatttaaggggaaatgaggaatccttccttgagtaagttttggaatagagtgactgaaataggataactgatgcaactcatccaatgcatatgttaagtaaggaaatcaatagatggaatagaatcagcagtgagtttaaaatggcatccatgtatctttccaatatgtgcaggacaccacttttccatgaattggattgccttggtagtgatagtgcccttggtgattctaatagtccttgtgttatggtttaagaagcactggtgagttttttgtatttcctcctctcctcccagtttcttatttcacattccatttttcactctcttagcttttctctattgactactccaccttatcctcagttaccacctccaacttattcagggtttctcccattcctgttccttcacagctcatatcaggacatcctgtgagactcttccccctgactcccccattgtgttaagaacccagcaacccaggagcctagtacaatatagtgatgccatcccgtcgactctccatttaaattgtttctctttctgcataataaacatttgttaataaaaaccaaattctaatttggaatgtttttcttggaatctccactttttatatagcactcaaccttcaaagcccatttctgatgtcatttcctccaacgatcttccttgacccatactgaacccagagagcccctcaactgttatgtgcatgcaacacttcctaccctgtgttgcagctacttgagtctttcatttcacatttttaagcttttaggaggcaaggatcatgataaatgtgcctttcctgcatatgataggctcagtgaaacacaaaacaaaacatcatatctaagttggattaactgtcatgttgacaatttttctcattatattccttcccaatgttgttgtagcattggaaatattcatgcttctctttttgtttaaatgcttcatacctagcagataaattccactctcctttagctccctcattttctccttaaacatttaaggaaaagtgcagccttgtgccttgatacacttattttctaaacacagagaattacaaatgcaaatagacagagagtgtgcaagttacatgtgatgatcagtggtgcagatatgtcacagaatacttaagaaaaggaacaagagatttggtcctcagggttggagtgggatcaaggcaaccgttgtgctgtatatggaagacaaatttcctaaaggactgtgttatctgagatagccttggctaaactttagtccctttaggtttcattcactaattcaactaatattttttgggtactgagttgagacaggcacatacagcttctaaactggtggtggataaaacaaatgagattgtgtccactctcctgaagttgcaatgtttgcttcctcaatctgtccatgtttcttttctattgttttcatgtttttatttatttcaaaaaattttaaatagattttattgatgtataatttatagtaaaatttgaatctttaagtgtacagttctatttattttgataattatataagcccatgtaaacaaaatcacaatcaagatattaaatgtcataacccctcatacctgtttgtagtcaagcacaccaacaacctggcattcttgcaactactgatatactttatgtcacttaagcttaaacttgtattttctatgattttattaagatggaaataaacatatctacttggtattgccttttatttttcttcagtttagcatatggttgtaagtaatccatgctgctcagtgtattagtaaccattccttttcattgctgaatagaattccatgtatgaatataactttttttggaggaggaggtggcttcccagatctggcagggcctttgtctccttgtgcccactcacccagcttgggtcctgcttccattgctcatttccttggcaccaggaggccctaggtgactctcctgcacccttagtcaccctgtgacctgcagatacagagagatcaatagagaggattctaggacacccaggaaactgggaaataaagccaaaaatatataaaattaaacttatttttgggagaagagacaagatggctgactagatgcagccaggaagagcttctcccaccaagagacaagaccatcaagaagagcagtatactctgagaagatcattggaaagaaggcattgagagtggatggagggagaatgcagaccctgggctgaaggggggaagaagctgcgaaccctacacagggttgccaaacactaggacttattcttggtcctcagtggcttctagggaaggggttggttaaataggcatgaagtggcccactctatccatgaacctctggaatcctagctgcaggagacccccacgacccacacagacatttgagttggcagggaaagctacttgcagagttggcagggacaggactccagcttctatggaacccagagggtttggtgtgagaacagctacagtggagcatggttagggatgtccattacccgaggattggcatgctcctctaggtggctttgacccctttgttgactatcagacctggacagaatagggctaccatgatatttggatggggctagactgatctgagccccccaccccatgtctgctggcctctcccaggatccctgcctgtctgtacctgcttgcagcacagcctcagatgttcaactgaggcagttctcagagaccactgccataggtctttcactggctgaccccaccaaaccaccaaatagcttctgtcactggcctcacatcagcatgcacctgccttcagcacccacaactgctttgctggcatacatacacagacatcactgccttgctaccacttctgtgcacatatgcatggagacctcaccactgctgcactgtccccactagcctacatgcgtgcatgtaccctgccacactaccatccctggtgtacatgtgcatggaccttctaccctgctgccccaccaccaccagcatgtgagtgagtgtggaccccaccatgccactgccccactgctgccagtacatgcatgtgagagcagacccccaccgccactgcccttatgaaatgcttttctgggacccccccaccctgcccaccataggagtgttgttgctagtggactgggagcaccttggccccctccagtgaagcaggtgctcagtctcaaggggccaaagaacaaggttctgggcctggtcccaggccctcagtcttagagcatatagcccaggagtgctgagctgagccatggccccctgaaatcatcaagaaataaagctagctcactgacccaaacctataccatagtcaaagcctcaagggcatcaaagaatataaaagcaaaaggccccatcaaaaggacagcaactttaaacgtttaaggaacatcagcccacatagatgagaaagaacccgtggaagaactcaggcagctctaaaagtcagagtgtcttcttacttccaaatgaacacactagctccccaacaatctttcctaaccagactaaaatggctgagataatagacatagattttagaatctggatggcaatgaaaatcatgagattcaggagaaagttgaaatggaatccaaagaatttaaggaatccagtaaaataatacaataactgaaagacaaaatagccattttaagaaagaactaagctgatttgacagagctaaaaaattcactaaagaattttataatacaattggaagtattaaaggcagaatagaccacactgagggaagaatctcagagctcaaagaccagttcttctaattaactcagtcacacaaaataaagaaaaaataatacagaaggatgaacaaaatctctgagaaatatgaggttatgtaaagagaccaaacctgtactcactgatgtccctgaaagagagagagagagagtgtgcaaacaattgaaaacatatatgaggatattgtccatgacaattttctcaacttcactatagaggtcaacattcaaattcaagaaattcagagaactcctgagagatactacactagaaaaccatcctcaagatacacaataataagattatccaaggtcaatgtgaaagaaaaaaatactaaaggcaaccagagaggaggagcaggtcacctacaaagggaccccaatcaagctaacagcatacctttcagcagaaaccttagaagccagaagacattgagggcctatattcagcattgttaaagaaaagaaattctgacccaaaatttcatatccagcccaagtaagatttatatgttaaagagaaattcagcaaagcaaatgctaaggaaatttattaccaccagacctgccttataagaagtcattaagggagtgctaaacatggaaataaaagactcttaccagccaccacataaatgcacttaagtaaacagaccagtgacactataaaggaactacacaattaagtctacataacaaccagctaacaataatgacaaaatcaacttctcacttatcaatatttaccttgaaggtaaatgagctaaacaccccaagacacagagtggcaagttgaataaagaaacaagaccctggctcatgcctgtaatcctaacactttgaggctaaggcaggccgattgcttgatcctatgagtttgagaccaacctaggcaacatggaaaaaccccatttctacaaaaaaaccaagaaaattagctaagaatggtggcacatgcctgtagtcccagctactgaggaggctgaggtaggaggatcacccaagcctggggaggtggaggctgcagtaagttgtgatcatgccactgcacttcggcctaagtaacagagtgagaccctgtctcacgttaaaaaagacaaaaaaaaaaaaaataaacaagacccacctgtaagttatcttcaagagacccatctcacatgcaaggacacccataggccaaaactaaaggatggcaaaaaatctgtcaagcatacaggaaagaaaaaagagcaggggttgctatttttatttcagacaaaataaactttaaaccaacaactgtcaaaaaggacatagaaaaacattatttaatgatagagggttgaatttaataagaagacttagctatcctcaatatatatgcacccaacacaggagcacacaggttcataaaacaagtacttagagacccacaaagaagcttatgttatcatacaataatggtggtagacttcaacacccaactgacagcattagatagatcatcaagagagaacactgataaaggtatttggaacttaaactcgacacttgaccaaatggacctaataaacattgacagaacgctccacccaaaaccaaaagaatatacattcttcccatgcctccagctttgttccttttgcttaggattgtcttggcaatgtgggctctttttgatttcatatgaaagatcaggtagtttttccaattctgtgaagaaagtcattggtagcttgatagggatggcattgaatttataaattactttgggcagtatggccattttcacgatattgattcttcctatcgatgagcatggaatgttcttccatttgtttgtgtcctcttttatttcattgagcagtggtttgtagttctccttgaagaggtccttcacatcccttgtaagttggattcctaggtattttattctctttgaagcgattgtgaatgggagttcactcatgatttggctctctgtttgtctgttattggtgtgtaggaaagcttgcgatttttgcacgttgattttgtatcctgagactttgctgaagttgcttatcagtttaaagagattttgggctgaggtgatggggttttctaaatatacaatcatgtcatctgcaaacagggacaatttgacttcctctgttcctaattaaataccctttatttctttctcttgcctgattgccctggccagacttccaatactatgttgaataggagtggtgagagagggcatccttgtcttgtgccagttttcaaagggaatgcttccagttttgcccattcagtatgatattggctgtgggtttgtcataaataggtcttagtattttgagatatgttccatcaatatctagtgtattgggagttttttagcatgaacagctgttgaattttgtcaaaggccttttctgcatctattgagataatgatgtggtttttttctttagttctgtttatgtgatggattacatttattgatttgcatatgttgaaccagccttgcatcccagggatgaagctgatttgatcatggtggataagctttttgatgtgctgctggattttgtttgccagtattttactgaggattttgcatcagtgttcatcggggacattggtctaaaactctcagcatcaggattatgctggcctcaaaaaatgagtaagggaggattccctctttttctattgattggaatagtttcagaaggaatggtaccagctcctctttgtacctttagtagaatttgtctgtgaatccatctggtcctggactttttttggttggcaggctattaattattgcctcaatttcagagcctgttattggtctattcagagattcaacttcttcctggtttactcttgggagggtgtgtgtgtcgaggaatttatccatttcttctagattttctagtttatttgcacagagatgtttatattatcctctgatggtagtttgtatttttgtgggatcagtggtgatatcccctttatcattttttattgtgtctatatgattcttctctcttttcttctttattagtcttgctagtggtctatcaattttgctgatcttttcaaaaagccaggtcctggattcattgatcttttgaagattttttttgtgtctttatctcattcaggtctgctctgatcttagttatttcttgtcttcggctagcttttgaatatgtttgctcttgcttctctagttcttttaatgtgatgttaggtgtcaattttagatctttcctgctttatcttgtgggcatttagtgctataaatttccctctacacactgctttacatgtgtcccagagattctggtacattgtgtctttgttctcattggtttcctagagcatctttatttctgccttcattttgttatttaccaagtagtcattcaggactacagtttccatgtagttgtgtggttttgagtgagtttcttaatcctgagttctaatttgattgcactgtggtccgagagacagtttgttgtgatttcttttctttttacatttgctgaggagtgctttacttccaattttgtggtcaattttagaataagtatgatgtggtgctgagaagaatatatactctgttgatttggggtagagagtaccgtagatgtctattaggtctgcttggtgcagagctgagttcaagtcctggatatccttgttaaccttctgtcttgttgatctgcctaatattgacaatggaatgttaaagtctcccattattattgtgtgggagtataagtctcttcgtaggtctgtaaggactcattatatgaatctgggtgctcctgttttgggtgcatatatatttaggatagttagctcttctttttgaattgattcctttaccattatgtaatggccttctttgtctctctctctctcttttttttttttttttgagacagagtctcactctgtcacccaggctggagtacagtggtgcgatctcagctcactgcaagctcttcctcctgtgttcataccattctcctgcttgagcctcctgagtagctgggactacaggtgcccaccaccatgcccagctaattttttgtatttttaatatagatggggtttcaccatattagccaggatggtctcaattgcctgacttcatgatctgcccacctcagcctcccaaattgctggaattacaggtgtgagccaccgtgcctttgtctcttttgatctttattggttaaagtctgttttatcagagactgggattgcaacccctgctttttttttgctttccatttgcttggtagagtgtcctccatccctttattttgagcctatgtgtgtctctgcacttgagatgggtctcctgaatacagcacactgatgggtcttgactctttatccaatttgtcagtctgtgtcttttaattgtcatatttagctcatttatatttaaggttaatattaagtgtgaatttgattctgtctttatgatgttagctggttattttgctcattagtttatgcagtttcttcatagcatcaatggtctttacaatttggcatgtttttggagtgtctggtactggttgtttctttccatgtttagtgcttccttcaggagctcttgtaaggcaggtctggtggtgacaaaatctcttagcatttgcttgtctgtaaaggattttatttctccttcacttatgatgcttagtttgggtggatatgaaattctcggttgaaaattcttttctttaagaatgttgaatattggcccccactctcttctggctggtagtgtttctgcagagaaatcagctgttagtctcatgggcttccctttgtggaacccgacttttctttctggctgcccttaacgtttttccttcatttcaaccttcgtgagtctgataattatgtgtcttggggttgctcttctcatggagtatctttgtggtgttctctgtatttcctgaatttgaatgttggcctgccttgctaggttggggatgttctcctggataacatcatgagcagtgttttccagcttggttccattctccctgtcactttcaggtacacaaatcaaacatagatttttttcttttcacatagtaccatatatgcagaatacacctgtcttctgcatcaatcaagctggtagttgcagaccggagctcttcctattcagccatcttgggggaagtcgcaattttcaacacattcaaaaaaaccaaaatcataccaaccacactcttgcaccacagcacaacaaaaatagaaatcaagatcaagaagagctctgaaaaactatacaattaaatagcaattaaacaatttctggtcctgaaagacttttgggtaaacaacgaagtcagaaatcaggaaattgttctaacaaatgaaaacaaagatactacatgccagagtctctgagacacagctaaaacagagttaagaggaaagtttatagcactaaatgcccacatcaaaaagttagaaagatctcaaattagtaacataatctcacatctaaaggaactagaaaaataaggaaaaaaaacctaaagctagcagaagacaagaaataaccaaaatcagagctgaactaaataaaactgagaactgaaaactatatagaagataaacaaaaaaattggtaatttgaaggaataaataaaattggtagaccacagactagaataataaaggagaaaaagatgagaaaatccacataaacccaatcagaaatgacaaagggggcattgccaccaacctcatagaaatacaaaaaccactcagatactgttatgaacacctctatgcacgtaaactatgaaacctagaagaaatggaaaaattcttgaaaatatacattcccaaaattgaaccaggaagaaattgaaaccctgaacagatcaataatgagttccagagttgaatcagtaataaaaaggttgccaatcagaaaaagccttggaacagagagatccatggctgaattctaacaggtatataaagaaaagctggtatcaattctttgaaaactattccaaaaattgagggggagtgattattccctaactaattctatgagataagtatcattgtgataccaaaacctggtagagacacaataaaaaaagaaaacattagctaattttcctgaagaatatatatgcaaaaatcctcaacaaaatattagcaaagcaaatccaggatcatatcaaaaagctaatctgctattatcaaatgggcttcatccctgggactcaaggttggttcaacatacagaaatcagtaaatgattcatcacataagcagaactaaaaacaaaaaacacatgattatctcaacagatgcagaacagtcttttgataaaattccacatctcttcaggttaaaaacccttaagaaactaggcattaaaggaacacacctcaaaacaattagaggcatctatgacaaacccacagccaacaacgtactgaataaacaaaagcttgaagcattccctttgaaaactagaacaaggcaatgatgtccactcttaccactgctattcaacataatactggaagtcctagccagagcaatcagggaagagaaaggcattcaaataaaaagagaggaagttaaattatctctttcttcagacaatatgaatctatacctagaaaaccccatagtctctgtgcaaagggttctaaatctgataaacaattttagttaatcttcaaggtataaaatcaatgtacaagaatcagtagcttctctatatatcaataaagtccagaccgagagctaaatcaagaacacaatcacattcactataatcacaaaaagaataaaatgcttaggaatatagctaaccaagaaagtgaaagatctctacaacaagaattataaagcactgctaaaaaatcagagattatacaactgaatgtaaaaatatttcatgctcatggattgaaagaattaatgttgttaaaattatcatattgctgaaagcaattcagagactcagtgctactcctatcaaagtaccaatgacattttttcatagaatttgaaaaaactattctaaaattcatacagaatcaaaaaagagtctggggggtgcaaccaagatggccgaataggaacagctctagtctagagctcccagcatgagcgacacagaagacgggtgatttctgcatttccaactgaggtacccggctcatctcactgaggagtgtcagaaagtgggtgcaggacagtgggtgcagttcacccagcgagagccaaagcaggatgaggcatcgcctcacctgggaagcacaaggggtcagggaattccttttcctaatcaaagaaaggggtgacagatagcaccaggaaaatcgggtcattcccaccctaatactgtgcttttccaatggtcttagcaaagggcacacaaggagattatatcccatgccaggcctggagggtcctatgcccacggagcctcactcattgctagcacagcagtctgagatcaaactgcaaggcagcagagaggctgggggaggggcgcccaccattgcctaggcttgagtaggtaaacaaaggccaggaagctcgaatggggtgtagcccaccgcagctcaaggaggcctgcctgcctctgtagactccatgtctgggggcagggcacagccaaaccaaaggcagcagaatcctctgcagacttaaatgttcctgtctgacagcttggaagagagtagtggttctcccagcatgcagctggagatctgagaatggacagagtgactcctcaagtgggtcactgatgcccaagtagcctaactgggaggcacctcccagtaggggcagactgacacctcacacggcagggtactcctctgagacaaaacttccagaggaataatcggcagcaacatttgctgttcaccaatatctgctgttctgcagcctctgctgctgatacccaggaaaacagggtctggagtagacctccagcaaactccaacagacctgctgctgagggtcctgactgttagaaggaaaactaacaaacagtaaggacatccacaccaaaaccccatctgtatgtcaccatcatcaaagaccaaaggtagataaaaccacaaagatggggaaaaaacagagcagaaaaactggaaactctaaaaatcagagtgcctctcctcctccaagggaacgcagctcctcaccagcagtggaacaaagcaggatggagaatgaccttgacgagttgagagaagaaggcatcagacgatcaaactactccgagctaaaagaggaagttcgaactcatcgcaaagaagttaaaaatcttgaaaaaaaattagatgaatggctaactagaataagcaatgcagagaagtctttaaaggaccggatggagctgaaaacaaaggcaagagaactacatgatgaataacaagcctcagtagccgatgggatcaagtggaagaaagggtatcagtgatggaagatcaaatgaatgaaacgaagtgagaagtttagacgaaaaagaataaaaagaaattaacaaagcctccaagaaatatgggactatgtgaaaagaccaaatctatgtctgattggtgtacctgaaagtgacagggagaatggaaccaagttggaaaacactctgcaggatattatccaggagaacttccccaatctagcaaggcaggccaacattcaaactcaggaaatacagagaatgccacaaagatactcctcaagaagagcaactccaagacacataattgtcagattcaccaaagttgaaatgaaggaaaaaatgttaagggcagccagagagaaaggtggggttacccacaaagggaagcccatcagactaacagctgatctctcagcagaaactctacaagccagaagagagtgggggccaatattcaacattcttaaagaaaagaattttcaaaccagaatttcatatgtagccaaactaagcttcataagtgaaggagaaataaaatagtttacagacaagcaaatgctgagagattttgtcatcaccaggcctgccctacaagagctcccgaaggaagcactaaacatgaaaaggaacaagtggtaccagccactgcaaaaacatgccaaattgtaaagactgtcgaggctaggaagaaactgcatcaactaatgagcaaaataaccacctaacatcataatgacaggatcaaattcacacataacaatattaactttaaatgtaaatgggctaaatgctccaattaaaagacacatactggcaaattggatagagtcaagacccatcagtgtgctgtattcaggaaacccatagcaagtgcagagacacacataggctcaaaataaagggatggaggaagacctaataagcaaatggaaaacaaaaaaaggcagtggttgcaaacctagtctctgataaaacagactttaaaccaacaaagatcaaaagagacaaagaaagccattacataatggtaaagggatcaattcagcaagaagagctaactatcctaaatatttatccacccaatacaggagcacccagattcataaagcaagtgattagaggcctagaaagagacttagactcccacacaataataatgggagattttaacaccccactgtcaacattagacaggtcaacgagacagaaagttaacaaggatatccaggaactgaactcagctctgcaccaagtggacctaatagacatctacagaactctccaccccaaatcaacagaatagacattcttctcagcaccacaccgcacttattccaaaattgaccacatagttggaagtaaaggtctccttagcaaatgtaaaagaacagaaattataacaaactgtctctcagaccacagtgcaatgaaactaggattcagcataaagaaattcactcaaagccgctcaactacatggaaaccgagcaacctgctcctgaatgactactgggtacataatgaaatgaaggcagaaataaagatgttctttgaaaccaacgagaacaaagacacaacataccagaatctctgggacacattcaaggcagtgtgtagagggaaatttatagcactaaatgcccactagagaaagcaggaaagatctaaaattgacaccataacatcacaattaaaagaactagagaagcaagagcaaacacattcaaaagctagcagaaggcaagaaataactaagatcagagctgaactgaaggaaataagagacacaaaaacccttcaaaaaatcaatgaatccaggagctgcttttttgaaaagatcaacaaaattgacagactgctagcaagactaatacagaagaaaagagagcagaatcaaatagacgcaataaaagatgataaaggggatatcaccactgatcccacagaaatacagactaccatcagagaataatactataaatacctctacgcaaataaactagaaagtctagaagaaatggataaattcctcgacacgtacacccccccaagactaaatcaggaagaatttgaatccctgaatagaccaataacaggctctgaaattgaggcaataattaatagcttaccaatcaaaaaaagtccaggacccaatggattcactgccaaattctaccagaagtacaaggaggagctggtaccattccttctgaaattattccaatcaatagaaaaagagggaatcctacctaactcattttatgaggccagcattatcctgataccaaaacctggcagagacataacaaaaaaagacaattttagaccaatatccctgacgaacatcgatgcagaaattctcaataaaatactggcaaaccgaattcagcagcatatcaaaaaccttatccaccctgataaagtgggcttcatcttgaaaagtcctttgacaaaattcaacaacccttcatgctaaaaactctcaataaattaggtattgatgggatgtatctcaaaataataagagctatttatgacaaacccacaggcaatatcatactgaatgggcaaaacctggaagcattcccttggaaaactggcacaagacagggatgccctctctcaccactcctattcaacatagtgttggaagttctggccagggcaattaggcaggagaaggaaataaagggtattgaattaggaaaagaggaagtcaaattgtccctgtttgcagatgacatgattgtatatctagaaaaccccatcgtctcagccccaaatctccttaagctgataggcaacttcagcaaagtctcaggatacaaagtcaatgtgcaaaaatcacaagcattcttacacaccagtaacagacagagagccaaaccatgagtgaactcccattcacaattgcttcaaaaagaataaaatacctaggaatccaacttacaagggatgtgaaggacctcttcaaggagaactacaaaccactgctcagtgaaataaaagaagatacaaacaaatggaagaacattccatgctcatgggtaggaagaatcaatatcatgaaaatggccatactgcccaaggtaatttgtagattcaatgccatctccatcaagttaccaatgactttcttcacagaattggaaaaaactactttaaagttcatatggaaccaaaaagagcccgcattgccaagtcaatcctaagccaaaagaataaagctggaggcatcacactacctgacttcaaactatactacaaggctacagtaaccaaaacagcatggtactggtaccaaaacagagatatagaacaatggaacagaacagagccctcagaaataacgccacatatctataaccatctgatctttgacaaagctgacaaaaaccagcaatggggaaaggattccctatttaataaatggtgctgggaaaactggttagccatatgtagaaagctgaaactggatcccttccttacaccttatacaaaaatcaattcaagatggattaaagacttacatgttagacctgaaaccgtaaaaaccctagaagaaaacctaggcaataccattcaggacataggcatgggcaaggacttcatgtctaaaacaccaaaagcaatggcaacaaaagccaaaattgacaaatgggatctaattaaactaaagagcttctgcacagcaaaagaaactaccatcagagtgaacaggcaacctacagaatgggagaaaacttttgcaacctactcatctgacaaagggctaatatccagaatctactatgaactcaaacaaatttacaagaaaaaaacaaacaaccccatcaaaaagtgggcaaaggatatgaacagacacttctcaaaagaagacctttatgcagccaaaggacacatgaaaaaacggtcatcatcactggccatcagagaaatgcaaatcaaaaccacaatgagataccatctcacagcctttagaatggcgatcattaaaaggtcaggaaacaagaggtgtggagaggatgtggagaaataggaacacttttacactgttggtgggactgtaaactagttcagccattgtggaggtcagtgtggcgattcctcagggatctagaactagaaataccatttgacccagccatcccattactggttatatacccaaaggattataaatcatgctgctataaagacacatgcacacatatgtttattgaggcactattcacaatagcaaagacttggaaccaacccaaatgtctaacaatgatagactggattaagaaaatgtggcacatatacaccatggaatactatgcagccataaaaaatgatgagttcatgtcctttgtagggacatggatgaaactggaaaccacctttctcagcaaactattgcaaggacaaaaaacaaaacactgcatgttctcactcaccggtgggaattgaacaatgagaacacatggacacaggaaggggaatatcacataccggggcctgttgtggggtggggggaagggggagggatagcattaggagatatacctaatattaaataacgagtcaatggctgcagcacaacaacatggcacatgtatacatatgtaactaacctgcacattgtgcacatataccctaaaacttaacgtataataataaaaaaaaaagagtctgaacacccaaagcaatcttaagcaaaaagaacaaagctggaagcaccacactatctgacttcaaactatagtacaaggctacagtaaccaaaacagcatggtactagtagaaaaacaaacacatagataaatgaacaggttagagaacccagaaataaatctgcacaccttacaaccatctgatctttgacaatgtcaacagtaagaagcaatggggtgaagacttcctattcaataaatggtgttgtgctaactggctagccatatgcagaatattgaaaatttacatcttcctttcaccacatacaaaagttaattcaagatggattaaagacttaaatgtaaaacctaaaactacagaaatcttggaagaaaacctgtgaaattccattctggacataggccctggcaaggatttcatgatgaagacttcaaaagaaactgcaatgtaaacaaaaattaaatggaacttaattaaactaaagagtttctgcacagcaaacaaaattgtcaacagaataaacagacaacttacagaatggaagaaaatacttgcaaactatgtgtatgacaaagatctaatacccagcatctataaggaacttaaattaacaagcaaaaaaccacaacctccttaaaaaataggcaaagaatatcaacagacacttctcaaaagaagacatatacatagccaacaagcatatttaaaaattctcaacatcactaatcatcagagaaatgcaaatcaaaaccacaatgagataccatctcacaccagtcagaatggctattattaaaatgtcaaaaaagtaacagattctagcaaggttgcagagaaaagagaatacttacactctgctggtgggaatgtaaattagttcagccactgtgaagggcagtttggtgatttctcaaagaacttaaaacagaacttaaaaactacctatgggtactatgctcattacctgtgtgacgaaacattctgtacaccaagcccccacaacatgcaatttacccatgtaacaaaccttcaaatgtagctgccgaacctaaaataagaggtggaaaaaaataatttttaaaaatctatctggtctttaatgggtatgtagtatatttttagtttgagactttttaaataaaatctatttttgtttgttttgtttttaatgtgttttttgtttgtttgcttttgtttagttacctgcttagactaattcaaactggggtgaagtgatatctcattgtggttttgatttgcatttccctactgattagtcatgttgagcatattatatttacctattggtcattcgtatgtcttcttttcagagttgtctaatcaggtcttttgccctttaaaatttaaataacttacttttttgccattgagctctttgagttccttatatattctggatcttaatccctttggatacgtagtttgcagatattttctaccattctgtagcttatttcctttgctgtgcagagcttgttagttagataaaatttcatttgtctattttgttttgtattaccagtgcttttgaggtcttatcgaaaatcttttcccagaccaacatgaagcattcccctatattttcttcttgtagtttcacaattttgggtcttacatttaagtctttaacccatcttcatttgattttgtatatggtgagatatagggctctagttttgttcttctgcatatggatattcagttttcccagcactatttattgaagagactgttctgtccccaatgtgtgtcttggcacttttgttaaaaataggttggctgtaaaggcatggatttatttctgggttatatattctgttccgctgatctatgtatctgtttttataccagtaccatgctgttttggttactatagttttgtagtatattttgaaatcaggtagtgtgatttctacagatttgccctttttgctcaagattactttggcttttggggtctttgatggttcaatattaatttagaatcatcttttctatttctatgaagtatgtcattggtattttgatagagattacattgaatctgtagattgctttgggtagtatgggcattttaacaatgccaattatttcaatctgtgaacataaaatacgatatgatttggccctgtgtccccacccaaatctcatcttgtagccaccataattcccatatgttgtgggagggacctagtgggagataattgaatcatggggacaagtctttcctgtgctgttctcatgttagtgaatgagtctcatgagatatgatagttttaaaaacagtagtttccatgcacaaactctcttctctcttgctgctgctgtgaaagaagtgccttttaccttctgccatgactgtgaggccaccccagctacgtggaactgtgagtccaaataaacctctttcttttgtaaattatctagtctcagatatgtctttattagcaggacgaaaatgaactagtacaaaatacctttccaataatttttgtccttttcaatttctttcatcagtgttttatagatttttttgtagagtttttacctcctttgttacatttattcttaagtatcttatattttgtagctattgtacatggaattgttttcttgatttatttttcagatattttgatgttggtgaaggaaatattactgctttttgtatatgcatttaatatcctgaaattttactgaatttattagtgctcatggctttttgatgaagtaattagtattttctgtatataaagttgtgatctccacaaacagggataatttgacttccttctttttaattgtatgccccgtatttctttctcttgcctaattaccctggctaggagttccagtacaatgttgactagaagtagtgaaagtggacatctttgcttagttccagattttagagaaatttctttcatcttttccccattttgttttatattagctgtgggtttctcatatgtggcttttattgtgtttaggtacgttccttatatacctaatttattgataacttttatcatagtgggatgttgaattctatcaaatgctttctctgtttctattgaaatgattacgtgcttttgtcctttattctattaatgtgatgcagtacatttattaatctgaatactttgaatcatccttgcatccctggaatgaatgccacttgattttaaaggctggtcttttaagtattctgttaaatttagtttgctagcattttcttgaggatttttgcatgcgtttcattagggatattggcctgtagtttttcctattttattttatttttttgagaaagtgtctcactctgtcacccaagctggagtgcagtggcacaatcacatctcaccacagcctcaacctcctgggcttgggtgatcctccaacctcagcctcctgagtagctaggactacaggcgcccaccaccatttttggctaatatttgtactttttttagagacaaggtttcatcatgttgacaggctggtcttgaactcctgggctcaagtgatccaccctccttggcttcccaaagtgctaggattacaggcatgagcaaccacttctgtcctgtaattttcttttttgtttttgttgtgtcatctagtttttatatcgggctaatgaatgctagtctcaaataataagtctggacaaatttactctcattcagtttttttgggggcagtttgagaagaacaggcattagttatttttaaaatatttggtagaattcagcagtgaggtcatcagctcctgtgctttctttaatgggagactttaaactattgtttcaatcttgttactcattattggcctgtttgaattttctgtttcttcatgattcaatcttgtggttgtatgagtccaggcatttacctatttcttctaagttttacaatttgttttcatatagttgttcataatggtctcttatggtcctttgtatttatattatattatttgtaaagtctccttttatatctatgattttatttttttgactcctctttttttcttaatttgtctagttaaaagtttgtcaggttttgtttttgatttttgagacagagtctagctccattgcccaggctggagtgcagtggcacgatctctgctcactgcaacctctgcctcccaggttcaagtgattctcctgattcagcctccctagtggctggaattacaggggcctgccaccacaatcagctaatttttgtatttttagtagagatggggtttcaccatgttgctcaggctggtgttgaactcttaacaagtgatccaccctcctcggcctcccaaagtgctgggattaagtggtgagccaccgtgcctggccaagtgttttatattttgaaaaaacgaagtcttctttgttattcttttgtgttgttttttaaggctctatttaatttatttctgtttttacatttatcgttgcttctcttctactagttttggcttgatgttgttcttgtttttctagttccttgagcaacatgaggttatctgaaatctttctactttttaaatgtaagcctttattgctgtagacttccctcttagaaatgcttttgctgtattccataggttttgttttgttttttttatttttttatttttttattattatactttaagttttagggtaatgtgcacattgtgcatgttagttacgtatgtatacatgtgccatgctggtgcgcggcacccactaactcgtcatctagcattaggtatatctcccaatgctatccctaccccctccccccaccccacaacagtccccagagtgtgatgttccccttcctgtgtccatgtgatctctttgttcaattcccacctatgagtgagaatatgcggtgtttggttttttgttcttgcgatagtttactgagaatgatgatttccaatttcatccatgtccctacaaaggacatgaactcatcattttttatggctgcatagtattccatggtgtatatatgccacattttcttaatccagtctatcattgttggacatttgggttggttccaagtctttgctattgtgaataatgccgcaataaacacacgtgtgcatgtgtctttatagcagcatgatttataatcctttgggtatatactcagtaatgggatggctgggtcaaatggtatttctagttctagatccctgaggaatcgccacactgacttccacaatggctgaactagtttacagtcccatcaacagtgtaaaagtgttcctatttctccacatcctccccagcacctgttgtttcctgactttttaatgattgccattctaactggtgtgagatggtatctcattgtggttttgatttgcatttctctgatggccagtgatgatgagcattttttaatgtgttttttggctgcataaaggtcttcttttgagaagtgtctgttcatgtcctttgcccactttttgatggggttgtttgtttttttcttgtaaatttgtttgagttcattgtagattctcgatattagccctttgtcagatgagtaggttgcaaaaattttctcccattttgtaggttgcctgttcactctgatggtagtttcttttgctgtgcagaagctctttagtttaattagatcccatttgtcaattttggcttttgttgccattgcttttggtgttttagacatgaagtccttgcccatgcctatgtcctgaatggtattgcctaggttttcttgtagggtttttatggttttagctctaacgtttaagtctttaatccatcttgaattaatttttgtatgaggtgtaaggaagggatccagtttcagctttctacatatggctagccaattatcccagcaccatttattaaatggggaatcctttccccattgctggtttttctcaggtttgtcaaagatcagatagttgtagatatgtggcattaattttgagggctctgttctgttccattgatctatatctctgttttggttccagtaccatgctgttttggttactgtagccttgtagtatagtttgaagtcaggtagtgtgatgcctccagctttgttcttttggcttacgattgacttggcaatgcgggctcttttttggttccatatgaactaaaaatagttttttccaattctgtgaagaaaggcattggtagcttgatggggatggcattgaatctgtaaattaccttgggcagtatggccattttcatgatattgattcttcctacccatgagcatggaatgttcttccatttgtttttatcctcttttatttcattgagcagtggtttgtagttctccttgaagaggtccttcacatcccttgtaagttggattcctaggtattttattctctttaaagcaattgtgaatgggatttcactcatgatttggctctctgtttgtctgttgttggtgtataagaatgcttgtgatttttgtacattgattttgtatcctgagattttgctgaagttgcttatctgcttaaggagattttgggctgagacaatggggttttctagatatacaatcatgtcgtctgcaaacagggacaatttgacttcctcttttcctaattgaataccctttatttccttctcctgcctaattgccctggccagaacttccaacactatgttgaataggagtggtgagagagggcatccttgtcttgtgccagttttcaaagtgaatacttccagtttttgcccattcagtatgatattggctgtgggtttgtcatagatagctcttattattttgaaatacgtcccatcaatacctaatttattgagagttttgagcatgaagggttgttgaattttgtcaaaggtcttttctgcatctattgagataatcatgtggtttttgtctttggctctgtttatatgctggattacatttattgatttgcgtatattgaaccagccttgcatcccagggatgaagcccacttgatcatggtggataagctttttgatgtgctgctggatttggtttggcagtattttattgaggatttttgcatcaatgttcatcaaggatattgattggtctaaaattctcttttttggttgtgtctctgcccggctttggtatcagaatgatgctggcctcataaaatgagttagggaggattccctctttttctattgattggaatagtttcagaaggaatggtaccagttcctccttgtacctctggtagaattcggctgtgaatccatctggtcctggactcttttttgttggtaaggtattgattattgccacaatttcatatcctgttattggtctattcagagattcaacttcttcctggtttagtcttgggagagtgtatgtgtcgaggaatttatccatttcttcttgattttctagtttatttgcgtagaggtgtttgtagtattctctgatggtagtttgtatttccgtgggatcggtggtgatatcccctttatcatttttttattgtgtctatttgattcttctctctttttttctttattagtcttgctagtggtctatctattttgttgatcctttcaaaaaaccagctcctggattcattaattttttgaagggtttttgtgtctctatttccttcagttctgctctgattttagttatctcttgccttctgctagcttttgaatgtgtttgctcttgcttttctagttcttttaactgtgatgttaggatgtcaattttggatgtttcctgctttctcttgtgggcatttagtgctataaatttccctctacacactgctttgaatgcgtcccagagattctggtatgttgtgtgtttgttcttgttcatttcaaagaacatctttatttctgccttcatttcattatgtacccagtagtcattcagaagcaggttgttcagtttccatgtagttgtgcggttttgagtgagattcttaattctgagttctaatttgattgcactgtggtctgagagatagtttgttataatttctgttcctttacatttgctgaggagagctttactgccaactatgtgatcaattttggaataggtgtggtgtggtgctgaaaaaaatgtatattctgttgatttggggtggagagttctgtagatgtctattaggtccgcttggggcagagctgagttcaattcctgggtatccttgttgactttctgtcttgttgatctgtctaatcttgacagtggggtgttaaagtctcccattattaatgtgtgggagtctaagtctctttgtaggtcactcaggacttgctttatggatctgggtgctcctgtattgggtgcatatatatttaggatagttaactcttcttgttgaattgatccctttaccattctgtaatggtcttctttgtctcttttgatctttattagtttaaagtcggttttatcagagactaggattgcaatccctgcctttttttgttttccatttgcttggtagatcttcctccatccttttatttttagcctgtgtgtgtctctgcacgtgagatgggtttcctgaatacagcacactgatgggtcttgactctttatccaatttgccagtctgtgtcttctaattggagcatttagtccatttacatttaaagttaatattgttatgtgtgaatttgatcctgtcattatgatgttaggtggttattttgttcattagttgatgcagtttcttcctagtcttgatggtctttacattttggcatgattttgcagcagcttgtaccggttgttcctttccatgtttagcgcttccttcaggagctcttttagggcaggcctgttggtgacaaaatctttcagcatttgcttgtctgtaaggtattttatttctccttcacttatgaagcttagtttggctggatatttaattctgggttgaaaattcttttctttaagaatgttgaatattggcccccactctcttctggcttgtagggtttctgccaagagatccgctgttagtctgatgggcttccctttgagggtaacccgacctttctctctggctgcccttaacattttttccttcatttcaactttggtgaatctgacaattatgtgtcttggagttgctcttctcgaggagtatctttgtggtgttctctgtatttcctgaatctgaacgttggcctgccttgctagattggggaagttctcctggataatatcctgcagagtgttttccaacttggttccattctccccgtcactttcaggtacaccaatcagacgtagatttggtcttttcacatagtcccatatttcttggaggctttgttgatttctttttattcttttttctctaaacttcccttctcacttcatttcattcatttcatcttccatcaatgataccctttcttccagttgatcgcatcagctcctgaggcttctgcattcttcacgtagttcttgagccttggttgtcagctccatcactcctttaagcacttctctgtgttggttattctagttatacattcttctaaatttttttccaagttttcaacttctttgcctttggtttgaatgtcctcctgtagctcagagtaatttgatcgtctgaagccttcttctctcagctcgtcaaagtcgttctctgtccagctttgttccattgctggtgaggaactgcgttcctttggaggaggagaggcgctctgctttttagagtttccagtttttctgttctgttttttccccatctttgtggttttatctattttggtctttgatgatggtgatgtacagatgagtttttgatgtggatgtcctttctgtttgttagttttccttctaacagacaggaccctcagctgcaggtctgttggagtaccctgccgtgtgaggtgtcagtgtgcccctgctggggggtgcctcccagttaggctgctcgggagtcaggggtcagggacccgcttgaggaggcagtctgcccgttctcagatctccagctgcgtactgggagaaccactgctctcttcaaagctgtcagacagggacatttaagtctgcagaggttacagctttttgtttgtctgtgccctgcccccagaggtggagcctacagaggcaggcaggcctccttgagctgtggtgggctccacccagttcgagcttcctggctgctttgtttacctaagcaagcctgggcaatggtgggcgcccctgcctcagcctcactgccgccttgcagtttgatctcagactgcggtgctagcaatcagctagactctgtgggcgtaggaccctccgaagcaggtgtgggatataatctcatggtgtgccgttttttaagtccgttggaaaagcgcagtattcgggtgggagtgacccgattttccaggtgccgtccgtcacccctttctttgactaggaaagggaactccctgaccccttgcacttcccgagtgaggcaatgcctcaccccgcttcagctggcacacggtgtgcgcacccactgacctgcacccactgtctggcactccctagtgagacgaacccggtacctcagatggaaatgcagaaatcacccatcttctgcgtcactcaggctgggagctgtagaccggagctcttcctatttggccatcttggctcctccccctccataggtttttgtgtgttgtgttcccatttccattttttcaagaaatattgaaattttctttttgatttttttcactgaaccactggttgttcagctgtatgttgtctaatttccatgtatttgtagtttccaaagttcctcctcttattgatttctagttctataccattattgttgaaaaaaacattttttgatttttaattttttaatatgttaagacttttattgcagcttaacataagatatatcctggagaatgttccatgtactgtggagaagaatgtatattctgcaactactggatagaatgttctttaagcatctgttaggttcatttggtcaggagttcagtttaactctcattttttgttgatattctgtctggattatctgtttatcactgaagataagatgttcaagtgttatactattgttgtattgtacgctatatctccctgtaggcctattagcatttgctttatatatttgtatgttcaattgttgggtacatgcatatttataattgttatattctctttctgtatttacccctgtatcagtttttaattagcatatttgtctttttttacagtttttttggcttaaagtctattatgtctgatataagtatagctacttctgctccattttgctctccatttgcatggaatatctttttctatcccttcactttcagtctatttgtgttcttctgcgtaaagtgaggctcttataggcagcatatagttgagtctttaaaaaaatcaatttagctactttatgttgtttaataggaaaatttaatccatttacattcaaggtaactaccaataggtaaggaggtactactgacattttgttacttctttcctagttattttgtagatccttccttcttttctaccactcttgctatatccttttgtggttaagtgatattctccaacattatgctttgattcactgctgtttatctttagtgtatctattataggtttttactttgtggttatcatgaggctttgaaatacatattttgtttacatcaagttgttttattttattttaaatttcctacttttattttaagtccaggggtacatgtgcaggatgtataggcttattacataggaaaacgtgtgccatggtggtttactgcacagatcatcccattacccaggtattaaccccagcatccattagctattcttcctgatcttctccctcctctcatccaccacactcaggccccagtgtgtgttgctccccaccatgtgtccatgtataacaagttattttaagctaataggaacttaattttaatcagaaaagaaaggaaacaaaaatattgtacactctaactcctttcttctcaagtattttgaattttcatgtcccaatttacatcttttgtatggtttatcttttaataaagtattttagttatcatttttaagagctttgtatttttgtattcatactaaagttataagtggtttacataccataattacaatgttagatatcccaaatttatctgtattcctacatttattcatttatttaaaggcagggtcttactctatcacccaggctggagtgctgtgactcaatcttggctcactgcaacctctgcctcctaggctcaaggaattatcctatttcagcctcttgaatagctgggactacagacacaagccaccatggctggctatttattttttttctgtttttttttttttgtgtgtgtgtgttttctcgtaagatgcggttttgccatgttgcccaggctgaactcaaatccttgggttttttagtcaatccacttgcctcagctttctaaagtggtggggttacagccatgagctaccatgcctggcctgtatacttacctttaccagtgagttttataccttttgatattttcttgttacacattagcattttttttctttcaagttgaagaacttcctttgttatttcttgtaagacagatctggtggtgttgcatttacttggcttttgtttgtctggaagagcctttatctctccttcatttctgaaggacatatttgccagttactttattcttggttggcattctttttctttcagcattctgaatatattgccctacactttttggcctgcaaggtttctgctgaaaagtctgctgccaggcatatcaacattcccttacatgttatttgcttcttttctattactgcttttagaatgctctgtttttatttcgagagtcaaagggtatgtcttgaggtagttccatttgtgttgaatatgattgatggcctttgaccttcctttacttgagcatttacatctttttccaggattgtaaaattttcttttatgatttctgtgaataagccgtctatttccttgtctttttctgtaccctcttgaactccaatgacccaagtatttgctctttcaatagtatcccatagactccatgaacttcacttgtttctttttttttattctttttcccactttggccatatttcaaatagtctatcttagatttcactgatttttttttcttctgcttgattaattctgctgttaatgctcgtattgcatttcttatttcattaattgtatcttttagctctaggatttctgttagatttttcattactttagtccctgttaaacttctctgataaatttctgaactgcttttctctgtgttttactgaaattcactaagcttccttaacaagctattctgaattccttgagagataatacttctgcatcactttagggtttgtcactggcatcgtgttttgtctgtttggtgaggtcaaaattccctggttgttcttgatgcttgtgtatgtacattgatgtatttgctctgaaaaaaggtatttatttcagtcttcacagtctggctttgtttgtgcctgtttttcttcactgggcctctccagaatttaagcaaacctaatgttagatcgcatgactgctgcagccatttcagcactagaggaaactctaagcctaggttcactgtgagtctcatgagggctgtcaggttggcatggagcccttatgtagaaggacgtggtgaagacccaaggagggtgcccaagctgtgtgaggaagctggccagggacctaagtccaggagcctgtctcattggctcagatgtgggaggagctggagctgagactgggtcccctcaggatctgctttgggacaaaggctggtggtcccaccaaggagtttcagaaaaatgggtgattgcctggctatgagataagggtaagtctttctctaggtatccttgtgtgagcagtaataaactcagatcacagctgagtgggtctggagccaaattacagggtaagtttcagattcactgccaacatttaacggacagaagagattttctgccaagttattagtgtgcatgattcctcctagacacattggtagatgtttttggttcaagactcaagacaaaattggagtgtagccaagccttttgttaacaaggatgtctccaagtttgaaacctgcagtatcattggtgggtctgctatccaggtgctggtctgcaatcccaaagctgcctacctagggccttgattccactgaattttcacagattactccctgattcctgaggcttctacagagagacttttgtctgtggatgagtagataattcttggagttgggggatatgagtgggttacctcctattccaccatctttctgacatagtgtttgtatttctattctgactataacatatatagtgggattagatgaatacataattacattgattcatcggaaaccaagattttccgtgtgggagaaaagagaagtagaagtgagactgatgagttaaacacatagttttgaatttgaagtgaaagtgtaaatatgagcttgtgatgatatttatcctcaaaaaaatttccacaggctaatttttttttaggttttcccccttagaaggcctagaaactaggattaacccagtaatttccgtatcacattttttccaactaacaaggaccagagatccttgctaatttctagtgtagggcaagaagtttcggcaagaaccttgagcaactttgccacatcaggaagtaatacagtcttgatgacagaggtgtattgatggagctcaggaagaattgacttgaaaagaatctcattgtcacagataggacaatttgagcttcaataaaacaactactatgatagatacaagcatttcaaataagctgaaattcatgagtatacaattatgcaaaaaaatattgatcacttgtggagaatgccatataaagcattcattctcttgagaacgggcaaataaatagaaagaataactatcttattaccttgattccatcaaggtaataagatagttactgaagataatgtctctttaggaatattctaagtaataagtgaagaatgtttaatatatttttctttcacccctaggccattcattcataagatatgacccagggcactttgaggtccctgactccttttctgagggtccatagttcccaataaatcctaacaatactaccaggttaaatttttaattctattcttctcatgattttatgttgttctccagagactacatggaatatgatattgcatcagattggatgccaaagcagatatgaaaatgtagctgttttcttattaagccagacattaaagagaattgccaaaatactaaataaataaataaatagatgcagctgtaaagccacgcttctcactacatttttgaaaaacatttaaaaataaagatgtaatttttgttgatatgtaatccatgtttaattgtagagaagtttataaatattttaattattagtttattagtttttacttctaagatatatctagctatctgtctggacatacattttatacacacacacacacacacacacacgcacacacacaatttccatcacacagagaaaagcttttctggtgatctcaatctttttttattgtggtaaaatacacatatataagatttaccattttgccatttttaagtgtacaattctgtgttattaagtacattcacactgttgtgccaccttcaatataacttgtacatatgtaaaatgtgtaagagttgagacctattaatctagtaatgaatgaagaccattaagcattatatgcctcttgtgagatatacatagtataataaaacctgaatctgagcacatcacaaatctatttatttattttttattttttcttgagacggagtcttgctctgttgcccaagctggagtgcagtggcgcgatctcggctcactgcaagctccgcctcccgggttcatgccattctcctgcctcagcctccctgagtagctgggactacaggtgcccaccatcatgcccggctaattttttttgtagttttagtagagatggggtttcaccgtgttagccaggatggtctcaatcaactgacctcgtgatccgcccgcctcggcctcccaaagtgctgggattacaggcgtgagccaccatgcccggcccacaaatctatttatatatagtaaataaagagaacaaaggggcatgttaaatgacattatagctatgtaatcagcaaaatccaaactgtggatgattttacaagaagaatgatgaaatttcttaaaaaacaaattctaggataaaaatgagagaagaatcctacagattaaaaacggtttatgaaacatcaactaattttaatgcatgaatcttatttttattccaatttaatgaataaactgtaaaaacaattcatgagacaattggaaaaaatttgaatgctgaacaactatatatttaatgatattaagaaatcttttatttggggatgtcataatgtaatttcggctatatgttaaaaaagtctatatgttacagaaagacatgttgaaatagttaaggatgaaatgatacaatatctaaaatttattttaatcatgagttgataattttttcaaaactgtctaatgagcaaaggatagtttttatactgctttatgtttgcacatataaaattattattattattattattatttttggagacagggtcttgctctgtcacccaggctggagtgcagtggcacaatcttggctcactgcaacctccacttgctgggttcaagtgattctcctgcctcagccttccaagtagctgggactacagccatgcaccaccatgcccggctaatttttgtatttttagtagagacagatttttgccatgttggtcagcctggtctcaaactcctgacctcaggtgatccacctgcctcggcctcccaaagtgctgggattacaggcgtgagccaccacgcttggcccctataaaattattaattaatggaccagcgctggctggtccattttacagagtgcagattggtctattttacagagcactgattggtgcattttacaaacctcttgctagctacagagcactgattggtgtgtttttacagagcattgattggtgcattttacaaaactcttgctagctacagaaaagttataggagtccccactcaacccagggagtgcagctggcttcacctctcactatgaccctttaaaagctagactatagctgtagtaagccaagggtggttttctccaaaggagtttggaacagaaataaagaaaacataatattttttggctaagtggaaactattttagcaagatgaggagaatctacaatatcaactaaaaagaaaaaaaaatggaagaaagtagatgactctgaaagaaaacacttaacttggcagttacagcaactcagatagtttttactgagaaaatgaatttgcagctaagagatcttgccagagatctacaaatagggaacactgtcctcaaggaacttacaaagagaaggggaggaaaaacaaaaataactccaatgctgtgagtaaagtgtaaagtggatctctgcatagttccttttattatagcactaggaaagtcttcctgaggaagaaacatctcagctgagccttcagggctagaaattcatccaggcagagacagagaagaaattacatccaaaaggctctgcatctctgcaaatacagtgattctggcataattatatttggttttctttgacaaatgatatttaaccagaagcagaaaaagaaaagttttaaattaggaagccaaagggagtcaccattaagtcatatgtattatatttcatttattttgagacacattttttctaacattttaatgtgtgtaaaatcagggtgaatcacactgttagtacagtctcataataaatttacagttttaagtacttttttgctgatgtttaaataataatttattttaaaatacatgaaaactctgatttcatcaaatttgaaaatcatttgaaatatgataagattgacttttgggctgatatcttgggcttcttggtacttattgcgaatgggagaggagacatgatgatggctcatgggatattctgatatgacctgttaaaaacagaagaacaaaagagctccacataaaagatgtttgtacacccatgctcatagtaatattattcatgatagttaaaagtgaaagcaacccaagtgtccatagatagacgaatggatgaacaaaatgtgttatacgcatatggtggaatattgttcagtttcaaaaaaggaaggaagccctttcatatgctacaatgaggatattacgctaaatgaaataagtcacaaaaagacaaatatttacaattccacttatataaggtacctagaatagtagaattcatagagacagaatgtagagctgtcctaaccagggcctggagaagaggggaaatgggaacttgcagcgtaataggaatagagtttcagtcttgcaagacagaagagttttgaagattgcttgcacaacaatgtgaacacacttaacaccactgaactgtacacttaaaaatggtaaagatggtaaaccttatgttatgtgtattttaacagaattttttttaagtccacataaaacgaatgggagtttggggaaaggatatggtgaaaggattgaggataaacaatttttaaatagataaaatggtaaaaaacagtggaagggtgaacagaaaagacatattaaaaaaaaaaacaacaccacccacaactcaccggcgcctcatataccataatgcaaggcatagcaaaaggagcaaggaaggcactattattgccaaaacaattgagccaatggaggtggggtttcctggcaattgagagaggacaaaaggatgtcacttcagatactatacacagaattccttggccttcctttgcccacctacttccaatgtatggtcatttccaacttcctctctcatattcctggccaccatatatccatcctttgatcccctctacccagtttacaggtccttaattcctacatttttgataacttggattatctatttttactcctgttgggattggggtcagggaaatcaatcaatcaatctctccctctttcatctctcaagctctatccttccccagtgcctccctctatgcctgaagatcacttcctggcatttccagcctgggtccctgctatttcttactccagtagaggatgatgtcctggccctctaaactgctgtgcttcacccgacaggacaggccagccgcctccccatctgccacatccagggttgctcggagataccatgtccagttagcattgggcaggatgtcccctagctgagtgccctgctgctcctgctcaccccgcatccacatcacccacacgggctttgggtagaatcctgagacatggcacacaagctgcagacggccaggtccaggactggggccactggacagccaggcctcaggcttcactaaggcaggaaggagaaaaaaaagtgtcatgttataactcgagttcagaggttatgaactcagaaacctacaagcttggacagcgaccccattcactcctttgggaaccaaataacttgttatttaagctcctatcctttgatatcttcctactcttgatcttagaggaggtggtgggaagtgaaataacagcaggactaaccttgtctttgcagatctgcttttcctgcattgaggacgcccaagagatatcgggggcaggtttcatagaggagaattctcacagtttccatgataccttgatattgtatgattagtgcacagaatttctgtgccctgctgccaccttctggggaaggcacacatgaagcattcttgacactcaggaaatccaatcctcctagagctcccctcaggaagcttactatggcacctccagaatgtagctcacagcctgctatgccctggatctcaaaggggtctatgtagagggaaaagagagcaagttattaaacacaaataagaaaaaaaggcatgagaaaagaacctaggattttagattttggtggggataaagttatttggtgtaaaatgatgatgagctaaaaactaatgataacatttagacattttcagaaacaagagcattgagaaaaatgtgcatgccatgaagacaaaggtatgtgataaaggagtacagtgagtatgaaaggagaggtgaggcagagcgtggtgcagtcaggagtgcagcattccagagtgagtggaggttcccagcaagaggagggtgcggggaggagatgattgaaaagatggattaggggagaaagccacacgttagatcactcaggcacagggtaggagaagtaagggcccttgaggagaagtttcacataagacaaaccagcagctggttacaaagtggaagacagtctgagagtaagagaagagtgcagaggcagcaagaatctgaaatatgaggtgtgatggtgtgaagctggagtgagagactactcaagaaaagcatgtgcgtgcatttgggtaaaatagaaaggaagggagaagcatcagagagagcaacactttgcaaaaaagagagaaaagagagtccttgagactcttcccagttcggtggactagactcacatttcatctggaaatcaccggcaaagtcttgtacttctcgagcgaatccaaagatgtagactcggaatatctcctctaactcagcaacctccttatcactaaagttacctttagaccaaggcttcaggaatatggcagtgcctgagtcgctatcccagccatgaatctgcaaatcatccaaccagcctgagccttgagtttgtgcccaggtactattggtaaaggacgaggtctggataacatgaaaggaggtcggcccctggaaggctgtgaagagtggaaaagcaagatggtgaagataaagagagaagcaggagacaattaggaagaatgggaatgaaaatgatttagaaaacaagaacctagagtctaaagaacacaggaagtctgacacatttgactgccttaaggcttcaggttctagtcccaaccaccagaatggttgaagagggcgggagacagaaagacccagacccttagtgtccaattgcacctaggaaagccctgcttgctttttaaaatccaaacccctgcaccagtgctgggagctgccagagtgactcttaccatgttcactgttaccaccaggaaagagaacagctaacagttgaaatggcagcagcagcatttcactgggagatgcaacttcttactggcagagctggtatttgatctccaatttcagcaaagcttttcctcagtaccctgtagtgacttcttctctcttccaactgccaaatctcttcctcatccaaactgcttttcagagaaaaatgtgcctcccacagccctgagcctcttacctcaccttttatttccctcaagccctgatttttagagtttctcttaccccctgacttccagccgctctccctgtcctcaccttccttcatacctccttggaaaagcccccatatgattctgcattttcatttctccttccttagcctttattcccacatctataggataagatggtgatattaagcaattgctaagcttttagtctgctttaacccagaaggatgcttttagtttatccacctcacttagtttctccccctcccagctgccttatcttctctggcactctgttactgcctcttcccaatgttttcaatgtcttctctttcttatattgccctctttgtctggtgttccttaccccacattctaaactggctgtgagtgtgtgtgtgtgtgtgtctgtgtctgtgtgtgtatgtgtatgtgtgtgtaccgatattcacttacataaaggcaaagcatttaaaacatttcctgtactatagcactacaaaccaaaatgccagaatcaaatataataaagactgaagtttataattcaaacaaaattatactatagcaatatttgagggaaaaaatgacaattgctgctagcatgtgaccttctcttcaggagctcttggcctatggagttttgagtaacaaatattcatgacataaacagaactctaggagctatgctatgtgcacctccagcctaagtcaaagataactttttgggtaagttgccataagcggcagagcttttttcttctcttgtaatatctcataactgctgtctacatttgggcattcatatcaccacatgtggtattaataatagagcaaagaggttgcttttttttatacaaaaagttcagagatacttgttttaagatgtcacctcctatcttcttgtatgcccatgaaaaattttaagaagaatttttggattttctccatttaggaaaaaaatcacatatttgcagagatctatgtatcatgcagaaatgtggcagcaatcgattccgagtaaagagggttatcactaactaattctattatgtggataagaaaatgcaaagtgagtagtgtagtgttctctattcctggggacactactggttcacaacattaataagactggttcacaacattaataagactagccttcatccaagattattactgcctaattgataaatacagagcagctctccaaatttcaatgtatgtatctcttaacaacattcccagactccaatacatgacagaattgaatctactttttggataacagccatagacaacagcagagacttaacattggctgtcacagacatgaacattactatttttgtgtttatacgttttttattttgggtagaacacccaaaacagagctcaggaaaagctggtagacaacctgggctattttccattaaaataagtgagttcttgcaagactcccaaagttgaattgctacaaactaaatctgaaatgttcctcattttaaaaaataatccaaaagagttggcagggcacaggagactatcacaacactagccccacactcatcaaacttttttttttcttgagacagagtcttgctctgttgcccaggctggagtgcagtggcatgatctcggctcactgcagcctctgcctcttgggttcaagtgattctcctgcctcagcctcctaagtagctggaactgtaggcatgtgccactatgcccagctaatgtttgtatttttagtagagactgtgtttcaccatgttggccaggatggtcttgatctcctgaccttgtgatctgcccacctcagcctcccaaagtgccaggattacaggcatgatccaccacgcccggccactcatcaaatttttaaacaagttaggcctaaaacatgagaacagtaatccagtcaaaattatgttaccagaaaggaatattagtattgccaaagactgagttagaaattgagttgtgcctgagagatggaagatagaggaagaggtcttggtttcagtttcaacagcgtgtaagtttttgccaaatatgtgcagaatgtctacctaaataacaaacagaaaaagaggctctctaaaagaaaaagatatttatttgagaatagagcattgcaatgggaatatatatgcaatagtaaacgatgtgcatactcagggaggtaaaggaaggcaaattttttaaagaaaaaaaattaagagaattacatatttgttttgaaataattatccttgcctacaaatatcaataacaacggtgacaccagtcagaggttaaacagacagttgctggcagggcagatgtctttgaggcatttttcatgtaaggttgtgatggccttttcaaggttgtggtttttgcagagtctttcgtggtagttgttcttattaggtatacaagtgtgagaaccctctctgcatagtcttccctagctctgccagttttttttttttttccttagtgactccgttttgtttctggcaacatttgcaagaagatgactgtaaatttaaatttagttaggaaaatcgccagacaaaatgaaaattttgtaattcttcaaatgtcatatttgagggaaaaatttactttatacactgtcttttccctgaaaagtggagcaattgagatagtttgaaaggtctagaaattaaccaggttttcaagttacattctttctcacatatcatatccagtatttcatatttcctttctgtttcaaaagtacagaaatggacatggcagtgtccacagtgtccacatagcaatgtcacacatttccatactacactgtgactatgcttgtccagctctatggcatggtttgtatttccactctgcattagtaccattgttgcttgacatatgaaacctgtcttctagggaaagattatccctagtcccaatatttgtttcaaattaaaaaatcaacatgtgcacacttctaaatcgaagctttaattgatgcttctaactcatactctcctatgggcctaaagaccatgtacacttctgataagctcatcaagacacaggttttctcagatattgattgcataatctcacttatatgtggaatcttaaaagagttgagtacacagaaatagagaataaaacagtgattaccaggggcaggaagcaggtggtggtgggaggaaatgaggagatgtaggtcaaagaatacaaagtagccgatgagtagtcccttctgaattgcagaggatacattccaagacttctggtggatgcccaaaaccacaaatagtgctgaacctgattgctgccaattggagcccctttctgttcatgtcctccacccacaaatttgatggctttttaagatcttaactaagcacttaccacacactctgatcataacctttgcaatttgaggtgtgatggtaaaatttgtacaattcccttttccccatcacaattttatggaaagaagattcctttttactgtagaacatagcaacatcagcatattttgtttctttccttattaagtcaaaaacttaacaccttttcatttaaaggaagcacttaatggcttctctttggcatatttgaattacaagcatcactacttctgtgctttggggctgttattaagtaaaataagggttacttgaacacaagcactggagaccaggacagctgatctgataactgagccagctactaagtgactaagggttgggtagcatatacagtggagatatgataattaaggggtgatttgcatccttgtccagatgaagtaggctggcctgaaatttcatcatactacttagaatggtgcacaattgaaaacttatagattatttatttctggaattgcccatttaatattttcagactgccattgaccatgggtaactaaaactgtggaaaacaaaaccatgaattaaggaggggtctactgtatattataaataaaaaaatattttgaccttgctaccagtatccatgttaaattaacttattacatttaatattttatctatagatccttttggattttctaatgatatctctaaatatgaaagtttcattagatttttttcaattgttataccttttattatattttttgcccattttccctggcctatgacctccagtaaaatgataaatagaattattgatagctactttttttttttaaacagagtcttgctctgtcactcaggctggagtgcaatggtgcgatgtcagctcactgcgacctccacctcctgggttcaagcaagtctcctgcctcagcctcccaagtagctgacattacaggcatctgctaccatgtgtggctattttttttgtatttttagtagagacggggtttcactatgttggataagctggtctcaaactcctgacctcaggtgatccacctgtgttggcctcccaaagtgctgggattacaggcgtgagccaccgcacctggcccagataagattttttaagaggttaggtggttccaagatggctgaagaggaacagctccagtctatagctcccagcgtgagtgacgcagaagagggtgatttctgcgtttccaactgtggtacccggttcatctcactggggcttgtcagacagtgggtgcaggacagtgggtgtagcccaccaagcatgagacgaaacagggtgaggcatcacctcaccagggaagtgcaaggggtcagggaattcccttttcaagccaagggaagctgtgacagatggcacctggaaaatcgggtcactcccgccctaatactgagcttttccaatacttagcaaacggcacaccaggagattacatcctgcgcctggctcagagggtcccatgcccatggagcctcactcattgctagcacagcagtctgagatcgaactgcaaggctgaagcaaggatgggggagaggcacctgccattgctgaggcttgggtaggtaaacaaagtggccaggaagctcgaactgggtgtggcccaccgcagctcaaggaggcctgcctgcctctagagactccacctctgggggcggggcatagctgaacaaaaggcagcagaaacctctgcagacttaaatgtccctgtctgacagctttgaagagagtagtggttctcccagcacggagtttgagatctgagaatggtcagactgcctcctcaagtgggtctctgacccccgagtagcctaactgggaggcaccccccagcaggggcagactgacacctcacacggctgggtattcctctgagatgaagcttccagaggaacgatcaggcagcaacatttgctgttcagcaatatctgctgttctccagcctctgctgctgatatccaggcaaacagggtctggagtggacctccagcaaactccaatcaacctgcagctgagggtcctgactgttagaatgaaaactaacaaaagaaaggacatccacataaaagccccatctgtacatcaccatcatcaaagatcaaaggtagataaaaccacaaagatggggaaaaaacagagcagaaaaactggaaactctaaaattcagagcacctctctccctccaaaggaacacagctcctcaacagcaatggaacaaagctggacagagaatgactttgacaagttgagagaagaaggcttcagatgatcaaacttctcagagctaaaggaggaagttggaacccatcacaaagaagctaaaatccttgaaaaaagattagatgaatggctaactagaataaccagtttagagaagcccttaaatgacctgatggagctgaaaaccatggcacgagaactacatgacgaatgcacaagcttcagtagctgatttgatcaactggaagaaaggatatcagtgatggaagatccaatgaatgaaacaaagcaagaagagaaatttagagaaaaaagaataaaaagaaatgaatgaagcctccaagaaatatgggatgatgtgaaaagaccaaatctatgtctaactggtgtacctgaaagtgatggggacagtggaaccaagttggaaaacaatctgcaagatattatccaggagaacttccccaacctagcaaggcaggccaacattcacattcaggaaatacagataatgccacaaagatactccttgagaagagcaaccccaagacacataattttcagattcaccaaagttgaaatgaaggaaaaaatgttaagggcagccagagagaaaggtcaggttacccacaaagggaagcccatcagactaacagctgatctgttggcagaaactctgcaagccagaagagagtgggggccaatattcaacattcttaaagaaaataattttcaacccagaatttcatatccagccaaactaaggttcataagtgaaggagaaataaaatcctttatagacaagcaaatgctgagagattttgtcaccaccaggcctgccctacatgagctcctgaaggaagcactaaacatggaaaggaacaactggtaccagccacgcaaaaacatgccaaattgtaaagaccatcgatgctaggaagaaactgcatcaactaacgagtaaaataaccagctaacatcataatgacaggatcaaattcacacataacaatattaaccttaaatgtaaatgggctaaatgctccaattaaaagacacagactggaaattggataaagagtcaagacccatcagtgtgctgtattcaggagacccatctcacatacagagacacacatgggctcaaaataaagggatgggggaagatctaccaagcaaatggaaaacaaaaaaaaggcaggggttgcaatcctagtctcggataaaacagactttaagccaagaaagatcaaaagagacaaagaaggccattacataatggtaaaggggtcaatttaacaagaacagctaattatcctaaatatatatgcacccaatacaggagcacccagattcataaagcaagtccttagagacctacaaagagacttagactcccacacaataataatgggagactttaacaccccactgtagacattagacagattaacaagagagaaagttaacaaagatatccaggaattgaactcagctctgcaccaagtggacctaatcggcatctacagaactatctgccccaaaccaacagaatatacattcttctcagcaccacatcacacttattccaaaattgaccacatagttggaagtaaaggtctcctcagcaaatgtaaaagaacagaaattgttacaaactgtctctcagatcacagtgcaatcaaactagaactcagtgttaagaaactcactcaaaaccactcaactacatggaaattgaacaactggctcctgagtgactactgggtacctaatgaaatgaaggcagaaataaagatgttctttgaaaccaatgagaacaaacacacaacataccagaatctctgggaaacatttaaagcagcctgtagagggaaatttatagcactaaatgcccacaagagaaagcaagaaagatctaaaattgacatcctaatttcacaattaaaagaactagagaagcaagagcaaacacattcaaaagctagcagaaggcaagaaataactaagatcggagcagaactgaaggagatggagacacaaaaaaaccttcaaaaaatcaatgaatccaggagcttgtttttggaaaagatcaacaaaattgatagaacgctagcaagactaataaagaggaaaatagagaagaatcaaatagatgcaataaaaaatgaaaaaggggatatcaccaccaatcccacagaaatataaactgccatcagagaatactataaacacctctacgcaaataaactagaaaatctagaagaaatggataaattcctggacacatacaacctcccaagactaaatcaggaagaagttgaatctctgaacctaccaataacaggctctgaaattgaggcaataattaatagcttactgaccaaagaaagtccaggacctgacggattcacagccgagttgtaccagaggtacaaagcggagctgctaccattccttctgaaactattccaatcaatagaaaaagagggaatcctctctaactcattttatgaggccagcatcatccagataccaaaacctggcagagacacaaccaaaaaagagaattttagaccaatacctttgatgaacatcgatgcaaaaatcctcaataaaatactggcaaaccaaatccagcagcacaccaaaaagtttatccaccacgatcaagtgggcttcttccccggggtgcaaggctggttcaacatatgcaaattaataattgtaatccagcatataaacagaactaaagaaaaaaaccacatgattatctcaatagatgcagaaaaggcctttgacaaaattcaacagcccttcatgctaaaaactctcaataaactaggtatttatgggatgtatctcaaaataataaaagctatttatgacaaacccacagccaacatcatattgaatgggcaaaaattggaagcattccctttgaaaactgtcacaagacagggatgccctctttcaccactcctattcaacatagtattggaagttctggccagggcaatcaggcaggagaaagaaacaaagggtattccattaggaaaagaggaagtcaaattgttcctgtttgcagatgacatgatcgtatatttagaaaaccccatcttctcagcccaaaatatccttaagctgataaggacattcagcaaagtctcaggatacaaaatcaatgtgcaaaaatcacaagcattcctatacaccaataacagacaaacagagagtcaattcatgagtgaactcccattcacaattgcttcaaagagaataaaatacctaggaattcaacttacaaaggatgtgaaggacctcttcaagaagaacttcaaaacactgctcaatgaaataaaagaggacacaaacaaatggaagaacatttcatgctcatgggtaggaagaatcaatatcatgaaaatggccatactgcccaaggtaatttatagattcaatgccatcctcatcaaactacgagtgactttcttcacagaattggaaagaactactttaaagttcatatggaactaaagaagagcccgcattgccaagacaatcctaagccaatagaaaaaagctggaggcatcatgctaccttacttcaaagtatgctacaaggctacaataaccaaaacagcatggtactggcaccaaaacaaagacatagaccaatggaacagaacagagccctcagaaataataccacacatctacaaccatctgacctttgacaaagctgacaaaaacaagaaatggggaaaggattctctatttaataaatggctctgggaaaactgtctagccatatgtagaaagctgaaactggatcccttccttacaccttatacaaaaatcaattcaagatggactaaagacttacatgttagacctaaaaccataaaaaccctagaagaaaacctaggcaataccattcaggacataggcatgggcaaggacttcatgtctaaaacaccaaaagcaatggcaacaaaagccaaaattgacaaatgggatctaattaaactaaagagcttctgcacagcaaaagaaactaccatcagagtgaacaggcaacctacagaatgggagaaaatttttgcaatctactcatctgacaaagggctaatatccagaatcgacaatgaactcaaacaaatttacaagaaaaaaacaaacaaccccatcaaaaagtgggcgaaggatatgaacagacacttctcaaaagaagatctttatgtagccaacagacacatgaaaaaatgctcatcatcattggccatcagagaaatgcaaatcaaaaccacaaagagataccatctcacaccagttagaatggcaatcatcaaaatgtcaagaaagtacaggtgctgcagaggatgtggagaaataggaatacttttacatggttggtgggaccataaactagttcaaccattgtggaagtcagtgtggcgattcctcaaggatctagaactagaaataccatttcaccaagccatcccattacttgctatatacccaaaggattataaatcgtgctgctataaagacacatgcacacatatgtttattgtggcactattcacaatagcaaagacttggaaccaacccaaatgtccatcagtgatagactggattaagaaaacgtggcacatatacaccatggaatactatgcagccataaaaaatgatgagttcatgtcctttgtagagatatggatgaaactgaaaatcatcattcccagcaaactatcggaaagacaaaaaaccaaagaccgcatgttctcactcataggtgggaattgaacaatgagaacacatggacacagtaaggggaacatcacacaccgggggcctgtcgtggggtgggggaaggggggagggatagcattaggagatatacctaatgttaaatgacgagttaatggatgcagcacactaacatggcacatgaaaacctatgtaacaagcctgcatattgtacatgtgtaccctagaacttaatgtataataataaaaaaaaagaaaatgaagatgaggaggaaaaaaattaaaaaaaaaaagattcctttggattttccaagtatacaatcaaaaaaatttaagaggttaacaagtgtcttattttctgggagtgttttgtgtaaacttttcttaccagattaagaatattctcttctaaattagtttaaagtttttttattattattatggagaactgtttatataggtgcttatattattttattttattatgtacttatttattcatttaatttttttttttttttgaaatggagtcttgctctgtcgttcaggctggagtgcagcggagcaatcgcagctcactgcaaactccgcctcccaggttcaagcgatgcttctgccccagcctctccagtagctgggactacaggtgcccaccaccacgcctggctaatttttgtgttttttttttttgtagagatagggtttcaccatgttgtccagcctggtcttgaactcctgagctcaagtgatccacccgccttggcctcccaaaatgctggggttacaggtgtcagccactgtgcctggccaggtcttacattttaattgttaatccaccgtgagttaatttttgtaaatggtgaaaggcagaaatgcagttttattcttctgcatatggttatctgttttcccagaaccatttattggatattgagttatttatcaattactcatttttgttcactttgttaaagaacagttggttgaaaatatgatgctttatttttgatttctctattaagttccattggtctatcttgttaatttttgtatcagtaccatgctgttttggttattgtagcctaatagtaaagtttgaagtcaggtagtgtgattcctctggctttgttcttttccctttggattgctttggctatctgggcttatttttggttccatatgaattttgggatagtttttttctaagtctgtgaaaaatgatgttggtgatttgatacaaataacatcaaatctgtagattgctttgggcagtatgaacattttaataatattgattcttcaaatccatgagcatgaaatgtttttccatttgtttgtgtcccgtatgatttctttcagcagtgttttgtaattctctttggagagatcttttgcccctttcgttaaaggtattcatagatattttactttttttggtggatactgcaaatggggttgcattctttatttggctctcagcttgaatgttattggtgtatagtaatgctactaggtttagtacattgactttgtatctgaagttttattgaaattgtttgtccactccaggagacttttggtggaatctttagagttttctacagataaaactatatcatcagtgaagagaggtaatttgacttcctatttagttatttggatgctttgcattttgttctcttgactaattgctttggcttggtcttccagcactatgttgaataagagtggtgagagtgggcatccttgtcttgttccagttcttaagaggaatgcttttggtcattcagtatgatgttggctatgggtttgttgtatatggcttttattattttgaagtaagttcctttgatgcctggtttgttgacggtccagggacttttttggttggtaagtttttattgctgattcagttatggaactcgttattgctctgtttagcgtttcaaattcttcctggttctatcttgagagattatgtgtttgcaggaatttacccatttcctctagattttatagtttgtgtgcatagaggtatttatagtagtttctgggggatcttttgtatttctgtgggatcagttgtaatgtcaccttttccatttctgcttatgcttatttggatcatgtctctttctttttttttaacacttttatcttaagtttaggggtacttgcacaggcttgttacatagataaacttctgtcatgaaggtttgtggtacagattatttaatcacccaggtattcagcttagtacccactagttttttttcctgatcctctccctcctcctaccctgtaacctccaataggccccagtgtgtgttgttcccctctatgtgtccatgtattctcatcattttgctcccacttatgagaacatgtggtattttgatttctgttcctgtgtagtttgctaaggataatggcctttatttagctccatccatgtcctataaagggcattatctcattcctttttgtggctgcatagtattccatggtgtatatgtaccacgttttctttatccagtctatcattaatggacatttaggttgactccatgtcttcactattgtgaataatgctgcaataaacattcacgtgcatgtgtctttataatagaacaatttatatttccctgggtatatacccagtaatgggattgttgggttgagtggtatttctgtttttagctctttgaggaattgccccactttcttccacaatggttgaactaatttatactcccaccaacagtgtataagtgttcctttttctccactacttcaccagcatctgttatttttggactttttaataatagccattctgactgatgtgagatggtatctcattgtggttttgatttgcatttctctaatgataagtgatgttgagctttctcttatatgattgttggccacatgtatgtcttctttcgagaagtgtctgttcatgtccttttcccactttttgatgggactctttttcttgtaaatttatttaagttccttataagtgctggatttaaactttattagatcttgttgagcttccttatcatccagattctgagtcctatatctgtcatttcatacatttcagtctggttaggttcattgctaaggttcattttcaaggtaacaaaacacctcggactttttgaattgctggagttattgtgttgattccttctcgtgagggagctggcacttctttttctttttgaatttgttgccatttggatgaggtttatatatacatatattatttttatatatatatatatatatatatatatatatatatatatgtacacacacacacacacacacacacacacacatatgagaatgagttctttcagagagccaaggctctgtacaggttcctaggttgtagatagtttcctgcagtgggtttcacaggcaatgagtgttggtgaaatgcacttttgtttggtgatgtacttcaggctgcagtcaagtagatggtgcttaacagcagggctgacagtctcttagtcagctgtacacttgttttgtttttcagcgtgtttgtagcaatgctctgggaagaggagatgggggtcaatatatggctccctctacaagtccattcccaggtttgggtaacatcaccttcgattactggtgctttgtccactttttctttgtcatttttctgtgtctactttaatatctttcattaatattttgtaattttcagtgcattagtctttttttttttttgagatggagtctcactctgtcaccaggctggagtgcagtggcacgatctcggcttactgcaacctctgcctcccaggttcaagcgattctcctgcctcagcctcctgagtagctgggattacaggtgcgtggcaccacacccagctaattttagtatttttagtagagacggagtttcaccatcttggccaggatggtctggatcccttgacctcgtgatccacctgcctcggccttccaaagtgctgggattacaagcatgagccactgcgcacctctttgattaagcttattcctgagtattttattctttttatgctactataaatgagattgttttcttaatttcctttcatgatagtttattgtgtatagaaacaccattgatatgtgtgtgttgatttttgtgttctacatctttactgaatttattaattctaatcatttttcttgttgagtattaagggttttctgtgtacatcatcatgtagattttataaatcatatcgattttgtagattattgctgcagacagataattttaccttttactttagattttgatgccttttatttctttttcttatctacttgctctgtctagggcttctaacactgtgttgaatagaagtagcaagagtggacattcacgccttgtcctggatcttagagaaaagattgattatgatgttagctgtgggattttcatatatgccctttattatgggatttttttttggatatgacaccaaaagcaccagcaacaaaagcaaaaatagacaaacaggattgtttcaaactaaaaagcttctgtgcagcaaaggagacaatagagtgaagaggcaacctacagaaggggagaaaatacgtgtaaactgctgggtgccgtgtctcacgcctgtaatcccagcactttgggaggccaacgtgggcagatcacctgaggtcaggagtttgagaccagcctggccaacatgatgaaaccccatttctactaaaaatacaaaaaattagttgggtgtggtggcaggcgcctgtaatcccagctactcaggaggctgggacagaagaatcgcttgaacctgggaggcagaggttgcagtgaactgagattgtgccactgcacaccaacctgggcaacaagagtgaaactccatcaaaaaaaaaaaaaagatagtatttgcaaaccatatgtctgaaaagggtttaatagtcaaaatacataaagaactcatgcaacacaatagcaaaacaaaacaaaacaaaacaaaaccctaattaaaaatgaacaaagaatctgaatagacacttttcttaaaaaaaaagcatacaaatagccaatagatatattaaaaggtgcgcaatatcactaatcatcagagacatgcaaatcaaaaccatagttagatatcccctcctacctgttaggatggctattgttaaaaatacaaaagaaaagaagtgttgatgaggatgtggagaaaaaagaacccttgtattcttttggtagccatgtaaattggtatatccattatggcaaacagtacgcaggtgcctcaaaaaattaaaaatagaattaccatatgttccagcaatcccacttttgtaaatatgtccaaaggaaatagaatcactatctcaaagagctatctgcactctcatgttcattgcagcactattcacaatagcaaaaatgtgaaaaaaaaactgagtgttcattgacatatgaatggataaataaaatttgatatatataatggatgttattcaatctttaaaaagaaaatcctgccatttgtaacaacacaggtgaaactggaggacattatgtgaagtgaaataagccagaaacagaaagaaaaatgctgcataatcttgtgtgaggattcgaaagtagtcaaactcatggaaacagaatagaatggggatttccagggattgcagtaagggagaagtgaggagatgttggtaaaaggtagatagttccagttatgcaagataagttctggaggtctaatgcacggcaatgtgactatagtgaacaatactctattgtgtagtatttgaattgctaagagggaggatcttagatgttctcactataaaaaataagaaaaaatggccaggcgcggtggctcatgcctgtaatcccagcactttgggaggctgaggcgagcggatcacgaggtcaggagatcgagaccatcctggctaacacagtgaaactccatctctactaaaaatacaaaaaataaaaataaaaaataaaaaaattagccgggcatactggctggtgcctgtactcccatctactcgggaggctgaggcaggagaatggtgtgaacctgggaggcggagcttgcagtgagccaagatcatgctgtgtccggaattggtgggttcttggtctcactgacttcaagaatgaagccgcggaccctcacggtgagtgttacagttcttaaaggcggtgtgtccggagtttgttccttctgatgttcagatgtgctgggagtttcttccttctggtgggttcgtggtctcgctggctcaggagtgaagctgcagaccttcgcaatgagtgttacagctcttaaggcagcacgtctggagttgctcgttcctcccggtgggctcgtggtctcgctggcttcaggagtgaagctgcagaccttcgaggtgagtgttacagctcataaaggcagtgtggacccaaagagtgagcagtagcaagatttattgcaaagagcgaaagaacaaagcttccacagtgtggaaggggaccccagcgggttgccactgctagctgggggcagcctgcttttattcttttatctggccccacccacatcctgctgattggtagagccaagtagtctgttttgacagggtgctgattggcgagtttacaatccctgagctagacacaaatgttctccacgtccccaccagattagctagatacagagtgtggacacaaaggttctccaaggccccaccagagtagctagatacagagtgttcattggtgcattcacaaaccctgagctagacacagggtgctgattggtgtgtttacaaaccttgagctagagacagaatgccaattggtgtatttacaatccctgagctagacataaagattctccacgttcccaccagactcaggatcccagctggcttcacctagtggatcctgcactggggctgcagatggagctgcctgccagtcccccgccgtgaacccgcactcctcagcccttgggtggttgatgggactgggcgccgtggagcagggggctgcgctcatcggggaggctccggccgcacaggagcccatggagggggtgggagtctcaggcatggcgggctgcaggtcccgagccctgccccacggggaggcagctaaggcctggtgagaaatcaagcacagcgctggtgggctggcactgctgggggatccagtacaccctccgcagccgctggcccgggtgctaagtctctcattgctcggggccggcagggccggctggctgctccgagtgcaggcccgccaagcccacgcccaccctgcactccagctggaaccgcccgcagcccaggttcccgctcgcgcctctccctccacacctccctgcaagctgagggagccggctccggccttggccagcccagaaaggggctcctacagtgcagcggcgggctgaaaggcttctcaagtgctgccaaagtgggagcccaggcagaggaggcaccgagagcaagcaaggtctgtgaggactgccagcacgctgtcacctctcaatgccactgcactccagcctgggtgacagagtgagaatccgtctcaaaaagaaaaaaaaaagaaaaaatgtaactatgtgaagtgatggatatggtaataacttgactgtggcgattacttcacagtatatacatatatcataacatcatgttgtgcaccttaagtatacccaatttaaaaaaaattaaggtagaatataaatatataatttatcatcttcaccattttaaaatgtacagctcagtggcaataaatacttttttatactcaatttttatttgttggttgtacctcactaaagctgaaaaatatatttgaataaatgttaaagaaatgtaagtatacacaaacaagaaaataagtatgtagaaattgcaatctttcctgaaaataagccaaaagaataaaactcgtatttaaattctttgaaaaaaaattagcagatggaacattttcttacatttgtacaaatgtactcaaaaattttattttaaatcaattttaatgagttatctcccataaatttcttacatagaaagattaagttgtccaaaggcacatcttataatatgttttgagagaattttctggttttggcaaaaggggatctgcgaatgtgactggggcagcctcaggaaaacataagagggggaatttctttttaaaagagacctctggccacagacactaagtatttttagtaaaaaatgtggttaggcactgatggaatattttttttttttttttttttgagacggagtctttctctgttgcccaggctggagtgcagtggcacaatcttggctcactgcaacctctgcctcccaggttcaagcgattctcttgcctcagcttctcgaatagctgggaccataggcacgtgccaccacgccctgctaatttttttgtatttttagtagagaggggtttcaccgtgttagccaggatggtctcgatctcctgacctcctgatctgcctgcctcggcctcccaaagtgctgggattacaggagtgagccactgtgcctggcccactgatggaattttgtaaagcaggaaggaaataattgtaccatataatgttacaaaaattaaaattaatatatattttttctttggaatttatcttctggataattttaatttagaaggaatttcattattttgttaatgattatggggttgctgtcagacattatttctactttgctcagtaatttgcagttatctgtgagttctgtagcctcaaaattaaaaactagggtgtggttatctttacttacataaaagtgcaattttttgtggaataacaacattgttttattctctaaatatactttgctttattttactttgcaaatgattttaaaattctccctcttttttttttttttgaaacagagtcacactctcctgcccaggatgtagtacagtggcacaatctcagttccctgcaatctccacctcctaggctcaagcaattctcctgcctcagcctcctgagtagctggaattacaggcacacatcatggcacctggctaatttttactttttttggtagagacagggtttcgccatgttggcaaggctggtctcgaattcgcctgccttggcttcccaaagtgctgggattacgggtgtgagccaccgtgcctggcctgattcaaaaattctaatggccggggcacggttgctcacgcctgtaatctcagcactttgggaggccgaggcgggtggatcacgaggtcaggagatcgagaccgtcttggtcaacatgaagaaaccctgtctctactaaaaatacaaaaaatagatggtcatggtggctcttgcctgtaatcccagctgctcaggaggctgaggcaggagagtcgtttgaaccagggattcgggggttgcaatgagccgagatcctgtggctgcactgcagcctgggcaacagagcaagacagcaagactccgtctcaggaaaaaacaaaacaaaacaaaaattctaaggaatacagttacttgtagtattgatgtttggcataggtcattccaatctttttattatgtattattgtttttgtaggttattatatattctcttgaatatttatctttgtttactgttcataattttattatgaaaccaattttggatttacatagcgatttatagatagatttacatagcaacttatagctttgaatgtatttgtagaaaagaaagagtattgaaaataaatgtagacattctactcaggaaacaaggaaactttttttaatctttctttaaccttagaatttagaaatgataataagatgaaactaggtttggacctttttcttttgtttttttgtttgcagtttttttttctttttttaattatttttatttatttatttattttttattattatactttaagttctagggtacatgtgcacaacgtgaaggtttgttacatatgtatacatgtgtcgtgttggtgtgctgcacccattaactcgtcatttacattaggtatatctcttaatgctatccctcccccctccccccaccccacaacaggccccggtgtgtgatgttcctcttcctgtgtccatgtgttctcattgttcaattcccacctatgagtgagaacatgcggtgtttggttttttgtccttgtgatagtttgctgagaatgatggtttccagcttcatccatgtccctacaaaggacatgaactcatcattttttatggctgcatagtatttcatggtgtatatgtgccacattttcttaatccagtctatcattgttggacatttgggttggttccaagtctttgttattgtgaatagtgccacaataaacatacgtgtgcatgtgtctttatagcaacatgatttataatcctttgtgtatatacccagtaatgggatggctgggtcaaatggtatttctagttctagatccctgaggaatcgccaccctgacttccacaatggttgaactagtttacagtcccaccaacagtgtaaaagtgttcctatttctccacatcctctccagcacctctccagcacagaaagagtattgaaaataaatgtagatattctactcaggaaacaaggaaactttttatctttcttttaacttagaatttagaaatgataataagatgaaactaggtttggacctttttcaataattctgaccaccgttcagtaaatttactgtataagaaaagtagaggtttggggttgaattttgttttattttttcccaaagatgtctatctctgatatcttagggcctggcaattgtttttggtcataggtgggctctgaataaatgtcagttgaacaaatatataaaaaccaaacaaaggaatgttatttcaactgccttcagagtgtcacaacaaatatcaatgaaaaagaaacaaaaataaagactcaattaattctccagtgaacacttcagtcctctaaaggtacttccctgtgtccacccaaactgctctctaaccgaacgcaagcaaggggactccaatgctctcaggccatgaactaatgaggagcttcatgcctgccctcagtctttcttcatacccagggcaaagctggcccacagacttccagacatgctttcttcgtttgggtagagatggggatccaagtcccctatgtatttcctccagagccacctgtaaaacatgggtgaaatctacctgccctctagctcaccacttccaggtcaagcccactcatggaccactttaacagtgacagttggcttgactggggaaactgaaaaaaaggagctttctttagattttctgataatctgcattgccattgctgctcctttttcagcagcacagagagttggattatggagggttgcctgcttattatgcagctacatccaggatgctgagtgtcttctgatcatgatcaagagcagagtgaagagatacaggcggaggagaggatcacagagaggtagaagtgggagactggtggataggtcactaggcaccactgagtgactaccatcacttctcacagattagattattgacaagccaagtatggatttgatttctacaaggccaaaagctactccatcttctagccagaccacagactttgttttaagccaggactcgttttccagcagttagtgcaattagggaggttgggaggatgtacacaagagcagggagaaaatctggagccagacagtgagagaggcagaggggaaatgaaaaaccccgtgggaggctgcagcaaagcgaggcagtgtggcttctctgacagggaagtcagcagagggagaggtttgtctgtctgtacagcaagggaagtcagacgagagtgcaagagggtgtggagaggggtactgatatctgaattattagggcaggtgtcctgccaaggaatccctcctttaacagagcttcaatgctgctcctgttcctcctcttcgagggtctctgctgtcctggggaaaatacagcaggtaagaagagtgcaggtggaaagatacctatggtagggcaccagagggctgagaggaagctctggggaggtcctgggggaagggagcagtactcttctaggatgcccttggaatatgcctttcaggctagttccaggcagagaattcttgctctcagtctcagtttttgtctctgattttggagaaaggaagctggccccacaggaaaagggtattggagtatgtacaagctacctaactgtctctcatctctgggttccttttttccctttggcatcacttttcccatccctttacattctctctacttgtcatttccctctctctcagctccccaggctctacaatcctatcatctagcagcagaggagcagctgtccttccgcatgctccaaacttcctcctttgccaaccacagctgggcacacagtgagggctcaggatggctgggtgacctgcagactcatggctgggacactgtcttgggcaccatccgctttctgaagccctggtcccatggaaacttcagcaagcaggagctgaaaaacttacagtcactgttccagttatacttccatagttttatccagatagtgcaagcttctgctggtcaatttcagcttgaatgtaagttcgttgctctaagctgataatttgcctgggaacaccaactattttccaaagtggaagatagtatatagactctgaccatcatttaaccttactaaccttgttccccactctctgactcccactccctcctctgcttcacccttcaccaccacccacactccaccatatacacaaaagggcctgcatgtacatatctcaacatgaatatagcttcatgtctggctctttggaatgattgtctcctctggatcttctgcccctcattcctgccctcagactcagcctttctcaaccctctttctgcccttctttatcctttgcctgagtgttgacatggactggcctgtacctaaccactttcacgtgaattatttatgaccaatctcctatcttcctgatagccttcccatctaccactttcccattagttatttcaaagtatctttattatcttcaaattttcttcccacaaattttcttcccctttgccagtaaactctagtctccatatgatttcccagcaaatttttcttcccttgaatctctttactgtctaaattgtttgtttttcttccttgtcattctttccataatgatctctcttccctgtccactctcagaccccttcgagatccagatattagctggctgtagaatgaatgccccacaaatcttcttaaatatggcatatcaagggtcagatttcctgagtttccaaggaatttcctgggagccatctccaggagcagggatccgggcccagaacatctgtaaagtgctcaatcgctacctagatattaaggaaatactgcaaagccttcttggtcacacctgccctcgatttctagcggggctcatggaagcaggggagtcagaactgaaacggaaaggtgagcccaactctctctctcccctcttgttcctagtactataactctcatatttgaatttgcctctcatcatcattttgaaagacatagtgagagactagagaatgagatgtgtgggttcaggactgtttcttagacaagagaaagaagtgattactaaatcactcttagtattattacaaaggcacctgagtctctgagctctggcctggggtgcccttcaaaattccattttttttctatcttcttcttcctagtgaagccagaggcctggctgtcctgtggccccagtcctggccctggccgtctgcagcttgtgtgccatgtctcaggattctacccaaagcccgtgtgggtgatgtggatgcggggtgagcaggagcagcggggcactcagcgaggggacgtcctgcctaatgctgacgagacatggtatctccgagcaaccctggatgtggcggctggggaggcagctggcctgtcctgtcgggtgaaacacagcagtctagggggccatgatctaatcatccattggggtgagaaacagctgaggctctgctgggaaataatgaaaatagccctggggcttttgagtgtggggctgaggaaatgggtaggaatgctaggtacaagaagggtaaaactgggacaatcaaaataaagaaggatagagtatgacagtagttaaattttaagaaaatggaagtagagaattagacatactaacagaaaaaggaggaggaactagtgatttagtgggagagggttgggaggagatcacagacaaaggatcaggaggaattgaaatgagggctttggaaaacccagatgaaaattctaggaaggtcccacccttgtgaaatgggaaatctcagcttggtggaatagagtattttagggttggtattcttattctatccccaaccaggtggatattccatctttctcatcctgatctgtttgactgtgatagttaccctggtcatattggttgtagttgactcacggttaaaaaaacagaggtgagctttttcttgttctttgtttcttcagcctgtaatcaattcattcctttttcctctatcttctttttttttttctctgcctttcccctttatttcctttctttgagattaatatcctcctcttttcccacagttcaaataagaacattctttctccccacacacccagccctgtctttctcatgggagccaacactcaggacaccaagaattcaagacatcagttctgcttggcacaagtatcgtggatcaaaaacagagtattgaagaagtggaagacacgcctaaaccaactctggtgacatttgctttaccttatacataaaatccttgtctgcatcttcttaaacaccgtccatgtcccataagggaagcatgcttttatttaaacagtttatactagcaaagatactgacccctttaggaatactttttccccatcttccagagattttttttttcctgctttggctacatatccatcattgtttatttttgaaactataatccagatacttctttttcatggattcccgagatcacccaattgatagctcttctgtactccccaaattgaactgatcttcacaagcacattcatctcttcctactctgaacagtagttatttaggtttttgctctttttttttttttaatctcagttgcttgaaagtaggatttaggtatttgtgtctgtattcatgaccaaaaatcttatctgaattcagggccagcttcataagcatgtgacctgtgcagacacatggaatcgtatgctctgcaagaacccatgctagatttaatgctctgctgttgtcatcttggaatccttagtcattttcaaacaagagatattgtattttcatttttcactgaccccacaaattatatagctgattcagtgtgaatgtaatatttctcaataaatgctgactgaaataaatttgttgttagatcaagtgatgttgttttactccattccctctctgatatctctgcgtcatttgactgtgctcttgctaaaactcttcctctctccctttgtctctcaccagaggtgaaggaggcagatgaatgcctcagactccacagtgattggaatgttaatgaatcttcagggatatttaggaaaataataacataagggctatctcctgtcaaagtgaggagaggtttgaactggcattcctacttccctgagccatgtgtatctatgtgtgtctttgtggatgtgcatattcatgggagaaattcatattttgagcaattagaaggaggacatagggtttgagccagagctaaggcaatgaagcaaaagagtgattggatgccagttaagctacatgcttgtttttatttattagtgtggcttattattgttattattatttacagtttgtttttctttagtttttatatggattcttacatttacataatttacttcatttgagatacatttggttgagcctactggagagcaggaatctggtagaagtgcagtgtttatatacatgtccagtcaacccctcccttggatggaataattgagcagattgtgcatatttcttttctttttttttgagatggagtctcgatctgttggccaggctggagtgcagtggctggtgtcagatcactgcaagctccacctccggggttcatgccgttctcctgcctcagcctccccagtagctgggactacaggcgcctgtcaccactcccggctattttttttttaatttttagtagagacggggtttcgccatgttagccaggatggtcttgatctcctgacctcgtgatccaccccccttggcctcccaaagtgctgggattacaggcgtgagccaccgcgcccagccggttgtgcagattttttagtggaagttataaacttcatgctgggcattttcaagtaatcagcaagggtcccatgatcacacatccaggctgcactcttactcatgaacaaggtatgaggccagccttttcactgttgagagtccatcaccttccatatggcacatccaatcagtctatctagagtcaggccactagaatcgggagaggaaggtagaatcttaaagggttgtttggagcaacaaatgatgaaaggggtcagggttccatcacgtggtataactctacaggggaggacctctattagaatgaagaggttcaagtaggaacctttccgtttttggcacaaggtgtgcgaaaggcacaagctatacctctggagacaatagatctgggctgcctgcacatatacatccttcttccccataaaatgacatagaaggattaaaaaaagagccagaggacaaataatcatgttataagaagtatcagctcaacaggacttgaggaagtttaaatatcagtccataatagtgtcagaaaagctctaggtcttccctgtactgtggtaaacctttgggctgggtattttcattataactcctggatgatggtgtacttaggagcaaaatatgttgctaaattgcccccaagacagagatgttacctaatactattaacaacttcattctggctgaaactctgggacatgggacttattctagaagaccagtgtagtttttttcaatagctaagtataccaatttccattcccaccaggtataaaacaaagaaggtaatctgctttactgacagtacacatctggagctttctgcattctgttgtaaatacaaatacagtaagaatgaaagtaggtagttttggaaaaagagatggagggggtattcttgatgattgctaaatttctctagaaatagcattcattgtttgagggagaatgcattgggactggtggaatcagaatatgctggggccaaagctcagaagagtgcagtagccacatgccccggataaaccatgacctaacctgtctgctatcttctatctaatgaatgagataagctgaggatatgggagacttgataagtggagcaacataaactgaaacaggggtccaggcctccaagtgtatcacctacaaacaaagaaagatgcagcacctgctctggagcttgtgttcagatgagtaggtatagttaagggcacatgactccttcttcaggttatgcatagaaggtacctatagatttttgaaacttagactttactcttaaggaattagaactagttcataatctttcctcattaaccattttctcactctcacttccttatgtaaatggttccattatcttactagtttctcaagatccaaactttcttctgtttgcttcattattcttcctgaatagcaagttaccaaagccttctttataagttttcttatgttccattccctcttccttgatcctgccccacgtgtgaacaccactgccaaaaatgtaatgcaaattactaaacccattatcaacaacgccataatgatcacttattgagcatgagatcttattacagcaaataagagaagtatttgttttatagttaaagctgattgaataataaaaatcatttgaatggggcctgctgtaggccaggcactcttctccatactttatacatattaactcatgcaatcttcttatcaatcctgagcatataatattattcccattttatatatgaggagacaaaagtcacataattagtaagtatgtaggcaattttgctccaaaattcctgctttaactactatactgcacattcactaaataactattggaagcttgttatgtgataaatgctattatggtaatgaaatgcattaaagacacaagaatgaattaccctgtcttcacagaacttacgttggcagtgggggagctggaaagacagacaataagtaaacaaagatgttgtattaaggtgatgacatattaagattgataactgaatcaggggaaaggaatcagagagaaattatttattatttcaaaatttgtggacagaatgggccactccgagaatatggcctttgagaagggaacgaaagcaagaacaaacacaagtcatgcagtatctggggtaagaacatttccggcaaaggaactacaaagaaaagcagcattgtaatggctgttttctaaaacctgtgagaattccagtttagatggattggagtaagtgatagggaacataggaaatgaggccagtgagttcaagtcagatcacaaaggggacttataggctattgtacagtgattaaaagccattgcagggttttgttgttgttgttgttcacagtaatatactttactcaattgttataagctgtaaatagcttaaaagtttccttgactctgaaaaacaaaacaaaggattagtagtgttttaagcaaagttaaaaagattgcttcagttttttttttatagaacatctttctttaaattttattattattatacttcaagttttagggtacatgtacacaatgtgcaggtttgttacgtacgtatacatgtgctatgttggtgtgctgcacccattaactcgtcatttaacattaggtatatctcctaatgctatccctcccccctccccccacctcacaacagtccccagcgtgtgatgttccccttcctgtgtccatgtgatctcattgttcaattcccacctatgagtgagaacatgcggtgtttggttttttgtccttgcgatagtttgctgagaatgatggtttccagtttcatccatgtccctacaaaggacatgaactcatcattttttatggctgcatagtattccatggtgtatatgtgccacattttcttaatccagtctaacattgttggacatttgggttggttccaagtctttgctattgtgaatagtgcctcaataaacatatgtgtgcatgtgtctttatagcagcatgatttataatcctttgggtatatacccagtaatgggatggctgggtcaaatggtatttctagttctagatccctgaggaattgccacactgacttccacaatggttgaactagtttacagtcccaccaacagtgtaaaagtgttcctgtttctccacatcctctccagcacctgttgtttcctgactttttaatgatggccattctaactgctgtgagatggtatctcattgtggttttgatttgcatttctctgatggccagtgacgatgagcattttctcatgtgttttttggctgcataaatgtcttcttttgagaagtgtctgttcatatccttagcccacttgttgatggggttgtttttttcttgtaaatttgtttgagttcattgtagattctggatattagccctttgtcagatgagtaggttgcaaaaattttctcccattttgtaggttgcctgttcactttgatggtagtttcttttgctgtgcagaagctctttagtttaattagatcccatttgtcaattttggcttttgttgccattgcttttggtgttttagacatgaagtccttgcccatgcctgtgtctgaatggtattgcctaggttttcttctagggttgttatggttttaggtctaacatgtaagtctttaatccatcttgaattaatttttgaggttttaaataaatgtataatggttgttaggtaaaaggagatgagttccaagaagcagagagtagaatagtggttgtcagggcctaaggggaggagtcaatggagagattgtggtcaatgggcacaaagtatgtaagataaataagttctggagatctactatgcagcacaatacctatggctaacaatgctgtgttgcattcttaacatttgctaagagagatcttgtgttaagtgttcttacaacatacacaaaacattactattactaataaagggggcgcgaagagacttagggctttgatagtggtgatgttattatgggtgtatacttacctttatactcattgagttatatacattaaatattaacagctttttacatgtcaattctaccttaatactgtggttaacaaaaggagatgagttcttaaacacaagtgtcacctgggtttgccatctgtgactacaaaatgggacatgatcatatctacactcaaattacagtgatcccaatctgcttatcttcacgagtacaaacttgtccccactaaaagtatgcctcacctgtgcttacaacccagtctttctgaatcaattaccaagtaacacctgctttgtttattcctctccagttcatgcttccctttgccagctgtctgctgttctcagggttgcttggaacagcttcataactgctggtgcccatggcaagaagatgatattgattccaaggtagctaccacctatttctccaaagcttctgtccagtcctcagatggctttatgtagggatgctccatacagagacacggggtaagggctatgcttcctttctttgactcctttgatacccagttctggaagttgcttggcttgtgttgtcttcttaaagcctctatgtacaataacatcagattctgactctcatccaactcttgggtttcagtgaagaatctcttagcaactttcccttcagttttggttgactggccaaatcagctagttgagtcatagcagtaattacacaggttgcaaaggattaacataaactcaatttgttaaaatacctaaaaaatattctcaatttctgtcactcttaatgtaagtgccttgcttaccactgtatatcatatgtatgcaccccagtgcccagattatacatttaaagaaaagagaaattccatcaagggaggtagtgggttccatttcttgtatcaagaaaaaatttagattacaaacattcttcaaataaagaacaatcattctaaaatacgaaataaaaatattgagtttccaagcaaatttgtttgaatgttatagtatgtttaatttttatcattttaataaatagtagtgttttttatatatagatgccattctattgattatgggttatttatatgcatatttgtttgcataagcaggcaagactaaagagaggaaataataagcacagtaatcataaaaatgtatatattgaaaccaatacaaggagattgtttaccagcaataattatctatataattatcaagtgaaaaagtggtcctacaattaatcatagatggtcattgttgaacagaacactatgcaccagttaccattgttggaggaggactccaactacatggagagtagcaaagatcatcaccatgccaaccaccaggaatagaagtccaaacacttctgctcaatgcatcttgatatcagagtttccaagaaaaacacaacacagtcaggtcctggataggacgacactttacttacatagagaagagatagagcaaaatcactttcaatagtgtgtgtcagtcccccttggcctgtgggtccctcctggcagcccacacagttggcctgcatacatctctcttgtgctgcagcagaaggaacctttccttttccccttggagcctgaaataatgaaagctgggagtgtgctacaaaattgggccatttatgcacacactgaagcaaaaggaggagcacacgttgagtctgaaatagggaaagactttcccacataggaaaataaccctggcaaaagctgtgggggctctttgtctcctggtaagaaaatgttcctggcctaaggcccattcttatgtggccaagtcgtggagttgaaagaccacatgcataagaccacctttccttacagcccatcccgactcttgtctggccactgaaacagcagataaaacatatcaaatcacccaccttgatttggaggtgtagttgggtcccatggagcccctgaacaacttgtgcagatgcagtttgctcaaacagcctcttttattttgaaagagcactttaccagagtcaataggggaactaaaagcaggagaatggttctcatcagaaatcccaagtgcttggattaaggtaaacaaagaggtaaaaaatgagtctgggtggactgtggatacatagctgacaaggacctcactgagtgtggatattcagcttggtttctaccctccctgaattaatgatatacacatagcaagtggtgttggccataacacaactaacaccctgttctgccagcagataactgagggctattcagttatccatcacccccttggctagtgaatttagtggctctttttcttaggtggctcaggcagtaatgttaactaactccactaccgtcaatggcaaattgtgtattgtttttccaggatatgtatgcctatgttggatttgaggacacagacagtagaatagagccagctgtcagtttgttcacctggcagggtgtctcaggcaaccctgtgatgtgtcagaaatgtggatacttccaatttagtgtcattttgaaaaagcatgtgagaccctcacattcccaagcagcatgcccatgtgagggtgacaggcactcggaagtctagtatgccccacagaggaacatataatccaggggagcacatgtaacgctggtcagggagcagttgtgttaagagtctgtagtccagctgtacaaagctttgtttgatttaaccatgagttaggtggcagggtaaagttgtccaatgtatggaccttcccatactatctgtgatgtttaatttgcctttgatttttttttttttttacctctgattttatgtctgtggaagaggtaagcttcactcatggagtattgggtgccctgcatatttgaactgcactgagggtgtcactctttgactggaacctctggtgaccattagaaatcctatcaggctgctgatgggccacacatggactatttgaggaaattcagaggaaaccttctgaatatgtgggtggcagatccaatagcccttcaaatctccacctgctgctactgctgggctgatcttcaccaatatgttgtcaatccaggcactccaactacattattatacccataacacacagggtattattaaggaacaaaagggagagagattagtatccctactgactacctgcttctggtacttccagtggctggaggcattcctgtctattagttggggtgctagaagtctgagtattgcaacccaggctgtctgtcccagtcctgaggctactacctctgtgggtactcattattcttgttacttgatccagatcttttggacttgaatatccagtccagaaaggaaggtcacaccctgcacaaacttgacgtgtcggttaatgtcacctatgagaatggaagaattggatttcccagtgcctctagagtgatcttagcaccctgacagtcccgggttgaatcgcccaccccagtacaatcccataaaaaggaggactccaggagtaagtattgccttcagattagagtcagagaggtggtatttagggtacaatggcaacctgaactgcaagccatctctaagacttacactctaaggggaaagaaaaaggaaaaaaaatggttgggaatactgagtttgagattttgaagtcccagcctgatacggcttggctgtgtccccacacaaatctcatcttgaattgtagctcccctaatccccatatgttgtgggagggacctggtgggaggtaactggatcacgggggtggatctttcccatgctgttctcatgatggtgaataagtctcatgagatctgatggatgtataaagaggagttcccctgcacaagtcctctcttgcctgccatgatgtaagaagtctttttgctcttccttcatcttccaccatgattatgaggcctccccagccacgtggaactgtgaatccattaaacctctttttctttacaaattacccagtctcgagtatctctttatcagcagcatgaaaatagactaatacacagtctaatcaaatggctgcctgatctaccgtgacatccaccatatcaaacaccaagctcagtgtttcccaacatgcattgcagtatcctcttccccctacaatcaagtgcagtgtttagtgtccattctgtcctagccagatgtgggtaccaccccactagcaagccttatttgttgtcctatttttaattgttacagtctatttttaattgtttcattgtttgatgaattgtcctatttttaattgtttcattgtttgatggttccattgacttgcaagtaagagtgcacataataagccctctgcattttatgagagtgcatccactgttgttttgcttgggcagtgaaagatgttccttggtcagagtaaagcctcatatattatctataaacatgacacagtttttttcaaagggggctatggtgattctagcatccacattttgggctgtaatagcaataccacatcctgaataggtgtcaatagtggcaaggcaccaccagtgcttgtggctaggggccatgggtttgatgtggtcaatctgtcaaattcaggcaggtcctagtctttggttaatttgtccttatttttctctttggatgatgtgggtccattagcaagagccaccatgtccttaatcttggcattttctaggatcatctcttggatcttatccaatttttatatgtaagatgaatgtccattttaagtccatataggtagtaggctgcctattggccacaagcttcatcagcaggagagttaatttatattttgttttaaaagatcatttgttatatgagtagcatttttgttcctcacaggcagcaagatgctgccatagttcctgtccacacacaggcaatcctttaatagttcagtcatttagctgaccttctcctggtcaggaagctaggcagttctctgcttgggattcaagagcctatggaacatccttcgaaatatagctggaggtagctaagtccctgcagctcttgaattatgttcacctgaaaagttagcaccacaggatgctgccaagatttatcacttgtgcactttggagtagtggtctgagccatacctggacccacttgagccacagctggattgccaaggaactctgctgtggagtgcagagagctttgaaatctttctgtccccagggtcctggcattctgggcatgtgatggttggagcagccctgaagatctcagaaatgcctttgggatcattcttccattgtcttaatatcgccaggcttctgcccattcatactagtcttcttattagaaggtttcttggccacatccttggtgtcctctccttaactatcttttttattttttacagcatagccaggttgagaattttcccaatctttaatgtctgcttctcttttgattatacattctatctttttttccctttcttttcacattttactgtaagcagccaagagtagcccgactacatcccaacaccttgcttagagatttattccaccaaatatcttttttcattgtttgtaagttctgccttccacagaacactagggcataaatagaatttaatgaagttctttgccattttataacagggcttttttttccttcaggttccaataatattttcctaattttggtctgagatcttatcagaacagcctttactgtaaatatttctgccaatatttttctgatcacaatcacttaggtaatctctaagaagacaaaggctttctctacatctcttctcttcttctgagccctcacgggaatcacccttaataccctgttcacagcaatataggcgtttttttgcattaacatccaaactcttccagtttctacttactactcaattccaaagctgcttccacaactttatgtatttattaaagcagcactccacttctcagtatcaatttctgtctcagtatgttcaatctactgtaacaaaatgccttcgactaggtgatttataaacaacagaaatttattgctcacagttctgaaggctgggaagtccaagatcaaggcaccagcagatttgacgagggcatgttttttccttcaaagattgtgccttttgctatgttttcacatggtagaagaaagagataagctccctcaggcctcaataagtctgcaacagggaaagataatcccacacaaagatggtaagcctggtacaggctgtgtgggttgtttgtctcttggtaaggaagtgttccaggcccagtgaccattgttttatagcctattgtggatcaaaagaccaactgaatgagactgcccttcccaaaacaagtacatgtaataaacaaacacaaagtaaagtcaatggtatgttctaatatttaaattagccaagtgtaaaaagcagaactaactttgcaacttaaatcttcatatccaaataatgctattcaaagtatggtccaaggaatagagtcagactaagaaatatttgttactcattgttgaaaagataagtatagaaattgagagtaaatgtttagaaacttttatggaaattcaacaaagtcattttacatctgttaatttaatagtaagaaagaaaggtcttgtatgtcctgtttcatttcatttttaaaataatatgtttcactgtgttttacaaagtctacagtaaatctgggagaaaacactggtctttcaccttaggtaggttgagaagcacagacatagaatgtgagaagggggacatggtcaggtgtaaagagtacatgggtagaatgtacaggcctttgtgtgtcatgctacagtatttggatgtgatctcataagaagggaaaaaaacatgtgtttataaactatttttaagaacttcttagaaaaattgatcttgagaatgtgtccaagtttattaacatgttatcaaggaaatcagcaaagtgataaaaaaatatttaagaagtgcaagtttcagagatgttataaatttggagcggtgacaaatttaaaaaataacacttaaagaataatagtgattatagttttatttgaaaggttcataaaggcataatagccaagtaatttatttacacagagcatctgtagcaaaattatctgagatgccagatgtgcagccataaagtcagaagaaaaaaataaaagaagcaaatgagagatacaaggtatctaaaattgaaatgacgtgttttcactctcaaaactagcataagatataaaaatgtatttaacagagatctgaaaaaacaatttctgatttcaaagatgccattgccaggggatttcttctactctgagctataaacaaaaatgctgatgacacagttcactcatttcttactttaggactattttacttttttttttttttgacacatggaagagtttttgagagtagctacaatgtttgcatcttagaaacataactgagtctgcaacatagaggttgatttgaagggtgtcaacaaagagacgtctgggctgggtgttgtggctcatgccagtaattccagcatttagggaggtagctgaaaagatagcttgagcccaggcattccagacctgacggggcaacatggtgagactattctccacaaaaaggaaaaaaggacaaaaaacagatttctgtagtaatcagggtgcaaattaagaaaggattgaactaaaaataacagtggtaaatgttgctatacaatttaacctaaactagaagttaaacaatacattatggaaaaaatgccaacctgtattaaaatcaagaagaattattctctatgagctctacattaattcattcttgaaacattaccaaaataatataaagagttacaaaatggtctcagcagtcactgtgatggaattagggaaaggctctgagcctgtactggggcccttagtacatcttacatcaggaccatctgtatgggaatagcactaccatggaaaaggtttgtcttatgggcccccaagataagaagtctgagagtattaataaaagaaacagaaagttaaacattattgggattttgctcttgtcaaataaagaggcaaatagaaagttggtaattattaaaagtaattttccagtttttcagtttaagtactgcctatgataaagatgatggtccctattttaaagagaggtacagtttcatcaaaaaagctgataatatttaataaataggctgtcaaaatacatacgtcatgggaaaatagagaaagaactgaagtatatttttaaattttcagatagaaattaagggaatacaagatgtcagaaaagaactggttactgtaattagcattactgtaaatttctatgtttgtcaagaatatgagaaaattttagttttttccctaagaaagaggctattgaaagattccagttcaataacttaagacatctttggaaagaagaagaaaaattataaattgataatcatacattaaagagaacatctagaggagaacactagagtccagcagagaattgatgggaaagatctgaggtacagaaggagaagaaagtgagtagccagcttcactgagttgggccaggagcccaaaggggcttggtatagcacatgcttttcagagcccgaaagctccctgtctagagctttgagaagtgatcctgaccttagcagcagcacaaactctgtccttagcattgcaagggaagaacaaaatcccctctcactggcagagtgacctctgtgcttggcttcatgaacagagaatgaagcccttctgcccctccacacatcctctctttctgttaccagagactggaacaaatgagccaggaggctacctgtctgggggaatgagtcacagctacgactccactagcagcacaggctccacagctgggattttcccctcttcatggtgctgtagtgctgctgctacagggagcaagagagattgaaagcagtgtgtctcggggtgttggggactaccccataccacagacaccaccaacactagcccacattatttgggacctaaagagtcattccactactgctattgccatggcccttaccatgctggctgcccagaaacctgagaacctgctcacctacctggctcactgctaccactatagtcatccaagcaagccacttgggggctcaagaatcagcccactggtaaccgccaacacaggtgccagggtacaccaccctggggcacaaagataggcatgcacagcccgctgctgctactactgaagcctgaagactgaccctcctggcttcctagtccccagcacaacgtcaccacaacctccactaataactaaaacctaaccctgataggaaaccacagatacccctagcattgtttagagccaaagatatgcagagactatactactgcatacacccagagtcaaatccaaactgcccagaccaatcgatagatatgtcttcaggaaaaagttgtctctacaaaagtaaattcaaaaaataaaagaaagcaactgttacatcagatgtgcagatatcaatgtaaggacacaataaacatgaaaaagcaagaaaatacgacaccttcaagggaatgcaatcattctctagcaataagtattaataaaaacatctcagaaatctcagataaataatttaaacattgattttaaagaggatcactgagataaaagaaaattctgaaaaatattactgagaagtcaaaaaaatagtttatcgatgggaaatttgccaaatacatagatatttttaaaaagaaccaaacaaaaatcttggaactaaaaaattcattgaagaaaatgcaaaatacatttgaaatcttcaggaataaatgagatgaggcagaaaaaaatatcagaactttaagaaaagtctttgaaacaactcagacaaagttaaagaaaaagagttaaaaagatgagcaaagcgtttattacatttgggagtaaataaagtgaccaaatatatggcttatcactatccccaagtatattagtcagttctcacactgttaataaagacttgcctgaaactgggtaatctataaagaaaaagaggtttaatggattcatagttccacatgtgggggaggcctcgcaaacatgacagaaggcaaaggagaaacaaaggcatgtcttacatggtgacaggcaagagagcttgtgcaggggaactgctgtttataaaaccatcagatcttgtgaggcttattcactatcatgagaccagcacaggaaaaacctgctcccatgattcaattacctcccactgggtccctccctcaacacatggggattatgggaactacaattcaagatgagatttgggtggggacactaccaaaccatatcactgagagtgaaaagaaaatgaaagaattagaaaatctatttattatttataaatagtataaattattatttatgactggtataaaattagaaaacctatttatttttgtaaaataataggtgaaaatctctgaagtatagaaagaaatttaaacattcaagtatagggttcaattatccccagccaggtacacgacaaaaaggtcttcttcatggcacattacaatcagaccatctaagggaaatgataaagagcaaatcccccaaacagcagaagtctctagtcactcataaaggaaactgtattgtgtccggaactggtaggttcttggtctcactgacttcaagaatgaagccgcggaccctcgcggtgagtgttacagttcttaaaggcggcgtgtctggagtttgttccttctgatgttcagatgtgttcggagtttcttccttctggtgggttcgtggtctcgctggctcaggagtgaagcttcagaccttcgcaatgagtgttacagctcttagggcagcgcgtctagagttgttcgttcctcccggtgggctcgtggtctcgctggcttcaggagtgaagctgcagaccttcctggtgagtgttacagctcgtaaaggcagtgtggacccaaagagtgagcagtattaagattgattgcaaagagcgaaagaacaaagcttccacaatatggaaggggacccgagtgggttgccactgctggctccaggcagcctgcttttattctcttatctggccccacccacatgctgctgatggtagagccaagtggtctcttttgacagggtgctgattggtgcgtttacaatccctgagctagacacaaaggttctccacctccccaccagattagctagatacagagtgtggacacaaaggttctccaagtccccaccagagtagctagatacagtgtgtcaattggtgcattcacaaaccctgagctagacacagggtgctaattggtgtgtttacaaaccttgagctagatacagagtgctgattggtgtatttacaatccctgagctagacataaaggttctccaaggccccaccagagtagctagatacagaatgtccattggtgcattcacaaaccctgagctagacacagggtgctgattggtgtgtttacaaaccttgagctagatacagagtaccgattggtgtatttacaatccctgagctagacataaagattctccacgtccccaccagactcaggagcccagctggcttcacccagtggataccgcacctgggctgcaggtgcagctgcctgctagcccgcaccctgtgcccacactcctcagcccttgggtggtcgatgggactgtgcgcagtggagcaggaggtggtgctcgtcagggaggctcgggccgcacaggagcccatggagggggtgggaggctcaggcatggctggctgcaggtcccgagccttgcccctcgggaaggcagctaaggtcgggtgagaaatccagcgcagcgcagtgagccggcactgctgggggacccagtacaccctccgcagccgctggcccgggtgctaagcccctcattgcaggggagggggggccaccaagtccacgcccaccccgaaccccagctggcctgcaagcgtgtgcggcgcccggttcccgctcgcgcctctccctccacacctccctgcaagctgagggagctggctctggccttggccagcccagaaaggggctcccacagtgcagtggtgggctgaagggctccacagtgcagtggtgggctgaagggctcctcaagtgctgccaaattgggagcccaggcagaggaggcgccaagagcgagcgagtgctgtaaggactgccagcacgctgtcacctctcactgtcaggttaacagtagatttatgagcagaaatctcacaggtcagaagagaatacaaagtgtgaaagaaaaaaagaaaaaccatgccagccagggaaactataccaagcaaaattattctgcataaatgaaggagaaatactctttcctagaaaagcaaatattgaggaaaatttgttaacactaggcatgtcctataagaaatgctccagggattaaagcctataacaagttaacaaaaaaaaaaaatgctcaactgagtactaaacctggaagtgaaaggagcacatttaccatcatgaaaacacacaaaagtataaaatacactggtaaagcaatcatacaaaagaggaagagaaaggactcaaacggtaccaatctagaaatccaccaaaccacaatgacaaacaaggaaaaaataaaggaatgaagaatatacaaaccaccagaaaataatttataatgtgacaggaataaagcctcacatgtcaatgataattttgaatttaaatagattaaattctacacttatgagatatagaatggctaaatggataaaattacataatccaattatatgctgcttacaaaaatctcaccttactggtaaatacacataaagatagaaactaaaagaatgtaaaaagataagccacacaaatggaaaccaaatactagtaggagaaggtattcttatatccagtaaaacaaacttttagtcaagaacactagaaaaagacaaataatataatgataatcaatccagcaagaagatataacaattttgaaaatatgtgcatgcaacattggatcacccagatccataaagccaatattactaaatcagaagggagagatttagtgcaatacaataatagtgaaaacttcaacccctgactctcagcattaaatatatcatatagcaagaaaatcaacaaagaaacaatagctttacatcagactttaggccaatgggcctaagagatgtttacagaacaggtcacctgactgctgcagaatacaggttcttttcattagcacatggaatattctccatggtagaccatatattagaccacaaaacaagtctcaacaaattaaaaataatcaaaattatatcaagcatcttctcatgccacgttggaatgaaaccagaaatcaatactaagataagctttggaaactatacaaatacacgaaaattaaagaacatgctcctgaatgaccactgggtcaatgaataaattaacatggaaataaaaaaatttcttgaaataactgaaaatggaaacagaacataccaaaacctgtggggcacagcaaaagtagtattgagggaagtttatagcaagaaatgtccaaatcaaaagagtagaaatattacaaattaatcaagcaacacaacacagagaactaaaaaagatctaattaaaccccaaattagcagaacaaaaataatagagattaaagcaaaataaaatcaaatagaggctgcccaaaatacagaagaccaatgaaatgaaaaattgcttctttgaaaagataagcaaaatgggtaaaccactagatagactaaccaggaaaagaagagagaagattcaaagaaacaaaatcaaaaatgaaaatggagacattaaaatgaatatcacaaaaatacaaaagatcatcagagattattatgaacatctatatgctgacaaacaggaaaacctagaggaaatagagaattcctggaaatacacaggcaactgagattgaatcaggaagaaatagtaaaccagagtagatcaataatgagtagtagattgaatcaggaataaaagatcttccaacaaagaaaaactgaattgcatggattcgcagctgaattgtgccaaatgtaaaaagaagaactaaaaccaatttctgccattattccaaaaaattgaagaggagagaattctccctagtcattcaatgaggccagcatctccctgatacccaaactagaaaaggatacaacaaaaaacaaaactatagaccaatatctctaatgaacatagtcataaaaattctcaagaacaaaactagcaaacaaaactcaatagcacatccaaaaggtaatcaaaagtgggtaataaattccactgctatcaagtgggatttatatcagagatgcaaagttagttcaacatacacaactgaataaacatgatacaacatatcaacaaactgaaaaccatacaatcatcccagtagatacagagaaatcatatgataaaattcaacactcttttatgatagaaactctctacaaaccaagcatagaaggaactaccccaaaataataaaagccgtatatgacaaactcacaggtaacatcatattgaatgaagaaaagttgaaaacattccaagaacttgaacaagacaatgatgcccattttcacaactcctattcaacgtagtactagaggccctagccagagaaaatcaggcaaggaaaagaagtaaaaggtattcaaaatggaaaagaggaagtcaaattgtttctctttgctcttaatatgatcttatatctggaaaaacttaaagactccaccaagaaactcattagtttggtaaatacattcagtaataacgcaggatacaaaatgaagatatgaaattagtagcatttctatgcaccaataatgatttaaccaagaaagaaatcaagaaagccaccccatttacaatagctagaaaaacaatgaaatacttaacaaataactttagccaagaaggtgaaagatctctacagtgaaaactctaaaacgctaatgaaagaaattgtaggtgacacaaacaaattgaaaaatatcctattttcacatatcagaaggattaatatccttaaaataaccatattgccccaaataatctacagattcaatgcaatctttgtgaaaatacaaatgtcattctttatagtatcagagaaaacaatcctaacattcatatggatccaaaaaagagcctaaattgctgaagcaagcctgactggaggcatcacattatgtgacttcaaaatgtatcacaaggctatcataatgaaaaacagcatgaaatttttataaatatataaacacagaccagtggagctaataaaaaaactcagaactaaaggcatatgttaatagcaaactaatcttctacaaacctgacaagaacttacattggggaaaagacaccctattcaataaatggtgctgagaaaattggatagccacatgtggaaaaataaaactggatcactgtctctcatcttattaaaacaactcaagatggggattaaagacttaaacataaaccccaaaactataaaaatactagagaaaactctcctgtacattggtttaggcaaataatttatcactaagaccttaaatgcacaggtgacaaaaataaaaatagacacatgggactatattaaactaaagagattctgcatagaaaaagaaataacaaagtgtagagacaatctgttaaatgggagaaaatatttgcaatctactcatctggcaggggactaatatccagaatatacaaggaactcaaacaactcaacaggcaaaagaaaataatctcatgaaaaagtgggcaaagaacatgaatagacatttttcaaaagaagacatacaaatgggtaacaggatttgaaaaaaaagtttaacatcaccaatcaccagaaaaatgcaaatcaaaaccgcagtggacacaatcttatcccattcagaattgctattattaagaagtaaaacaaacaaataaaaaacagatgttgacaaagatatggagaaaaaggaactctcatatactgctggtgggaatgtaaactagcactcgagctatgaaaacaatatgaagatttctcaaaaaataacaatagaattactgttcgatctagaaatcccactatgggtatctacccagaggaatagaaaccaatatatcaaagggatacatgaacttgcatgtttcttgcagcattattcacaatagctaagatatggaatcaacctaaacatccatcaatggatgaatggataaagaaaatgtgatatgtatacacaaaatggaatactgtttggccctgaaaaagaatgatattatgtcatttgcagcaagatggatggaactggagatcatcctaagtgaaataattcaggcacaaaaagacaaatatcacatgttatcacttatatcacttatatcactaaaaaatttgatcacattgagatggagaggggaaagattgatgagagactaggaagggtgagctggaggaagaagcaggataaagggaggagttctaaagggtgcaaacataacagtaagatagaagaaacaaattcaatgtttgatggcagggtagagtgcttatacttaacatttttgtaacaaaaatgctataaaaatgttttatattcaggtgatcaacaccctaaatacaaaacctgtgggggacagcaaaagtagtgttgagagaagtttatagcaagaaatgtctaaatcaaaagagtagaaatattacaaattaatcaagcaacacaacacaaagaaccaaaagagatctaattaaaccctaaattagcagaacaaaaataatagagattaaagcaaaataaaatcaaatagaggctgcccaaaatacagaagaccatacaaatggctaatatgatttgaaaaaaatgctcaatgtcactaatcaccagataaatgcaaatcaaaaccgtaatggacacaatcttatcccattcagaatggctattattaaaaagtaaaacaaacaaataaacaaaaacagatgttgacaaagatatggaaaaaaaggaactctcatatactgctggtgggaatgtaaactagcacgcacactatgaaaacaatatgaagatttctcaaaaaataaaaataaaattactgtttgacctagaaatcccactactgggtatctactcagaggaatagaaactaatatgtcaaagggatacctgaacttgcatgtttattgcagcattattcacaatagctaagatatggaatcaacctaaacatccatcaatggacttgattactatgcattctatacatgtaacaaaatttcacatgtacctcatacatatgtagaaacaaaaaatatggtggctagatttacaattctaaaataatcatcgcccaatcagtgtatccctcacccaggaaataaacttttcgtgtctggattaatctcttagctacatggaactttattctaaaataatttttatctcatagaaagttgcctatcatcttgattcggaaaacataacagctttctctcttaatttctctatctaaatttgcctgaaattagtaaatgtttgaagtgaaccgtgccagaaatgtagacttatatatgtgttcttctttgaaaacattgttgagggctgcttaattttgaattaggggtagggattctcaactttccatttaaaaggtgatcatttggaataaaagaaaactgtttatttggtcctcaccttggtcttgttttaagaaatagtataactctctgtaggtctttatttgcttattcataaaataaggccacctggtacattatctttatcttacaaacaaatacaaacaaaaaaaaaaaccctttcatttttctgagagtagaaatacaggccctatgaacatgtggtatttatgctgcggtaaaaaagccatgcaatgtttttactgtcaagatactgggttaatctctaataatccagttgttgcttctgattgggtgccaaatgttttgttagaaattgtagagggattacagaaaggagataagaaatctctgagatctagagacctgaaccacattaccaaccaacttgacctagttgacataaaccttcaatctaacaacagcagaaagcataatcttttcaatagcacaaggaacatcaccaagaaagtctatattccgagtcattaaacaaaccttaatatatttaaaaggattgaaatcatatgaagaatgttctgtcaccataatacaattaagcaagaacacaaaaataggaaatgttctggaaaactccatatatttggaaatgcaaataaccgctgagtcaaagaaaattaaaataaaaactggaaagtattttgagttagaagaaattgaaaatacaacatatacaatttgtgagatgcagttaatgcaatgcacagagcacaaattatagctttaaataattaaaattttaaaaagcaatgtgtaaaattaataagctaagcttctaccttaaataactggaaaaaagaagaacatattaaatccaaaacaagtggaaagaaaaaaataacaacaggggaaataaatgagttaaagatgtaaaaaattagaaaagaattatttaaatcaaaagataactcttttgaaaaatcattaatacagttacatctctatccaggcaaaaaagagaagacacaagttactaatatcaggaatgaaagggaggagattgtactattcatgcagatttcataaatatgataaaagtaataaaacaactttatgctggtaagttcagcaacttagattaaagaaatttattaaaagaaacaacctagagtcactcaaaaatatatagataatctacataaccttatatatctttgaaaaacgtgaatttataattgaaatacctaacacaatgaaactatatgtccagatggctttattgttgaattctactaaatgtataagaagaaataatgctaattcatcacaatgttttccaaaacgatagaagcgtaaagaaaattaagatgagtcatagacttaagggtaatagatgaaattataaactttctaaaggaaaacatagaaaaattcgagtgactgagttaagcaaagatttttttagatagtacacaaaaaaataagagccattaaaatttgatcttcagaaaacactattgagaagcagaaaatgcagagcagggatggggagaaaatatttgtaaaacatatatatctcataaaggattagtggccgaggaatattctttaaaaaactcctatatctcgataacaggaaaaacaattcaataacaaaaacagagcaaaatatttgaatacactttttatcaaagaagatatgtaagtggctcataagttcatgaaaagatgctcatcatcattagtcaaatgctaattgaaaccacaatgaggcattactacacaaccgctagaatgcttaaaataataataataaaaaaaccctgaaagtactgtcttagtgcaggctgctacaacaaataatcaaatactgggtggcttacaaaatatagaaatttatctcccagagttctagagactggaatttccagatccgagtagcagcctagtcaggttctggtgagggtcctcttctttgctgcagactgccatcttcttgtagtatcttcacatagtgtaatactacgtgagaggggctctggtggtcccctttataaggacactaatcttgttaatgagggctccactcttacgacctaattccctcccaaaggccccagctcctaacaccatcacattgaaggttaggatttcaaaatacaaactttgggaagagacaaaaattcagtctattgaagcaccaagtgttgaagaggatgtggaacaaaaggagcttttgtacatcactgataaaattgcaaaacggtacagctatttttgaaacagttttgaaaacagtttggtagtattttacaaagttaaacatacctagtacgttattgattgatttattgtaaatgtgaatacatgtaacctagtaatctcattcctagttatttacttaagagaaataaagacaattttcttcacaaagacttgtatgtgaatattctcagcagccttatttataagagccccaaactgggagcaatccaaatgtcctttgactggtgaattcagaaataaattgtgtcataatcatacaatagagtacttctcagccataaaaagatgggggaaaaaacgctattaatatactcaacaaaatggatgactaacaagagcattatgctaagtgaaataactcagacacaagactgcataatgtatttctatactgcaatgcatatttttatttatatgaaattataaaaaaagacatgttatagtgacagaagattagtgcttgcctggggtcatggatgggggaaaggtttgagtgctaagtgtgcatggaaacttttggagtgatgaaattgaaatatcttgaatgtggtgttggttacatgactgtatccacttgtgaaaatttgatctttacatttaaaatgaatgaatttgaattttatgtattattgctagcaattcaaaagagtagttggactctcaaagttagacaaagatccctttcttctgggtgaattccgcccgtctacttaataagcagatgtgattattcaatatttgggtaggtgcaaattaagattgatttacaataaaaatgtatctgaagaaaattaaaaggtattacctgtgcaatattttgaagaatttttcttctcctttgtgtttagtgagtgtatgatattctactttattgtcatataatttatttaaaatatatcctgttgttatatttagtgtagtattgtttgaaatttactttaaaatattttacgtagcacatcgtatttgtaaattaatcttgaatttacaacatagagacagctgttcaccaagtcaatcattctattaaggaatacttccagcagctgttagttagcatttttaaacaccctattgaggagctcccattcaagaggaagattaggaaatggcaagaactttaaatattgctacctaaatctatactagataatacattgttctgcatcaaagatgcttgaaataatttttaatatgtgaaatcacaggtatatcaaagtctgaagtctaaagatatctactcatacttgtgggatgctgtaggagaattagagaagcccaggaacctaacatccttcagaatactgcccaaggcttaggattattttagttccatattttactatataacaggctctgtcttctaacctgaccagagtttgactgactatcaaggatactatattgtcatatctgcctggagagaaccctttaaacagagaacataatagaaaatttacatatattgacttaaacaaaatatgtgtccatttccttccatgaaaaagatacattcagagaggggcagtttataacttgtaggacagctttatgacatttttagggctcaggtttatgatattttattttctgagagacttaacatgtgaattccttctcatggtttaaaagagaccactggtgttttggccatcacatccacattttagttagggaggtagagaagataaatattattcccccaccgccaccactttaaagacattttctggggcatatgacattttcacttacattccacacttacaagacatcttagttacattctagctgcaaaggagctggggagtatctttatttaggttaatcatttgcccattaaaaattgaggatttcctttactaagaaagataataaatgggacattttaggaccaactagaaatctaagccctgatacattataacacagagttattttaaaaaagaactttaaacaaatgcaaattagaatcgaattttcttatcattggtaaatgttataaacaaaaacttatagaaaaggccatatttattaaagattctttatatacctatcattcaatattaagaactagacaagaatggttattattttactattaactctaatataccattgaaaatcctatgaaatatttggagacaagaaaaacaagtgaaatataaagatttgaagagaaattaaaatgttcattattataggacagtatcgtcatttgaaagttgtcaaatacaagtagtggataaactattggcactaaaataattcagtaaatttgttggacactaaatgaacaaacaaaagtgaagtgcatttctttatgtaattaactattaatagaaagtaaaatccaatatggatattacaaaaagtagcaatacaaactataaattatctagagattaatttaataaatattttttaatcactattaaataattattgtcatataaaaatatcttaaatggaaaaaatggttcacatttttggatgactgaatttaatgttataaaaagactgacttccctaaatttcaggtgaccagtgtatgtgtgtgcttgtgtgtaggtgctacttgaaaatgttaagcctaatttgaaaaagaagacatagtgagcaaagttactctgtaaggtacaaaggatactgcaaagaaatggcaatataaaaaatggaacgtcaagcagtgaagagcatcaggaatctgtgctttatagtattcaatgacatcccattctggcgtggccaggggaaatatagtgctacctaaattcctaaaatttaactttattcatagaaaaatattctaaaggaaatactgaactttcagaagaaaaaacctgagataattttggagttgaatttgtgggtaaaatagcaaattttttttcttttttttttattatactttaagttttagggtacatatgcataacgtgcaggttagttacatatgtatacatatgccatgttggtgtgctgcacccattaactccattaactcgtcatttaacattaggtatatgtcctaatgctatcctccccctgccccccaccccacaacaggccccagtgtgtgatgttccccttactgtgtccatgtgttctcattgttcaattcccacctatgagtgagaacgtgcagtgtttggttttctgttcttgcaatagtttcctgagaatgatggtttccagcttcatccatgtccctacaagggacatgaactcatcattttttatggctgcatggtattccatggtgtatatgtgccacattttcttaatccagtctatcattgttggacatttggcttggttccaagtctttgctattctgaatagtgccacaataaacacacgtgtgcatgtgtctttatagcagcatgatttataatcctttggatatatacccagtaatgggatggctgggtcaaatggcatttctagttctagatccctgaggaatcgccacactgacttccacaagtttacagtcccaccaacagtgtaaaatgttcctatttctccacatcctctccagcacctgttgtttcctgactttttaatgattaccattctaactggtgtgagatggtatctcactgtggtttggattggcatttctctgatggccagtgatcatgagcattttttcatgtgtcttttggctgcataaacgtcttcttttgagaagtgtctgttcatatacttcacccacttgttgatggggttttttttgttgtaaatttgtttgagttcattgtcgattctggatattagccctttgtcagatgagtagattacaaaaattttctcccattctgtatgttgcatgttcagtctgacagtagtttcttttgctgtgcagaagctcttcagtttaattagatcccatttgtcaattttggcttttgttgccattgcttttggtgttttagacatgaagtccatccctgggatgcaaggcttgttcaacatatgcaaaccaataaacgtaatccagcatataaacagaaccaatgacaaaaaccacatgattatctcaatagatgcagaaaaggcctttgataaaattcaacaatgcttcatgctaaaaactctcaataaattaggtattgatgggagtatctcaaaataataagagctatctatgacaaacccacagccaatatcatactgaatgggaaaaactggaagcccattccctttgaaaactggcacaagacggggatgccctctctcaccactcctattcaacatagtgttggaagttctggccagggcaatcaggcaggagaaggaaataaatggtattcaattaggaaaagaggaagtcaaattgtccctgtttgcagatgacatgattgtatatctagaaaccccattgtctcagcccaaaatctccttaagctgataggcaacttcagcaaagtctcaggatacaaaatcaatgtgcaaaaatcacaagcattcttatacaccaataacagacaaacagagagccaaatcatgagtgaactcccattcacaattgcttcaaagagaataaaatacctaggaatccaacttataagggatgtgaaggacctcttcaaggagaactacaaaccactgctcaacgcaataaaagaggatacaaacaaaaggaagaacattccatgctcatgggtgggaagaacgaatatcatgaaaatggccatattgcccaaggtaatttatagattcaatgccatccccaccaaactaccaatgactttcttcacagaattggaaaaaactactttaaagttcatatggaaccaaaaaagagcccacaacaccaagtcaatcgtaagccaaaagaacaaagctggaggcatcacactacctgacttcaaactatactacaaggctacagtaaccaaaacagcatggtactggtaccaaaacagagatatagaccaatggaacagaacagagccctcagaaataatgccgcatatctacaaccatctgatctttgacaaacctgacaaaaacaagaaatggggaaacgattccctatttaataaatggtgccgggaaaatggctagccatatggccagtttcagaaagctgaaactggatcccttccttacaccttatacaaaaattaattcaagatggattaaagacttaaatgttagacctgaaaccataaaaaccctagaagaaaacctaggtaataccattcaggacacaggcatcggcaaggacttcatgtctaaaacaccaaaagcaatggcaacaaaagccaaaattgacaaatgggatctaattaaacaaaagagcttctgcacagcaaaagaaactatcatcagagtgaacagggaacctacagaatgggagaaaattttcgcaatctactcatctgacaaagggctaatatccagaatcgacaatgaactcaaacaaatttacaacaaaaaaacaaacaaccccatcaacaagtgggcaaaggatatgaacagacacttctcaaaagaagacgtttatgcagccaaaagacacatgaaaaaatgctcatggtcactggccatcagagaaatgccaatcaaaaccacaatgagataccatctcacaccagttagaatggcaatcattaaaaagtcaggaaacaacaggtgctggagaggatgtggagaaataggaacacttttacactgttggtgggactgtaaacttgtggaagtcagtgtggtgattcctcagggatctagaactagaaataccatttgacccagccatcccattactgggtatatacccaaaggattataagtcatgctgctataaagacacatgcacacgtgtgtttattgcggcactattcacaatagcaaagacttggaaccaagccaaatgtccaaaaaatgatagactggattaagaaaatgtggcatatatgcaccatggaatactatgcagccataaaaaatgatgagttcatgtcccttgtagggacatggatgaagctggaaaccatcattctcaggaaactattgcaaggacagaaaaccaaacactgcatgttctcactcataggtgggaattgaacattgagaacacatggacacaggaaggggaacatcacacactggggcctgttgtggggtagggggcagggaagggatagcattaggagatataactaatgttaaatgaggagttaatgggtgcagcacaccaacatggcacatgtatacatatgtaactaacctgcacgttgtgcacatgtaccctaaaacttaaagtgtaattttaaaaaaagtaaaaataaataagaattttcaaatgaaaaacaaagaaacaaaaaacacccaccatatcaaatattaaaactgacaagtgttctaatatttatatattggaatcattgcaaataatcaaatttcatattaaaataagtatttttattaaaaatctattttctaaaaacagaaaatattgagaagagatgcattattttacacttttttgaatctgttataatatctggctgaacagaagaaagttggattccctatttgcttctgcattcaatttttgaaatatgctgttttagttgaagtaaagaaaatccagacttccacagatgggtaatttgaaaggggaggagaattttaatagtctttttatatagttgtggatatttttctttgatactacaccatgatctgaaaaatgataaagtttttcaaaggatattttccatgagcattctgaaaacttgtcagtgaactttctgtactgctacattaaaatccatcggtctatcttgcacactgaatggatctttctcctatgtataattttgtaaacattattaattggtcatttggaaaatattggttcactgtgttatgtagatcttccaaatgttgatatatttctttatacagtataaaaatcacattagttaatatcaccaattttcccagaaaagcctttaaatattgagacgctatcaagctcacagtactagatacaggttctctaaaaatctaattttcacttgatccattgccaattttatcattggcaacgaatactattagttattagccttgaatagacaggctcactttattcatttttgagaaatatagtttgtcagtcactctttcaaataaaaatggtgatttatgaaagataaaaataaactagttcagcacaccactcaatcacccaagtgtttttccacaaccgctgtatttcagtatgtaacagaagtgtcttatgaacacttactatttgggcacaaaaatcattaaaaagaggtatacttaagaatcaagaggtaataaaattaataatttttactacaaaaaaatttgaaaaaattgcatgtgtaataaatatgtacagactttttcttgtcatcattctttaaacaattaggtataacaactattgacatagtattaaatattataagtaatctagagataatttaaagtatacaggagtatgtgcataggttttatgcaactaccatgccattttatattaggtacctgagcattcacaaatgttggtatctgtaagagttctggaaccaatccctatggatacagagggatgtctgtaacagaaaaatatgtaataaaggatgttagagtcccttagtttcaaggtacataattagctgttgggagataattgtccatgtgtctcacttttctgctcattgtgggagcactctggaacctttgtttcagaatattttttccttagtgtttctgtggtgacagagagagataattgtctttttctttgaggtgtgcttattgtccatataaaagtttcaacttccctatgtcaaagcagatttgtgctagacaatgttaaatggccaagcaaaattgtatttaagtgcatttcaatagagaagcaagaccaaaacttggtatgaactaggcaacttcatctaaacaaggggcaagatggtttttaaaggttgaggtaagctagtagagaagtatatcacagagaagtaatatcaaaagggaaaataaggctttgatatctttgtgacaggaagtagaagttttgtgactttagtgaggcatccactgaagttaggcttctactctcccatagaaagtgggagataagggtactgtcttgtttgaagattacatttcaaagagatggctctcaggtccttaagaaaaatatttctggattctaaaattaacaaaggcttttaaaaagatttatatcacgggggggcagagaaataatttacaattacaagttttctaaagtaaaccttttaagaaaaaggagggcaggtgcctggagtcaggaaaaagcctgtctaaagttcagtcaagcctaaggaaacattaaggctgttgtagtcactaagctctgggttcctgtcctgtaatatacatcactatatgtgcagatgttccagccctcttcacatttttctctgggaactgatgctcagcacaaattttgatagtctggctaccactattactgtgagtaataagctgtcgtctgtcttgaacccaggagtcttctactggcatccatgaaattgaagcagacctaactttgtaggcttgcaacaagtgcaaaatctcagactctttaaagttcttcaaatttgattgtgtaagtaaaagtgaagatttttgaaagttttgtgggcagttcatgtacatgaagaaagaactcagtagccaggccttgtgagggaaacagaacaactcaacaactccagagtatcttagctgtagcatcttgttagtcatctacttagggaattatcactggatgatttccagccctctgtgtctctactcaagattttaaatcctgggtaagagaaaaggatttgctctgctggggtcacaagtccaaacttgtggtcaaaataatgatttcctgtaatttgtttccccagttggttttcatggagtcaggaaaagaatgttccctgaaagagggatactgacataatgaaaatagtagatctccattaaattgtactgctgggctttacagcaatgtccacgtgtttgaatacacagacatatctcttcctttatatagttcccaaaatgtccattcttaatctaataaaatattattaaataaacacctactgtgtgttagccacttttctagtcaagggatacattagtgaacaaaggcacagaatcctgcctttaaggagtttatattctagtgcacagaaagaaaaaattaaacaggacaaatcagtaaattatgtcatatattagaggtaatatgtactatagaaaaaaagtggattgaggtaaaagagtagaggtgttaggagtgatgattttaaatagggtattcaggaaggcttcattaagaaggggacatttaagccaagacatgaaagaggtgagcaagttagcagttgcacacataactgggaaagaatactcagttcaaaggcttgaagcctagagcatgcctggggttttagggagtccgaggagactgtggttctaatggagagatggagggagagaggaggagcagatggattcagagagttatctgtggggataaaggtggcagcagagtgaccagctaggaggcagttgcagtaatctatgatactccaaagttaggaaaaagatgtgaatagagaatgactcttggattaagcatcataaaataaaatgaaatgtattcactgctcctaaaaggagactacctaaagtcacaaattagccataggaaggacctctggctgatgttcatgtgctttagtcctgtcatagtaccatctcagaattacacagtctttggactaggtggaaaatttaagtaccaccatcatactgttaaataacatgaatttttgaagaaatttaaagagaaaaattttaaaaactggaaataccattaggggttataatcctcagttcttccatggattatggtgatataattgctgtttacttacgcttaatctactcatttgctcttgccatttttttaacttcatttagcttagtagttgattggcatttggtgtaaatatttattccagacaactcagacttcttggagaaactgccttgatgagattgtgggtgtcttcttttatctctaggcagggtagtgcagtaaggcctacaaaaagattctcaacattctatgtcttcttctcatcttaataatataattgtaacctaatttctatctgatgctcagataagccaccctaagtgacaccatattcctttattttcttggttgttcaatagcttcggaaacctgaaatgaccagtggtctcattgatccaatgactgcttttgaggaagtgctctttctttgggctccaaattctgtaggccaataaagaattgggttagggcagagagaaatggttgagaagaaaatattttgagtaaaattagacattgatggttgagagttgaaccctctacctcactcctagggttcccagaagcccaccatatatcaattactgtttcatattaaatgtttcttccacaattgatggcatacttcggtttcgagatgttttattgaagttaacaacatactgagttctcaataggtcatttcagcaaatgtaagacagagttgcaccaaactgaagaaaatacaaaatatcagggttaataagtattgttgtcatctagcagtgagcagtagccaggtgagcttttatgaggcaaataaatcttgccgagtatatagatagctttccccactgccaacatagcctttgtccatcgtcccattaatccacactaggtagtctgcgaagaatgatgactgatgatctgaaagtaggttaccgcatctactggattattaagattgtttgtgttttggggtcttactgatgaacatttatatagatacaatcatattcaaggattatccttagaggtttttctatctatttcttcccccaaatatcctttttccattggtctgacttttgtcctaggtttgtcagaatatggcaagactaaagcaactttccaggatttgttgtaaacctcatccttggatgtctcttcacacagataaagtgagaaaacacatgttctgcctggagggctgccccctgatggttcaggtccatctctgactgaccacagagtctacttcttcttggtattgctatatcaagcagaactagctggaaaccaggcttcggagttttcgtgccgagtcatacgttcatggaaaaactcccttaaatgccatagtttcagactgaaagaagagtaggagtatgatttttaaaaaatatgcgttctgggattggaaactatatgtacatatcatatacttgtgtatttgggtatttttttttttttacctcagtcacatatatacttcttccaattcattaaaagttgctgattatacccagttttgtcgttatacatgtcaaatatctgataatgttgtctggtcacttagtttctacccaggaccagtaagtagtaagccagaaactttttaaaaacatttttaaaatttttatttcagtagtttttggggaacaggtggtgtttggttacatgattaagttcttcagtggtgatttctgagattttggtgcacccatcacaggagcagcgtacactgtacccagtgtgtagtctttcatacctcaccccctcccaaccttcccttccagtcccaaaagtccattgtatcattatgcctttgcatcctcatagcttaactcccactggtatgtgagaacatgtgatgtttggttttccattcctgagttactttacttagaataatggtctccaactccatccaggttgctgcaaataccattattttctttctttttatggctaagtagtatcagaagctttttcttaaataaaaataaatacttgctaaaagagccagaattttaatctgacatcagtattgtagtatgaaaaatatttttctttattattcagtttcttttagttctttattagttgcaaaaataattaactatataatacaaagggaataattttaaaaaaataagcaaatttagtttttaaataataaaaaactcaaaagaaccatttacattttgaactttttgttaaaacacctatatttataataaaatattaattatttaacatttatattaatgtatgattttagaaaaacaatgggcaaaaaaataaaaaatcttacaatctcttctttcaagagagcttttctttctccacgataaaaatgtgatattcacatcattaaaagtgaaagaaaactatgacttcctcttttgagacatgaaataggtactttcctaggttacttaatggagacagcaaacataaattgttttccttaaaattaacactctattgagaccatcctggctcacacagtgaaaccccgtctctactaaaaatacaaaaaattagccaggtgtggtggtgggcacctgtagtcccagctactcgggaggctgaggcaggagaatggagtgaacccgggaggcggagcttgcagtgagcggagatcgtgccactgcactccagcctgggtgacagagagagactccgtcaaaaaaaaaaaaaaaaaaatatatatatatatatatatatatacacacacacatatataaacgctctagttaaatgtaacattttatggaacacttcaagtaatcttccttttcaagcttacattttcattgtttattactgaaaagataacagctgctatggaataccctgctagaggaagtatactgtcagtcctaatttcaaattttttttctttaatttttccatttttctagtgagaaaaccaccttcaaatcaccagaagcaattttcacaggtaggtagacacacttgaaaaggtatataggggagaccctaaagttgcaagtgtatatttgccttacaagtgatttgtcccattgttttggggaagaaaaagggtaattagatctttcaacttttttcttgtacatatttagtggatacagtgtaatgttttgatacatgtacacataatgttatgatcagattgtcttctgagtgtttgaaaggagcagctcttgtgtagtagacacttatccccatggagaaaagagtgatggaaacttggggtctgaagtgcttacacaacagaacaaattctgactcagtgttcctgaggaaagctggtggttcatacagaattgtgaatgggaattaaaggggggcatagttgaaacctggagagaacaagtgtgagagagtcttaatttacctgggagaaattaatatccctcaaagaaccatgcctgttgggaagcccattgtgatgagtgagaaaaccagcactgcctgtcagccatgtaggaacaatcctactgatgatactgtggatggcaggggggacagcagcacatagtgaaccagctgtctcttgtctttggtatggaaaccactctgattccccttttaaatgtgtaaatccacagggtttccaaaaaattcaggatagaacgcctcgagaaggaaatagtgaatttcttgctaacaaggcatctggggggttgaggaataacttggacaaacaagagttgtaactgcatacacatctttcttaaattgtagaacagtttataaaatcatatggacttagttttaaatgaaaactaataaaccatacacccacaatatcagcacacttccttttgcatgttacatttctattctcatctatagaaagatacttttttacatagttaaaaataaacaatagaatcctcacatctgcccaacaggaccaggtaattggttgattaatccaaagtccgacagagttgaaaataaaaaagatgactcatatactaaaatgttttattttaactcgaaccacttaaaatgcatatatttgtgagtatattcatgtaaggataaagcttttactttgagtgttagactacttgatcagagttcttcagagtacctctagcataagcccactcataatgcattacattcattttggtgaatttaaatataagcagcaacttagagacacttgcaaatccaaatgaataagacttctagaatttttgattttattttcttcctcatatttagtctttccaaaatactcacctggctgtattaaaaacagatgatttttttgcaatcatactttattagtttacatagtacaaaattacattttccaagtagaataaatagaataataaatacaactgttagaagtggttttgagcacaacccccaacccccaccccaacttgtaggggcagaaatatgcaggcacaatggaaagtaatctgttaaacattaagtgttcattgtgccatgaaaatcagtcagggtgctttacagagggaagagagcatgctgtttagtttgttatataaaaatattttggccaggggcggtggctcactcctgtaattccagcgcttggagagggtgaggcaggcagatctcttgaggacaggagtttgaaaccagcctagccaacatggtgaaaccccgtctctacaaaaaatacaaaaattagccgggcatggtggtgggcgcctgtggtcccagctactcgggaggctgaagctcgagaatagcttgaatccgggaggtggaggctgcagtgagccaagatggagccactgcactccagcctgggtgacgggagtgaaaccctgtctcaaaagaaatttacatgtaatttttacagtcatacaaataatgatatttcagcttttcatttgtgagatgtaacataattttaacaaatcagcacattttaattctagtatggattagtctctgggcagaacaaggatctcattaacagtaattgacttcctcaactagaatccaagcactgtgagaatgagatagttcacatatttactcaaaaagaaatcttagttgtccagatgaaaaaattcaaggcttatcggttttaatattttaaataacatttcttaaactatgtagcttgaaaatgatagtttagatttaaaatcaatcctgatcagtgtttataattcaaattatattattgtaatcaataaaatgttaggttcttataaatattacactatgatcatcatagagattatggccatttttgcctcttcagagcactgtgttctatttgatcatcaagcatgaggatgtcattattgtttatttttagtctattattaaataaaattacatgatctgaaagacttaaacatatagctaagtgaaaacaaaacaagaactataaataaaaataatgtttaacttatgattaacataataaaaatctagaattatcaacaattctgataatatcgactaacatttttgtcactgtaaaacccagtttcttagtattaatatcttgttctccatttaaggcccagtttaaattttctctcccttcttttcatcccacttattctaaatctttttctctttcagatggaataaatactcattcctttgattctcataacttttatttatattcttttaactgatctgattatcttgcattatcatatttttttcagtgttcatcttttcacctaaccagtcccaaccaccataaactatacatttgttcattttaatttttcctctttgaatgggacttggtacacagatgaatgtattactagatgaatgcctgtgtcccggataaaaatgcttccagccacttcagactctgttctgtctctccaactggaaaagcaattcttgtccttatatttgttttggttgtgtctttccttgaaaaactctcatgcttttctctttttgccaagtgtgaattctttttttttaactagacatcggacaatatagagtgtagattgacttgcattttttttctttttggaataggagaaaataaaaaggcaagagtagtcttaatccttggaaacactgtctgagacatgcattaaaggtcagaaaattttatgcctttgatttcttctgtgactatcctagttaattttattattattttttgttagctcatcttggttgccacaggcattccaagaactcttttcaatgcagtttttacctctttgttcctcagactgtagacaagaggattaagtaagggagtaaccactgtgtaggtcactgccaccaactgatccttgtctgaggcagacttggacttgggccgcagatagatgatagaggcacagccatagtggacaaagaccacagtgagatgtgaggcacaggtgacaaaggccttcttgccctcagctgaggggatcttcaggatggtgttaactatgaagccataggatatgagaattaacagaaaaggcaccataattaccaggatgctgaggctaaataaagccagctctttcacatgggtgtcagtgcaggctaacttgataacaggtgccatgtcacagaaatagtggttaaccctgttggggccacaaaaacgcatgtcacaaatgaggttggtggccaccaaagcaataaagaaacctgtggctcctgagagagaaatcaactccaaccccagccttttgtttatgatgagtgtgtacctcagagggtgacaaattgctacatagcgatcatatcccatcacagcaatgaggaggcagttggtgcaagcaaagccaaggaaaaagaacagctgggtggcacaggccatgaaggagatggtcttggtgtctgagagcaggtggaccagcagctgagggatgatgacaaaagtgtagcaggactcagaaaatgaaaggatgaatagaaagccatacatgggagtgtggagagtccagctgaagcgaataacggccatgatggtcacattggccaccaggattgtcaagtataggagaagaaagatgacaaaaagcagcagctggagctcccccaggctggagaaacccaccaggatgaactgtgtaaccacagtggttttgttgaaacctcgcatctgaagaacttctttctgtgaatgtgtttgaaagagacaaaatgataactatctttgtaatacgaacattttcccaacgccttcactttaagtatattgtcagattcataatgttaacatctctttctcttagtctatctagatatagatgcctctttaatgatttttccaaaaaattattaatctgaagtaacagagaaatgattatctacagtatataaataccccaaagttaaaaggatccaactatttgacaaagtgaaaaaattctctcagtttaagaagaacattacctttaatatggaatagaaaatttaaattatttttcttgtactgtatgtatttggtccttggatgtatattctatcctagtaatttggtttcttttcctaatatcacaatccatttaattagcattattgtaaaggcattaaaacactgtgcctcagagcatgaactcaggaacaagaatgctagagtttgaaacttgcctttgccacttagagctatgaacttcagtgtgacaatttttgtcatttgtaagacataaataataatagaacatattatataggattgttgtaagaacttagatggtatcagtaaagtacttgaaacagcacttaacacatagtaactccaatctatgtgtcagctgcttatgcagttaaggagttgagaggcaaatatcctggtttgatatttgaacactagaagagcctggatccttaaatcaggacagagtttccctgcatttgactcaagtgttcttatccttgagattctttttttaaaattgagcaaaaatttaaatatgcacagttaaagaaagaactgttacttttacattaaaaattattttctttttaatttattttttattagcaactaataattatacctatttatggggtattatgtgatgtttggatacatgtatacattgtgggatgattaaatcagggttctttaataaatctgcccttttttatctaaggagtctatagcctttgaattctctttcatgattctatgacatttgttctgtatgtttctggaactgtgaagggacccaagtgtaaaacctgggtcacaaggttatgatgtatctgtctatgaaacaaaatagattgatgatcactagggataggggtggagcaggaagacttaggttaagtcttctctctattgaagaaaggagaggaaataacgtgaacacaccttgaatctcttgcaaataaaatagccagaaagtgctaaatggaaagagaagagcctatctcaggaacttcccattattttatagtctaggttattcctcctcccagagccctaatcatttttactttcagaatcagcttcagagattcaggagaaaccagatgtaatatttgcacaactcaacatctcccaacttgttggctactttaatggctataagatgttaggagttgtttgacatgacacacttgagtttgttcattgttcatcatctctgttgtggtttatttgcttaggtagttccacacacctgctacaacagctttttcctttgtttcagaccttgcggaaaataagatgctttcatgtaagctacttcattttgactctaacatcctggtagatagcaaaagtgtctgtcgctatctcacgggtaaggcaagtgtactaagtgccctctcctgggcagagtaacccatgaacatcctcatccttgcatttaacatgttgcattgaacatacctctttatatatttggcactctttatagattataagcttctcacatgcaggaataactttttttaattaaaaaatgagtccctagaatatagcatagtttgtgatttatcatgtgctcaaatattttgacttgattagaatgtcaaaaattaaataatatgctcaagtggtagatccagtatcagaattcaagtcttctaacttcaaatccactttctacttaccaaattttatggcacccattctttaccttgtgagaagatgatctgctcatatactttttccaagggatgaaagcatagttccacactgcttgttcaagtaaaacttatttgaacatttctgacttttctggaataatccaattttaattatccaagtttttgaattatcaaattttacaatatgagctttattctctttaatcttaatattagggagataagaaaaaaaggaaaaaggggtaggaaagtaggcaaatcaacgatatgattgatgaaaacactcaaaggcaaggaaggtcaagaggatgttgcttaatttcaatcacagttttatgaaccaaacaaattgcaatgtctccctaaatcattagcttctttttctgtgactttcatgagaattttgttaaataaaaaatatcctctaatcttgtcctcaacactagtactgcttctatatcttttttcaggttgcttcatatacttgttttgcccattgtttttcatattcaaatccctgtcatgtcccatgttctccaataaaccttccctgcctactagagtccatacagatgctcattctcttcatagcccttagggactttaaaatcatgcatgtatgggaacaggttaggtaatatcttgtattactttataaccaatttgcctatgtggatgcctcataaccttacttacattctttctttgaggcagggggcatagcaacgtattctaaacaatccgcagcagctgcaaacaatactgggcacacaacagacactaattaaatacttaataaattatggcatggtttatttcattcttcttttgtttatgagttttctcatctgctatttttttctggaatatattctttgttctactataatccgtattatttttaaatttttattttttttcaaaatcatcttttactttaaaaggagaaatctacactgctgagttttcctgcctttctagttacttctgttgcttgacatctcagcatttaaatttgatatattaagatggggtgggtgtatatgtgtgtgtgtgtatatgtgtctgtatctgtgtgtgtgcactctagcagagtggaagctttttcatttcaagaaaagagcaaataaagttttactttggctcagaatcctttttataacaataaaaactgctaatgaattcacacctgaatttttggtagaaaatatttctttctttagatagtaactgacaaagaaaatagacttacctacttatgttcaaaagatcaaggaagctgccatatgactttgttgacttatactctggtaggtcttttaaatattgaaagaggcagcagaaagtcagttgtaaaatcaatgacatctgtcccagaagattaaataattggaagaaaatgcactgaagcatttcataaagggcttatatggagacttagttgtccaaacctttttaagtcttaaataagagctgaaaatagtctggtttatattaatatgtcaattcagattctatataattgtatttgagagtatttatagccttctgggattctaaggagtaaggccttctgagacttagtaagagctaaaacaagagaggaaataaatgactaaccttgaacaaggacatattatttaataccagaagaaaaaaattcaaatggcaataaaacattccatctctgcaaagaactacaagtgtagataagtattctagtgattttctgacatcaagactacaaaatgttgaaatgccaggagaagccaaattctccaaccaggagatatacaataaaactgccttgataaggcctaagagatgaaggcaggggttggtgagtgctggttgcttggcacttttgggaattacctaagtagtctctgttctcctaaatcaaaacctggaaaaaattcaaataactggatttcttaattctgtcctaggaattattgtaaggaaatgactttcatcgaggaaccattcattgagtaccatgatttagagagcaggatatgagacattatagtgatacaaagataaaatgtatgaattgatctgctctagtagagaagacagatgtaagcagagctatgtgtaataagaggtaaaatgatgttcagtgttattgtgacattaacaacagggcagctacaaggatgagcacccccagcagggattgccttgaccacagccagggccgagattcagcaaatgacttcatgaataaaaacattctggaataataagccaagtagaaatataagaattaggaataaaaagtcaatactgaatgttttgtgcctaggacactcaaaggaaaaaggagtgaggaggaagactgagaactatttcttgtagtggcttggctggtcacatgagaaaactttctgaaaacaaaattgctgggaaaaattttaaaaaattttagaaacctattgacaagttgaaggtatagtaggaatttttaggccaaaatctgaagaaaataaaatccacagaggaacactggaaattgcttctattctgaggacatttatttacctgtatgaaattacaaactcaagctgttgatttgagagaatcacaggatgaggctgacagaaattaaagaccagaatctaacaaatgggggagtcaaaaattagatattaaagagattttctagttcccatatttgatgctttgacatctggaccttcctgaccaggaaaaaagcagccctcctagggttagcagattcatagagatagaaaatgaatttaatttcatgtgcagtcaaacacatccagaagtcatacccacaattgcctatttttgcatgctcttggactgagggacactacttccttttcctaatccccctggggccaggtgtcagacaactaaggatagtcctggaacccagtgaaactgtttaaactatccaattctaaacctgctcaaaaccacttaccctgctttgcctcaaaaccactctgaagactctccaaaacttccctttgctttctgcctacaactgaactttgtgctctggtgttctccccatggcacttcatgtgtggcgtgatatgccaccttcccactcaggcactcaggaactctaagtaatcaactatctttcaatggcagtcatctctctatctgctggtcttaccctatctgaataaaaataaagtcaacattttaaaacatattgaaggagaggaacaaagttgaagaactgacactatctgacctcaagacttactgcaaagctacagtaatcaagacagtgtggtattggtgaaagaacagataaatagaaaaataaatgggctatcaagccatgaaaagtcattgaggaaccttaaatgtgtattgctaagtggaaaagagctgtctgaaaagcctatatactatgtggtcccaactacatgacattctggaaaaagcaacactattgaaataataaaatgaccactggtgttttccaaggattcagagaaggaggggagggaaaaataaatggggcccaaggcatttttaggacagtgaaactattctgtataatactctaattgtgactatgacattttgcagttctgaaaatatgtagcatgaacccaaatgtaaaatatatggacttaggttaataataatgtgtcaacattggtttaataaataaaaaatgtacagcaataattcagaatgttaataataagaaaaactgtgcaggagagggaggccacaacggaatttgatattttctgtgtattttttctgtgaacctaaatatgctctaaaaataaagtctactgaagaaaaacaaaaagaaaataagattctcatagacatgatatgtagagatacaggccaagcacagtatgattcaatttatataatgatcaaggaaaggtcacaatgatggtggtggaagtcaggatagaagttatatttataaatgcaatacctgagaggcagtacaatggaaccctacagtgttctcataatatttaatttatttattagatatgttcaatttgtaacactttcatcaagctaaacccttatgatttacgtcccatttagtgtatatattatgttttaagatatttactttagttaaagtatactaattagagtttctctttggggaatagtgagctagaaatttttagtcagcccacagactagtaatcactagaaaaactacaaaaatattggaaaaaaatcagatcgaaggcatcagactttaacaaagaattagtattcaggaaaagcaactaacctgtgatgttgtcctggtatttgggagtaattagctcccaaggagaatctgacaattccggaagatattaccaaaaagtcaagaagttgagacacaccgaggtaaagagcaaatatatagatcatgaaccactgaggagtgatggggctggtgaacttcctgaactttgaattgacatttaaaagggaatataataggaacaagattaaacaagaaaaaaatcaactttcacaggagccaaattcagtttcaaacagccttaaaccctggtaaacttgagccaagactccatttaatactctagctccataagtccctactctgaaaagtagatcatagcagcattttgggtctcttgggatctgaacaggttaatagacagtatatcttatgatccctagaaggcctgggtatttttctttatccagtacacagataccttagtagatggcaaagttctctagggaaatattggaacaagattctcaggatataattgtggccatgctgttgtgtctttcctcaaaattccccatctaccctcacaatcaaggggttttgggtttgggaattggtgttgaattggcaattgggtgcaaagacatgaattaaaacactataacttacttttatctgtttgtcagacatgaaataaatgtcaaacatcactttagttgtctgctcatctgcttcattgctagaaatcccttatccattagccatttctacagatctctgcaggacaaaactcctctcattactctgaacttcaggtttatattggttattttccatatcttctccctgacagtttcatgtaacttcctgccttccatcctctggaatactattattcttattgatagcagtgagctcaatttctctgtagcatcatcaccgtcatctctagtttactaatgacaatttctttagaacacttcaaagatgtttatattctcatcaccaatgcatttattgatgcatttattaatgcattggtaggtggttattttccaggtcctctgtgtaacagtggttagggattagccaaggaggctgtccaaataattcattccaacacatctatttctctgaatctttgaattaattcctttacaatgagttaaaagatgtgacatttccaacttattaaccatagatcaatgtttagtttaggttttgtttagtctattagctagcaaaatattaatgccacttttgcatgcatgagccaatatattaaattctgaagcccaagggaatactcccatagcaatgagtttgactgaattccaatttatgtttcctctcccttggctaaagtctttacaattctttcctaaatataccctcctaactcccacagatacaaattggaaggcattttattattattattaattattattatttttgcagtttatctgttctcagagccaaccttgtacctctttatctgattatactgtgatccagttcctgttgtaggcctagaaacaaagttaagtgatggggcaattatttcaggtgcaatccctgagtcttttatgttgagtatggttgattaccttagatgtggtgtgtgcgcctgtgtgtgtgtcgggggacgggggcggtaagggtaatttaaaggaaatgggtcatcaagatctcagcctcacatggatctgctgaaaaatgtccatttcatgtctcaaagtcttacttcttattgagcagaagagcgctggtcaggctgatatttgacagctaaatgtataatcagtttgtgtgcttcatccttagccatggctgccctaaacccctggatgatccttcacatctgtcataaaaacattttgattttttcatacatcttttaaaatgcttattacttataatttccctttcgtctatgcttactctctatgatagtaaagaaaagccagaagcttaataaatctctgaaatctctgttattgccaacaaccacatgatcacccaatgtcttgccctccctctgtaattcatcctatgctattactggtgtttatttgttaattaaaatggtattaggtcaggagtttgagaccaacctggccaacatgacgaaacctcatctctactaaaaacacaaaaaaattagctgggcgtggtggtgtgtgcttgtggtccgagctactcgggaggctgaggcaggagaatcgcttgaacctgggagacggaagttgcagtgaggggagatcatgccactgcactccagcctgggtgacagagcaagactccgtctcaaaaaaaaaaaaaaaaaaaaagtattaaatgctgggatgaattcatccttttttaccccaacactgaagttatttgtgtactcattagatatacaagcaacccaatccaagaatcccattttcaggaatttttttcttaagttactcttgctcctaatcactgtttcagccaaggtccactagaaaataaatagcacatcaaaataaaggaaattcaagagataggaaaataccatagacaatggaataatctgggattagcagcatcccagtttttgtgtcttctattttataccctgttactgtgatccatatagatcagtcttctgagaaagaaagctgggtgaagaatggtagagagtagatctggagtgaatgtgtacatactgaacattagcataatattgactaaaaattctgacaaaagtagtacaacaaaagaaaatttcaagccaatctcttgaccatgggtgctgtgaaaattaagcagaagctttaagaactatggcacagctctaccatcctgttttttctcccttagtgagaacagcatgctattctcttaagggctgtttcttcagtcttaatcacagaataaaaaaaaagaagaactgtttgagggttgatacccacacacaacttgtttaagaaacagagctttgtggttctaagccatttagggtttagcattatttgctattggagaataacctaacaaaggctgactaatagagatgtaaaaattaaataactaaaggcttacaaaaccccaaggaatgtggtatagatgtattaaaatgcgatatactaggttttataataaaattaccaatgatagaggggttcggtttataatgcatgaattttaattctttaggaagccaccagtatatatatacacacacactcagaactaaagaaagaaatgcaaaaatctagaatcatttgggaggtttaacaaatcttttaaaataataccattatctatacatacataatatatttatggctgtatatttccttctaagtactattttagttaaatatcacaagttttcatgtacaatatttaaaaatattttcttgtgaaaatatgatttctttttaagctattatttacaagtgtgctttctaagttttaaacatatggggattttctttttttctttttttattatactttaagttctggggtacatgtgcagaacatgcagtttttttacatagctatacacgtgccatggtggtttgctgcacccatcaacccgtaacctacattaggtatttctcctaatgctatccctctcctagcccctcacccccaaacaggccccagtgtgtgatgttctcctccctgtgttcatgtgttctcattgttcaactcccacttatgaatgagaacatgcggtgtttggtttcctgttcttgtgttagtttgctgagaatgatggtttccagcttcatccatgtccctgcaaaggacatgaactcatcctttcttatggttgcatagtattccatggtttatacaaacatatggggattttctagtaatatattctactcattttgtatgcaataaatatttattgcctgttatgtaccaggcacccttccagctctagacatgcatcagtgagcaaaatgaataaatatccccataaatatatttatagctacatatttctaagtacttccttttcaacatagaaaggaagaaagaccaaaagctgcatgtagagccttaaagttatataggacttgaatagttaaaataggaaacaaatttaccatacagataccaacaaaaccaaaaatttgttctttttaaaaaactgataaaattgacaaatctctagtgagactgattagtgaaacaaaagcccaatatacattatcagaaatgataaaagagctataactaccgatgctgcagagaataacaaggtaataaggacatactgtaaaggttattatatatagctatgtataatttagatgaaaataaatgaaatgttaataaaataaacaatagcatgatttattctggaagaaattggggacttatacaaacatttaacaactgaattatttgtaaacttttcaataaagaaatatccagtccccaatggcttcaacaaagattataaaatttcagtgattcagaaatatttcagagaatatacaagaaaaatcccaagtcgttttatgaggccagaaaaccttgattttaaaccttaatgggaattgttaagagagataaaaatttcataccaatattcattcataagtacatacaaaatacacacacacacacatatggcatatctaaattgtctcagtctgagtaagtgtgggtgtgtgtgagtgcaccttgtgatggaatggtgttctgctcaggcttggctcctgctttgtgcttcggccttccagaaccctgaactgaaataaacgagttggaaaatgaatgaaaaatacaaattatttcaacataaaaatttgtaaatatacaataatgatacaaatgcatgacaataaatgatgtgatctgaaagtgttcagcaagcctgccatatttgtgattgttttgaactgccttgtggtaggaggtgctctttacaatgttctcttggcaagcatttattccttaatttaacctccactactatgatcaccattacttactgattcacctaaaattgggtaaataataatcatggttttattaatcttttgcaaatgtatggatagcttaaatttattacaatatttattgtaatattattattatagtaagtgaggacttactgtacaaaggtgatttgggagggttcattaatttcaccattatcctgttctgtcttttttactccctcttccactttagctatctttcttttgctgttaagtagcaagccccccatggacactctagggttggggaggcattgtgaataaccctggccatcacttgttgacctaatatgtgtaaggaaattacttaaaactttacacaaattgtcttattcaatcttcatgatcagaaaataaaagaaaaatcttatgtcaattgtgaggtaataaatggtatgtgtgaaagtcaggatagaagtcctgggttcccccttacagtgtcaatgggagcaatggaattcttgatctagacagccctcacatgaggacctacaagacttcccgctgggcagggtttgcaatacttgcccagatcatgggttgaatgatatatcagggaagtttataagttttgtctgggcatagaaccaagtggcctaagtactaatcttgcctgccaccaagcagctgtgtgggacattggacaagctatattacctctctagccttggttttcttctctgtgaaatagagattacataaagacatttgacatctttgagattctaaaacactcctggataagatatctgactagctagtctatgcttggggagaaatagaagaaaaggttgactccaaggaatggctacagggaaagaccaagtggatttacctagctagggagttgagagatgatacaaaattatcctgcctcctgcatactttgcctggatctgggcactagcatacgaccaatttccatacgacagaccctgagaccttctgggctgtaaaaagccttataaccatctgatgctacttttgctagcctttagaggtttgttcatcagaatctgccaatttctgcacaggagaacacagggaaaatagaaatagagtggctaactggttatgcggaactagtgtcctatatttgatcagcactaatattattggcttttcaatttctttattttatatgtgggcatattcttaacttttgggaatattcaagatagtgaagattgctgagaaagtaacgtttttggtttttctctttcagttccttcaagttcaatgcatcatagcaattaggaagtattagcattggtttctagtaagggttaggatatattactgcaggttagagatgttctgtaagcaaaacaccctgagccaatagggagaaagaactgactcaaggtcagcatgaaacagggacagagaaaagtttaacaatctggaccaaggtccaaacaagtaaataaaaaatgcagtaaatcctgacacaggtatcaaaacaaagaaacagccaagccagaggcaaatgcatcattttgttatgcagaatgaagtgaaatgggtaagaatataactttaacatttgtgttatacttaatataagaaccaaataaatgagacaagaaaaagtcctttgctgtaggaaaagggcattggcattcattcactgcagatttagcttcggaaactataaaatgggagacagaagatcaagcactcatgtaggaggaggagtcagtgcccagtcagtaccaagtgaagtgtgcctgggaacaacatggccataaacagcactacgcagtttaaactgtgttatcaagaaaagcatcaggtttcccagctggtgacagactccgaaatccattcactttactcttagggtgcataatgccgtccccattaatgactacaaaaataaaacatttaaaataaataaatatattaagaagacaataaacttttatctgtactcttaccacccagatatcaatcacttctattaacatgttcacatatgtttctgacttttctatgtgcacaatgtggagaaaagtgttttatggcaggcctgaactgctgctattcttaggaaagcctgcttgcaagattggcctttggctagcatctgaaaacttggctggcaaacagttccttacacaaatctttccctaattgataaggtggctcactgtccctctctgtttgtacaaagaatgcagaattttggtacatgctaggcagaggttgccaatgtggccaattctgaatgaagtcctcggacactgcgtttctagtgggttttcctgggtagaaacataacctagaggttgctgcatttttgttgatggggaaggatgcactatgactcttatggggagaggtagcatgagaagtctgcacatagattcctgcagacactgcctgtgtcttatgatttgactgtgtggtgaattctcattaagctcctgtaatcaatgttagctgtgaagacaaccctattgtgagtcttgagtcctggtagtaaagatttgaatgtggggttggtcttgggaactccccaacaaaacaaacacatgtaatatttttaaaagaagagatcgtattatctgcacctttttctacctttttatatagaatacatagaaaatatgtttctacatcaataaatatagacctataataatattataaattattgcatagcattatagttagtcttggacttttaagttttgcacattttaccacactgtgacttaaaaaaaaaccttgagatatagcaacacttaatcctcattcattattacttatgaggaaatttaaaaaatagtaattctgggtctacatacatttttatgatttttgatacatattgtaaactggcctttagaaagcatgagccagtgtccacataaagataatgtgtaatcaaaagtctcatttcccatttccagaatgataaaatcattaatttaaaaaaatttgattgatgcagcagagtgataattctttgttttaaattgcattacttcaattgtttacacaattggacatttttcaaaattcttctaaaaattgactatgatttcttcttttagaaatgcttatttatttatatttttcatttaaaaattatttggctttacttttatgatatttacatttctacaattaaagaagtaattatctttcataagtgtttaaaatattttttactcatttttaaattttatttaatagccacttcatattgttatataaataatttacacattcaaatcttttccttcatgcttacaacttctattttcatgtttgtagagctcgtctatatttagagaactctccaacattagatgcatttacctaaatttgctttttaactcactataattttgaaaatatggtaatatctgatctattttattttagattatattttgaacataatctaattggcatcctttagtattaaacaatgtgcatacttctatttctttaaaaaataacctttcctcactaattgaatattacttttgaatattccattctgtgctttgatttgtgtgtctagtttgctctaatatcacactggttgagtagttattttatagtacattttactttctgacagagcaaatcttatcactattctttagaagctctttgaatattaattcatgaccaatcttgcacttgaaattcaaggatatttatcatattatcaaaaataggtatttgattggaatttatttaaactttgaagtgaaataaggattaattggcatctctaaaattgagaaatcctatccttgtataatgcgtgcctgtattcattcatttttcattctgtatccccgtgaaaagtttttaatggctttactttaataggccttcactagaaattgttaaatgtgtttctggaagttttgtgagctgactaatttaaatttcctatttttcaaccactttccatctacctcacacaggtgtgttgagctccctaaaggatctcatatatctcacaccttacactgtcctcttgctttcctcatttgtaggctgttcagggatttactcttaaactatcagagttctgtggtctctagcacatccccctactttgttgaatcactgctctgtggagtttgtctcttctacaggatccttctgagctgtttccccactgatgaccagtgatgtcccctttggtctcttgccaaccacagatacaacatctgctttgtagcggtgttatcatatcctttgggaatgattactgcattatgttttaacaccagttctttcgaccttgctaataaagattaatcaagggaaatgaacaactttcctaagacatgaaagtgaagaagcaaaaaaagtggaatcaaattccaaaaggttttcatgatggaaagttaaaagtagagtggaatggttgggaaaatcatctcaggaatcacagaagttttggcggaaggacataggaagtcacctcagtgagcattgagagaggatgaaatcaatgcacggaagttgagtcagttagtgtcagataatttgctagagaatgactgtttctaaaatcttattttgccatgatttaattactctcttcccattacatgtagtcatactatattttctggttgttagagtcaacatcctattaaaaacaggtacacttgtgctacagacacattttattttatttttattttttcaggctggagtgcagtgagacaatcatagctcactgcagcctcaaactcctgaactcaagcaattctcctacctcagcctcccgagtagctgggactacagatgtgccaccatgccgagctaatttatgtatttatttactttagagatgagtggggagtcattttgttgtccaggctggtcttaaactcctggcctcaagttatcctcctgcctcagcttctccagtagctgggattacaggtgtgagccaccatgcccagccacagacacaatttaatcatgaaggattataagaagaggatagctccacacatttttaaatgtgcatgggcacttttgtgaggagttgccttttcaaatgaaagacccctactgtcctagaaaagtgtccaggactcagctagcctatgtaaaacaaaatgttttattttacaatggctgactttctaaaaatgtgtgaaactattaatatctcactaaaatcagcaggaaataaactgatttagaataattatgagttggatttttttctttgagtattactacccaatcaacctaattaactcttgatctttgtaaccgttaagactgttatatattaatgcataggaaaacaaactgtatttttaaataacctctcactaacttgtgtggaagatatagccttgatgtggaagactctttattgctaaacacaaacctactcatcttcctcggtataaatgcctctatgacagttatttacctgttgcccctcagcaataccccattcttctatattttgcaaattgccttttctttaactctcttgccttctggcttttgatacttgttaagcactgaagaaaaggatgtaagagaaggtgcttcccttctgcttccagctcctgttggcatttcctagtagcagtagatagctgtggatccagcctccagtttcctttaacactcttagtgccaaacttgctgtgttccctcagaggtaccagccacatccatgccaatcctctgggtaatcatgatactactgcatgctcccacacccactgggtatacccaatagatcacttaatcaactgtcaggccactcccactcctggaaggttctagcattggacctccaaggtttcttctctgaactcctggtttctattaacaccacattttctccatccattgcaagtctaagcagagtagccattttccttaattatacaaatgtgttgccctagcatccctttttgcttttacaaattttcacattcgtgtaagcaattccctatattaaaccagttctgtttgaaataattgccattacttttgttttattggcttacccttgagagttagagcctcggacataacagaatataacattgaatattgagtgtgggtaagcactttagacttccatgatatgtgggagaaacggaatgctcagcataagatggagtgagtaacggaagtggtactctggtagagattcaggattggcacatgttctcacaagtgggagctaaacagtgggtacgcatggacataaagatggaaatggcagacactgaggaccccaaatgagggagggtgagagggaggttagtgctgaaaaattatctatcgggtacaatgttcactgtttgggtgatgagtacactagaagcccaaacctcagcatcacacaatatatctatataacaaacctgcacatgtgcctgttgaatctattttaaaaaaagcttttggtttgaggaagagaaagtaggatcaattgaaggagtaggaggctggcaatctaggggagagatttgggaagagatatctaaaggtgatagttggagtggaatgtgattcttcgctaaatgtttttttgaaaataaaaatttacctgaggggttggatattggtttatatggggcactgaaaacagctattatgtgggttgccttctgttagtgtaaatgtgtacttgtcctggcacttggtgagagctgtcttatgatttcaattgtcatcgatctgttcaatgttaaaggatgtcttagaaaatcagtccaaagccagcctcataggctacaaatctagttttagtgctattataattttttgtggccaaggtaacagaaattccataatcctaacttggtataaccttttcttttccctataatgtattcaaaaacatatatacataatagactatgatgaatgggaattcaaaacatcaatttattattattggttgagtttatgcttaaaattcaagtgcagatttctctgtaagctaaacaggcatatgtggcctagttagaaggcctaagggactttactctcttgctaaggccaactacaaatctaaaaatcttacccctctcctgcaaattacatatgactggacaatagagatatgtgtgtatgcacatgtgtgaaaagaggaacagagagagcaagatgttcagttttaacacatgggagtgcaggccaatgtcaacttaatttttcattcacatggaaaaaataagaggaaggataaaatgccaggagagtcattttttatattcatgaccttttaacacaaagtggtcctgcccacttgtcagtgttgtggtgtcagacaccaaaataatagtatccacagctcttggaaagaaacaaggtttaaaagactatttaacatcaagagtatatcttcccacagatgacccatggcttaatcttctattgttaaaggtgaatacttacacattctactccattaattaaaaaaatcacagtcttttcaagaggtcatgtgagagcattataaaagcattaataaagaaaatatatcgtgtggactcagtattaattcttttactcattagtaggcaagtcattttatttgtctgagtgtcatttttctcatctacaaaatgtcgattataatatatacttcccagtgtgctctaaagtttagatgaagtaacattgttaaactgtagcacacaatgcagctataaataattaacacattaaacttagctcatttacttttcatgaatcaacttgatatgctagaggcctggggtgggcaatatggccaaggaaaagacaacttgggggccactgaaattaattttgcatcaatctctgattttcaacccccatcagcagcaagtttcaagcttcaaagaaatggaagaaatgcaattgacttacattcttggcagatgtcctacaataaacagggcctgtgaacaaaacattccacttccttccctcccgcttcaacccctaaaaggtatatgtcattgatgaacagactggtggaagaaaaggaaaaaacttagaaacaaaattaatcaatcaggagacttttattgacaggtggatcttgaaaagagttataatgtgagaaatcatcactattgtgaaacttcctttttgctatcactcctgcagaaaggacagagaatgaaaacatcaaattcaagttctggtctcaaatgtacatgcaaactcttgagttgcatggagaacagattgattgttccatatgcaaagctttccacttggtgaatatttagtgaaattgacttaatctttagcttgccttgcctggtcatttagggtgattttaggtcttgtcattaatccattcccctgtgaatatcttcagggtatgctgaccaaatttagatgtatgcctctgcatacaaaattagaaatgatcgtatttcattaatacactagaagcatggaaatggataacttttcataattttctaacttgaacattatttggatatatcttatgcatcaaacattgtaatgacgcttaacaagtatctcatttaacaattgtgatctgatgattttggaagactgaggtccagaaaacatctagaaaaatcctagagatagaagcttcctgagtccctagaacagagagttagggttgtgccacattttgcagggttagttttcagcctggctgagattgggtgacagtatttactacagcctaaaattctgaacatacatatgcttccccataatagccatctgttattttttaatcaattgaaaaaaaaccttcaataggcaataattcatgtgagtgtggcaagcagttgtgtttttctaaattttatatttggagatgacgggaaatgatctatgttcctgaacataatgctcaattgcttgatcatttgtgttctgggaattttttcctgtataacttgtggtttacggtttggaaagagcagaatcaagatttttttttttttttttttttgagacagagtcttactctgtcgcccaggctggagtgcagtgacacaatctcactgcaaccttctgcctcctgggttcaagtgattctcctgtctcaggctcctgagtagctgagattacaggagcccaccacagcatccggttaatttgtatattttttggtagagataggatttcactatgttggccaggctggtcttgaagtcctgacctcaggtgatccacctgtctcagccttccaaaatgctgggattacagtcgtgagaaactgcacctggccagaatcaagattaattttacaacagggaaattgttcttctcacaattttacaaagagctgatttgaactctttatttctcaagctataaatcattgggttgaacaatggagttataatagtgtaggatactgatattagagcatcctggcttgaggagtagttggactgaggccttaagtagataaaggaggcacagccatagtggacagtgacaataatgaggtgagatacacaggtagaaaaagctttgcacctacccagtgtggaaggaaactgaagtatggcagagaggatgtgaacataggacaccaagatcaacaataaggggatagccaggaccaatgtacagagcatgaagatgacaatctgactaaagtggttatggtgagatgccagcttgaggacaggagcaatgtcacagaagaagtgatgtagttgattggaggaataaaaaggcaggtgaaataccaaggatgtgatgatctgtgcaacagtgaagccacaggcacaggcagcagccactagtcccatacacaccccatgtcccattagcactgagtagcgcagtgggttacagatggctatgtaacgatcataacccatgactgccagcagaaaggagtgagagcagccaaggaagaggaaggaaaacatttggatggcacagcccaggaaagaaatggtcttcttctgggacagcaggtcaaccagcatcttgggtacaatgatgaaggtgtagcaaatctcagagcaagagaggatggcaaggaagaagtacatggggatatgaagggccctgtccaggacaatggtggaaatgatgattgcattggtgcccagagtgaacaggtagaggagcaggaagataacaaagagcagctgctgcagcctggccagggatgagaagccgaggaagatgacctctctcaccacagtctcattgacccgctccatggagcatacatcatcaggagacaattagggagagaagaggagtcagcaccaaaagaaatccctgaaaataagtagattcacagaaaattgatcatcagcatgcgttctcagattttgaattctgaactcatcttttctcctccctcttgtttttccaagacatgatgttgctaaaatcaggcaagcgttcatcaaactttgagtctccatagtagttctaacaccatagccaaaaagttcttgatacttttcagattcaaacaacccttcacctccattcccatcatgaggctgttgctcagattcttagcctttcactcttagattatcttgcaaagcttgtctttttggctttcttgtatctgtattctctcaaaaaaaaaatctttcctacagttgcaaagtaaaccatctgaatataccatagttgtctataactcaatggttcttaattgtcattctcgttacacctaaacccccactcttagttttagtctctccataacttagattcaaattctcttcaacactgttttccttacctcctggcaggaattgtcagttatacttttattatgctggctcttgtttctgtagctttatcctggtcccaaagtggaaaaaaattatcttcttctttctattcattaatttcaaggtggaatatgtctacactttctctaatacctatatcagagtctttcctctggcagggaggaccacaatctttcttcaggattctgtccattttcatcacttcccttaaatccttctgaagtcacattatggctcctttcccatggccactcttacatctctgcaacccatctttcccgtgatgacagcctaatcttttaaaatataaattgtattatattgcttcactcttcaaaaattttacttatggctatagtatagagttcaaactcttcattgtttccaaaagtctttcctttggggaaataaactatctttctgtgctcattttcagaccttaatgggatgtatcactattgcccaaatatgttctggacttccttgcctctattccttgctcatattagtttgttattcacttttatttacagatattttatggttcttctgaagcctccattaaaatctacctttttgaaagctttcttgctaccttctggtgaaaggagttgtttcttcctcagaactcacataagacattgattgccaaaattatgtgatatttacttaattttgaaatattctaatgaattgttcttatatctattttctctaccagattttgtacctttgaggaaaatatgtgaattttatgtatctttgcctgagttctcctctgcatctagaaccatattaagtacttaacaccttctcaataaataattgttatataaataagagagttaacttgccacattatctattttattcatgggcagcacagacttcttagaactcacagagtaagagaattc